(19) United States
(12) Patent Application Publication
(10) Pub. No.: US 2024/0042021 A1
(43) Pub. Date: Feb. 8, 2024

(54) IMMUNOMODULATORY PROTEINS AND RELATED METHODS

(71) Applicant: Flagship Pioneering Innovations VII, LLC, Cambridge, MA (US)

(72) Inventors: Ellen Lovisa Larsdotter AFZELIUS, Belmont, MA (US); Hozefa Saifuddin BANDUKWALA, Southborough, MA (US); Ryan Edward KOHN, Cambridge, MA (US); Sofia Marques Tuna Ribeiro BRITES BOSS, Boston, MA (US); Michael Horne ASKENASE, Somerville, MA (US)

(21) Appl. No.: 18/362,410

(22) Filed: Jul. 31, 2023

Related U.S. Application Data
(60) Provisional application No. 63/394,155, filed on Aug. 1, 2022.
(60) Provisional application No. 63/483,419, filed on Feb. 6, 2023.
(60) Provisional application No. 63/502,864, filed on May 17, 2023.

(51) Int. Cl. A61K 39/395; A61P 37/02; C07K 14/715

(52) U.S. Cl. CPC A61K 39/3955; A61P 37/02; C07K 14/7155; A61K 2039/505

(57) ABSTRACT

Provided herein are immunomodulatory proteins and compositions (e.g., pharmaceutical compositions) comprising the same; as well as methods of making the immunomodulatory proteins and compositions. The immunomodulatory proteins provided herein are useful in pharmaceutical compositions and methods of treating IL-10 responsive diseases.

### RELATED APPLICATIONS

This application claims priority to U.S. Ser. No. 63/394,155, filed Aug. 1, 2022, U.S. Ser. No. 63/483,419, filed Feb. 6, 2023, and U.S. Ser. No. 63/502,864, filed May 17, 2023, the entire contents of each of which is incorporated herein by reference.

### SEQUENCE LISTING

The instant application contains a Sequence Listing which has been submitted electronically in XML format and is hereby incorporated by reference in its entirety. Said XML copy, created on Jul. 28, 2023, is named 0062801_00004US01_SL.xml and is 594,889 bytes in size.

### 1. FIELD

This disclosure relates to immunomodulatory polypeptides and proteins that bind to the human interleukin-10 receptor (hIL-10R) and polynucleotides encoding the immunomodulatory polypeptides and proteins. The disclosure further relates to methods of making and utilizing the same.

### 2. BACKGROUND

Human IL-10 (hIL-10) is a pleiotropic immunoregulatory cytokine that mainly targets antigen presenting cells, such as monocytes and macrophages, inhibiting the release of pro-inflammatory cytokines such as TNFα, IL-1β, IL-6, and IL-8. hIL-10 also interferes with antigen presentation by reducing the expression of major histocompatibility complex (MHC)-II and costimulatory molecules. hIL-10 further functions to suppress T cell-responses by inhibiting the expression of IL-12 and IL-23, cytokines needed for CD4+ T cell differentiation. In addition to these anti-inflammatory properties, hIL-10 is also known to have pro-inflammatory functions. For example, hIL-10 has been shown to stimulate CD8+ T cells and promote the survival, proliferation, and differentiation of B cells.

### 3. SUMMARY

Provided herein are, inter alia, immunomodulatory polypeptides and polynucleotides encoding the same; fusions and conjugates comprising the immunomodulatory polypeptides; methods of manufacturing; pharmaceutical compositions; and methods of use including e.g., methods of treating pro-inflammatory diseases (e.g., autoimmune diseases), methods of promoting an immune response, methods of treating viral infections, and diagnostics.

Accordingly, in one aspect provided herein are isolated polypeptides or proteins comprising an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-454, wherein the polypeptide or protein specifically binds the human IL-10 Receptor (hIL-10R) (e.g., hIL-10Rα).

In some embodiments, the isolated polypeptide or protein consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-454. In some embodiments, the isolated polypeptide or protein comprises or consists of an amino acid sequence at least about 85% identical to the amino acid sequence of any one of SEQ ID NOS: 108-454. In some embodiments, the isolated polypeptide or protein comprises or consists of an amino acid sequence at least about 90% identical to the amino acid sequence of any one of SEQ ID NOS: 108-454. In some embodiments, the isolated polypeptide or protein comprises or consists of an amino acid sequence at least about 95% identical to the amino acid sequence of any one of SEQ ID NOS: 108-454. In some embodiments, the isolated polypeptide or protein comprises or consists of an amino acid sequence 100% identical to the amino acid sequence of any one of SEQ ID NOS: 108-454.

In some embodiments, the isolated polypeptide or protein comprises or consists of an amino acid sequence at least about 85% identical to the amino acid sequence of any one of SEQ ID NOS: 108, 111-119 or 122-126. In some embodiments, the isolated polypeptide or protein comprises or consists of an amino acid sequence at least about 90% identical to the amino acid sequence of any one of SEQ ID NOS: 108, 111-119 or 122-126. In some embodiments, the isolated polypeptide or protein comprises or consists of an amino acid sequence at least about 95% identical to the amino acid sequence of any one of SEQ ID NOS: 108, 111-119 or 122-126. In some embodiments, the isolated polypeptide or protein comprises or consists of an amino acid sequence 100% identical to the amino acid sequence of any one of SEQ ID NOS: 108, 111-119 or 122-126.

In some embodiments, the isolated polypeptide or protein comprises or consists of an amino acid sequence at least about 85% identical to the amino acid sequence of any one of SEQ ID NOS: 112-115 or 123-126. In some embodiments, the isolated polypeptide or protein comprises or consists of an amino acid sequence at least about 90% identical to the amino acid sequence of any one of SEQ ID NOS: 112-115 or 123-126. In some embodiments, the isolated polypeptide or protein comprises or consists of an amino acid sequence at least about 95% identical to the amino acid sequence of any one of SEQ ID NOS: 112-115 or 123-126. In some embodiments, the isolated polypeptide or protein comprises or consists of an amino acid sequence 100% identical to the amino acid sequence of any one of SEQ ID NOS: 112-115 or 123-126.

In some embodiments, the isolated polypeptide or protein comprises or consists of an amino acid sequence at least about 85% identical to the amino acid sequence of any one of SEQ ID NO: 114. In some embodiments, the isolated polypeptide or protein comprises or consists of an amino acid sequence at least about 90% identical to the amino acid sequence of any one of SEQ ID NO: 114. In some embodiments, the isolated polypeptide or protein comprises or consists of an amino acid sequence at least about 95% identical to the amino acid sequence of any one of SEQ ID NO: 114. In some embodiments, the isolated polypeptide or protein comprises or consists of an amino acid sequence 100% identical to the amino acid sequence of any one of SEQ ID NO: 114.

In some embodiments, the isolated polypeptide or protein comprises or consists of an amino acid sequence at least about 85% identical to the amino acid sequence of any one of SEQ ID NO: 125. In some embodiments, the isolated polypeptide or protein comprises or consists of an amino acid sequence at least about 90% identical to the amino acid sequence of any one of SEQ ID NO: 125. In some embodiments, the isolated polypeptide or protein comprises or consists of an amino acid sequence at least about 95% identical to the amino acid sequence of any one of SEQ ID NO: 125. In some embodiments, the isolated polypeptide or protein comprises or consists of an amino acid sequence 100% identical to the amino acid sequence of any one of SEQ ID NO: 125.

In one aspect, provided herein are isolated polypeptides or proteins comprising an amino acid sequence at least about 85% identical to the amino acid sequence of any one of SEQ ID NOS: 119-126 or 291-454, wherein the polypeptide or protein specifically binds the hIL-10R (e.g., hIL-10Rα).

In some embodiments, the isolated polypeptide or protein consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 119-126 or 291-454. In some embodiments, the isolated polypeptide or protein comprises or consists of an amino acid sequence at least about 85% identical to the amino acid sequence of any one of SEQ ID NOS: 119-126 or 291-454. In some embodiments, the isolated polypeptide or protein comprises or consists of an amino acid sequence at least about 90% identical to the amino acid sequence of any one of SEQ ID NOS: 119-126 or 291-454. In some embodiments, the isolated polypeptide or protein comprises or consists of an amino acid sequence at least about 95% identical to the amino acid sequence of any one of SEQ ID NOS: 119-126 or 291-454. In some embodiments, the isolated polypeptide or protein comprises or consists of an amino acid sequence 100% identical to the amino acid sequence of any one of SEQ ID NOS: 119-126 or 291-454.

In one aspect, provided herein are isolated polypeptides or proteins comprising an amino acid sequence at least about 85% identical to the amino acid sequence of any one of SEQ ID NOS: 108-118 or 127-290, wherein the polypeptide or protein specifically binds the hIL-10R (e.g., hIL-10Rα).

In some embodiments, the isolated polypeptide or protein consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-118 or 127-290. In some embodiments, the isolated polypeptide or protein comprises or consists of an amino acid sequence at least about 85% identical to the amino acid sequence of any one of SEQ ID NOS: 108-118 or 127-290. In some embodiments, the isolated polypeptide or protein comprises or consists of an amino acid sequence at least about 90% identical to the amino acid sequence of any one of SEQ ID NOS: 108-118 or 127-290. In some embodiments, the isolated polypeptide or protein comprises or consists of an amino acid sequence at least about 95% identical to the amino acid sequence of any one of SEQ ID NOS: 108-118 or 127-290. In some embodiments, the isolated polypeptide or protein comprises or consists of an amino acid sequence 100% identical to the amino acid sequence of any one of SEQ ID NOS: 108-118 or 127-290.

It should be understood that any of the following embodiments are applicable to any of the foregoing aspects related to isolated polypeptides and proteins.

In some embodiments, the isolated polypeptide or protein (a) binds to a cell expressing the hIL-10R (e.g., hIL-10Rα) on the surface with an EC50 of less than about 500 μM, 400 μM, 300 μM, 200 μM, 100 μM, 50 μM, 40 μM, 30 μM, 20 μM, 10 μM, 9 μM, 8 μM, 7 μM, 6 μM, 5 μM, 4 μM, 3 μM, 2 μM, 1 μM, 0.9 μM, 0.8 μM, 0.7 μM, 0.6 μM, 0.5 μM, 0.4 μM, 0.3 μM, 0.2 μM, or 0.1 μM; or (b) binds to a cell expressing the hIL-10R (e.g., hIL-10Rα) on the surface with an EC50 of from about 500 μM-0.1 μM, 400 μM-0.1 μM, 300 μM-0.1 μM, 200 μM-0.1 μM, 100 μM-0.1 μM, 50 μM-0.1 μM, 25 μM-0.1 μM, 10 μM-0.1 μM, 5 μM-0.1 μM, or 1 μM-0.1 μM, 500 μM-0.5, 400 μM-0.5, 300 μM-0.5, 200 μM-0.5, 100 μM-0.5, 50 μM-0.5, 25 μM-0.5, 10 μM-0.5, 5 μM-0.5, or 1 μM-0.5 μM. In some embodiments, EC50 is determined by the assay described in Example 3 herein.

In some embodiments, the isolated polypeptide or protein (a) binds to a cell expressing the hIL-10R (e.g., hIL-10Rα) on the surface with an EC50 that is at least about 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 140-fold, or 150-fold higher than that of a reference hIL-10 polypeptide (e.g., SEQ ID NO: 2); or (b) binds to a cell expressing the hIL-10R (e.g., hIL-10Rα) on the surface with an EC50 that is from about 10-150-fold, 20-150-fold, 30-150-fold, 40-150-fold, 50-150-fold, 60-150-fold, 70-150-fold, 80-150-fold, 90-150-fold, 100-150-fold, 110-150-fold, 120-150-fold, 130-150-fold, or 140-150-fold, higher than that of a reference hIL-10 protein or polypeptide (e.g., SEQ ID NO: 2). In some embodiments, EC50 is determined by the assay described in Example 3 herein.

In some embodiments, the polypeptide or protein specifically binds hIL-10Rα. In some embodiments, the polypeptide or protein specifically binds hIL-10Rβ. In some embodiments, the polypeptide or protein specifically binds hIL-10Rα and hIL-10Rβ.

In some embodiments, the polypeptide or protein is a hIL-10R (e.g., hIL-10Rα, hIL-10Rβ) agonist. In some embodiments, the polypeptide or protein is a hIL-10R (e.g., hIL-10Rα, hIL-10Rβ) antagonist.

In some embodiments, the isolated polypeptide or protein further comprises a homologous or heterologous signal peptide operably connected to the N-terminus of the polypeptide or protein.

In some embodiments, the isolated polypeptide or protein is an isolated protein.

In one aspect, provided herein are polypeptides comprising a polypeptide or protein described herein operably connected to a heterologous moiety.

In one aspect, provided herein are fusion polypeptides or proteins comprising a polypeptide or protein described herein operably connected to a heterologous polypeptide or protein.

In some embodiments, the heterologous polypeptide or protein comprises a half-life extension polypeptide or protein.

In some embodiments, the heterologous polypeptide or protein comprises an immunoglobulin (Ig) (e.g., a human Ig (hIg), murine Ig (mIg)) Fc region. In some embodiments, the Ig (e.g., hIg, mIg) Fc region comprises at least a portion of a hinge region, a CH2 region, and a CH3 region. In some embodiments, the Ig (e.g., hIg, mIg) Fc region comprises a hinge region, a CH2 region, and a CH3 region.

In some embodiments, the Ig is a hIg. In some embodiments, the hIg is a human IgG (hIgG). In some embodiments, the hIgG is hIgG1 or hIgG4.

In some embodiments, the Ig is a mIg. In some embodiments, the mIg is a mIgG1. In some embodiments, the mIg is a mIgG2a.

In some embodiments, the Ig (e.g., hIg, mIg) Fc region comprises one or more amino acid substitutions relative to a reference Ig (e.g., hIg, mIg) Fc region that reduces or abolishes one or more of the following effector functions relative to the reference hIg Fc region: antibody dependent cell mediated cytotoxicity (ADCC), complement dependent cytotoxicity (CDC), and/or affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the Ig (e.g., hIg, mIg) Fc region does not substantially mediate ADCC, does not substantially mediate CDC, and/or does not bind to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the Ig is s hIgG4 and the amino acid sequence of the Fc region comprises an amino acid substitution at amino acid position S228, an amino acid substitution at amino acid position F234, and/or an amino acid substitution at amino acid position L235, numbering according to the EU index of Kabat. In some embodiments, the Ig is hIgG4 and the amino acid sequence of the Fc region comprises a proline at amino acid position S228, an alanine at amino acid position F234, and/or an alanine at amino acid position L235, numbering according to EU index of Kabat.

In some embodiments, the Ig is hIgG1 and the amino acid sequence of the Fc region comprises an amino acid substitution at amino acid position L234, and/or an amino acid substitution at amino acid position L235, numbering according to the EU index of Kabat. In some embodiments, the Ig is hIgG1 and the amino acid sequence of the Fc region comprises an alanine at amino acid position L234 and/or an alanine at amino acid position L235, numbering according to the EU index of Kabat. In some embodiments, the Ig is hIgG1 and the amino acid sequence of the Fc region comprises an alanine at amino acid position L234, an alanine at amino acid position L235, and/or a glycine at position P329 numbering according to the EU index of Kabat.

In some embodiments, the Ig is mIgG2a and the amino acid sequence of the Fc region comprises an amino acid substitution at amino acid position L234, and/or an amino acid substitution at amino acid position L235, numbering according to the EU index of Kabat. In some embodiments, the Ig is mIgG2a and the amino acid sequence of the Fc region comprises a proline (or alanine) at amino acid position L234 and/or a proline (or alanine) at amino acid position L235, numbering according to the EU index of Kabat. In some embodiments, the Ig is mIgG2a and the amino acid sequence of the Fc region comprises a proline (or alanine) at amino acid position L234, a proline (or alanine) at amino acid position L235, and/or a glycine at position P329 numbering according to the EU index of Kabat.

In some embodiments, the polypeptide or protein described herein is directly operably connected to the heterologous polypeptide or protein through a peptide bond.

In some embodiments, the polypeptide or protein described herein is indirectly operably connected to the heterologous polypeptide or protein through a peptide linker. In some embodiments, the amino acid sequence of the peptide linker comprises or consists of glycine or glycine and serine amino acid residues. In some embodiments, the amino acid of the peptide linker comprises or consists of (a) the amino acid sequence of any one of SEQ ID NOS: 49-57; or (b) the amino acid sequence of any one of SEQ ID NOS: 49-57 comprising or consisting of 1, 2, or 3 amino acid substitutions.

In some embodiments, the fusion polypeptide or protein comprises from N- to C-terminus: the polypeptide or protein described herein and the heterologous polypeptide or protein. In some embodiments, the fusion polypeptide or protein comprises from N- to C-terminus: the polypeptide or protein described herein, a peptide linker, and the heterologous polypeptide or protein. In some embodiments, the fusion polypeptide or protein comprises from N- to C-terminus: a signal peptide, the polypeptide or protein described herein, a peptide linker, and the heterologous polypeptide or protein. In some embodiments, the fusion polypeptide or protein comprises from N- to C-terminus: the heterologous polypeptide or protein and the polypeptide or protein described herein. In some embodiments, the fusion polypeptide or protein comprises from N- to C-terminus: the heterologous polypeptide or protein, a peptide linker, and the polypeptide or protein described herein. In some embodiments, the fusion polypeptide or protein comprises from N- to C-terminus: a signal peptide, the heterologous polypeptide or protein, a peptide linker, and the isolated polypeptide or protein described herein.

In one aspect, provided herein are fusion proteins comprising a first polypeptide or protein and a second polypeptide or protein, wherein the first polypeptide or protein comprises a first Ig (e.g., hIg, mIg) Fc region operably connected to a first polypeptide or protein described herein (e.g., a first immunomodulatory protein described herein); and wherein the second polypeptide or protein comprises a second Ig (e.g., hIg, mIg) Fc region operably connected to a second polypeptide or protein described herein (e.g., a second immunomodulatory protein described herein).

In some embodiments, the first Fc region and the second Fc region associate to form a dimer.

In some embodiments, the first polypeptide or protein comprises an amino acid sequence at least about 90%, 91%, 92%, 93%, 94%, 95%, 96%, 97%, 98%, 99%, or 100% identical to the amino acid sequence of the second polypeptide or protein.

In some embodiments, the first Ig (e.g., hIg, mIg) Fc region comprises at least a portion of a hinge region, a CH2 region, and a CH3 region; and the second Ig (e.g., hIg, mIg) Fc region comprises at least a portion of a hinge region, a CH2 region, and a CH3 region. In some embodiments, the first Ig (e.g., hIg, mIg) Fc region comprises a hinge region, a CH2 region, and a CH3 region; and the second Ig (e.g., hIg, mIg) Fc region comprises a hinge region, a CH2 region, and a CH3 region.

In some embodiments, the Ig of the first Ig Fc region is a hIg and the Ig of the second Ig Fc region is a hIg. In some embodiments, the hIg of the first hIg Fc region is a hIgG and the hIg of the second hIg Fc region is a hIgG. In some embodiments, the hIgG of the first hIg Fc region is hIgG4 and the hIgG of the first hIg Fc region is hIgG4. In some embodiments, the hIgG of the first hIg Fc region is hIgG1 and the hIgG of the first hIg Fc region is hIgG1.

In some embodiments, the Ig of the first Ig Fc region is a hIg and the Ig of the second Ig Fc region is a mIg. In some embodiments, the mIg of the first mIg Fc region is a mIgG and the mIg of the second mIg Fc region is a mIgG. In some embodiments, the mIg of the first mIg Fc region is a mIgG1 and the mIg of the second mIg Fc region is a mIgG1. In some embodiments, the mIg of the first mIg Fc region is a mIgG2A and the mIg of the second mIg Fc region is a mIgG2a.

In some embodiments, the first Ig (e.g., hIg, mIg) Fc region and the second Ig (e.g., hIg, mIg) Fc region each comprises one or more amino acid substitutions relative to a reference Ig (e.g., hIg, mIg) Fc region that reduces or abolishes one or more of the following effector functions relative to the reference Ig (e.g., hIg, mIg) Fc region: ADCC, CDC, and/or binding affinity to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the fusion protein does not substantially mediate ADCC, does not substantially mediate CDC, and/or does not bind to one or more Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

In some embodiments, the Ig of the first Ig Fc region and the second Ig Fc region is hIgG4 and the amino acid sequence of the first Fc region and the second Fc region each comprise an amino acid substitution at amino acid position S228, an amino acid substitution at amino acid position F234, and/or an amino acid substitution at amino acid position E235, numbering according to the EU index of Kabat. In some embodiments, the Ig of the first Ig Fc region and the second Ig Fc region is hIgG4 and the amino acid sequence of the first Fc region and the second Fc region each comprise a proline at amino acid position S228, an alanine at amino acid position F234, and/or an alanine at amino acid position E235, numbering according to EU index of Kabat.

In some embodiments, the Ig of the first Ig Fc region and the second Ig Fc region is hIgG1 and the amino acid sequence of the first Fc region and the second Fc region each comprise an amino acid substitution at amino acid position L234, and/or an amino acid substitution at amino acid position L235, numbering according to the EU index of Kabat. In some embodiments, the Ig of the first Ig Fc region and the second Ig Fc region is hIgG1 and the amino acid sequence of the first Fc region and the second Fc region each comprise an alanine at amino acid position L234 and/or an alanine at amino acid position L235, numbering according to the EU index of Kabat. In some embodiments, the Ig of the first Ig Fc region and the second Ig Fc region is hIgG1 and the amino acid sequence of the first Fc region and the second Fc region each comprise a proline (or alanine) at amino acid position L234 and/or a proline (or alanine) at amino acid position L235, numbering according to the EU index of Kabat. In some embodiments, the Ig of the first Ig Fc region and the second Ig Fc region is hIgG1 and the amino acid sequence of the first Fc region and the second Fc region each comprise a proline (or alanine) at amino acid position L234 a proline (or alanine) at amino acid position L235, and/or a glycine at amino acid position P329, numbering according to the EU index of Kabat.

In some embodiments, the Ig of the first Ig Fc region and the second Ig Fc region is mIgG2a and the amino acid sequence of the first Fc region and the second Fc region each comprise an amino acid substitution at amino acid position L234, and/or an amino acid substitution at amino acid position L235, numbering according to the EU index of Kabat.

In some embodiments, the first Ig (e.g., hIg, mIg) Fc region comprises an amino acid sequence at least about 90%, 91%, 92%, 93%, 94%, 95%, 96%, 97%, 98%, 99%, or 100% identical to the amino acid sequence of the second Ig (e.g., hIg, mIg) Fc region.

In some embodiments, the first polypeptide or protein comprises from N- to C-terminus: the first Ig (e.g., hIg, mIg) Fc region and the first polypeptide or protein described herein (e.g., an immunomodulatory protein described herein); and the second polypeptide or protein comprises from N- to C-terminus: the second Ig (e.g., hIg, mIg) Fc region and the second polypeptide or protein described herein (e.g., an immunomodulatory protein described herein).

In some embodiments, the first polypeptide or protein comprises from N- to C-terminus: the first Ig (e.g., hIg, mIg) Fc region, a first peptide linker, and the first protein or polypeptide described herein (e.g., an immunomodulatory protein described herein); and the second polypeptide or protein comprises from N- to C-terminus: the second Ig (e.g., hIg, mIg) Fc region, a second peptide linker, and the second protein or polypeptide described herein (e.g., an immunomodulatory protein described herein).

In some embodiments, the first polypeptide or protein comprises from N- to C-terminus: the first polypeptide or protein described herein (e.g., an immunomodulatory protein described herein) and the first Ig (e.g., hIg, mIg) Fc region; and the second polypeptide or protein comprises from N- to C-terminus: the second polypeptide or protein described herein (e.g., an immunomodulatory protein described herein) and the second Ig (e.g., hIg, mIg) Fc region.

In some embodiments, the first polypeptide or protein comprises from N- to C-terminus: the first polypeptide or protein described herein (e.g., an immunomodulatory protein described herein), a first peptide linker, and the first Ig (e.g., hIg, mIg) Fc region; and the second polypeptide or protein comprises from N- to C-terminus: the second polypeptide or protein described herein (e.g., an immunomodulatory protein described herein), a second peptide linker, and the second Ig (e.g., hIg, mIg) Fc region.

In some embodiments, the amino acid sequence of the first peptide linker and the second peptide linker each comprises or consists of glycine or glycine and serine amino acid residues. In some embodiments, the amino acid of the first peptide linker and the second peptide linker each comprises or consists of (a) the amino acid sequence of any one of SEQ ID NOS: 49-57; or (b) the amino acid sequence of any one of SEQ ID NOS: 49-57 comprising or consisting of 1, 2, or 3 amino acid substitutions.

In some embodiments, the polypeptide or fusion protein comprises an amino acid sequence at least 85% identical to the amino acid sequence of any one of SEQ ID NO: 58, 61-69, or 72-79. In some embodiments, the polypeptide or fusion protein comprises an amino acid sequence at least 90% identical to the amino acid sequence of any one of SEQ ID NO: 58, 61-69, or 72-79. In some embodiments, the polypeptide or fusion protein comprises an amino acid sequence at least 95% identical to the amino acid sequence of any one of SEQ ID NO: 58, 61-69, or 72-79. In some embodiments, the polypeptide or fusion protein comprises an amino acid sequence 100% identical to SEQ ID NO: 58, 61-69, or 72-79. In some embodiments, the polypeptide or fusion protein comprises an amino acid sequence at least 85% identical to the amino acid sequence of any one of SEQ ID NO: 62-65 or 73-76. In some embodiments, the polypeptide or fusion protein comprises an amino acid sequence at least 90% identical to the amino acid sequence of any one of SEQ ID NO: 62-65 or 73-76. In some embodiments, the polypeptide or fusion protein comprises an amino acid sequence at least 95% identical to the amino acid sequence of any one of SEQ ID NO: 62-65 or 73-76. In some embodiments, the polypeptide or fusion protein comprises an amino acid sequence 100% identical to the amino acid sequence of any one of SEQ ID NO: 62-65 or 73-76.

In one aspect, provided herein are conjugates comprising a polypeptide or protein described herein (e.g., an immunomodulatory polypeptide or protein described herein) operably connected to a heterologous moiety.

In one aspect, provided herein are immunogenic peptides or proteins comprising (a) at least a portion of a polypeptide or protein described herein (e.g., an immunomodulatory polypeptide or protein described herein); and/or (b) a polypeptide or protein described herein (e.g., an immunomodulatory polypeptide or protein described herein) comprising at least one amino acid variation (e.g., substitution, addition, deletion), and wherein the immunogenic peptide or protein does not specifically bind the hIL-10R (e.g., hIL-10Rα) or binds the hIL-10R (e.g., hIL-10Rα) with lower affinity relative to a reference polypeptide or protein.

In some embodiments, the immunogenic peptide or protein comprises at least about 10, 15, 20, 30, 40, 50, 60, 70, 80, 90, 100, 150, 200, or 250 amino acids. In some embodiments, the immunogenic peptide or protein comprises from about 10-250, 10-200, 10-100, 10-90, 10-80, 10-70, 10-60, 10-50, 10-40, 10-30, 10-20, 10-250, 20-250, 30-250, 40-250, 50-250, 60-250, 70-250, 80-250, 90-250, 100-250, 10-200, 20-200, 30-200, 40-200, 50-200, 60-200, 70-200, 80-200, 90-200, or 100-200 amino acids.

In some embodiments, the amino acid sequence of the immunogenic peptide or protein comprises at least 2, 3, 4, 5, 6, 7, 8, 9, 10, or more amino acid variations (e.g., substitutions, additions, deletions) relative to a reference polypeptide or protein.

In some embodiments, the immunogenic peptide or protein comprises an amino acid sequence that is at least about 80%, 85%, 90%, 91%, 92%, 93%, 94%, 95%, 96%, 97%, 98%, 99% or 100% identical to a contiguous stretch of at least about 10, 15, 20, 30, 40, 50, 60, 70, 80, 90, 100, 150, or 200 amino acids of any one of SEQ ID NOS: 108-454.

In some embodiments, the immunogenic peptide or protein comprises an amino acid sequence that, other than the at least one amino acid variation (e.g., substitution, addition, deletion), is at least about 80%, 85%, 90%, 91%, 92%, 93%, 94%, 95%, 96%, 97%, 98%, 99% or 100% identical to the amino acid sequence of any one of SEQ ID NOS: 108-454.

In some embodiments, wherein the immunogenic polypeptide or protein is formulated with an adjuvant.

In one aspect, provided herein are isolated antibodies that specifically bind to a protein or polypeptide described herein (e.g., an immunomodulatory protein or polypeptide described herein).

In one aspect, provided herein are polynucleotides encoding a polypeptide or protein described herein (e.g., an immunomodulatory protein or polypeptide described herein), a fusion polypeptide or protein described herein, a conjugate described herein, an immunogenic peptide or protein described herein, or an antibody described herein.

In some embodiments, the polynucleotide is RNA (e.g., mRNA) or DNA.

In one aspect, provided herein are mRNA polynucleotides encoding a polypeptide or protein described herein (e.g., an immunomodulatory protein or polypeptide described herein), a fusion polypeptide or protein described herein, a conjugate described herein, an immunogenic peptide or protein described herein, or an antibody described herein.

In some embodiments, the mRNA comprises a heterologous 5′-untranslated region (UTR), 3′-UTR, or both a 5′-UTR and 3′-UTR. In some embodiments, the mRNA comprises a poly(A) sequence. In some embodiments, the mRNA comprises a 5′ cap structure.

In some embodiments, the polynucleotide or the mRNA polynucleotide comprises at least one variant nucleotide. In some embodiments, the nucleic acid sequence of the polynucleotide or mRNA polynucleotide, respectively, is codon optimized.

In one aspect, provided herein are expression vectors comprising a polynucleotide described herein or an mRNA polynucleotide described herein. In some embodiments, the expression vector is a viral vector or a plasmid.

In one aspect, provided herein are cells (e.g., host cells) comprising a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, or a carrier described herein.

In one aspect, provided herein are carriers comprising a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, or a cell described herein. In some embodiments, the carrier is a lipid nanoparticle, liposome, lipoplex, or nanoliposome.

In one aspect, provided herein are lipid nanoparticles comprising a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, or a cell described herein.

In one aspect, provided herein are vaccine compositions comprising an immunogenic peptide or protein described herein (or a nucleic acid molecule encoding the same (or a vector encoding the nucleic acid molecule)).

In one aspect, provided herein are pharmaceutical compositions comprising a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein; and a pharmaceutically acceptable excipient.

In one aspect, provided herein are kits comprising a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein; and optionally instructions for using any one or more of the foregoing.

In one aspect, provided herein are methods of delivering a polypeptide, protein, fusion protein, conjugate, polynucleotide, mRNA polynucleotide, expression vector, cell, carrier, lipid nanoparticle, immunogenic peptide or protein, an antibody, a vaccine composition, or pharmaceutical composition to a subject, the method comprising administering to the subject a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein, to thereby deliver the polypeptide, protein, fusion polypeptide, fusion protein, conjugate, immunogenic peptide or protein, antibody, mRNA polynucleotide, expression vector, cell, carrier, lipid nanoparticle, the vaccine composition, or pharmaceutical composition to a subject.

In one aspect, provided herein are uses of a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein in medicine.

In one aspect, provided herein are methods of stimulating the IL-10 pathway in a subject in need thereof, the method comprising administering to the subject a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein, to thereby stimulate the IL-10 pathway in the subject.

In one aspect, provided herein are a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein for use in a method of stimulating the IL-10 pathway in a subject in need thereof, the method comprising administering to the subject the polypeptide or protein, the polypeptide or protein, the fusion polypeptide or protein, the fusion protein or protein, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby stimulate the IL-10 pathway in the subject.

In one aspect, provided herein are uses of a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein in the manufacture of a medicament for stimulating the IL-10 pathway in a subject in need thereof, comprising administering to the subject the polypeptide or protein, the polypeptide or protein, the fusion polypeptide or protein, the fusion protein or protein, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby stimulate the IL-10 pathway in the subject.

In one aspect, provided herein are method of treating, preventing, or ameliorating an IL-10 responsive disease in a subject in need thereof, the method comprising administering to the subject a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein, to thereby treat, prevent, or ameliorate the IL-10 responsive disease. In some embodiments, the IL-10 responsive disease is a proinflammatory disease. In some embodiments, the IL-10 responsive disease is an autoimmune disease. In some embodiments, the IL-10 responsive disease is a metabolic inflammatory disease.

In one aspect, provided herein are a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein for use in a method of treating, preventing, or ameliorating an IL-10 responsive disease in a subject in need thereof, the method comprising administering to the subject the polypeptide or protein, the polypeptide or protein, the fusion polypeptide or protein, the fusion protein, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby treat, prevent, or ameliorate the IL-10 responsive disease. In some embodiments, the IL-10 responsive disease is a proinflammatory disease. In some embodiments, the IL-10 responsive disease is an autoimmune disease. In some embodiments, the IL-10 responsive disease is a metabolic inflammatory disease.

In one aspect, provided herein are uses of a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein in the manufacture of a medicament for treating, preventing, or ameliorating an IL-10 responsive disease in a subject in need thereof, comprising administering to the subject the polypeptide or protein, the polypeptide or protein, the fusion polypeptide or protein, the fusion protein, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby treat, prevent, or ameliorate the IL-10 responsive disease. In some embodiments, the IL-10 responsive disease is a proinflammatory disease. In some embodiments, the IL-10 responsive disease is an autoimmune disease. In some embodiments, the IL-10 responsive disease is a metabolic inflammatory disease.

In one aspect, provided herein are methods of suppressing or preventing an immune response in a subject in need thereof, the method comprising administering to the subject a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein, to thereby suppress or prevent an immune response in the subject.

In one aspect, provided herein are a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein for use in a method of suppressing or preventing an immune response in a subject in need thereof, the method comprising administering to the subject the polypeptide or protein, the polypeptide or protein, the fusion polypeptide or protein, the fusion, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby suppress or prevent an immune response in the subject.

In one aspect, provided herein are uses of a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein in the manufacture of a medicament for suppressing or preventing an immune response in a subject in need thereof, comprising administering to the subject the polypeptide or protein, the polypeptide or protein, the fusion polypeptide or protein, the fusion, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby suppress or prevent an immune response in the subject.

In one aspect, provide herein are methods of inducing tolerance to an immunogen in a subject in need thereof, the method comprising administering to the subject a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein, to thereby induce tolerance to an immunogen in the subject.

In one aspect, provided herein are a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein for use in a method of inducing tolerance to an immunogen in a subject in need thereof, the method comprising administering to the subject the polypeptide or protein, the polypeptide or protein, the fusion polypeptide or protein, the fusion, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby induce tolerance to an immunogen in the subject.

In one aspect, provided herein are uses of a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein in the manufacture of a medicament for inducing tolerance to an immunogen in a subject in need thereof, comprising administering to the subject the polypeptide or protein, the polypeptide or protein, the fusion polypeptide or protein, the fusion, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby induce tolerance to an immunogen in the subject.

In one aspect, provided herein are methods of suppressing IgE expression in a subject in need thereof, the method comprising administering to the subject a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein, to thereby suppress IgE expression in the subject.

In one aspect, provided herein are a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein for use in a method of suppressing IgE expression in a subject in need thereof, the method comprising administering to the subject the polypeptide or protein, the polypeptide or protein, the fusion polypeptide or protein, the fusion, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby suppress IgE expression in the subject.

In one aspect, provided herein are uses of a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein in the manufacture of a medicament for suppressing IgE expression in a subject in need thereof, comprising administering to the subject the polypeptide or protein, the polypeptide or protein, the fusion polypeptide or protein, the fusion, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby suppress IgE expression in the subject.

In some embodiments, the level of one or more pro-inflammatory cytokine (e.g., IFNγ, TNFα, IL-17A) is suppressed (e.g., as measured by an assay described herein).

In some embodiments, the level of immunogen specific immunoglobulin is suppressed (e.g., as measured by an assay described herein).

In some embodiments, the level of IgE (e.g., immunogen specific IgE, e.g., allergen specific IgE) is suppressed (e.g., as measured by an assay described herein).

In some embodiments, the subject has an IL-10 responsive disease (e.g., an IL-10 responsive disease described herein (e.g., an autoimmune disease (e.g., GvHD, an allergic disease, etc.)).

In some embodiments, prior to the administering of the polypeptide or protein described herein (e.g., an immunomodulatory protein or polypeptide described herein), the fusion polypeptide or protein described herein, the conjugate described herein, the immunogenic peptide or protein described herein, the antibody described herein, the polynucleotide described herein, the mRNA described herein, the expression vector described herein, the cell described herein, the carrier described herein, the lipid nanoparticle described herein, the vaccine composition described herein, or the pharmaceutical composition described herein to the subject, the subject has been administered one or more immunosuppressive agent (e.g., one or more steroid (e.g., corticosteroid)).

In some embodiments, the IL-10 responsive disease is refractory to the one or more immunosuppressive agent (e.g., the one or more steroid (e.g., corticosteroid)).

In some embodiments, the polypeptide or protein described herein (e.g., an immunomodulatory protein or polypeptide described herein), the fusion polypeptide or protein described herein, the conjugate described herein, the immunogenic peptide or protein described herein, the antibody described herein, the polynucleotide described herein, the mRNA described herein, the expression vector described herein, the cell described herein, the carrier described herein, the lipid nanoparticle described herein, the vaccine composition described herein, or the pharmaceutical composition described herein is administered to the subject in combination with one or more immunosuppressive agent (e.g., one or more steroid (e.g., corticosteroid)).

In some embodiments, the immunogen is an allergen. In some embodiments, the subject has been administered an allergen-specific therapy (e.g., as described herein (e.g., the subject has been administered at least 1 (e.g., at least 2, 3, 4, 5, 6, 7, 8, 9, 10, or more doses (e.g., escalating doses) of an allergen)). In some embodiments, the allergen-specific therapy induced a degree of tolerance to the immunogen in the subject.

In some embodiments, the administration of the polypeptide or protein described herein (e.g., an immunomodulatory protein or polypeptide described herein), the fusion polypeptide or protein described herein, the conjugate described herein, the immunogenic peptide or protein described herein, the antibody described herein, the polynucleotide described herein, the mRNA described herein, the expression vector described herein, the cell described herein, the carrier described herein, the lipid nanoparticle described herein, the vaccine composition described herein, or the pharmaceutical composition described herein enhances the degree of tolerance induced by the immunogen-specific therapy.

In some embodiments, the subject has received an allogenic or autologous cell, tissue, or organ transplant. In some embodiments, the immunogen is part of an allogenic or autologous cell, tissue, or organ. In some embodiments, the subject has graft versus host disease. In some embodiments, the graft versus host disease is acute or chronic.

In one aspect, provided herein are methods of inducing an immune response in a subject in need thereof, the method comprising administering to the subject a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein, to thereby induce an immune response in the subject.

In one aspect, provided herein are a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein for use in a method of inducing an immune response in a subject in need thereof, the method comprising administering to the subject the polypeptide or protein, the polypeptide or protein, the fusion polypeptide or protein, the fusion, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby induce an immune response in the subject.

In one aspect, provided herein are uses of a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein in the manufacture of a medicament for inducing an immune response in a subject in need thereof, comprising administering to the subject the polypeptide or protein, the polypeptide or protein, the fusion polypeptide or protein, the fusion, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby induce an immune response in the subject.

In one aspect, provided herein are methods of preventing, treating, or ameliorating a viral infection in a subject in need thereof, the method comprising administering to the subject a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein, to thereby prevent, treat, or ameliorate the viral infection in the subject. In some embodiments, the viral infection is an infection with a parapoxvirus, cytomegalovirus, gammaherpesvirus, orf virus, pseudocowpox virus, betaherpesvirus, and Epstein-Barr virus.

In one aspect provided herein is a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein for use in a method of preventing, treating, or ameliorating a viral infection in a subject in need thereof, the method comprising administering to the subject the polypeptide or protein, the polypeptide or protein, the fusion polypeptide or protein, the fusion, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby prevent, treat, or ameliorate the viral infection in the subject. In some embodiments, the viral infection is an infection with a parapoxvirus, cytomegalovirus, gammaherpesvirus, orf virus, pseudocowpox virus, betaherpesvirus, and Epstein-Barr virus.

In one aspect, provided herein are uses of a polypeptide or protein described herein (e.g., an immunomodulatory protein or polypeptide described herein), a fusion polypeptide or protein described herein, a conjugate described herein, an immunogenic peptide or protein described herein, an antibody described herein, a polynucleotide described herein, an mRNA described herein, an expression vector described herein, a cell described herein, a carrier described herein, a lipid nanoparticle described herein, a vaccine composition described herein, or a pharmaceutical composition described herein in the manufacture of a medicament for preventing, treating, or ameliorating a viral infection in a subject in need thereof, comprising administering to the subject the polypeptide or protein, the polypeptide or protein, the fusion polypeptide or protein, the fusion, the conjugate, the immunogenic peptide or protein, the antibody, the polynucleotide, the mRNA polynucleotide, the expression vector, the cell, the carrier, the lipid nanoparticle, the vaccine composition, or the pharmaceutical composition, to thereby prevent, treat, or ameliorate the viral infection in the subject. In some embodiments, the viral infection is an infection with a parapoxvirus, cytomegalovirus, gammaherpesvirus, orf virus, pseudocowpox virus, betaherpesvirus, and Epstein-Barr virus.

In one aspect, provided herein are method of determining the presence of a virus in a subject, the method comprising (a) obtaining the sample from a subject or providing a sample that has been obtained from a subject, and (b) determining the presence or absence of a polypeptide or protein described herein (e.g., an immunomodulatory polypeptide or protein described herein) (or a fragment or variant thereof) or a polynucleotide encoding the polypeptide or protein described herein (e.g., an immunomodulatory polypeptide or protein described herein) (or a fragment or variant thereof) in the sample.

In some embodiments, the sample is a blood, cell, tissue, or saliva, or nasal swab. In some embodiments, the subject is a human. In some embodiments, an antibody described herein is utilized to determine the presence or absence of the polypeptide or protein described herein (e.g., an immunomodulatory polypeptide or protein described herein) (or a fragment or variant thereof). In some embodiments, the viral infection is an infection with a parapoxvirus, cytomegalovirus, gammaherpesvirus, orf virus, pseudocowpox virus, betaherpesvirus, and Epstein-Barr virus.

In one aspect, provided herein are methods of diagnosing a viral infection in a subject, the method comprising (a) obtaining a sample from a subject or providing a sample that has been obtained from a subject, (b) determining the presence or absence of a polypeptide or protein described herein (e.g., an immunomodulatory polypeptide or protein described herein) (or a fragment or variant thereof) or a polynucleotide encoding the polypeptide or protein described herein (e.g., an immunomodulatory polypeptide or protein described herein) (or a fragment or variant thereof), and (c) diagnosing the subject as having the viral infection if the polypeptide or protein described herein (e.g., an immunomodulatory polypeptide or protein described herein) (or a fragment or variant thereof) or a polynucleotide encoding the polypeptide or protein described herein (e.g., an immunomodulatory polypeptide or protein described herein) (or a fragment or variant thereof) is determined to be present in the sample in step (b). In some embodiments, the method is an in vitro method.

In some embodiments, the sample is a blood, cell, tissue, or saliva, or nasal swab. In some embodiments, the subject is a human. In some embodiments, an antibody described herein is utilized to determine the presence or absence of the polypeptide or protein described herein (e.g., an immunomodulatory polypeptide or protein described herein) (or a fragment or variant thereof). In some embodiments, the viral infection is an infection with a parapoxvirus, cytomegalovirus, gammaherpesvirus, orf virus, pseudocowpox virus, betaherpesvirus, and Epstein-Barr virus.

In one aspect, provided herein are methods of treating a viral infection in a subject, the method comprising (a) receiving testing results that determined the presence of a polypeptide or protein described herein (e.g., an immunomodulatory polypeptide or protein described herein) (or a fragment or variant thereof) or a polynucleotide encoding the polypeptide or protein described herein (e.g., an immunomodulatory polypeptide or protein described herein) (or a fragment or variant thereof) in a sample from the subject, (b) diagnosing the subject as having the viral infection, and (c) administering a therapeutic agent to treat the viral infection.

In some embodiments, the sample is a blood, cell, tissue, or saliva, or nasal swab. In some embodiments, the subject is a human. In some embodiments, an antibody described herein is utilized to determine the presence or absence of the polypeptide or protein described herein (e.g., an immunomodulatory polypeptide or protein described herein) (or a fragment or variant thereof). In some embodiments, the viral infection is an infection with a parapoxvirus, cytomegalovirus, gammaherpesvirus, orf virus, pseudocowpox virus, betaherpesvirus, and Epstein-Barr virus.

In one aspect, provided herein are therapeutic agents to treat the viral infection for use in a method of treating a viral infection in a subject, the method comprising (a) receiving testing results that determined the presence of a polypeptide or protein described herein (e.g., an immunomodulatory polypeptide or protein described herein) (or a fragment or variant thereof) or a polynucleotide encoding the polypeptide or protein described herein (e.g., an immunomodulatory polypeptide or protein described herein) (or a fragment or variant thereof) in a sample from the subject, (b) diagnosing the subject as having the viral infection, and (c) administering the therapeutic agent to treat the viral infection.

In some embodiments, the sample is a blood, cell, tissue, or saliva, or nasal swab. In some embodiments, the subject is a human. In some embodiments, an antibody described herein is utilized to determine the presence or absence of the polypeptide or protein described herein (e.g., an immunomodulatory polypeptide or protein described herein) (or a fragment or variant thereof). In some embodiments, the viral infection is an infection with a parapoxvirus, cytomegalovirus, gammaherpesvirus, orf virus, pseudocowpox virus, betaherpesvirus, and Epstein-Barr virus.

In one aspect, provided herein are uses of a therapeutic agent to treat the viral infection in the manufacture of a medicament for treating a viral infection in a subject, comprising (a) receiving testing results that determined the presence of a polypeptide or protein described herein (e.g., an immunomodulatory polypeptide or protein described herein) (or a fragment or variant thereof) or a polynucleotide encoding the polypeptide or protein described herein (e.g., an immunomodulatory polypeptide or protein described herein) (or a fragment or variant thereof) in a sample from the subject, (b) diagnosing the subject as having the viral infection, and (c) administering the therapeutic agent to treat the viral infection.

In some embodiments, the sample is a blood, cell, tissue, or saliva, or nasal swab. In some embodiments, the subject is a human. In some embodiments, an antibody described herein is utilized to determine the presence or absence of the polypeptide or protein described herein (e.g., an immunomodulatory polypeptide or protein described herein) (or a fragment or variant thereof). In some embodiments, the viral infection is an infection with a parapoxvirus, cytomegalovirus, gammaherpesvirus, orf virus, pseudocowpox virus, betaherpesvirus, and Epstein-Barr virus.

### 4. BRIEF DESCRIPTION OF THE FIGURES

FIG. 1 is a bar graph showing the level (ng/mL) of each of immunomodulatory fusion proteins 1-11 (IFPs-1-11) and a reference Fc-hIL-10 fusion protein expressed in vitro.

FIG. 2A is a line graph showing hIL-10R engagement by IFP-1 and a reference Fc-hIL-10 fusion protein in vitro. FIG. 2B is a line graph showing hIL-10R engagement by IFP-2 and a reference Fc-hIL-10 fusion protein in vitro. FIG. 2C is a line graph showing hIL-10R engagement by IFP-3 and a reference Fc-hIL-10 fusion protein in vitro. FIG. 2D is a line graph showing hIL-10R engagement by IFP-4 and a reference Fc-hIL-10 fusion protein in vitro. FIG. 2E is a line graph showing hIL-10R engagement by IFP-5 and a reference Fc-hIL-10 fusion protein in vitro. FIG. 2F is a line graph showing hIL-10R engagement by IFP-6 and a reference Fc-hIL-10 fusion protein in vitro. FIG. 2G is a line graph showing hIL-10R engagement by IFP-7 and a reference Fc-hIL-10 fusion protein in vitro. FIG. 2H is a line graph showing hIL-10R engagement by IFP-8 and a reference Fc-hIL-10 fusion protein in vitro. FIG. 2I is a line graph showing hIL-10R engagement by IFP-9 and a reference Fc-hIL-10 fusion protein in vitro. FIG. 2J is a line graph showing hIL-10R engagement by IFP-10 and a reference Fc-hIL-10 fusion protein in vitro. FIG. 2K is a line graph showing hIL-10R engagement by IFP-11 and a reference Fc-hIL-10 fusion protein in vitro.

FIG. 3A is a line graph showing hIL-10R engagement by a reference Fc-hIL-10 fusion protein in vitro in the presence and absence of an anti-IL-10Rα antibody that blocks binding of hIL-10 to hIL-10Rα. FIG. 3B is a line graph showing hIL-10R engagement by IFP-1 in vitro in the presence and absence of an anti-IL-10Rα antibody that blocks binding of hIL-10 to hIL-10Rα. FIG. 3C is a line graph showing hIL-10R engagement by IFP-2 in vitro in the presence and absence of an anti-IL-10Rα antibody that blocks binding of hIL-10 to hIL-10Rα. FIG. 3D is a line graph showing hIL-10R engagement by IFP-3 in vitro in the presence and absence of an anti-IL-10Rα antibody that blocks binding of hIL-10 to hIL-10Rα. FIG. 3E is a line graph showing hIL-10R engagement by IFP-4 in vitro in the presence and absence of an anti-IL-10Rα antibody that blocks binding of hIL-10 to hIL-10Rα. FIG. 3F is a line graph showing hIL-10R engagement by IFP-5 in vitro in the presence and absence of an anti-IL-10Rα antibody that blocks binding of hIL-10 to hIL-10Rα. FIG. 3G is a line graph showing hIL-10R engagement by IFP-6 in vitro in the presence and absence of an anti-IL-10Rα antibody that blocks binding of hIL-10 to hIL-10Rα. FIG. 3H is a line graph showing hIL-10R engagement by IFP-7 in vitro in the presence and absence of an anti-IL-10Rα antibody that blocks binding of hIL-10 to hIL-10Rα. FIG. 3I is a line graph showing hIL-10R engagement by IFP-8 in vitro in the presence and absence of an anti-IL-10Rα antibody that blocks binding of hIL-10 to hIL-10Rα. FIG. 3J is a line graph showing hIL-10R engagement by IFP-9 in vitro in the presence and absence of an anti-IL-10Rα antibody that blocks binding of hIL-10 to hIL-10Rα. FIG. 3K is a line graph showing hIL-10R engagement by IFP-10 in vitro in the presence and absence of an anti-IL-10Rα antibody that blocks binding of hIL-10 to hIL-10Rα. FIG. 3L is a line graph showing hIL-10R engagement by IFP-11 in vitro in the presence and absence of an anti-IL-10Rα antibody that blocks binding of hIL-10 to hIL-10Rα.

FIG. 4A is a radar chart showing the expression of IFN-γ, IL-1β, IL-8, IL-6, TNFα, and IL-4 from LPS-stimulated human peripheral blood mononuclear cells (hPBMCs) treated with a reference Fc-hIL-10 fusion protein or an Fc-GFP negative control (referred to in FIG. 4A as “Fc”). The scale indicates the percent of cytokine present relative to the level in the Fc-GFP control group. FIG. 4B is a radar chart showing the expression of IL-1β, IL-8, IL-6, and TNFα, from LPS-stimulated human peripheral blood mononuclear cells (hPBMCs) treated with IFP-2 or an Fc-GFP negative control (referred to in FIG. 4B as “Fc”). The scale indicates the percent of cytokine present relative to the level in the Fc-GFP control group. FIG. 4C is a radar chart showing the expression of IFN-γ, IL-1β, IL-8, IL-6, TNFα, and IL-4 from LPS-stimulated human peripheral blood mononuclear cells (hPBMCs) treated with IFP-5 or an Fc-GFP negative control (referred to in FIG. 4C as “Fc”). The scale indicates the percent of cytokine present relative to the level in the Fc-GFP control group. FIG. 4D is a radar chart showing the expression of IL-1β, IL-8, IL-6, and TNFα from LPS-stimulated human peripheral blood mononuclear cells (hPBMCs) treated with IFP-6 or an Fc-GFP negative control (referred to in FIG. 4D as “Fc”). The scale indicates the percent of cytokine present relative to the level in the Fc-GFP control group. FIG. 4E is a radar chart showing the expression of IFN-γ, IL-1β, IL-8, IL-6, TNFα, and IL-4 from LPS-stimulated human peripheral blood mononuclear cells (hPBMCs) treated with IFP-7 or an Fc-GFP negative control (referred to in FIG. 4E as “Fc”). The scale indicates the percent of cytokine present relative to the level in the Fc-GFP control group. FIG. 4F is a radar chart showing the expression of IL-13, IL-8, IL-6, and TNFα from LPS-stimulated human peripheral blood mononuclear cells (hPBMCs) treated with IFP-8 or an Fc-GFP negative control (referred to in FIG. 4F as “Fc”). The scale indicates the percent of cytokine present relative to the level in the Fc-GFP control group.

FIG. 5 is a series of line graphs showing the expression of IFNγ, TNFα, and IL-17a from stimulated human peripheral blood mononuclear cells (hPBMCs) treated with IFP-7 (bottom row) or a reference hIL-10 Fc fusion protein (top row) alone or in combination with dexamethasone.

FIG. 6 is a PCA plot showing the clustering of IMPs 1-175 (SEQ ID NOS: 108-454) by sequence and structural similarity and visualized by principal component analysis (PCA). “Sampled” indicates the 97 IMPs (IMPs 1-12, 21, 26-27, 31-32, 36, 38, 41, 49-50, 53, 55, 58, 62, 67-70, 72-73, 75-82, 84, 86-87, 89, 91, 93, 95, 97-100, 102-103, 106-109, 111-112, 115, 117-118, 120-127, 129-130, 132, 134-136, 138-158) that were generated and analyzed in e.g., Examples 1 and 3 herein. “Not Sampled” indicates the other 78 IMPs. The sequences are clustered in clusters 0-5 as denoted in the plot. The IMPs in each cluster is set forth in Example 2.

FIG. 7 is a bar graph showing the engraftment of human CD45+ immune cells (hCD45) in vivo in a xenograft mouse model. The mice were treated with dexamethasone (dexa) and IFP-7, dexamethasone (dexa) and a reference hIL-10 Fc fusion protein, dexamethasone (dexa) and human Fc protein, or an untreated control. The data is presented as the ratio of hCD45+ immune cells to murine CD45+ immune cells (mCD45). “*” indicates a P value of about 0.02. “**” indicates a P value of about 0.001-0.005.

FIG. 8A is a bar graph showing the percentage of human naïve (CD27+ and CD45Ra+) CD4+ FOXP3− T cells in vivo in a xenograft mouse model. The mice were treated with dexamethasone (dexa) and IFP-7, dexamethasone (dexa) and a reference hIL-10 Fc fusion protein, dexamethasone (dexa) and human Fc protein, or an untreated control. “*” indicates a P value of about 0.05. “****” indicates a P value of less than 0.0001. FIG. 8B is a bar graph showing the percentage of human effector (CD27− and CD45Ra−) CD4+ FOXP3− T cells in vivo in a xenograft mouse model of GVHD. The mice were treated with dexamethasone (dexa) and IFP-7, dexamethasone and a reference hIL-10 Fc fusion protein, dexamethasone and human Fc, or an untreated control. “**” indicates a P value of about 0.001. “***” indicates a P value of about 0.0009. “****” indicates a P value of less than 0.0001.

FIG. 9 is a bar graph showing the expression of IFNγ in vivo in a xenograft mouse model. The mice were treated with dexamethasone (dexa) and IFP-7, dexamethasone and a reference hIL-10 Fc fusion protein, dexamethasone and human Fc, or an untreated control. “*” indicates a P value of about 0.05.

FIG. 10 is a bar graph showing the relative body weight of mice in a xenograft mouse model of graft versus host disease (GVHD). The mice were treated with dexamethasone (dexa) and IFP-7, dexamethasone and a reference hIL-10 Fc fusion protein, an untreated control, or control mice that received no hPBMCs.

FIG. 11 is a bar graph showing the survival of mice in a xenograft mouse model of GVHD. The mice were treated with dexamethasone (dexa) and IFP-7, dexamethasone and a reference hIL-10 Fc fusion protein, an untreated control, or control mice that received no hPBMCs. “*” indicates a P value of about 0.02.

### 5. DETAILED DESCRIPTION

While hIL-10 has been evaluated as a therapeutic for the treatment of different autoimmune diseases, in each instance it has shown limited efficacy. The biologically active form of hIL-10 is an unstable homodimer, which in vivo exhibits a short half-life, easy degradation, and low potency. The inventors have, inter alia, identified immunomodulatory proteins and polypeptides that bind to the hIL-10R (e.g., hIL-10Rα) and that, inter alia, exhibit one or more improved properties over hIL-10, e.g., increased binding affinity for the hIL-10R (e.g., hIL-10Rα), increased potency, enhanced suppression of one or more pro-inflammatory cytokines, etc. Accordingly, the novel immunomodulatory proteins disclosed herein may be used for modulation of an immune response, and for treatment of diseases (e.g., IL-10 responsive diseases). As such, the current disclosure provides, inter alia, novel immunomodulatory proteins for use in pharmaceutical compositions for the treatment of diseases (e.g., IL-10 responsive diseases).

### 5.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

Use of the singular herein includes the plural unless specifically stated otherwise. For example, as used herein, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The term “about” refers to a value or composition that is within an acceptable error range for the particular value or composition as determined by one of ordinary skill in the art, which will depend in part on how the value or composition is measured or determined, i.e., the limitations of the measurement system. When particular values or compositions are provided herein, unless otherwise stated, the meaning of “about” should be assumed to be within an acceptable error range for that particular value or composition.

Where proteins and/or polypeptides are described herein, it is understood that polynucleotides (e.g., RNA (e.g., mRNA) or DNA nucleic acid molecules) encoding the protein or polypeptide are also provided herein.

Where proteins, polypeptides, nucleic acid molecules, vectors, carriers, etc. are described herein, it is understood that isolated forms of the proteins, polypeptides, nucleic acid molecules, vectors, carriers, etc. are also provided herein.

Where proteins, polypeptides, nucleic acid molecules, etc. are described herein, it is understood that recombinant forms of the proteins, polypeptides, nucleic acid molecules, etc. are also provided herein.

Where polypeptides or sets of polypeptides are described herein, it is understood that proteins comprising the polypeptides or sets of polypeptides folded into their three-dimensional structure (i.e., tertiary or quaternary structure) are also provided herein and vice versa.

As used herein, the term “adjuvant” refers to a substance that causes stimulation of the immune system of a subject when administered to the subject.

As used herein, the term “administering” refers to the physical introduction of an agent, e.g., a therapeutic agent (or a precursor of the therapeutic agent that is metabolized or altered within the body of the subject to produce the therapeutic agent in vivo) or vaccine to a subject, using any of the various methods and delivery systems known to those skilled in the art. Administering can also be performed, for example, once, a plurality of times, and/or over one or more extended periods.

As used herein, the term “affinity” refers to the strength of the binding of one protein (e.g., a Ligand) to another protein (e.g., a Receptor). The affinity of a protein is measured by the dissociation constant Kd, defined as [Ligand]×[Receptor]/[Ligand-Receptor] where [Ligand-Receptor] is the molar concentration of the Ligand-Receptor complex, [Ligand] is the molar concentration of the unbound Ligand and [Receptor] is the molar concentration of the unbound Receptor. The affinity constant Ka is defined by 1/Kd. Standard methods of measuring affinity are known to the person of ordinary skill in the art. Exemplary methods of measuring affinity are described herein, see for example, § 5.2.2.

As used herein, the term “antibody” or “antibodies” is used in the broadest sense and encompasses various immunoglobulin (Ig) (e.g., human Ig (hIg), murine Ig (mIg)) structures, including, but not limited to monoclonal antibodies, polyclonal antibodies, multispecific (e.g., bispecific, trispecific) antibodies, and antibody fragments so long as they exhibit the desired antigen-binding activity (i.e., antigen binding fragments or variants). The term antibody thus includes, for example, full-length antibodies; antigen-binding fragments of full-length antibodies; molecules comprising antibody CDRs, VH regions, and/or VL regions; and antibody-like scaffolds (e.g., fibronectins). Examples of antibodies include, without limitation, monoclonal antibodies, polyclonal antibodies, monospecific antibodies, multispecific antibodies, human antibodies, humanized antibodies, chimeric antibodies, camelized antibodies, intrabodies, affybodies, diabodies, tribodies, heteroconjugate antibodies, antibody-drug conjugates, single domain antibodies (e.g., VHH, (VHH)₂), single chain antibodies, single-chain Fvs (scFv; (scFv)₂), Fab fragments (e.g., Fab, single chain Fab (scFab), F(ab′)₂ fragments, disulfide-linked Fvs (sdFv), Fc fusions (e.g., Fab-Fc, scFv-Fc, VHH-Fc, (scFv)₂-Fc, (VHH)₂-Fc), and antigen-binding fragments of any of the above, and conjugates or fusion proteins comprising any of the above. Antibodies can be of Ig isotype (e.g., IgG, IgE, IgM, IgD, or IgA), any class (e.g., IgG₁, IgG₂, IgG₃, IgG₄, IgA₁ or IgA₂), or any subclass (e.g., IgG₂a or IgG₂b) of Ig). In certain embodiments, antibodies described herein are IgG antibodies, or a class (e.g., human IgG₁ or IgG₄) or subclass thereof. In certain embodiments, antibodies described herein are mIgG antibodies, or a class (e.g., mIgG1 or mIgG2a) or subclass thereof. In some embodiments, the antibody is a human, humanized, or chimeric IgG₁ or IgG₄ monoclonal antibody. In some embodiments, the term antibodies refers to a monoclonal or polyclonal antibody population. Antibodies described herein can be produced by any standard methods known in the art, e.g., recombinant production in host cells, see, e.g., § 5.5; or synthetic production.

The terms “CH1” and “CH1 region” are used interchangeably herein and refer to the first constant region of an immunoglobulin heavy chain. The amino acid sequence of an exemplary reference hIgG1 CH1 region is set forth in SEQ ID NO: 531; and the amino acid sequence of an exemplary reference hIgG4 CH1 region is set forth in SEQ ID NO: 534.

The terms “CH2” and “CH2 region” are used interchangeably herein and refer to the second constant region of an immunoglobulin heavy chain. The amino acid sequence of an exemplary reference hIgG1 CH2 region is set forth in SEQ ID NO: 13; and the amino acid sequence of an exemplary reference hIgG4 CH2 region is set forth in SEQ ID NO: 24.

The terms “CH3” and “CH3 region” are used interchangeably herein and refer to the third constant region of an immunoglobulin heavy chain. The amino acid sequence of an exemplary reference hIgG1 CH3 region is set forth in SEQ ID NO: 14; and the amino acid sequence of an exemplary reference hIgG4 CH3 region is set forth in SEQ ID NO: 25.

As used herein, the term “conjugation” refers to chemical conjugation of a protein or polypeptide with a moiety (e.g., small molecule, polypeptide, polynucleotide, carbohydrate, lipid, synthetic polymer (e.g., polymers of polyethylene glycol (PEG)), etc.). The moiety can be directly connected to the protein or polypeptide or indirectly connected through a linker, e.g., as described herein. Chemical conjugation methods are well known in the art, as are commercially available conjugation reagents and kits, with detailed instructions for their use readily available from the commercial suppliers.

As used herein, the term “derived from,” with reference to a polynucleotide refers to a polynucleotide that has at least 70% sequence identity to a reference polynucleotide (e.g., a naturally occurring polynucleotide) or a fragment thereof. The term “derived from,” with reference to a polypeptide or protein refers to a polypeptide or protein that comprises an amino acid sequence that has at least 70% sequence identity to the amino acid sequence of a reference polypeptide or protein (e.g., a naturally occurring polypeptide or protein). The term “derived from” as used herein does not denote any specific process or method for obtaining the polynucleotide, polypeptide, or protein. For example, the polynucleotide, polypeptide, or protein can be recombinantly produced or chemically synthesized.

As used herein, the term “diagnosing” or “diagnosis” refers to a determination of the presence, absence, severity, or course of treatment of a disease (e.g., an infection, e.g., a viral infection). The term “diagnosing” encompasses an initial determination as well as subsequent determinations (e.g., monitoring) after the initial determination.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

The term “EC50” or “half maximal effective concentration” is a measure of potency of an agent (e.g., an immunomodulatory protein or polypeptide described herein) and refers to the concentration of the agent (e.g., an immunomodulatory protein or polypeptide described herein) required to induce a response halfway between baseline and maximal response after a particular exposure period. Assays to measure the EC50 of a protein or polypeptide are standard in the art, see, also, e.g., § 5.2.2. In some embodiments, the EC50 is measured according to the methods described in Example 3 herein.

The term “effector function” when used in reference to an antibody refers to those biological activities attributable to the Fc region of an antibody, which therefore vary with the antibody isotype. Antibody effector functions include, but are not limited to, antibody-dependent cell-mediated cytotoxicity (ADCC), antibody-dependent cellular phagocytosis (ADCP), complement dependent cytotoxicity (CDC), Fc receptor binding (e.g., FcγRI, FcγRIIa, FcγRIIc, FcγRIIIa, and/or FcγRIIIb (e.g., FcγRI, FcγIIa, and/or FcγIIIa)), and C1q binding.

As used herein, the term “Fc region” refers to the C-terminal region of an Ig heavy chain that comprises from N- to C-terminus at least a CH2 region operably connected to a CH3 region. In some embodiments, the Fc region comprises an Ig hinge region or at least a portion of an Ig hinge region operably connected to the N-terminus of the CH2 region. In some embodiments, the Fc region is engineered relative to a reference Fc region, see, e.g., § 5.3.1.3. Additional examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, the entire contents of which is incorporated by reference herein for all purposes).

The term “functional variant” as used herein in reference to a polypeptide or protein refers to a polypeptide or protein that comprises at least one but no more than 15%, not more than 12%, no more than 10%, no more than 8% amino acid variation (e.g., substitution, deletion, addition) compared to the amino acid sequence of a reference polypeptide or protein, wherein the polypeptide or protein retains at least one particular function of the reference polypeptide or protein. Not all functions of the reference polypeptide or protein (e.g., wild type) need be retained by the functional variant of the protein. In some instances, one or more functions are selectively reduced or eliminated. In some embodiments, the reference polypeptide or protein is a wild type protein. For example, a functional variant of a hIL-10 polypeptide or protein can refer to a hIL-10 protein comprising one or more amino acid substitution as compared to a reference hIL-10 protein (e.g., wild type) that retains the ability to specifically bind the hIL-10R.

The term “functional fragment” as used herein in reference to a polypeptide or protein refers to a fragment of a reference polypeptide or protein that retains at least one particular function. Not all functions of the reference polypeptide or protein need be retained by a functional fragment of the polypeptide or protein. In some instances, one or more functions are selectively reduced or eliminated. In some embodiments, the reference polypeptide or protein is a wild type protein. For example, a functional fragment of hIL-10 can refer to a fragment of hIL-10 that retains the ability to specifically bind the IL-10R.

As used herein, the term “fuse” and grammatical equivalents thereof refer to the operable connection of at least a first polypeptide to a second polypeptide, wherein the first and second polypeptides are not naturally found operably connected together. For example, the first and second polypeptides are derived from different proteins. The term fuse encompasses both a direct connection of the at least two polypeptides through a peptide bond, and the indirect connection through a linker (e.g., a peptide linker).

As used herein, the term “fusion protein” and grammatical equivalents thereof refers to a protein that comprises at least one polypeptide operably connected to another polypeptide, wherein the first and second polypeptides are different and not naturally found operably connected together. For example, the first and second polypeptides of the fusion protein are each derived from different proteins. The at least two polypeptides of the fusion protein can be directly operably connected through a peptide bond; or can be indirectly operably connected through a linker (e.g., a peptide linker). Therefore, for example, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “half-life extension moiety” refers to a moiety (e.g., small molecule, polypeptide, polynucleotide, carbohydrate, lipid, synthetic polymer (e.g., polymers of PEG), etc.) that when conjugated or otherwise operably connected (e.g., fused) to a polypeptide or protein (the subject polypeptide or protein), increases the half-life of the subject polypeptide or protein in vivo when administered to a subject (e.g., a human subject). The pharmacokinetic properties of the polypeptide or protein can be evaluated utilizing in vivo models known in the art.

As used herein, the term “half-life extension polypeptide” or “half-life extension protein” refers to a polypeptide or protein that when operably connected to another polypeptide or protein (the subject polypeptide or protein), increases the half-life of the subject polypeptide or protein in vivo when administered to a subject (e.g., a human subject). The pharmacokinetic properties of the polypeptide or protein can be evaluated utilizing in vivo models known in the art.

As used herein, the term “heterologous”, when used to describe a first element in reference to a second element means that the first element and second element do not exist in nature disposed as described. For example, a polypeptide comprising a “heterologous moiety” means a polypeptide that is joined to a moiety (e.g., small molecule, polypeptide, polynucleotide, carbohydrate, lipid, synthetic polymer (e.g., polymers of PEG), etc.) that is not joined to the polypeptide in nature. In one embodiment, the heterologous moiety is not derived from a polypeptide or protein comprising or consisting of the amino acid sequence of any one of SEQ ID NOS: 108-454. For example, a non-limiting example of a heterologous moiety is a heterologous polypeptide (as defined herein). In one embodiment, the heterologous polypeptide is a polypeptide derived from a polypeptide or protein other than a polypeptide or protein comprising or consisting of the amino acid sequence of any one of SEQ ID NOS: 108-454. For example, a non-limiting example of a heterologous polypeptide, as described herein, is a human Ig Fc region.

As used, herein the term “heterologous signal peptide” refers to a signal peptide that is not operably connected to a subject polypeptide or protein in nature. For example, in reference to a polypeptide comprising a signal peptide from human IL-2 operably connected to human IL-12, the human IL-2 signal peptide would constitute a heterologous signal peptide. The terms “signal peptide” and “signal sequence” are used interchangeably herein.

The terms “hinge” or “hinge region” are used interchangeably herein and refer to the hinge region of an immunoglobulin heavy chain. The amino acid sequence of an exemplary reference hIgG1 hinge region is set forth in SEQ ID NO: 12; and the amino acid sequence of an exemplary reference hIgG4 hinge region is set forth in SEQ ID NO: 22.

As used herein, the term “homologous signal peptide” refers to a signal peptide that is operably connected to a subject polypeptide or protein in nature. For example, in reference to a polypeptide comprising a signal peptide from human IL-2 operably connected to human IL-2, the human IL-2 signal peptide would constitute a homologous signal peptide.

As used herein, the term “human interleukin 10” or “hIL-10” refers to the human immunomodulatory cytokine that mediates signaling through the human IL-10 Receptor. The amino acid sequence of an exemplary reference mature hIL-10 protein is set forth in SEQ ID NO: 2.

As used herein, the term “human IL-10 Receptor” or “hIL-10R” refers to the human heterodimeric cell surface complex comprised of hIL-10Rα and hIL-10Rβ, through which hIL-10 mediates signaling.

As used herein, the term “human IL-10 Receptor α” or “hIL-10Rα” refers to the alpha (α) subunit of the hIL-10 Receptor. The amino acid sequence of an exemplary reference mature hIL-10Rα polypeptide is set forth in SEQ ID NO: 4.

As used herein, the term “human IL-10 Receptor β” or “hIL-10Rβ” refers to the beta (β) subunit of the hIL-10 Receptor. The amino acid sequence of an exemplary reference mature hIL-10Rβ polypeptide is set forth in SEQ ID NO: 6.

As used herein, the term “variant Ig Fc fusion polypeptide or protein” refers to a fusion polypeptide or protein comprising an immunomodulatory polypeptide or protein described herein and an Ig Fc region, wherein the Ig Fc region comprises one or more variation (e.g., one or more amino acid substitution, deletion, or addition)) that decreases or abolishes one or more Fc effector function, relative to a reference Ig Fc fusion protein that does not comprise the one or more variation.

As used herein, the term “IL-10 responsive disease” refers to a disease in which one or more symptom is reduced or ameliorated through stimulation of the IL-10 pathway.

As used herein, the term “immunogen” refers to a substance that is capable of inducing an immune response (e.g., an adaptive immune response) in a human. An immunogen may have one or more isoforms, sequence variants, or splice variants that have equivalent biological and immunological activity, and are thus also considered for the purposes of this disclosure to be immunogenic equivalents of the immunogen.

As used herein, the term “immunogenic peptide or protein” refers to a peptide or protein that comprises an immunogen.

As used herein, the term “in combination with” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed. In some embodiments, administration of two or more agents or treatments in combination is such that the reduction in a symptom, or other parameter related to the condition is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, and intramuscular routes. The therapeutic agents can be administered by the same route or by different routes.

As used herein, the term “isolated” with reference to a polypeptide, protein, or polynucleotide refers to a polypeptide, protein, or polynucleotide that is substantially free of other cellular components with which it is associated in the natural state.

As used herein, the term “moiety” is used generically to describe any macro or micro molecule that can be operably connected to a polypeptide or protein described herein. Exemplary moieties include, but are not limited small molecules, polypeptides, polynucleotides (e.g., DNA, RNA), carbohydrates, lipids, synthetic polymers (e.g., polymers of PEG).

As used herein, the term “modified nucleotide,” “nucleotide modification,” or use of the term “modification” and the like in reference to a nucleotide or nucleic acid sequence refers to a nucleotide comprising a chemical modification, e.g., a modified sugar moiety, a modified nucleobase, and/or a modified internucleoside linkage, or any combination thereof. Exemplary modifications are provided herein, see, e.g., § 5.4.4.2. In certain embodiments of the instant disclosure, inclusion of a deoxynucleotide—which is acknowledged as a naturally occurring form of nucleotide—if present within an RNA molecule is considered to constitute a modified nucleotide.

As used herein, the term “obtaining a sample” refers to the acquisition of a sample. The term includes the direct acquisition from a subject and the indirect acquisition through one or more third parties wherein one of the third parties directly acquired the sample from the subject.

As used herein, the term “operably connected” refers to the linkage of two moieties in a functional relationship. For example, a polypeptide is operably connected to another polypeptide when they are linked (either directly or indirectly via a peptide linker) in frame such that both polypeptides are functional (e.g., a fusion protein or polypeptide described herein). Or for example, a transcription regulatory polynucleotide e.g., a promoter, enhancer, or other expression control element is operably linked to a polynucleotide that encodes a protein if it affects the transcription of the polynucleotide that encodes the protein. The term “operably connected” can also refer to the conjugation of a moiety to e.g., a polynucleotide or polypeptide (e.g., the conjugation of a PEG polymer to a protein or polypeptide).

The determination of “percent identity” between two sequences (e.g., peptide or protein (amino acid sequences) or polynucleotide (nucleic acid sequences)) can be accomplished using a mathematical algorithm. A specific, non-limiting example of a mathematical algorithm utilized for the comparison of two sequences is the algorithm of Karlin S & Altschul SF (1990) PNAS 87: 2264-2268, modified as in Karlin S & Altschul SF (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance intended for use in contact with the tissues of human beings and/or non-human animals, and without excessive toxicity, irritation, allergic response, or other problem or complication, commensurate with a reasonable therapeutic benefit/risk ratio.

As used herein, the term “poly(A) sequence,” refers to a sequence of adenosine nucleotides, typically located at the 3′-end of a coding linear RNA, of up to about 1000 adenosine nucleotides. In some embodiments, the poly(A) sequence is essentially homopolymeric, e.g., a poly(A) sequence of e.g., 100 adenosine nucleotides having essentially the length of 100 nucleotides. In other embodiments, the poly(A) sequence may be interrupted by at least one nucleotide different from an adenosine nucleotide, e.g., a poly(A) sequence of e.g., 100 adenosine nucleotides may have a length of more than 100 nucleotides (comprising 100 adenosine nucleotides and in addition said at least one nucleotide—or a stretch of nucleotides—different from an adenosine nucleotide). It has to be understood that “poly(A) sequence” as defined herein typically relates to mRNA—however in the context of the invention, the term likewise relates to corresponding sequences in a DNA molecule (e.g., a “poly(T) sequence”).

The terms “polynucleotide” and “nucleic acid molecule” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA polynucleotides encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” are used interchangeably herein.

As used herein, the term “protein” refers to one or more polypeptides folded into its three-dimensional structure. Where polypeptides are contemplated herein, it should be understood that proteins comprising the polypeptides (i.e., the polypeptides folded unto their three-dimensional structure) are also provided herein. In some embodiments, an immunomodulatory polypeptide or protein herein is an immunomodulatory protein.

A “prophylactic” treatment is a treatment administered to a subject who does not exhibit signs of a disease or exhibits only early signs for the purpose of decreasing the risk of developing pathology.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester linkage found between the nucleotides of an unmodified nucleic acid molecule.

As used herein, the term “sample” encompass a variety of biological specimens obtained from a subject. Exemplary sample types include, e.g., blood and other liquid samples of biological origin (including, but not limited to, whole-blood, peripheral blood mononuclear cells (PBMCs), serum, plasma, urine, saliva, amniotic fluid, stool, synovial fluid, etc.), nasopharyngeal swabs, solid tissue samples such as biopsies (or cells derived therefrom and the progeny thereof), tissue cultures (or cells derived therefrom and the progeny thereof), and cell cultures (or cells derived therefrom and the progeny thereof). The term also includes samples that have been manipulated in any way after their procurement from a subject, such as by centrifugation, filtration, washing, precipitation, dialysis, chromatography, lysis, treatment with reagents, enriched for certain cell populations, refrigeration, freezing, staining, etc.

As used herein, the term “translatable RNA” refers to any RNA that encodes at least one polypeptide and can be translated to produce the encoded protein in vitro, in vivo, in situ or ex vivo. A translatable RNA may be an mRNA or a circular RNA encoding a polypeptide.

The term “(scFv)₂” as used herein refers to an antibody that comprises a first and a second scFv operably connected (e.g., via a peptide linker). The first and second scFv can specifically bind the same or different antigens. In some embodiments, the first and second scFv are operably connected by a peptide linker.

The term “scFv-Fc” as used herein refers to an antibody that comprises a scFv operably linked (e.g., via a peptide linker) to an Fc domain or subunit of an Fc domain. In some embodiments, a scFv is operably connected to only a first Fc domain of a first and a second Fc domain pair. In some embodiments, a first scFv is operably connected to a first Fc domain and a second scFv is operably connected to a second Fc domain of a first and second Fc domain pair.

The term “(scFv)₂-Fc” as used herein refers to a (scFv)₂ operably linked (e.g., via a peptide linker) to an Fc domain or a subunit of an Fc domain. In some embodiments, a (scFv)₂ is operably connected to only a first Fc domain of a first and a second Fc domain pair. In some embodiments, a first (scFv)₂ is operably connected to a first Fc domain and a second (scFv)₂ is operably connected to a second Fc domain of a first and second Fc domain pair.

As used herein, the term “single domain antibody” or “sdAb” refers to an antibody having a single monomeric variable antibody domain. A sdAb is able to specifically bind to a specific antigen. A VHH (as defined herein) is an example of a sdAb.

As used herein, the term “signal peptide” or “signal sequence” refers to a sequence (e.g., an amino acid sequence) that can direct the transport or localization of a protein to a certain organelle, cell compartment, or extracellular export. The term encompasses both the signal sequence peptide and the nucleic acid sequence encoding the signal peptide. Thus, references to a signal peptide in the context of a nucleic acid refers to the nucleic acid sequence encoding the signal peptide.

As used herein, the term “specifically binds” refers to preferential interaction, i.e., significantly higher binding affinity, between a first protein (e.g., a ligand) and a second protein (e.g., the ligand's cognate receptor) relative to other amino acid sequences. Herein, when a first protein or polypeptide is said to “specifically bind” to a second protein or polypeptide, it is understood that the first protein or polypeptide specifically binds to an epitope of the second protein or polypeptide. The term “epitope” refers to the portion of the second protein or polypeptide that the first protein or polypeptide specifically recognizes. The term specifically binds includes molecules that are cross reactive with the same epitope of a different species. For example, an antibody that specifically binds human IL-10 may be cross reactive with IL-10 of another species (e.g., cynomolgus, murine, etc.), and still be considered herein to specifically bind human IL-10. A protein can specifically bind more than one different protein.

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

As used herein, the term “therapeutically effective amount” of a therapeutic agent refers to any amount of the therapeutic agent that, when used alone or in combination with another therapeutic agent, improves a disease condition, e.g., protects a subject against the onset of a disease (or infection); improves a symptom of disease or infection, e.g., decreases severity of disease or infection symptoms, decreases frequency or duration of disease or infection symptoms, increases disease or infection symptom-free periods; prevents or reduces impairment or disability due to the disease or infection; or promotes disease (or infection) regression. The ability of a therapeutic agent to improve a disease condition can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease or infection and/or symptom(s) associated therewith or obtaining a desired pharmacologic and/or physiologic effect. It will be appreciated that, although not precluded, treating a disease or infection does not require that the disease or infection, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease or infection. In some embodiments, the effect is preventative, i.e., the effect protects or prevents an occurrence or reoccurrence of a disease or infection. To this end, the presently disclosed methods comprise administering a therapeutically effective amount of a composition as described herein.

The terms “VL” and “VL domain” are used interchangeably to refer to the light chain variable region of an antibody.

The terms “VH” and “VH domain” are used interchangeably to refer to the heavy chain variable region of an antibody.

The term “VHH” as used herein refers to a type of single domain antibody (sdAb) that has a single monomeric heavy chain variable antibody domain (VH). Such antibodies can be found in or produced from camelid mammals (e.g., camels, llamas) which are naturally devoid of light chains or synthetically produced.

The term “(VHH)₂” as used herein refers to an antibody that comprises a first and a second VHH operably connected (e.g., via a peptide linker). The first and the second VHH can specifically bind the same or different antigens. In some embodiments, the first and second VHH are operably connected by a peptide linker.

The term “VHH-Fc” as used herein refers to an antibody that comprises a VHH operably linked (e.g., via a peptide linker) to an Fc domain or a subunit of an Fc domain. In some embodiments, a VHH is operably connected to only a first Fc domain of a first and a second Fc domain pair. In some embodiments, a first VHH is operably connected to a first Fc domain and a second VHH is operably connected to a second Fc domain of a first Fc and a second Fc pair.

The term “(VHH)₂-Fc” as used herein refers to (VHH)₂ operably linked (e.g., via a peptide linker) to an Fc domain or a subunit of an Fc domain. In some embodiments, a (VHH)₂ is operably connected to only a first Fc domain of a first and a second Fc domain pair. In some embodiments, a first (VHH)₂ is operably connected to a first Fc domain and a second (VHH)₂ is operably connected to a second Fc domain of a first Fc and a second Fc pair.

As used herein, the term “5′-untranslated region” or “5′-UTR” refers to a part of a nucleic acid molecule located 5′ (i.e., “upstream”) of a coding sequence and which is not translated into protein. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g., by enzymatic or post-transcriptional addition of a 5′-cap structure.

As used herein the term “3′-untranslated region” or “3′-UTR” refers to a part of a nucleic acid molecule located 3′ (i.e., downstream) of a coding sequence and which is not translated into protein. A 3′-UTR may located between a coding sequence and an (optional) terminal poly(A) sequence of a nucleic acid sequence. A 3′-UTR may comprise elements for controlling gene expression, also called regulatory elements. Such regulatory elements may be, e.g., ribosomal binding sites, miRNA binding sites etc.

### 5.2 Immunomodulatory Proteins

IL-10 is the founding member of the IL-10 cytokine family, which includes IL-19, IL-20, IL-22, IL-24, and IL-26. hIL-10 is an important immunoregulatory cytokine that functions in part, to suppress inflammatory immune responses. hIL-10 potently inhibits the production of pro-inflammatory cytokines such as IFN-γ, TNFα, IL-1β, and IL-6 in several cell types and prevents dendritic cell maturation in part by inhibiting the expression of IL-12. hIL-10 also inhibits the expression of MHC and co-stimulatory molecules important for cell-mediated immunity. While, largely functioning as an anti-inflammatory cytokine, hIL-10 is pleiotropic in nature, known to also mediate pro-inflammatory effects, including the stimulation of IFN-γ and granzyme B production by CD8+ T cells.

The cellular responses induced by hIL-10 require the specific recognition and assembly of the hIL-10 receptor (hIL-10R). The hIL-10R comprises two different subunits, a hIL-10Rα subunit and a hIL-10Rβ subunit. While similar in overall architecture, hIL-10Rβ exhibits lower affinity for hIL-10 relative to the a subunit. Due to this difference in affinity of hIL-10Rα and hIL-10Rβ for hIL-10, each receptor subunit has a different function in activating hIL-10 cellular responses. More specifically, hIL-10Rα generally functions as the hIL-10 binding subunit which controls cell specificity and cellular targeting of hIL-10 to immune cells that selectively express the hIL-10Rα subunit. While the hIL-10Rβ subunit functions as a sensor, which effectively senses hIL-10 bound to the IL-10Rα and activates signaling based on the kinetics of the hIL-10/hIL-10Rα interaction.

The amino acid sequence of a reference immature hIL-10 polypeptide and mature hIL-10 polypeptide is set forth in SEQ ID NOS: 1 and 2, respectively. The amino acid sequence of a reference immature hIL-10Rα polypeptide and mature hIL-10Rα polypeptide is set forth in SEQ ID NOS: 3 and 4, respectively. The amino acid sequence of a reference immature hIL-10β polypeptide and mature hIL-10β polypeptide is set forth in SEQ ID NOS: 5 and 6, respectively. See Table 1, herein.

TABLE 1
The Amino Acid Sequence of Reference hIL-10, hIL-10Rα, and hIL-10Rβ
Polypeptides

| | | SEQ ID |
|---|---|---|
| Description | Amino Acid Sequence | NO |
| hIL-10 | MHSSALLCCLVLLTGVRASPGQGTQSENSCTHFPGNLPNMLRDLRD | 1 |
| (Immature-Signal | AFSRVKTFFQMKDQLDNLLLKESLLEDFKGYLGCQALSEMIQFYLE | |
| Peptide Underlined) | EVMPQAENQDPDIKAHVNSLGENLKILRLRLRRCHRFLPCENKSKA | |
| | VEQVKNAFNKLQEKGIYKAMSEFDIFINYIEAYMTMKIRN | |
| hIL-10 | SPGQGTQSENSCTHFPGNLPNMLRDLRDAFSRVKTFFQMKDQLDNL | 2 |
| (Mature-No Signal | LLKESLLEDFKGYLGCQALSEMIQFYLEEVMPQAENQDPDIKAHVN | |
| Peptide) | SLGENLKTLRLRLRRCHRFLPCENKSKAVEQVKNAFNKLQEKGIYK | |
| | AMSEFDIFINYIEAYMTMKIRN | |
| hIL-10Rα | MLPCLVVLLAALLSLRLGSDAHGTELPSPPSVWFEAEFFHHILHWT | 3 |
| (Immature-Signal | PIPNQSESTCYEVALLRYGIESWNSISNCSQTLSYDLIAVILDLYH | |
| Peptide Underlined) | SNGYRARVRAVDGSRHSNWTVTNTRFSVDEVTLTVGSVNLEIHNGF | |
| | ILGKIQLPRPKMAPANDTYESIFSHFREYEIAIRKVPGNFTFTHKK | |
| | VKHENFSLLTSGEVGEFCVQVKPSVASRSNKGMWSKEECISLTRQY | |
| | FTVTNVIIFFAFVLLLSGALAYCLALQLYVRRRKKLPSVLLFKKPS | |
| | PFIFISQRPSPETQDTIHPLDEEAFLKVSPELKNLDLHGSTDSGFG | |
| | STKPSLQTEEPQFLLPDPHPQADRTLGNREPPVLGDSCSSGSSNST | |
| | DSGICLQEPSLSPSTGPTWEQQVGSNSRGQDDSGIDLVQNSEGRAG | |
| | DTQGGSALGHHSPPEPEVPGEEDPAAVAFQGYLRQTRCAEKATKTG | |
| | CLEEESPLTDGLGPKFGRCLVDEAGLHPPALAKGYLKQDPLEMTLA | |
| | SSGAPIGQWNQPTEEWSLLALSSCSDLGISDWSFAHDLAPLGCVAA | |
| | PGGLLGSFNSDLVTLPLISSLQSSE | |
| hIL-10Rα | HGTELPSPPSVWFEAEFFHHILHWTPIPNQSESTCYEVALLRYGIE | 4 |
| (Mature-No Signal | SWNSISNCSQTLSYDLTAVTLDLYHSNGYRARVRAVDGSRHSNWTV | |
| Peptide) | TNTRFSVDEVTLTVGSVNLEIHNGFILGKIQLPRPKMAPANDTYES | |
| | IFSHFREYEIAIRKVPGNFIFTHKKVKHENFSLLISGEVGEFCVQV | |
| | KPSVASRSNKGMWSKEECISLTRQYFTVINVIIFFAFVLLLSGALA | |
| | YCLALQLYVRRRKKLPSVLLFKKPSPFIFISQRPSPETQDTIHPLD | |
| | EEAFLKVSPELKNLDLHGSTDSGFGSTKPSLQTEEPQFLLPDPHPQ | |
| | ADRILGNREPPVLGDSCSSGSSNSTDSGICLQEPSLSPSTGPTWEQ | |
| | QVGSNSRGQDDSGIDLVQNSEGRAGDTQGGSALGHHSPPEPEVPGE | |
| | EDPAAVAFQGYLRQTRCAEKATKIGCLEEESPLTDGLGPKFGRCLV | |
| | DEAGLHPPALAKGYLKQDPLEMTLASSGAPTGQWNQPTEEWSLLAL | |
| | SSCSDLGISDWSFAHDLAPLGCVAAPGGLLGSFNSDLVTLPLISSL | |
| | QSSE | |
| hIL-10Rβ | MAWSLGSWLGGCLLVSALGMVPPPENVRMNSVNFKNILQWESPAFA | 5 |
| (Immature-Signal | KGNLTFTAQYLSYRIFQDKCMNTTLTECDFSSLSKYGDHTLRVRAE | |
| Peptide Underlined) | FADEHSDWVNITFCPVDDTIIGPPGMQVEVLADSLHMRFLAPKIEN | |
| | EYETWTMKNVYNSWTYNVQYWKNGTDEKFQITPQYDFEVLRNLEPW | |
| | TTYCVQVRGFLPDRNKAGEWSEPVCEQTTHDETVPSWMVAVILMAS | |
| | VFMVCLALLGCFALLWCVYKKTKYAFSPRNSLPQHLKEFLGHPHHN | |
| | TLLFFSFPLSDENDVEDKLSVIAEDSESGKQNPGDSCSLGTPPGQG | |
| | PQS | |
| hIL-10Rβ | MVPPPENVRMNSVNFKNILQWESPAFAKGNLTFTAQYLSYRIFQDK | 6 |
| (Mature-No Signal | CMNTTLTECDFSSLSKYGDHTLRVRAEFADEHSDWVNITFCPVDDT | |
| Peptide) | IIGPPGMQVEVLADSLHMRFLAPKIENEYETWTMKNVYNSWTYNVQ | |
| | YWKNGTDEKFQITPQYDFEVLRNLEPWTTYCVQVRGFLPDRNKAGE | |
| | WSEPVCEQTTHDETVPSWMVAVILMASVEMVCLALLGCFALLWCVY | |
| | KKTKYAFSPRNSLPQHLKEFLGHPHHNTLLFFSFPLSDENDVFDKL | |
| | SVIAEDSESGKQNPGDSCSLGTPPGQGPQS | |

In one aspect, the present disclosure provides immunomodulatory proteins and polypeptides (and functional fragments and variants thereof) that specifically bind the hIL-10R (e.g., hIL-10Rα). The amino acid sequence of the immunomodulatory proteins and polypeptides provided herein is set forth in Table 2. The amino acid sequence of the immature form of the immunomodulatory proteins and polypeptides (i.e., containing the native signal peptide) is set forth in SEQ ID NOS: 108-118 or 127-290. The amino acid sequence of the mature form of the immunomodulatory proteins and polypeptides (i.e., lacking the native signal peptide) is set forth in SEQ ID NOS: 119-126 or 291-454.

The signal peptides have been computationally predicted using standard methods (see, e.g., Teufel, F., Almagro Armenteros, J. J., Johansen, A. R. et al. SignalP 6.0 predicts all five types of signal peptides using protein language models. Nat Biotechnol (2022). https://doi.org/10.1038/s41587-021-01156-3, the entire contents of which is incorporated by reference herein for all purposes). A person of ordinary skill in the art would know how to experimentally identify and/or validate a computationally predicted signal peptide using standard methods known in the art, e.g., expression of the immunomodulatory protein from a host cell and sequencing of the intracellular form and the extracellular form of the expressed protein (see, e.g., Zhang Z, Henzel W J. Signal peptide prediction based on analysis of experimentally verified cleavage sites. Protein Sci. 2004; 13(10):2819-2824. doi:10.1110/ps.04682504, the entire contents of which is incorporated by reference herein for all purposes).

TABLE 2
The Amino Acid Sequence of Immunomodulatory Proteins

| | | SEQ ID |
|---|---|---|
| Description | Amino Acid Sequence | NO |
| Immunomodulatory | MGKRAFVVSVAMALLGIYVITNTVNARHCMFGDSLRNSPDMKNML | 108 |
| Protein (IMP)-1 | QDLRGGYSGSGIKRTFQGKDTLDSMLLTQSLLDDFKGYLGCQALS | |
| with signal peptide | EMIQFYLEEVMPQAENHGPTDSVKQLGEKLHTLNQKFGECPRWFP | |
| | CYYNTTPAVENVKSVFSKLQERGVYKAMSEFDIFINYIETYTTMK | |
| IMP-2 | MARRLTVASCGSVSLLAAFAAVLLIGCQLESGEALPLGSRSADSR | 109 |
| with signal peptide | SVDGQRVPAPQNNYPGLLRDLRLGYEGFKQKVTDSHPDETLLGSS | |
| | RLAGDLKGPLRCQALSEMIQFLLQVVLPDAENSRQDLRSQFSTLG | |
| | DRITGLRQQLRRDPTVFPCESRSDGVSDLRSAYTRLGSTGAEKVL | |
| | SEFDIFINYIEAYVTSV | |
| IMP-3 | MSNNKILVCAVIILTYTLYTDAYCVEYAESDEDRQQCSSSSNFPA | 110 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVFNMLQERGVYKAMSEFDIFINYIES | |
| | YMTTKM | |
| IMP-4 | MANVVYVVLVISIMMANIHVSKTYCTSCSHHQCTEDENQKQDCED | 111 |
| with signal peptide | ANHSLPHMLRELRAAFGKVKTFFQMKDQLHSLLLTQSLLDDFKGY | |
| | LGCQALSEMIQFYLEEVMPQAENHGPEEHDNSLSEHGPDVKEHVN | |
| | SLGEKLKTLRLRLRRCHRFLPCENKSKAVEKVKRVFSELQERGVY | |
| | KAMSEFDIFINYIETYMTT | |
| IMP-5 | MQGLQLLRGLLCCGVFAAASSRSPKNKPSIDCNPQTGDFVNMLKS | 112 |
| with signal peptide | MRQDYSRIRDTLHDRDKLHSSLLTGALLDEMMGYSGCRTTLLLME | |
| | HYLDTWYPAAYRHHLYDNQTLVVVDRMGSTLVALLKAMVQCPMLA | |
| | CGAPSPAMDKMLQQEAKMKKYTGVYKGISETDLLLGYLELYMMKF | |
| | KR | |
| IMP-6 | MRRRRSFGIVVSGAIRTLLMVAVVAVSVRGHEHKVPPACDPVHGN | 113 |
| with signal peptide | LAGIFKELRAIYASIREALQKKDTVYYTSLFNDRVLQEMLSPMGC | |
| | RVTNELMEHYLDGVLPRAAHFDYDNSTLNGLHAFTSSMQALYQHM | |
| | LKCPALACTGKTPAWMYFLEVEHKLNPWRGTAKAAAEADLLLNYL | |
| | ETFLLQF | |
| IMP-7 | MGSRRLSRCSFATAVCLVAIVAAVAAKGRDSKPSPACDPMHGALA | 114 |
| with signal peptide | GIFKELRTTYRSVREALQTKDTVYYVSLFHEQLLQEMLSPVGCRV | |
| | TNELMQHYLDGVLPRAFHCGYDNATLNALHALSSSLSTLYQHMLK | |
| | CPALACTGQTPAWTQFLDTEHKLDPWKGTVKATAEMDLLLNYLET | |
| | FLLQS | |
| IMP-8 | MLSVMVSSSLVLIVFFLGASEEAKPATTTIKNTKPQCRPEDYATR | 115 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDTLFSRLEEYLHSRK | |
| IMP-9 | MALAHQLPVWIFSIWILYFTLPLSEERVLPLRGNCKLLLQDTVIP | 116 |
| with signal peptide | NLLYSMRSIFQDIKPYFQGKDSLNNLLLSGQLLEDLQSPIGCDAL | |
| | SEMIQFYLEEVMPQAEIHHPKHKNSVMQLGETLHTLISQLQECTA | |
| | LFPCKHKSLGAQKIKEEVSKLGQYGIIKAVAEFDIFINYMESYFG | |
| | VK | |
| IMP-10 | MRRRRRSFGIIVAGAIGTLLMMAVVVLSAHDHEHKEVPPACDPVH | 117 |
| with signal peptide | GNLAGIFKELRATYASIREGLQKKDTVYYTSLFNDRVLHEMLSPM | |
| | GCRVTNELMEHYLDGVLPRASHLDYDNSTLNGLHVFASSMQALYQ | |
| | HMLKCPALACTGKTPAWMYFLEVEHKLNPWRGTAKAAAEADLLLN | |
| | YLETFLLQF | |
| IMP-11 | MRRRRSFGIVVAGAIGTLLMMAVVVFSAHEHKEVPPACDPVHGNL | 118 |
| with signal peptide | AGIFKELRATYASIREGLQKKDTVYYTSLFNDRVLQEMLSPMGCR | |
| | VTNELMEHYLDGVLPRALHLDYDNSTLNGLHAFASSMQALYQHML | |
| | KCPALACTGKTPAWMYFLEVEHKLNPWRGTAKAAAEADLLLNYLE | |
| | TFLLQF | |
| IMP-12 | MSKNKILVCVVIILTYTLYTDAYCVEYEESEEDRQQCSSSNFPAS | 127 |
| with signal peptide | LPHMLRELRAAFGKVKTFFQMKDQLNSMLLTQSLLDDFKGYLGCQ | |
| | ALSEMIQFYLEEVMPQAENHGPDIKEHVNSLGEKLKILRLRLRRC | |
| | HRFLPCENKSKAVEQVKRVFNMLQERGVYKAMSEFDIFINYIESY | |
| | MTTKP | |
| IMP-13 | MSNNKILVCVVIILTYTLYTDAYCVEYEESEEDRQQCSSSNFPAS | 128 |
| with signal peptide | LPHMLRELRAAFGKVKIFFQMKDQLNSMLLTQSLLDDFKGYLGCQ | |
| | ALSEMIQFYLEEVMPQAENHGPDIKEHVNSLGEKLKTLRLRLRRC | |
| | HRFLPCENKSKAVEQVKRVFNMLQERGVYKAMSEFDIFINYIESY | |
| | MTTKT | |
| IMP-14 | MPNNKILVCAVIILTYTLYTDAYCVEYEESEEDRQQCSSSNFPAS | 129 |
| with signal peptide | LPHMLRELRAAFGKVKTFFQMKDQLNSMLLTQSLLDDFKGYLGCQ | |
| | ALSEMIQFYLEEVMPQAENHGPDIKEHVNSLGEKLKTLRLRLRRC | |
| | HRFLPCENKSKAVEQVKRVFNMLQERGVYKAMSEFDIFINYIESY | |
| | MTTKS | |
| IMP-15 | MSNKKILVCVVIILTYTLYTDAYCVEYKESEEDRQQCSSSSFPAS | 130 |
| with signal peptide | LPHMLRELRAAFGKVKTFFQMKDQLNSMLLTQSLLDDFKGYLGCQ | |
| | ALSEMIQFYLEEVMPQAENHGPDIKEHVNSLGEKLKTLRLRLRRC | |
| | HRFLPCENKSKAVEQVKRVFNMLQERGVYKAMSEFDIFINYIESY | |
| | MTTK | |
| IMP-16 | MSNNKILVCVAIILTYTLYTDAYCVEYAESDEDKQQCSGSNFPAS | 131 |
| with signal peptide | LPHMLRELRAAFGKVKTFFQMKDQLNSMLLTQSLLDDFKGYLGCQ | |
| | ALSEMIQFYLEEVMPQAENHGPDIKEHVNSLGEKLKTLRLRLRRC | |
| | HRFLPCENKSKAVEQVKRVFNMLQERGVYKAMSEFDIFINYIESY | |
| | MTTKM | |
| IMP-17 | MSKNKVLVCFVIILTYTLYTDAYCVEYEESEEDKQQCGSNGGPAS | 132 |
| with signal peptide | LPHMLRELRAAFGKVKTFFQMKDQLNSMLLTQSLLDDEKGYLGCQ | |
| | ALSEMIQFYLEEVMPQAENHGPDIKEHVNSLGEKLKTLRLRLRRC | |
| | HRFLPCENKSKAVEQVKRVFNMLQERGVYKAMSEFDIFINYIESY | |
| | MTTKM | |
| IMP-18 | MSNNKILVCAVIILTYTLYTDAYCVEYAESDEDRQQCSGSNFPAS | 133 |
| with signal peptide | LPHMLRELRAAFGKVKTFFQMKDQLNSMLLTQSLLDDEKGYLGCQ | |
| | ALSEMIQFYLEEVMPQAENHGPDIKEHVNSLGEKLKTLRLRLRRC | |
| | HRFLPCENKSKAVEQVKRVENMLQERGVYKAMSEFDIFINYIESY | |
| | MTTKM | |
| IMP-19 | MSNNKILLCVAIILTYTLYTDAYCVEYEESEEDKQQCSSSSNFPA | 134 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-20 | MSNNKILVCAVIILTYTLYTDAYCIQYEESEEDKQQCSSSNFPAS | 135 |
| with signal peptide | LPHMLRELRAAFGKVKTFFQMKDQLNSMLLTQSLLDDEKGYLGCQ | |
| | ALSEMIRFYLEEVMPQAENHGPDIKEHVNSLGEKLKTLRLRLRRC | |
| | HRFLPCENKSKAVEQVKRVENMLQERGVYKAMSEFDIFINYIESY | |
| | MTTKM | |
| IMP-21 | MSKNKFLVCVVIILTYTLYTDAYCVEYEESEEDRQQCSSSNFPAS | 136 |
| with signal peptide | LPHMLRELRAAFGKVKTFFQMKDQLNSMLLTQSLLDDFKGYLGCQ | |
| | ALSEMIQFYLEEVMPQAENHGPDIKEHVNSLREKLKTLRLRLRRC | |
| | HRFLPCENKSKAVEQVKRVFNMLQERGVYKAMSEFDIFINYIESY | |
| | MTTKT | |
| IMP-22 | MSKNKILVCFVIILTYTLYTDAYCVEYEESEEDKQQCGSSSNFPA | 137 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVFNMLQERGVYKAMSEFDIFINYIES | |
| | YMTTKM | |
| IMP-23 | MSNNKILVCVAIILTYTLYTDAYCVEYAESDEDKQQCSGSNFPAS | 138 |
| with signal peptide | LPHMLRELRAAFGKVKTFFQMKDQLNSMLLTQSLLDDFKGYLGCQ | |
| | ALSEMIQFYLEEVMPRAENHGPDIKEHVNSLGEKLKTLRLRLRRC | |
| | HRFLPCENKSKAVEQVKRVFNMLQERGVYKAMSEFDIFINYIESY | |
| | MTTKM | |
| IMP-24 | MSNNKILVCVVIILTYTLYTDAYCVEYEESEEDRQQCSGSSNFPA | 139 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVFNMLQERGVYKAMSEFDIFINYIES | |
| | YMTTKM | |
| IMP-25 | MSNNKILVCAVIILTYTLYTDAYCVEYEESDEDRQQCSSSSNFPA | 140 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVFNMLQERGVYKAMSEFDIFINYIES | |
| | YMTTKM | |
| IMP-26 | MSKNKILVCVAIILTYTLYTDAYCVEYEESDEDKQQCSSSTGAPA | 141 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-27 | MSKNKILVCVAIILTYTLYTDAYCVEYEETKEDEQQCSSSSNFPA | 142 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-28 | MSKNKILVCVVIILTYTLYTDAYCVEYEESEEDRQQCSSSNFPAS | 143 |
| with signal peptide | LPHMLRELRAAFGKVKIFFQMKDQLNSMLLTQSLLDDFKGYLGCQ | |
| | ALSEMIQFYLEEVMPQAENHGPDIKEHVNSLGEKLKTLRLRLRRC | |
| | HRFLPCENKSKAVEQVKRVFNMLQERGDYKAMSEFDIFINYIESY | |
| | MTTKS | |
| IMP-29 | MSNNKILVCVVIILTYTLYTDAYCVEYEESEEDRQQCSSSSNFPA | 144 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCEDKSKAVEQVKRVFNMLQERGVYKAMSEFDILINYIES | |
| | YMTTKM | |
| IMP-30 | MSNNKILVCAVIILTYTLYTDAYCVEYEESDEDRQQCSSSSNFPA | 145 |
| with signal peptide | SLPHMLRELRAAFGKVKIFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVFNMLQERGVYKAMSEFDIFINYIES | |
| | YMATKM | |
| IMP-31 | MFRALLLCCLALLAGVWADNRYDGQDGNDCPTLPTSLPHMLHELR | 146 |
| with signal peptide | AAFSRVKTFFQMKDQLDNMLLDGSLLEDFKGYLGCQALSEMIQFY | |
| | LEEVMPQAENHSTDQEKDKVNSLGEKLKTLRVRLRRCHRFLPCEN | |
| | KSKAVEQVKSAFSKLQEKGVYKAMSEFDIFINYIEAYMTTKMKN | |
| IMP-32 | MSKNKILVCVAIILTYTLYTDAYCVEYLESREDEQQCSSSSNFPA | 147 |
| with signal peptide | SLPHMLRELRAAFGKVKIFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVFNMLQERGVYKAMSEFDIFINYIES | |
| | YMTTKM | |
| IMP-33 | MSKNKILVCVAIILTYTLYTDAYCVEYEESKEDEQQCSGSNGASA | 148 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKILRLRLRR | |
| | CHRFLPCENKSKAVEQVKRVFNMLQERGVYKAMSEFDIFINYIES | |
| | YMTTKM | |
| IMP-34 | MSKNKILVCVAIILTYTLYTDAYCVEYLESGEDEQQCGSSSNFPA | 149 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVFNMLQERGVYKAMSEFDIFINYIES | |
| | YMTTKM | |
| IMP-35 | MSKNKILVCVAIILTYTLYTDAYCVEYLESREDEQQCSGSNGASA | 150 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVFNMLQERGVYKAMSEFDIFINYIES | |
| | YMTTKM | |
| IMP-36 | MPGAALLYCLFFVTGVWAESENNCTHFPTSLPHMLHELRAAFSRV | 151 |
| with signal peptide | KTFFQMKDQLDNMLLNGSLLEDFKGYLGCQALSEMIQFYLEEVMP | |
| | QAENHSGGGGPDIKEHVNSLGEKLKTLRVRLRRCHRFLPCENKSK | |
| | AVEQVKSAFSKLQEKGVYKAMSEFDIFINYIEAYMTTKMKNKK | |
| IMP-37 | MSNNKILVCVAIILTYTLYTDAYCVEYLESDEDKQHCSSSNGASA | 152 |
| with signal peptide | SSPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVFNMLQERGVYKAMSEFDIFINYIES | |
| | YMTTKM | |
| IMP-38 | MSNNKILVCVAIILTYTLYTNAYCVEYLESEEDKQQCGSNGASSS | 153 |
| with signal peptide | SPHMLRELRAAFGKVKTFFQMKDQLNSMLLTQSLLDDFKGYLGCQ | |
| | ALSEMIQFYLEEVMPQAENHGPDVKEHVNSLAEKLKTLRLRLRRC | |
| | HRFLPCENKSKAVEQVKRVFNMLQERGVYKAMSEFDIFINYIESY | |
| | MTTKS | |
| IMP-39 | MSNNKILVCVAIILTYTLYTNAYCVEYLESEEDKQQCGSNGASSS | 154 |
| with signal peptide | SPHMLRELRAAFGKVKTFFQMKDQLNSMLLTQSLLDDEKGYLGCQ | |
| | ALSEMIQFYLEEVMPQAENHGPDVKEHVNSLGEKLKTLRLRLRRC | |
| | HRFLPCENKSKAVAQVKRVFNMLQERGVYKAMSEFDIFINYIESY | |
| | MTTKS | |
| IMP-40 | ILVCFVIILTYTLYTDAYCVEYEESEEDRQQCSSSNFPASLPHML | 155 |
| with signal peptide | RELRAAFGKVKTFFQMKDQLNSMLLTQSLLDDFKGYLGCQALSEM | |
| | IQFYLEEVMPQAENHGPDIKEHVNSLGEKLKTLRLRLRRCHRFLP | |
| | CENKSKAVEQVKRVFNMLQERGVYKAMSEFDIFINYIES | |
| IMP-41 | ILVCFVIILTYTLYTDAYCVEYEESEEDRQQCSSSNFPASLPHML | 156 |
| with signal peptide | RELRAAFGKVKIFFQMKDQLNSMLLTQSLLDDFKGYLGCQAFSEM | |
| | IQFYLEEVMPQAENHGPDIKEHVNSLGEKLKTLRLRLRRCHRFLP | |
| | CENKSKAVEQVKRVFNMLQERGVYKAMSEFDIFINYIES | |
| IMP-42 | LVCVAIILTYTLYTDAYCVEYLESREDEQQCSSSSNFPASLPHML | 157 |
| with signal peptide | RELRAAFGKVKTFFQMKDQLNSMLLTQSLLDDFKGYLGCQALSEM | |
| | IQFYLEEVMPQAENHGPDIKEHVNSLGEKLKTLRLRLRRCHRFLP | |
| | CENKSKAVEQVKRVFNMLQERGVYKAMSEFDIFINYIES | |
| IMP-43 | MGLRSGLTLQCLVILQCLVMLYLAPACKGVSNCGNLPHMLRDLRD | 158 |
| with signal peptide | AFSRVKTFFQMKDQLDNILLKESLLEDFKGYLGCQALSEMIQFYL | |
| | EEVMPQAENQDPHAKEHVNSLGENLKTLRLRLRRCHRFLPCENKS | |
| | KAVEQVKNAFSKLQEKGVYKAMSEFDIFINYIEAYMTMKIRR | |
| IMP-44 | LVCVAIILTYTLYTDAYCVEYLESREDEQQCGSSSNFPASLPHML | 159 |
| with signal peptide | RELRAAFGKVKTFFQMKDQLNSMLLTQSLLDDFKGYLGCQALSEM | |
| | IQFYLEEVMPQAENHGPDIKEHVNSLGEKLKTLRLRLRRCHRFLP | |
| | CENKSKAVEQVKRVFNMLQERGVYKAMSEFDIFINYIES | |
| IMP-45 | ILVCFVIILTYTLYTDAYCVEYEESEEDRQQCSSSNFPASLPHML | 160 |
| with signal peptide | RELRAAFGKVKTFFQMKDQLNSMLLTQSLLDDFKGYLGCQALSEM | |
| | IQFYLEEVMPQAENHGPDIKEHVNSPGEKLKTLRLRLRRCHRFLP | |
| | CENKSKAVEQVKRVFNMLQERGVYKAMSEFDIFINYIES | |
| IMP-46 | MANVIYVVLALNILLSQIHVSNPYCTSCSYRDCTEDEDQKQQCEG | 161 |
| with signal peptide | GLRSLPHMLRELRAAFGKVKTFFQMKDQLHSLLLTQSLLDDFKGY | |
| | LGCQALSEMIQFYLEEVMPQAENHGPDIKEHVNSLGEKLKTLRLR | |
| | LRRCHRFLPCENKSKAVEKVKRVFSELQERGVYKAMSEFDIFINY | |
| | IETYMT | |
| IMP-47 | ILVCFVIILTYTLYTDAYCVEYEESEEDRQQCSSSNFPASLPHMP | 162 |
| with signal peptide | RELRAAFGKVKTFFQMKDQLNSMLLTQSLLDDFKGYLGCQALSEM | |
| | IQFYLEEVMPQAENHGPDIKEHVNSLGEKLKTLRLRLRRCHRFLP | |
| | CENKSKAVEQVKRVFNMLQERGVYKAMSEFDIFINYIES | |
| IMP-48 | MANVIYVVLALNILLSQFHVSNPYCTSCSHRDCTEDDEQKQQCEG | 163 |
| with signal peptide | GSGGLGSLPHMLRELRAAFGKVKTFFQMKDQLHSLLLTQSLLDDF | |
| | KGYLGCQALSEMIQFYLEEVMPQAENHGPDIKEHVNSLGEKLKTL | |
| | RLRLRRCHRFLPCENKSKAVEKVKRVFSELQERGVYKAMSEFDIF | |
| | INYIETYMT | |
| IMP-49 | MERRLVVTLQCLVLLYLAPECGGTDQCDNFPQMLRDLRDAFSRVK | 164 |
| with signal peptide | TFFQTKDEVDNLLLKESLLEDFKGYLGCQALSEMIQFYLEEVMPQ | |
| | AENQDPGAKDHVNSLGENLKTLRLRLRRCHRFLPCENKSKAVEQI | |
| | KNAFNKLQEKGIYKAMSEFDIFINYIEAYMTIKAR | |
| IMP-50 | MERRLVVTLQCLVLLYLAPECGSTDQCDNFPQMLRDLRDAFSRVK | 165 |
| with signal peptide | TFFQTKDEVDNLLLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-51 | MERRLVVTLQCLVLLYLAPECGGTDQCDNFPQMLRDLRDAFSRVK | 166 |
| with signal peptide | TFFQTKDEVDNLLLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTMKAR | |
| IMP-52 | MGPRAGLALQCLLLLYLAPACKGVSNCGNLPHMLRDLRDAFSRVK | 167 |
| with signal peptide | TFFQMKDQLDNILLKESLLEDFKGYLGCQALSEMIQFYLEEVMPQ | |
| | AENQDPNAKEHVNSLGENLKTLRLRLRRCHRFLPCENKSKAVEQV | |
| | KNAFSKLQEKGVYKAMSEFDIFINYIEAYMTMKTRR | |
| IMP-53 | MERRLVVTLQCLVLLYLAPECGGTDQCDNFPQMLRDLRDAFSRVK | 168 |
| with signal peptide | TFFQTKDEVDNLLLKESLLEDFKGYLGCQALSEMIQFYLEEVMPQ | |
| | AENQDPEAKDHVNSLGENLKTIRLRLRRCHRFLPCENKSKAVEQI | |
| | KNAFNKLQEKGIYKAMSEFDIFINYIEAYMTIKAR | |
| IMP-54 | MERRLVVTLQCLVLLYLAPECGGTDQCDNFPQMLRDLRDAFSRVK | 169 |
| with signal peptide | TFFQTKDEVDSLLLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-55 | MERRLMVTLQCLVLLYLAPECGSTDQCDNFPQMLRDLRDAFSRVK | 170 |
| with signal peptide | TFFQTKDEVDNILLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-56 | MERRLVVTLQCLVLLYLAPECGGTDQCDNFPQMLRDLRDAFSRVK | 171 |
| with signal peptide | TFFQTKDEVDNLELKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-57 | MERRLVVTLQCLVLLYLAPECGGTDQCDNFPQMLRDLRDAFSRVK | 172 |
| with signal peptide | TFFQTKDEVDNLLLKESLLEDFKGYLGCQALSEMIQFYLEKVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-58 | MERRLMVTLQCLVLLYLAPECGSTDQCDNFPQMLRDLRDAFSRVK | 173 |
| with signal peptide | TFFQTKDAVDNLLLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-59 | MELRSGLTLQCLVMLQCLVMLYLAPACKGASNCGNLPHMLRDLRD | 174 |
| with signal peptide | AFSRVKTFFQMKDQLDNILLKESLLEDFRGYLGCQALSEMIQFYL | |
| | EEVMPQAENQDPHSKEHVNSLGENLKTLRLRLRRCHRFLPCENKG | |
| | KAVEQVKNAFSKLQEKGVYKAMSEFDIFINYIEAYMTMKLRR | |
| IMP-60 | MERRLVVTLQCLVLLYLAPECGGTDQCDNFPQMLRDLRDAFSRVK | 175 |
| with signal peptide | TFFQTKDEVDNLLLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFHIFINYIEAYMTIKAR | |
| IMP-61 | MERRLVVTLQCLVLLYLAPECGEMLRDLRDAFSRVKTFFQTKDEV | 176 |
| with signal peptide | DNLLLKESLLEDFKGYLGCQALSEMIQFYLEEVMPQAENQDPEAK | |
| | DHVNSLGENLKTLRLRLRRCHRFLPCENKSKAVEQIKNAFNKLQE | |
| | KGIYKAMSEFDIFINYIEAYMTIKAR | |
| IMP-62 | MELSLGLILHFLVFLCLAPACGRAETCGNIPHMLRDLRDAFSRVK | 177 |
| with signal peptide | TFFQMKDQLDNILLKESLLEDFKGYLGCQALSEMIQFYLEEVMPQ | |
| | AEAMSLKSQEHVNFLGENLNTLRLRLRRCHRFLPCENKSKAVEQV | |
| | KNAFSKLQEKGVYKAMSEFDIFINYIEAYMTMKLRR | |
| IMP-63 | MAYGKKIVAASLLVIPAYVVFTNATANNRAQKCFCFDGSNAGNSE | 178 |
| with signal peptide | ETNTAAFQKKCDSEIPESLPYMLRDLRNSSVQTRRYFQEKDEENS | |
| | PLLTQKLLEDFKGYLGCQALSEMIQFYLEEVMPQAEDSNPSAKDS | |
| | VTSLGEKLKTLRLRLRRCHRFLPCENKSKAVENLKSKFGDLGNQG | |
| | VHKAMSEFDIFINYIETYMTTKMK | |
| IMP-64 | MLSVMVSSSLVLIVFFLGASEEAKPATTTTIKNTKPQCRPEDYAT | 179 |
| with signal peptide | RLQDLRVTFHRVKPTLQREDDYSVWLDGTVVKGCWGCSVMDWLLR | |
| | RYLETVFPAGDHVYPGLKTELHSMRSTLESIYKDMRQCPLLGCGD | |
| | KSVISRLSQEAERKSDNGTRKGLSELDTLFSRLEEYLHSRK | |
| IMP-65 | MLSVMVSSSLVLIVFFLGASEEAKPATTTTIKNTKPQCRPEDYAT | 180 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-66 | MLSVMVSSSLVLIVFFLGASEEAKPATITIKNTKPQCRPEDYATR | 181 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDTLFSRLEEYLHSRK | |
| IMP-67 | MLSVMVSSSLVLIVFFLGASEEAKPATTTTIKNTKPQCRPEDYAT | 182 |
| with signal peptide | RLQDLRVTFHRVKPTLQREDDYSVWLDGTMVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-68 | MLSVMVSSSLVLIVFFLGASEEAKPATTTTIKNTKPQCRPEDYAT | 183 |
| with signal peptide | RLQDLRVTFHRVKSTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-69 | MLSVMVSSSLVLIVFFLGASEEAKPATTTTIKNTKPQCRPEDYAT | 184 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDMLFSRLEEYLHSRK | |
| IMP-70 | MLSVMVSSSLVLIVFFLGASEEAKPATTTTIKNTKPQCRPEDYAT | 185 |
| with signal peptide | RLQDLRVTFHRVKPTLQREDDYSVWLDGTVVKGCWGCSVMDWLLR | |
| | RYLEIVLPAGDHVYPGLKTELHSMRSTLESIYKDMRQCPLLGCGD | |
| | KSVISRLSQEAERKSDNGTRKGLSELDTLFSRLEEYLHSRK | |
| IMP-71 | MLSVMVSSSLVLIVFFLGASEEAKPAATTTIKNTKPQCRPEDYAT | 186 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-72 | MLSVMVSSSLVLIVFFLGASEEAKPATTTTINNTKPQCRPEDYAT | 187 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-73 | MLSVMVSSSLVLIVFFLGASEEAKPATTTTIKNTKPQCRPEDYAT | 188 |
| with signal peptide | RLQDLRVTFHRVKPTLQREDDYSVWLDGTVVKGCWGCSVMDWLLR | |
| | RYLEIVFPAGDHVYPGLKTELHSMHSTLESIYKDMRQCPLLGCGD | |
| | KSVISRLSQEAERKSDNGTRKGLSELDTLFSRLEEYLHSRK | |
| IMP-74 | MLSVMVSSSLVLIIFFLGASEEAKPATTTTIKNTKPQCRPEDYAT | 189 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGMRKGLSELDTLFSRLEEYLHSRK | |
| IMP-75 | MGSRPVRVCGSSNLLCLLLLILVVLVAVVIDRGCWASSKPPVDCD | 190 |
| with signal peptide | PIHGILSRIIKEVRTGYGSIKQALQSKDTVYYVSLFHESLLREML | |
| | SPVGCRVTNELMQHYLDGVLPRAFQCGYDNTTLSGLHSLVSSLDT | |
| | LYKHMLKCPALACTGQTPAWTQFLETEHKLDPWKGTIKATAEMDL | |
| | LVNYLETFLAQS | |
| IMP-76 | MLSVMVSSSLVLIVFFLGASEEAKPATTTTIKNTKPQCRPEDYAT | 191 |
| with signal peptide | RLQDLRVTFDRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDILFSRLEEYLHSRK | |
| IMP-77 | MLSVMVSSSLVLIVFFLGASEEAKPAATTTIKNTKPQCRPEDYAT | 192 |
| with signal peptide | RLQDLRVTFHRVKPTLQHEDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-78 | MLSVMVSSSLVLIVFFLGASEEAKPATTTTIKNTKPQCRPEDYAT | 193 |
| with signal peptide | RLQDLRVTFHRVKPTLQREDDYSVWLDGMVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-79 | MLSVMVSSSLVLIVFFLGASEEAKPATTITIKNTKPQCRPEDYAT | 194 |
| with signal peptide | RLQDLRVTFHRVKPTLQREDDYSVWLDGTVVKGCWGCSVVDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-80 | MLSVMVSSSLVLIVFFLGASEEAKPATTITIKNTKPQCRPEDYAT | 195 |
| with signal peptide | RLQDLRITFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-81 | MLSVMVSSSLVLIVFFLGASEEAKPATTTIKNTKPQCRPEDYATR | 196 |
| with signal peptide | LQDLRVTFHRIKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDTLFSRLEEYLHSRK | |
| IMP-82 | MLSVMVSSSLVLIVFFLGASEEAKPATTTTIKNTKPQCRPEDYAT | 197 |
| with signal peptide | RLQDLRVTFHRVKPTLQCEDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-83 | MLSVMVFSSLVLIVFFLGASEEAKPATTTTTIKNTKPQCRPEDYA | 198 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-84 | MLSVMVSSSLVLIVFFLGASEEAKPATTTTIKNTKPQCRPEDYAT | 199 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDALFSRLEEYLHSRK | |
| IMP-85 | MLSVMVSSSLVLIVFFLGASEEAKPATTTTIKNTKPQCRPEDYAT | 200 |
| with signal peptide | RLQDLRVTFHRVKPTLQREDDYSVWLDGTVVKGCWGCSVMDWLLR | |
| | RYLEILFPAGDHVYPGLKTELHSMRSTLESIYKDMRQCPLLGCGD | |
| | KSVISRLSQEAERKSDNGTRKGLSELDTLFSRLEEYLHSRK | |
| IMP-86 | MLSVMVSSSLVLIVFFLGASEEAKPATTTTTIKNTKPQCRPEDYA | 201 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGMRKGLSELDTLFSRLEEYLHSRK | |
| IMP-87 | MLSVMVSSSLVLIVFFLGASEEAKPATTTIKNTKPQCRPEDYAIR | 202 |
| with signal peptide | LQDLRVTFHRVKPTLQREDDYSVWLDGTVVKGCWGCSVMDWLLRR | |
| | YLEIVFPAGDHVYPRLKTELHSMRSTLESIYKDMQQCPLLGCGDK | |
| | SVISRLSQEAERKSDNGTRKGLSELDTLFSRLEEYLHSRK | |
| IMP-88 | MLSVMVSSSLVLIVFFLGASEEAKPAATTTTIKNTKPQCRPEDYA | 203 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-89 | MLSVMVSSSLVLIVFFLGASEEAKPAATTITTIKNTKPQCRPEDY | 204 |
| with signal peptide | ATRLQDLRVTFHRVKPTLQREDDYSVWLDGTVVKGCWGCSVMDWL | |
| | LRRYLEIVFPAGDHVYPGLKTELHSMRSTLESIYKDMRQCPLLGC | |
| | GDKSVISRLSQEAEKKSDNGTRKGLSELDTLFSRLEEYLHSRK | |
| IMP-90 | MLSVMVSSSLVLIVFFLGASEEAKPAATTTTIKNTKPQCRPEDYA | 205 |
| with signal peptide | SRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-91 | MLSVMVSSSLVLIVFFLGASEEAKPATTTTTIKNTKPQCRPEDYA | 206 |
| with signal peptide | TRLQDLRVTFHRVKPTLQREDDYSVWLDGTVVKGCWGCSVMDWLL | |
| | RRYLEIMFPAGDHVYPGLKTELHSMRSTLESIYKDMRQCPLLGCG | |
| | DKSVISRLSQEAERKSDNGTRKGLSELDTLFSRLEEYLHSRK | |
| IMP-92 | MLSVMVSSSLVLIVFFLGASEEAKSATTTIKNTKPRCRPEDYATR | 207 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDTLFSRLEEYLHSRK | |
| IMP-93 | MLSVMVSSSLVLIIFFLGASEEAKPATTTTIKNTKPQCRPEDYAT | 208 |
| with signal peptide | RLQDLRVTFHRVKPTLQREDDYSVWLDGTVVKGCWGCSVMDWLLR | |
| | RYLEIVFPAGDYVYPGLKTELHSMRSTLESIYKDMRQCPLLGCGD | |
| | KSVISRLSQEAERKSDNGMRKGLSELDTLFSRLEEYLHSRK | |
| IMP-94 | MLSVMVSSSLVLIVFFLGASEEAKPATTTIKNTKPQCRPEDYATR | 209 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDALFSRLEEYLHSRK | |
| IMP-95 | MLSVMVSSSLVLIVFFLGASEEAKPATTTTTIKNTKPRCRPEDYA | 210 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-96 | MLSVMVSSSLVLIVFFLGASEEAKPAATTTTTIKNTKPQCRPEDY | 211 |
| with signal peptide | ATRLQDLRVTFHRVKPTLQREDDYSVWLDGTVVKGCWGCSVMDWL | |
| | LRRYLEIVFPAGDHVYPGLKTELHSMRSTLESIYKDMRQCPLLGC | |
| | GDKSVISRLSQEAERKSDNGTRKGLSELDTLFSRLEEYLHSRK | |
| IMP-97 | MLSVMVSSSLVLIIFFLGASEEAKPATTTTTTIKNIKPQCRPEDY | 212 |
| with signal peptide | ATRLQDLRVTFHRVKPTLQREDDYSVWLDGTVVKGCWGCSVMDWL | |
| | LRRYLEIVFPAGDHVYPGLKTELHSMRSTLESIYKDMRQCPLLGC | |
| | GDKSVISRLSQEAERKSDNGTRKGLSELDTLFSRLEEYLHSRK | |
| IMP-98 | MLSVMVSSSLVLIVFFLGASEEAKPAATTTIKNTKPQCRPEDYAT | 213 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLNELDTLFSRLEEYLHSRK | |
| IMP-99 | MLSVMVSSSLVLIVFFLGASEEAKPATTTTTIKNTKPQCRPEDYA | 214 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDALFSRLEEYLHSRK | |
| IMP-100 | MLSVMVSSSLVLIVFFLGASEEAKPATTTTIKNTKPQCRPEDYAT | 215 |
| with signal peptide | RLQDLCVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-101 | MLSVMVSSSLVLIVFFLGASEEAKPATTTTTTTIKNTKPQCRPED | 216 |
| with signal peptide | YATRLQDLRVTFHRVKPTLQREDDYSVWLDGTVVKGCWGCSVMDW | |
| | LLRRYLEIVFPAGDHVYPGLKTELHSMRSTLESIYKDMRQCPLLG | |
| | CGDKSVISRLSQKAERKSDNGTRKGLSELDTLFSRLEEYLHSRK | |
| IMP-102 | MLSVMVSSSLVLIVFFLGASEEAKPAATTTTIKNTKPQCRPEDYA | 217 |
| with signal peptide | SRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKSLSELDTLFSRLEEYLHSRK | |
| IMP-103 | MLSVMVSSSLVLIVFFLGASEKAKSATTTIKNTKPQCRPEDYATR | 218 |
| with signal peptide | LQDLRVTFHRVKPTLQREDDYSVWLDGTVVKGCWGCSVMDWLLRR | |
| | YLEIVFPAGDHVYSGLKTELHSMRSTLESIYKDMRQCPLLGCGDK | |
| | SVISRLSQEAERKSDNGTRKGLSELDTLFSRLEEYLHSRK | |
| IMP-104 | MLSVMVSSSLVLIVFFLGASEEAKPAATTTTTTIKNTKPQCRPED | 219 |
| with signal peptide | YATRLQDLRVTFHRVKPTLQREDDYSVWLDGTVVKGCWGCSVMDW | |
| | LLRRYLEIVFPAGDHVYPGLKTELHSMRSTLESIYKDMRQCPLLG | |
| | CGDKSVISRLSQKAERKSDNGTRKGLSELDTLFSRLEEYLHSRK | |
| IMP-105 | MLSVMVSSSLVLIVFFLGASEEAKPAATTTTTMIKNTKPQCRPED | 220 |
| with signal peptide | YATRLQDLRVTFHRVKPTLQREDDYSVWLDGTVVKGCWGCSVMDW | |
| | LLRRYLEIVFPAGDHVYPGLKTELHSMRSTLESIYKDMRQCPLLG | |
| | CGDKSVISRLSQEAERKSDNGTRKGLSELDTLFSRLEEYLHSRK | |
| IMP-106 | MLSVMVSSSLVLIVFFLGASEEAKPATTTTTIKNTKPQCRPEDYA | 221 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMWQCPLLGCG | |
| | DKSVISRLSQEAERKSDNGTRKGLSELDTLFSRLEEYLHSRK | |
| IMP-107 | MLSVMVSSSLVLIVFFLGASEEAKPAATTTTIKNTKPQCRPEDYA | 222 |
| with signal peptide | TRLQDFRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-108 | MLSVMVSSSLVLIVFFLGASEEAKPATIKNTKPQCRPEDYATRLQ | 223 |
| with signal peptide | DLRVTFHRVKPTLQREDDYSVWLDGTVVKGCWGCSVMDWLLRRYL | |
| | EIVFPAGDHVYPGLKTELHSMRSTLESIYKDMRQCPLLGCGDKSV | |
| | ISRLSQEAERKSDNGTRKGLSELDTLFSRLEEYLHSRK | |
| IMP-109 | MLSVMVSSSLVLIVFFLGASEEAKPAATTTTTTTIKNTKPQCRPE | 224 |
| with signal peptide | DYATRLQDLRVTFHRVKPTLQREDDYSVWLDGTVVKGCWGCSVMD | |
| | WLLRRYLEIVFPAGDHVYPGLKTELHSMRSTLESIYKDMRQCPLL | |
| | GCGDKSVISRLSQEAERKSDNGTRKGLSELDTLFSRLEEYLHSRK | |
| IMP-110 | MLSVMVSSSLVLIVFFLGASEEAKPAATTTTTTMIKNTKPQCRPE | 225 |
| with signal peptide | DYATRLQDLRVTFHRVKPTLQREDDYSVWLDGTVVKGCWGCSVMD | |
| | WLLRRYLEIVFPAGDHVYPGLKTELHSMRSTLESIYKDMRQCPLL | |
| | GCGDKSVISRLSQEAERKSDNGTRKGLSELDTLFSRLEEYLHSRK | |
| IMP-111 | MLSVMVSSSLVLIVFFLGASEEAKPATTTTTIKNTKPQCRPEDYA | 226 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLRELDTLFSRLEEYLHSRK | |
| IMP-112 | MLSVMVSSSLVLIVFFLGASEEAKPAATTTTTTTTIKNTKPQCRP | 227 |
| with signal peptide | EDYATRLQDLRVTFHRVKPTLQREDDYSVWLDGTVVKGCWGCSVM | |
| | DWLLRRYLEIVFPAGDHVYPGLKTELHSMRSTLESIYKDMRQCPL | |
| | LGCGDKSVISRLSQEAERKSDNGTRKGLSELDTLFSRLEEYLHSR | |
| | K | |
| IMP-113 | MLSVMVSSSLVLIVFFLGASEEAKPAATTTTTTIKNTKPQCRPED | 228 |
| with signal peptide | YATRLQDLRVTFHRVKPTLQREDDYSVWLDGTVVKGCWRCSVMDW | |
| | LLRRYLEIVFPAGDHVYPGLKTELHSMRSTLESIYKDMRQCPLLG | |
| | CGDKSVISRLSQEAERKSDNGTRKGLSELDTLFSRLEEYLHSRK | |
| IMP-114 | MLSVMVSSSLVMIVFFLGASEEAKPATTTTTIKNTKPQCRPEDYA | 229 |
| with signal peptide | TRLQDLCVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDILFSRLEEYLHSRK | |
| IMP-115 | MLSVMVSSSLVLIVFFLGASEEAKPAAATTTTTTTTIKNTKPQCR | 230 |
| with signal peptide | PEDYATRLQDLRVTFHRVKPTLQREDDYSVWLDGTVVKGCWGCSV | |
| | MDWLLRRYLEIVFPAGDHVYPGLKTELHSMRSTLESIYKDMRQCP | |
| | LLGCGDKSVISRLSQEAERKSDNGTRKGLSELDTLFSRLEEYLHS | |
| | RK | |
| IMP-116 | MLSVMVSSSLVLIVFFLGASEEAKPAATTTTTTTIKNTKPQCRPE | 231 |
| with signal peptide | DYATRLQDLRVTFHRVKPTLQREDDYSVWLDGTVVKGCWGCSVMD | |
| | WLLRRYLEIVFPAGDHVYPGLKTELHSMRSTLESIYKDMRQCPLL | |
| | GCGDKSVISRLSQEAERKSDNGTRKGLSELDTLFIRLEEYLHSRK | |
| IMP-117 | MLSVMVSSSLVLIVFFLGASEEAKPATTTTTIKNTKPQCRPEDYA | 232 |
| with signal peptide | TRLQDLRVTFHRVKPTLQREDDYSVWLDGTVVKGCWGRSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-118 | MLSVMVSSSLVLIVFFLGASEEAKPATTTTTIKNTKPQCRPEDYA | 233 |
| with signal peptide | TRLQDLRVTFHRVKPTLQREDDYSVWLDGTVVKGCWGCSVMDWLL | |
| | RRYLEIVFPAGDHVYPGLKTELHSMRSTLESICKDMRQRPLLGCG | |
| | DKSVISRLSQEAERKSDNGTRKGLSELDTLFSRLEEYLHSRK | |
| IMP-119 | MLSVMVSSSLVLIVFFLGASEEAKPAATTTTTTTTTIKNTKPQCR | 234 |
| with signal peptide | PEDYATRLQDFRVTFHRVKPTLQREDDYSVWLDGTVVKGCWGCSV | |
| | MDWLLRRYLEIVFPAGDHVYPGLKTELHSMRSTLESIYKDMRQCP | |
| | LLGCGDKSVISRLSQEAERKSDNGTRKGLSELDTLFSRLEEYLHS | |
| | RK | |
| IMP-120 | MLSVMVSSSLVLIVFFLGASEEAKPATIKNTKPQCRPEDYATRLQ | 235 |
| with signal peptide | DLRVTFHRVKPTLPGHQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-121 | MKTNTKIILFCYVILSLYVFSCAIASAKKCDDVSFDYILKDLRSE | 236 |
| with signal peptide | FSKIKSFVQDNDQENMMLLSQSMLDKLTSRIGCKSLSDMIKFYLN | |
| | DVLPNAEKIEHMKNKITSIGEKLKSLKEKLISCDFLHCENHDEIK | |
| | TVKTIFNKLKDKGIYKAMGEFDIFINYLEKYIVKK | |
| IMP-122 | MKTNTKIILFCYVILSLYVFSCAIASAKKCNDVSFDYILKDLRSE | 237 |
| with signal peptide | FSKIKSFVQDNDQENMMLLSQSMLDKLISRIGCKSLSDMIKFYLN | |
| | DVLPNAEKIEHMKNKITSIGEKLKSLKEKLISCDFLHCENHDEIK | |
| | TVKTIFNKLKDKGIYKAMGEFDIFINYLEKYIVKK | |
| IMP-123 | MKTNIKIILFCYVIFLSLYVFSCVVASAKKCDDVSFDYILKDLRS | 238 |
| with signal peptide | EFSKIKSFVQDNDQENMMLLSQSMLDKLISRIGCKSLSDMIKFYL | |
| | NDVLPNAEKIEHMKNKITSIGEKLKSLKEKLISCDFLHCENHDEI | |
| | KTVKTIFNKLKDKGIYKAMGEFDIFINYLEKYIVKK | |
| IMP-124 | MKTNIKIILFCYVILSLYVFSCVVAYAKKCDDVSFDYILKDLRSE | 239 |
| with signal peptide | FSKIKSFVQNNDQENMMLLSQSMLNKLISCIGCKSLSDMIKFYLN | |
| | DVLPNAEKIEQIKNIITSIGEKLKSLKEKLISCDFLHCENNDEIK | |
| | TVKAIFNKLKDKGIYKAMGEFDIFINYVEKYIVKT | |
| IMP-125 | MKTSTKIILFCYVILSLYVFSCVVASAKKCDDVSFDYILKDLRSE | 240 |
| with signal peptide | FIKIKSFVQNNDQENMMLLSQSMLDKLISCIGCKSLSDMIKFYLN | |
| | DVLPNAEKIEQIKNIITSIGEKLKSLKEKLISCDFLHCENNDEIK | |
| | TVKAIFNKLKDKGIYKAMGEFDIFINYVEKYIVKT | |
| IMP-126 | MKTNTKIILFCYVILYVFSCTVASAKKCDDVSFDYILKDLRSEFS | 241 |
| with signal peptide | KIKSFVQNNDKENMMLLSQSMLDKLISCIGCKSLSDMIKFYLNDV | |
| | LPNAEKIEHIKNKITSIGEKLKSLKEKLISCDFLHCENHDEIKAV | |
| | KTIFNKLKDKGIYKAMGEFDIFINHLEKYIVKK | |
| IMP-127 | MKTSTKIILFCYVILSLYVFSCVVASAKKCDDVSFDYILKDLRSE | 242 |
| with signal peptide | FIKIKSFVQNNDQENMMLLSQSMLDKLISRIGCKSLSDMIKFYLN | |
| | DVLPNAEKIEQIKNIITSIGEKLKSLKEKLISCDFLHCENNDEIK | |
| | TVKAIFNKLKDKGIYKAMGEFDIFINYVEKYIVKT | |
| IMP-128 | MKTNTKIILFCYVILYLFSCTVASAKKCDDVSFDYILKDLRSEFS | 243 |
| with signal peptide | KIKSFVQNNDKENMMLLSQSMLDKLISCIGCKSLSDMIKFYLNDV | |
| | LPNAEKIEHIKNKITSIGEKLKSLKEKLISCDFLHCENHDEIKAV | |
| | KTIFNKLKDKGIYKAMGEFDIFINHLEKYIVKK | |
| IMP-129 | MLSVMVSSSLVLIVFFLGASEEAKPATTTTTIKNTKPQCRPEDYA | 244 |
| with signal peptide | TRLQDLRVTFHRVKPTLDDYSVWLDGTVVKGCWGCSVMDWLLRRY | |
| | LEIVFPAGDHVYPGLKTELHSMRSTLESIYKDMRQCPLLGCGDKA | |
| | VISRLSQEAERKSDNGTRKGLSELDTLFSRLEEYLHSRK | |
| IMP-130 | MKTNTKIILFCYVILYLFSCTVASAKKCDDVSFDYILKDLRSEFS | 245 |
| with signal peptide | KIKSFVQNNDKENMMLLSQSMLDKLTRCIGCKSLSDMIKFYLNDV | |
| | LPNAEKIEHIKNKITSIGEKLKSLKERLISCDFLHCENHDEIKAV | |
| | KTIFNELKDKGIYKAMGEFDIFINHLEKYIVKK | |
| IMP-131 | MERRLVVTLQCLVLLYLAPECGGTDQCDNFPQMLRDLRDAFSRVK | 246 |
| with signal peptide | TFFQTKDEVDNLLLKESLLEDFKGYLGCQALSEMIQFYLEEVMPQ | |
| | AENQDPEAKDHVNSLGENLKTLRLRLRRCHRFLPCENKSKAVEQI | |
| | KMPLTSCRKKEFTKP | |
| IMP-132 | MGSRRLSRCSLATAVCLVAIVVAVAAKGRDSKPSPACDPMHGALA | 247 |
| with signal peptide | GIFKELRTTYRSVRETLQTKDTVYYVSLFHEQLLQEMLSPVGCRV | |
| | TNELMQHYLDGVLPRAFHCGYDNTTLNALHELSSSLSTLYQHMLK | |
| | CPALACTGQTPAWTQFLDTEHKLDPWKGTVKATAEMDLLLNYLET | |
| | FLLQS | |
| IMP-133 | MLSVMVSSSLVLIVFLLGASEEAKPATTTIKNTKPQCRPEDYATR | 248 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCEAERKSDN | |
| | GTRKGLSELDTLFSRLEEYLHSRK | |
| IMP-134 | MRRRRSFGVVVVGAIGTLLMMAVVVLSAHDHEHKVPPACDPVHGN | 249 |
| with signal peptide | LAGIFKELRTIYTSIREGLQKKDTVYYTSLFNDRVLQEMLSPMGC | |
| | RVTNEIMEHYLDGVLPRASHLDYDNSTLNGLHAFASSMQALYQHM | |
| | LKCPALACTGKTPAWMYFLEVEHKLNPWRGTAKAAAEADLLLNYL | |
| | ETFLLQF | |
| IMP-135 | MGSRPARMCGLSNLLCLLLVVLVAVVIHRGCGASKPPVDCDPIHG | 250 |
| with signal peptide | TLSRIIKEVRIGYGSIKQALQSKDTVYYVSLFHENLLNEMLSPVG | |
| | CRVTNELMQHYLDGVLPRAFQCGYDNTTLDGLHSLVSSLDALYKH | |
| | MLKCPALACTGQTPAWTQFLETEHKLDPWKGTIKATAEMDLLVNY | |
| | LETFLAQS | |
| IMP-136 | MLSMMVSSSLVLIVFFLGASEEAKPATTTTTIKNTKPQCRPEDYA | 251 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISR | |
| IMP-137 | MLSVMVSSSLVLIVFFLGASEEAKPATTTIKNTKPQCRPEDYATR | 252 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCVSVSVAAL | |
| | SAQR | |
| IMP-138 | MININILSLLILILSIYANAIIDTCYDDQERERTKSNSISSVTPE | 253 |
| with signal peptide | MCKGLKQLVSTKLKDARQKEKSVRDYFTSRDNDLDFMLLQGVKET | |
| | HKKTCGCYVLYLLLSFYGKTIRDTIQSNKHKNLNTELTNLAVSVL | |
| | SLEDLLEACGITCNPKKDSLLKRIEEYMKEHGDDAIYKVIGEIEF | |
| | LFQAIEKHVY | |
| IMP-139 | MINISINILSLLILILSIYANSIIDMCYDDQERERTKSNSISSIT | 254 |
| with signal peptide | PDMCKGLKQLVATKLKDARQKEKLVNSYFTSRDNDLTYMLLQGVR | |
| | ETHKKPCGCYVLYLLLTFYRKTIKDTIQSKKHESINTELINLAVT | |
| | VLSLEDLLEACGITCNPKKDSLLKRIEGYTKEHGDDAIYKVIGEI | |
| | DFLFQAIERHVY | |
| IMP-140 | MININILSLLILILSIYANAIIDTCYDDQERERTKSNSISSVTPE | 255 |
| with signal peptide | MCKGLKQLVATKLKDARQKEKLVNDYFTGRDNDLSYMLLQGVRET | |
| | HKKPCGCYVLYLLLSFYRKTIRDTIQSNKHASINAELTNLAVSVL | |
| | SLEDLLDACGITCNPKKDSLLKRIEEYMKEHGDDAIYKLIGEIEF | |
| | LFQAIERHVYT | |
| IMP-141 | MININILSLLILILSIYANAIIDTYDEDEDEDSIKLSSIGSITPE | 256 |
| with signal peptide | MCKNLKQLVASKLKDIRQKEKSLRDYFTNLDDELDYMLLQGVGEN | |
| | HKKKCGCYILHLLLKFYSKTIRNTIQSEKHKNVNLELTNIAVSML | |
| | ALEDLLEKCGITCNPKKDPLLKRIEDYMKQHGDDGVNKAIAELEF | |
| | LFQMIEKQVYI | |
| IMP-142 | MARFIYVVLLCLVEDAAQSAAQCRKGTITSRLKMLRTAFEKVREF | 257 |
| with signal peptide | YEDRDEEETALASTEHLHGPESCSVIDELITHYTKCVIPAANEEE | |
| | GADLLSLDTLQVALENVKGLLANCQEEFGCKPPFSMRDYKKQYRQ | |
| | LNKEKNAGMIKAMGELGMLENGIEERVIGM | |
| IMP-143 | MMLALAMMLMALGPLSTNAMYVQHGSDYCTTTVHADIASAISGMR | 258 |
| with signal peptide | AEYDSGLGHYFKSLVPHPDNPYDTDDYKYMINNINSYNCHALQST | |
| | INALLGMYGYVDIDEPHQLAMMKLATHTMQAAMLLNKCAKQLGCY | |
| | HIPFDVETLHEAHPDDVMASLDTALNLMSMVTNEI | |
| IMP-144 | MARFIYVVLLCLVFDAAQSAAQCRKGTITSRLKMLRTAFEKVREF | 259 |
| with signal peptide | YEDSDEEETALASTEHLHGPESCSVIDELITHYTKCVIPAANEEE | |
| | GADLLSLDTLQVALENVKGLLANCQEEFGCKPPFSMRDYKKQYRQ | |
| | LNKEKNAGMIKAMGELGMLFNGIEERVIGM | |
| IMP-145 | MFQLEGIVLLVYLANWVSPATIKCVGMSTLFNPELIQLRRLFGDG | 260 |
| with signal peptide | IKDFFQNKDEDLDNAFLNEDVQRELASDCGCDHLMDMLSLYVNDT | |
| | IPKGMKTEDAPSGLGQMGQLMSSLYRKMDMCWSELGCSHNTRLTL | |
| | QEYADKKGGWDNKALGESDILFDALELFFSKIK | |
| IMP-146 | MMTTLALVMTLMALSTNAMYVQHGSDYCTTTVRADIASAISGMRA | 261 |
| with signal peptide | EYNNGLGDYFKSLAPHPNNPYDIDDYKYMINSTNSYNCHALQSTI | |
| | NALLGMYGYVDIDEPHQLAMMKLATHTMQTAMLLNKCAAQLGCYH | |
| | IPFDVETLHEAHPNDVMASLDTALNLMSMVTNEI | |
| IMP-147 | MITLALVMTLMALGPLSTNAMYARRSGDYCTTTVRADIASAISGM | 262 |
| with signal peptide | RAEYNSGLRDYFKSLVPHPDNPYDIDDYKYMLNNINSYNCHALQS | |
| | TINALLGMYGYVDIDESHQLAMMKLATHTMQTAMMLNKCAAQLGC | |
| | YHIPFDLETLHEAHPDDVMASLDTALNLMSMITNEI | |
| IMP-148 | MMTTLALVMTLMALATNAMYVQHGSDYCTTTVRADIASAISGMRA | 263 |
| with signal peptide | EYNNGLGDYFKSLAPHPNNPYDTDDYKYMINSTNSYNCHALQSTI | |
| | NALLGMYGYVDIDEPHQLAMMKLATHTMQTAMLLNKCAAQLGCYH | |
| | IPFDVETLHEAHPDDVMASLDTALNLMSMVTNEI | |
| IMP-149 | MSGTSNKKFVFLVAIAVAICMMSSVSSNVHSGTEDNPCINSKTVL | 264 |
| with signal peptide | NTLLNQIKQEYINNLLPYYKALTPKPVDVFDDSYTYSIQSTDYNC | |
| | YTIYETLNFLLGDVFPRATTDATVRLSLAKIATSSQQASMLMNLC | |
| | KKELACGPAPFDMIKLYHDTKEYGADNIMGTLDTPFQYFVIV | |
| IMP-150 | MLALAMVLMALGPLSTNAMYVQHGRGDYCTTTVRADIASAISGMR | 265 |
| with signal peptide | AEYDSGLGHYFKSLVPHPDNPYDTDDYKYMINNINSYNCHALQST | |
| | INALLGMYGYVDIDEPHQLAMMKLATHTMQTAMLLNKCAEQLGCY | |
| | HIPFDVEILHEAHPDDVMASLDTALNLMSMVTNEI | |
| IMP-151 | MFLAVLLTATIFFEARGAPATTPKDSCVYLIGQTPQLLRQLRNAY | 266 |
| with signal peptide | QAIIGADGSGVDEDDMPIYPSDVMNELASTSVACDAIKKVLIMNI | |
| | GILPNVTAAYPDKKSEVDEIGDNLSRLHQNIVNCRDFLKCEDLPH | |
| | WHQMAENYKEKPMQGFSEMDFVFQSVEKFLVAKDVKNMKTKRKH | |
| IMP-152 | MLKQIIVVCIVAMAAVFADDDPCTNVKTQLNTLFNQIKTEYDINL | 267 |
| with signal peptide | KTYYQSIAPSAFDPFNNTNYLYSVQGNDYKCYTIFETLSFLMGDV | |
| | YPRATTNESVRLSLAKVATSSTQGAMVMNLCRQQLGCGPPPFDAK | |
| | TLYDDRAEYGADDIMATLDTALAKFKLVLESENVV | |
| IMP-153 | MLKQIIVVCIVAMAAVFADDDPCTNVKTQLNTLFNQIKTEYDINL | 268 |
| with signal peptide | KTYYQSIAPSAFDPFNNTNYLYSVQGNDYKCYTIFETLSFLMGDV | |
| | YPRATTNESVRLSLAKVAISSTQGAMVMNLCREQLGCGPPPFDAK | |
| | TLYDDRAEYGADDIMATLDTALAKFKLVLESENVV | |
| IMP-154 | MITLALVMTLMALGPLSTNGVHARRRGDYCTTTVRADIASAISGM | 269 |
| with signal peptide | RAEYNSGLGDYFKSLVPHPDNPYDIDDYKHMIDNANSYNCHALQS | |
| | TINALLGMYGYVDIDEPHQLAMMKLATHTMQTAMLLNKCAAQLGC | |
| | YHIPFDLETLREAPPADVMASLDTALNLMSMITNEI | |
| IMP-155 | MDAQFLLLIVLALPASFAASLSTHYNNYDLTRIATIDKDVCKRVA | 270 |
| with signal peptide | QHINDDFVNMRKLYETQLKNYFQQLVPNPTDVFKDDSYMYMINGT | |
| | DYNCHIIYETMRFLSGDVFPFATETEAELQYMWKMMLGVSQLSAY | |
| | IGNCYQYFKCGPAPFDPQVLYHDRELFHADTVMAYLDTAFSHETL | |
| IMP-156 | MKLYFYCIFFYKIIVTISLNCGIEHNELNNIKNIFFKVRNVVQAD | 271 |
| with signal peptide | DVDHNLRILTPALLNNITVSETCFFIYDMFELYLNDVFVKYTNTA | |
| | LKLNILKSLSSVANNFLAIFNKVKKRRVKKNTVNVLEIKKLLLID | |
| | NNCKKLFSEIDIFLTWVMAKI | |
| IMP-157 | MKLYFYCIFFYKIIVTISLNCGIEHNELNNIKNIFFKVRNVVQAD | 272 |
| with signal peptide | DVDHNLRILTPALLNNITVSETCFFIYDMFELYLNDVFVKYINTA | |
| | LKLNILKSLSSVANNFLAIFNKVKKRRVKKNNVNVLEIKKLLLID | |
| | NNCKKLFSEIDIFLTWVMAKI | |
| IMP-158 | MFLAVLLTATIFFEARGAPATTPKDSCVYLIGQTPQLLRQLRNAY | 273 |
| with signal peptide | QAIIGADGSGVDEDDMPIYPSDVMNELASTSVACDAIKKVLIMNI | |
| | GILPNVTAAYPDKKSEVDEIGDNLSRLHQNIVNCVSRTQHLCYD | |
| IMP-159 | MFRASLLCCLVLLAGVWADNKYDSESGDDCPTLPTSLPHMLHELR | 274 |
| with signal peptide | AAFSRVKIFFQMKDQLDNMLLDGSLLEDFKGYLGCQALSEMIQFY | |
| | LEEVMPQAENHSPDQDKNKVNSLGEKLKTLRVRLRRCHRFLPCEN | |
| | KSKAVEQVKSAFSKLQEKGVYKAMSEFDIFINYIEAYMTTKMKN | |
| IMP-160 | MFRASLLCCLVLLAGVWADNKYDSESGNDCPTLPTSLPHMLHELR | 275 |
| with signal peptide | AAFSRVKTFFQMKDQLDNMLLDGSLLEDFKGYLGCQALSEMIQFY | |
| | LEEVMPQAENHSTGQEKDKVNSLGEKLKTLRVRLRRCHRFLPCEN | |
| | KSKAVEQVKSAFSKLQEKGVYKAMSEFDIFINYIEAYMTTKMKN | |
| IMP-161 | MFRASLLCCLVLLAGVWADNKYDSESGNDCPTLPTSLPHMLHELR | 276 |
| with signal peptide | AAFSRVKTFFQMKDQLDNMLLDGSLLEDFKGYLGCQALSEMIQFY | |
| | LEEVMPQAENHSTDQEKDKVNSLGEKLKILRVRLRRCHRFLPCEN | |
| | KSKAVEQVKSAFSKLQEKGVYKAMSEFDIFINYIEAYMTTKMKN | |
| IMP-162 | MFRASLLCCLVLLAGVWADNKYDSESGDDCPTLPTSLPHMLHELR | 277 |
| with signal peptide | AAFSRVKIFFQMKDQLDNMLLDGSLLEDFKGYLGCQALSEMIQFY | |
| | LEEVMPQAENHSTGQEKDKVNSLGEKLKTLRVRLRRCHRFLPCEN | |
| | KSKAVEQVKSAFSKLQEKGVYKAMSEFDIFINYIEAYMTTKMKN | |
| IMP-163 | MFRALLLCCLALLAGVWADNRYDGQDGNDCPTLPTSLPHMLHELR | 278 |
| with signal peptide | AAFSRVKTFFQMKDQLDNMLLDGSLLEDFKGYLGCQALSEMIQFY | |
| | LEEVMPQAENHSPDQDKNKVNSLGEKLKTLRVRLRRCHRFLPCEN | |
| | KSKAVEQVKSAFSKLQEKGVYKAMSEFDIFINYIEAYMTTKMKN | |
| IMP-164 | MLSVMVSSSLVLIVFFLGASEEAKPATTAIKNTKPQCRPEDYATR | 279 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDTLFSRLEEYLHSRK | |
| IMP-165 | MLSVMVSSSLVLIVFFLGAFEEAKPATTTTIKNTKPQCRPEDYAT | 280 |
| with signal peptide | RLQDLRVTFDRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAEKKSDNGTRKGLSELDTLFSRLEEYLHSRK | |
| IMP-166 | MLSVMVSSSLVLIVFFLGASEEAKPATTTITIKNTKPQCRPEDYA | 281 |
| with signal peptide | TRLQDLRVTFYRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-167 | MLSVMVSSSLVLIVFFLGASEEAKPATTTTTIKNTKPQCRPEDYA | 282 |
| with signal peptide | TRLQDLRVTFHRVKPTLDDYSVWLDGTVVKGCWGCSVMDWLLRRY | |
| | LEIVFPAGDHVYPGLKTELHSMRSTLESIYKDMRQCPLLGCGDKS | |
| | VISRLSQEAERKSDNGTRKGLSELDTLFSRLEEYLHSRK | |
| IMP-168 | MKTNTKIILFCYVIFLSLYVFSCVVASTKKCDDVSFDYILKDLRS | 283 |
| with signal peptide | EFSKIKSFVQDNDQENMMLLSQSMLDKLTSRIGCKSLSDMIKFYL | |
| | NDVLPNAEKIEHMKNKITSIGEKLKSLKEKLISCDFLHCENHDEI | |
| | KTVKTIFNKLKDKGIYKAMGEFDIFINYLEKYIVKK | |
| IMP-169 | MLSVMVSSSLVLIVFFLGASEEAKPATTTIKNTKPQCRPEDYATR | 284 |
| with signal peptide | LQDLRVTFHRVKPTLQREDDYSVWLDGDHVYPGLKTELHSMRSTL | |
| | ESIYKDMRQCPLLGCGDKSVISRLSQEAERKSDNGTRKGLSELDT | |
| | LFSRLEEYLHSRK | |
| IMP-170 | MSNFRILSLLIFSLIVHVNAMIGTCYDEDEEIERLKSNSISSITP | 285 |
| with signal peptide | GMCRNLKHSVMIRLIDARQIEASIRSYFTDGDNNLSFMLLQGIRE | |
| | ISKKKCGCYILNLMLRFYIQTIKHTILSNKHKDMNLELTNLAVTI | |
| | LSLESLLEKCGVTCNPVKDPLLTRIEEYTRKHGDNAIYKTIGELE | |
| | FLFDAIEKFV | |
| IMP-171 | MARFIYVVLLCLVFDAAQSAAQCRKGTITIRLKMLRTAFEKVREF | 286 |
| with signal peptide | YEDRDEEETALASTEHLHGPESCSVIDELITHYTKCVIPAANEEE | |
| | GADLRSLDTLQFALENVKGLLANCQEEFGCKPPFSMRDYKKQYRQ | |
| | LNKEKNAGMIKAMGELGMLFNGIEERVIGM | |
| IMP-172 | MARFIYVVLLCLVFDAAQSAAQCRKGTITIRLKMLRTAFEKVREF | 287 |
| with signal peptide | YEDRDEEETALASTEHLHGPESCSVIDELITHYTKCVIPAANEEE | |
| | GADLRSLDTLQFALENVKGLLANCQEEFGCKPPFSMRDYKKQYRQ | |
| | LNKEKNAGMIKAMGELGMLFNGIEERVNGM | |
| IMP-173 | MARFIYVVLLCLVEDAAQSAAQCRKGTITIRLKMLRTAFEKVREF | 288 |
| with signal peptide | YEDRDEEETALASTEHLHGPESCSVIDELITHYTKCVIPAANEEE | |
| | GADLLSLDTLQFALENVKGLLANCQEEFGCKPPFSMRDYKKQYRQ | |
| | LNKEKNAGMIKAMGELGMLFNGIEERVIGM | |
| IMP-174 | MARFIYVVLLCLVEDAAQSAAQCRKGTITSRLKMLRTAFEKVREF | 289 |
| with signal peptide | YEDRDEEETALASTEHLHGPESCSVIDELITHHTKCVIPAANEEE | |
| | GADLLSLDTLQVALENVKGLLANCQEEFGCKPPFSMRDYKKQYRQ | |
| | LNKEKNAGMIKAMGELGMLFNGIEERVIGM | |
| IMP-175 | MLSVMVSSSLVLIVFLLGASEEAKPATTTIKNTKPQCRPEDYATR | 290 |
| with signal peptide | LQDLRVTFHRVKPTLVGHVGDHVYPGLKTELHSMRSTLESIYKDM | |
| | RQCEAERKSDNGTRKGLSELDTLFSRLEEYLHSRK | |
| IMP-1 | RHCMFGDSLRNSPDMKNMLQDLRGGYSGSGIKRTFQGKDTLDSML | 119 |
| without signal | LTQSLLDDFKGYLGCQALSEMIQFYLEEVMPQAENHGPTDSVKQL | |
| peptide | GEKLHTLNQKFGECPRWFPCYYNTTPAVENVKSVFSKLQERGVYK | |
| | AMSEFDIFINYIETYTTMK | |
| IMP-2 | CQLESGEALPLGSRSADSRSVDGQRVPAPQNNYPGLLRDLRLGYE | 120 |
| without signal | GFKQKVTDSHPDETLLGSSRLAGDLKGPLRCQALSEMIQFLLQVV | |
| peptide | LPDAENSRQDLRSQFSTLGDRITGLRQQLRRDPTVFPCESRSDGV | |
| | SDLRSAYTRLGSTGAEKVLSEFDIFINYIEAYVTSV | |
| IMP-3 | YCVEYAESDEDRQQCSSSSNFPASLPHMLRELRAAFGKVKIFFQM | 121 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-4 | YCTSCSHHQCTEDENQKQDCEDANHSLPHMLRELRAAFGKVKTFF | 122 |
| without signal | QMKDQLHSLLLTQSLLDDFKGYLGCQALSEMIQFYLEEVMPQAEN | |
| peptide | HGPEEHDNSLSEHGPDVKEHVNSLGEKLKTLRLRLRRCHRFLPCE | |
| | NKSKAVEKVKRVFSELQERGVYKAMSEFDIFINYIETYMTT | |
| IMP-5 | RSPKNKPSIDCNPQTGDFVNMLKSMRQDYSRIRDTLHDRDKLHSS | 123 |
| without signal | LLTGALLDEMMGYSGCRTTLLLMEHYLDTWYPAAYRHHLYDNQTL | |
| peptide | VVVDRMGSTLVALLKAMVQCPMLACGAPSPAMDKMLQQEAKMKKY | |
| | TGVYKGISETDLLLGYLELYMMKFKR | |
| IMP-6 | HEHKVPPACDPVHGNLAGIFKELRAIYASIREALQKKDTVYYTSL | 124 |
| without signal | FNDRVLQEMLSPMGCRVTNELMEHYLDGVLPRAAHFDYDNSTLNG | |
| peptide | LHAFTSSMQALYQHMLKCPALACTGKTPAWMYFLEVEHKLNPWRG | |
| | TAKAAAEADLLLNYLETFLLQF | |
| IMP-7 | KGRDSKPSPACDPMHGALAGIFKELRTTYRSVREALQTKDTVYYV | 125 |
| without signal | SLFHEQLLQEMLSPVGCRVTNELMQHYLDGVLPRAFHCGYDNATL | |
| peptide | NALHALSSSLSTLYQHMLKCPALACTGQTPAWTQFLDTEHKLDPW | |
| | KGTVKATAEMDLLLNYLETFLLQS | |
| IMP-8 | ATTTIKNTKPQCRPEDYATRLQDLRVTFHRVKPTLQREDDYSVWL | 126 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DTLFSRLEEYLHSRK | |
| IMP-12 | YCVEYEESEEDRQQCSSSNFPASLPHMLRELRAAFGKVKTFFQMK | 291 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTKP | |
| IMP-13 | YCVEYEESEEDRQQCSSSNFPASLPHMLRELRAAFGKVKIFFQMK | 292 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTKT | |
| IMP-14 | YCVEYEESEEDRQQCSSSNFPASLPHMLRELRAAFGKVKTFFQMK | 293 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTKS | |
| IMP-15 | YCVEYKESEEDRQQCSSSSFPASLPHMLRELRAAFGKVKIFFQMK | 294 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTK | |
| IMP-16 | YCVEYAESDEDKQQCSGSNFPASLPHMLRELRAAFGKVKIFFQMK | 295 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTKM | |
| IMP-17 | YCVEYEESEEDKQQCGSNGGPASLPHMLRELRAAFGKVKTFFQMK | 296 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVFNM | |
| | LQERGVYKAMSEFDIFINYIESYMTTKM | |
| IMP-18 | YCVEYAESDEDRQQCSGSNFPASLPHMLRELRAAFGKVKIFFQMK | 297 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVFNM | |
| | LQERGVYKAMSEFDIFINYIESYMTTKM | |
| IMP-19 | YCVEYEESEEDKQQCSSSSNFPASLPHMLRELRAAFGKVKIFFQM | 298 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-20 | YCIQYEESEEDKQQCSSSNFPASLPHMLRELRAAFGKVKIFFQMK | 299 |
| without signal | DQLNSMLLTQSLLDDFKGYLGCQALSEMIRFYLEEVMPQAENHGP | |
| peptide | DIKEHVNSLGEKLKTLRLRLRRCHRFLPCENKSKAVEQVKRVFNM | |
| | LQERGVYKAMSEFDIFINYIESYMTTKM | |
| IMP-21 | YCVEYEESEEDRQQCSSSNFPASLPHMLRELRAAFGKVKTFFQMK | 300 |
| without signal | DQLNSMLLTQSLLDDFKGYLGCQALSEMIQFYLEEVMPQAENHGP | |
| peptide | DIKEHVNSLREKLKTLRLRLRRCHRFLPCENKSKAVEQVKRVFNM | |
| | LQERGVYKAMSEFDIFINYIESYMTTKT | |
| IMP-22 | YCVEYEESEEDKQQCGSSSNFPASLPHMLRELRAAFGKVKTFFQM | 301 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-23 | YCVEYAESDEDKQQCSGSNFPASLPHMLRELRAAFGKVKIFFQMK | 302 |
| without signal | DQLNSMLLTQSLLDDFKGYLGCQALSEMIQFYLEEVMPRAENHGP | |
| peptide | DIKEHVNSLGEKLKTLRLRLRRCHRFLPCENKSKAVEQVKRVFNM | |
| | LQERGVYKAMSEFDIFINYIESYMTTKM | |
| IMP-24 | YCVEYEESEEDRQQCSGSSNFPASLPHMLRELRAAFGKVKTFFQM | 303 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-25 | YCVEYEESDEDRQQCSSSSNFPASLPHMLRELRAAFGKVKIFFQM | 304 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-26 | YCVEYEESDEDKQQCSSSTGAPASLPHMLRELRAAFGKVKTFFQM | 305 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-27 | YCVEYEETKEDEQQCSSSSNFPASLPHMLRELRAAFGKVKIFFQM | 306 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-28 | YCVEYEESEEDRQQCSSSNFPASLPHMLRELRAAFGKVKTFFQMK | 307 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVFNM | |
| | LQERGDYKAMSEFDIFINYIESYMTTKS | |
| IMP-29 | YCVEYEESEEDRQQCSSSSNFPASLPHMLRELRAAFGKVKIFFQM | 308 |
| without signal | KDQLNSMLLTQSLLDDFKGYLGCQALSEMIQFYLEEVMPQAENHG | |
| peptide | PDIKEHVNSLGEKLKTLRLRLRRCHRFLPCEDKSKAVEQVKRVEN | |
| | MLQERGVYKAMSEFDILINYIESYMTTKM | |
| IMP-30 | YCVEYEESDEDRQQCSSSSNFPASLPHMLRELRAAFGKVKIFFQM | 309 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMATKM | |
| IMP-31 | DNRYDGQDGNDCPTLPTSLPHMLHELRAAFSRVKIFFQMKDQLDN | 310 |
| without signal | MLLDGSLLEDFKGYLGCQALSEMIQFYLEEVMPQAENHSTDQEKD | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-32 | YCVEYLESREDEQQCSSSSNFPASLPHMLRELRAAFGKVKTFFQM | 311 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-33 | YCVEYEESKEDEQQCSGSNGASASLPHMLRELRAAFGKVKIFFQM | 312 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-34 | YCVEYLESGEDEQQCGSSSNFPASLPHMLRELRAAFGKVKIFFQM | 313 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-35 | YCVEYLESREDEQQCSGSNGASASLPHMLRELRAAFGKVKIFFQM | 314 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-36 | ESENNCTHFPTSLPHMLHELRAAFSRVKIFFQMKDQLDNMLINGS | 315 |
| without signal | LLEDFKGYLGCQALSEMIQFYLEEVMPQAENHSGGGGPDIKEHVN | |
| peptide | SLGEKLKTLRVRLRRCHRFLPCENKSKAVEQVKSAFSKLQEKGVY | |
| | KAMSEFDIFINYIEAYMITKMKNKK | |
| IMP-37 | YCVEYLESDEDKQHCSSSNGASASSPHMLRELRAAFGKVKIFFQM | 316 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-38 | YCVEYLESEEDKQQCGSNGASSSSPHMLRELRAAFGKVKTFFQMK | 317 |
| without signal | DQLNSMLLTQSLLDDFKGYLGCQALSEMIQFYLEEVMPQAENHGP | |
| peptide | DVKEHVNSLAEKLKTLRLRLRRCHRFLPCENKSKAVEQVKRVENM | |
| | LQERGVYKAMSEFDIFINYIESYMTTKS | |
| IMP-39 | YCVEYLESEEDKQQCGSNGASSSSPHMLRELRAAFGKVKTFFQMK | 318 |
| without signal | DQLNSMLLTQSLLDDFKGYLGCQALSEMIQFYLEEVMPQAENHGP | |
| peptide | DVKEHVNSLGEKLKTLRLRLRRCHRFLPCENKSKAVAQVKRVFNM | |
| | LQERGVYKAMSEFDIFINYIESYMTTKS | |
| IMP-40 | YCVEYEESEEDRQQCSSSNFPASLPHMLRELRAAFGKVKTFFQMK | 319 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIES | |
| IMP-41 | YCVEYEESEEDRQQCSSSNFPASLPHMLRELRAAFGKVKTFFQMK | 320 |
| without signal | DQLNSMLLTQSLLDDFKGYLGCQAFSEMIQFYLEEVMPQAENHGP | |
| peptide | DIKEHVNSLGEKLKTLRLRLRRCHRFLPCENKSKAVEQVKRVFNM | |
| | LQERGVYKAMSEFDIFINYIES | |
| IMP-42 | YCVEYLESREDEQQCSSSSNFPASLPHMLRELRAAFGKVKIFFQM | 321 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIES | |
| IMP-43 | VSNCGNLPHMLRDLRDAFSRVKTFFQMKDQLDNILLKESLLEDFK | 322 |
| without signal | GYLGCQALSEMIQFYLEEVMPQAENQDPHAKEHVNSLGENLKTLR | |
| peptide | LRLRRCHRFLPCENKSKAVEQVKNAFSKLQEKGVYKAMSEFDIFI | |
| | NYIEAYMTMKIRR | |
| IMP-44 | YCVEYLESREDEQQCGSSSNFPASLPHMLRELRAAFGKVKIFFQM | 323 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIES | |
| IMP-45 | YCVEYEESEEDRQQCSSSNFPASLPHMLRELRAAFGKVKTFFQMK | 324 |
| without signal | DQLNSMLLTQSLLDDFKGYLGCQALSEMIQFYLEEVMPQAENHGP | |
| peptide | DIKEHVNSPGEKLKTLRLRLRRCHRFLPCENKSKAVEQVKRVFNM | |
| | LQERGVYKAMSEFDIFINYIES | |
| IMP-46 | YCTSCSYRDCTEDEDQKQQCEGGLRSLPHMLRELRAAFGKVKIFF | 325 |
| without signal | QMKDQLHSLLLTQSLLDDFKGYLGCQALSEMIQFYLEEVMPQAEN | |
| peptide | HGPDIKEHVNSLGEKLKTLRLRLRRCHRFLPCENKSKAVEKVKRV | |
| | FSELQERGVYKAMSEFDIFINYIETYMT | |
| IMP-47 | YCVEYEESEEDRQQCSSSNFPASLPHMPRELRAAFGKVKTFFQMK | 326 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIES | |
| IMP-48 | YCTSCSHRDCTEDDEQKQQCEGGSGGLGSLPHMLRELRAAFGKVK | 327 |
| without signal | TFFQMKDQLHSLLLTQSLLDDFKGYLGCQALSEMIQFYLEEVMPQ | |
| peptide | AENHGPDIKEHVNSLGEKLKTLRLRLRRCHRFLPCENKSKAVEKV | |
| | KRVFSELQERGVYKAMSEFDIFINYIETYMT | |
| IMP-49 | TDQCDNFPQMLRDLRDAFSRVKTFFQTKDEVDNLLLKESLLEDFK | 328 |
| without signal | GYLGCQALSEMIQFYLEEVMPQAENQDPGAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-50 | TDQCDNFPQMLRDLRDAFSRVKTFFQTKDEVDNLLLKESLLEDFK | 329 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-51 | TDQCDNFPQMLRDLRDAFSRVKTFFQTKDEVDNLLLKESLLEDFK | 330 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTMKAR | |
| IMP-52 | VSNCGNLPHMLRDLRDAFSRVKTFFQMKDQLDNILLKESLLEDFK | 331 |
| without signal | GYLGCQALSEMIQFYLEEVMPQAENQDPNAKEHVNSLGENLKTLR | |
| peptide | LRLRRCHRFLPCENKSKAVEQVKNAFSKLQEKGVYKAMSEFDIFI | |
| | NYIEAYMTMKTRR | |
| IMP-53 | TDQCDNFPQMLRDLRDAFSRVKTFFQTKDEVDNLLLKESLLEDFK | 332 |
| without signal | GYLGCQALSEMIQFYLEEVMPQAENQDPEAKDHVNSLGENLKTIR | |
| peptide | LRLRRCHRFLPCENKSKAVEQIKNAFNKLQEKGIYKAMSEFDIFI | |
| | NYIEAYMTIKAR | |
| IMP-54 | TDQCDNFPQMLRDLRDAFSRVKTFFQTKDEVDSLLLKESLLEDFK | 333 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-55 | TDQCDNFPQMLRDLRDAFSRVKTFFQTKDEVDNILLKESLLEDFK | 334 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-56 | TDQCDNFPQMLRDLRDAFSRVKTFFQTKDEVDNLFLKESLLEDFK | 335 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-57 | TDQCDNFPQMLRDLRDAFSRVKTFFQTKDEVDNLLLKESLLEDFK | 336 |
| without signal | GYLGCQALSEMIQFYLEKVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-58 | TDQCDNFPQMLRDLRDAFSRVKTFFQTKDAVDNLLLKESLLEDFK | 337 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-59 | ASNCGNLPHMLRDLRDAFSRVKIFFQMKDQLDNILLKESLLEDER | 338 |
| without signal | GYLGCQALSEMIQFYLEEVMPQAENQDPHSKEHVNSLGENLKTLR | |
| peptide | LRLRRCHRFLPCENKGKAVEQVKNAFSKLQEKGVYKAMSEFDIFI | |
| | NYIEAYMTMKLRR | |
| IMP-60 | TDQCDNFPQMLRDLRDAFSRVKTFFQTKDEVDNLLLKESLLEDFK | 339 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFHIFI | |
| | NYIEAYMTIKAR | |
| IMP-61 | EMLRDLRDAFSRVKTFFQTKDEVDNLLLKESLLEDFKGYLGCQAL | 340 |
| without signal | SEMIQFYLEEVMPQAENQDPEAKDHVNSLGENLKTLRLRLRRCHR | |
| peptide | FLPCENKSKAVEQIKNAFNKLQEKGIYKAMSEFDIFINYIEAYMT | |
| | IKAR | |
| IMP-62 | ETCGNIPHMLRDLRDAFSRVKTFFQMKDQLDNILLKESLLEDFKG | 341 |
| without signal | YLGCQALSEMIQFYLEEVMPQAEAMSLKSQEHVNFLGENLNTLRL | |
| peptide | RLRRCHRFLPCENKSKAVEQVKNAFSKLQEKGVYKAMSEFDIFIN | |
| | YIEAYMTMKLRR | |
| IMP-63 | TANNRAQKCFCFDGSNAGNSEETNTAAFQKKCDSEIPESLPYMLR | 342 |
| without signal | DLRNSSVQTRRYFQEKDEENSPLLTQKLLEDFKGYLGCQALSEMI | |
| peptide | QFYLEEVMPQAEDSNPSAKDSVTSLGEKLKILRLRLRRCHRFLPC | |
| | ENKSKAVENLKSKFGDLGNQGVHKAMSEFDIFINYIETYMTTKMK | |
| IMP-64 | ATTTTIKNTKPQCRPEDYATRLQDLRVTFHRVKPTLQREDDYSVW | 343 |
| without signal | LDGTVVKGCWGCSVMDWLLRRYLETVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-65 | ATTTTIKNTKPQCRPEDYATRLQDLRVTFHRVKPTLQREDDYSVW | 344 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-66 | ATTTTKNTKPQCRPEDYATRLQDLRVTFHRVKPTLQREDDYSVWL | 345 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DTLFSRLEEYLHSRK | |
| IMP-67 | ATTTTIKNTKPQCRPEDYATRLQDLRVTFHRVKPTLQREDDYSVW | 346 |
| without signal | LDGTMVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-68 | ATTTTIKNTKPQCRPEDYATRLQDLRVTFHRVKSTLQREDDYSVW | 347 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-69 | ATTTTIKNTKPQCRPEDYATRLQDLRVTFHRVKPTLQREDDYSVW | 348 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDMLFSRLEEYLHSRK | |
| IMP-70 | ATTTTIKNTKPQCRPEDYATRLQDLRVTFHRVKPTLQREDDYSVW | 349 |
| without signal | LDGTVVKGCWGCSVMDWLLRRYLEIVLPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-71 | AATTTIKNTKPQCRPEDYATRLQDLRVTFHRVKPTLQREDDYSVW | 350 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-72 | ATTTTINNTKPQCRPEDYATRLQDLRVTFHRVKPTLQREDDYSVW | 351 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-73 | ATTTTIKNTKPQCRPEDYATRLQDLRVTFHRVKPTLQREDDYSVW | 352 |
| without signal | LDGTVVKGCWGCSVMDWLLRRYLEIVFPAGDHVYPGLKTELHSMH | |
| peptide | STLESIYKDMRQCPLLGCGDKSVISRLSQEAERKSDNGTRKGLSE | |
| | LDTLFSRLEEYLHSRK | |
| IMP-74 | ATTTTIKNTKPQCRPEDYATRLQDLRVTFHRVKPTLQREDDYSVW | 353 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGMRKGLSE | |
| | LDTLFSRLEEYLHSRK | |
| IMP-75 | SKPPVDCDPIHGTLSRIIKEVRTGYGSIKQALQSKDTVYYVSLFH | 354 |
| without signal | ESLLREMLSPVGCRVTNELMQHYLDGVLPRAFQCGYDNTTLSGLH | |
| peptide | SLVSSLDTLYKHMLKCPALACTGQTPAWTQFLETEHKLDPWKGTI | |
| | KATAEMDLLVNYLETFLAQS | |
| IMP-76 | ATTTTIKNTKPQCRPEDYATRLQDLRVTFDRVKPTLQREDDYSVW | 355 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-77 | AATTTIKNTKPQCRPEDYATRLQDLRVTFHRVKPTLQHEDDYSVW | 356 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-78 | ATTTTIKNTKPQCRPEDYATRLQDLRVTFHRVKPTLQREDDYSVW | 357 |
| without signal | LDGMVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-79 | ATTTTIKNTKPQCRPEDYATRLQDLRVTFHRVKPTLQREDDYSVW | 358 |
| without signal | LDGTVVKGCWGCSVVDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-80 | ATTTTIKNTKPQCRPEDYATRLQDLRITFHRVKPTLQREDDYSVW | 359 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-81 | ATTTIKNTKPQCRPEDYATRLQDLRVTFHRIKPTLQREDDYSVWL | 360 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DTLFSRLEEYLHSRK | |
| IMP-82 | ATTTTIKNTKPQCRPEDYATRLQDLRVTFHRVKPTLQCEDDYSVW | 361 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-83 | ATTTTTIKNTKPQCRPEDYATRLQDLRVTFHRVKPTLQREDDYSV | 362 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-84 | ATTTTIKNTKPQCRPEDYATRLQDLRVTFHRVKPTLQREDDYSVW | 363 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDALFSRLEEYLHSRK | |
| IMP-85 | ATTTTIKNTKPQCRPEDYATRLQDLRVTFHRVKPTLQREDDYSVW | 364 |
| without signal | LDGTVVKGCWGCSVMDWLLRRYLEILFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-86 | ATTTTTIKNTKPQCRPEDYATRLQDLRVTFHRVKPTLQREDDYSV | 365 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGMRKGLS | |
| | ELDTLFSRLEEYLHSRK | |
| IMP-87 | ATTTIKNTKPQCRPEDYATRLQDLRVTFHRVKPTLQREDDYSVWL | 366 |
| without signal | DGTVVKGCWGCSVMDWLLRRYLEIVFPAGDHVYPRLKTELHSMRS | |
| peptide | TLESIYKDMQQCPLLGCGDKSVISRLSQEAERKSDNGTRKGLSEL | |
| | DTLFSRLEEYLHSRK | |
| IMP-88 | AATTTTIKNTKPQCRPEDYATRLQDLRVTFHRVKPTLQREDDYSV | 367 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-89 | AATTTTTIKNTKPQCRPEDYATRLQDLRVTFHRVKPTLQREDDYS | 368 |
| without signal | VWLDGTVVKGCWGCSVMDWLLRRYLEIVFPAGDHVYPGLKTELHS | |
| peptide | MRSTLESIYKDMRQCPLLGCGDKSVISRLSQEAEKKSDNGTRKGL | |
| | SELDTLFSRLEEYLHSRK | |
| IMP-90 | AATTTTIKNTKPQCRPEDYASRLQDLRVTFHRVKPTLQREDDYSV | 369 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-91 | ATTTTTIKNTKPQCRPEDYATRLQDLRVTFHRVKPTLQREDDYSV | 370 |
| without signal | WLDGTVVKGCWGCSVMDWLLRRYLEIMFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-92 | ATTTIKNTKPRCRPEDYATRLQDLRVTFHRVKPTLQREDDYSVWL | 371 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DTLFSRLEEYLHSRK | |
| IMP-93 | ATTTTIKNTKPQCRPEDYATRLQDLRVTFHRVKPTLQREDDYSVW | 372 |
| without signal | LDGTVVKGCWGCSVMDWLLRRYLEIVFPAGDYVYPGLKTELHSMR | |
| peptide | STLESIYKDMRQCPLLGCGDKSVISRLSQEAERKSDNGMRKGLSE | |
| | LDTLFSRLEEYLHSRK | |
| IMP-94 | ATTTIKNTKPQCRPEDYATRLQDLRVTFHRVKPTLQREDDYSVWL | 373 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGIRKGLSEL | |
| | DALFSRLEEYLHSRK | |
| IMP-95 | ATTTTTIKNTKPRCRPEDYATRLQDLRVTFHRVKPTLQREDDYSV | 374 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-96 | AATTTTTIKNTKPQCRPEDYATRLQDLRVTFHRVKPTLQREDDYS | 375 |
| without signal | VWLDGTVVKGCWGCSVMDWLLRRYLEIVFPAGDHVYPGLKTELHS | |
| peptide | MRSTLESIYKDMRQCPLLGCGDKSVISRLSQEAERKSDNGTRKGL | |
| | SELDTLFSRLEEYLHSRK | |
| IMP-97 | ATTTTTTIKNTKPQCRPEDYATRLQDLRVTFHRVKPTLQREDDYS | 376 |
| without signal | VWLDGTVVKGCWGCSVMDWLLRRYLEIVFPAGDHVYPGLKTELHS | |
| peptide | MRSTLESIYKDMRQCPLLGCGDKSVISRLSQEAERKSDNGTRKGL | |
| | SELDTLFSRLEEYLHSRK | |
| IMP-98 | AATTTIKNTKPQCRPEDYATRLQDLRVTFHRVKPTLQREDDYSVW | 377 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLNE | |
| | LDTLFSRLEEYLHSRK | |
| IMP-99 | ATTTTTIKNTKPQCRPEDYATRLQDLRVTFHRVKPTLQREDDYSV | 378 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDALFSRLEEYLHSRK | |
| IMP-100 | ATTTTIKNTKPQCRPEDYATRLQDLCVTFHRVKPTLQREDDYSVW | 379 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-101 | ATTTTTTTIKNTKPQCRPEDYATRLQDLRVTFHRVKPTLQREDDY | 380 |
| without signal | SVWLDGTVVKGCWGCSVMDWLLRRYLEIVFPAGDHVYPGLKTELH | |
| peptide | SMRSTLESIYKDMRQCPLLGCGDKSVISRLSQKAERKSDNGTRKG | |
| | LSELDTLFSRLEEYLHSRK | |
| IMP-102 | AATTTTIKNTKPQCRPEDYASRLQDLRVTFHRVKPTLQREDDYSV | 381 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKSLS | |
| | ELDTLFSRLEEYLHSRK | |
| IMP-103 | ATTTIKNTKPQCRPEDYATRLQDLRVTFHRVKPTLQREDDYSVWL | 382 |
| without signal | DGTVVKGCWGCSVMDWLLRRYLEIVFPAGDHVYSGLKTELHSMRS | |
| peptide | TLESIYKDMRQCPLLGCGDKSVISRLSQEAERKSDNGTRKGLSEL | |
| | DTLFSRLEEYLHSRK | |
| IMP-104 | ATTTTTTIKNTKPQCRPEDYATRLQDLRVTFHRVKPTLQREDDYS | 383 |
| without signal | VWLDGTVVKGCWGCSVMDWLLRRYLEIVFPAGDHVYPGLKTELHS | |
| peptide | MRSTLESIYKDMRQCPLLGCGDKSVISRLSQKAERKSDNGTRKGL | |
| | SELDTLFSRLEEYLHSRK | |
| IMP-105 | ATTTTTMIKNTKPQCRPEDYATRLQDLRVTFHRVKPTLQREDDYS | 384 |
| without signal | VWLDGTVVKGCWGCSVMDWLLRRYLEIVFPAGDHVYPGLKTELHS | |
| peptide | MRSTLESIYKDMRQCPLLGCGDKSVISRLSQEAERKSDNGTRKGL | |
| | SELDTLFSRLEEYLHSRK | |
| IMP-106 | ATTTTTIKNTKPQCRPEDYATRLQDLRVTFHRVKPTLQREDDYSV | 385 |
| without signal | WLDGTVVKGCWGCSVMDWLLRRYLEIVFPAGDHVYPGLKTELHSM | |
| peptide | RSTLESIYKDMWQCPLLGCGDKSVISRLSQEAERKSDNGTRKGLS | |
| | ELDTLFSRLEEYLHSRK | |
| IMP-107 | AATTTTIKNTKPQCRPEDYATRLQDFRVTFHRVKPTLQREDDYSV | 386 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-108 | ATIKNTKPQCRPEDYATRLQDLRVTFHRVKPTLQREDDYSVWLDG | 387 |
| without signal | TVVKGCWGCSVMDWLLRRYLEIVFPAGDHVYPGLKTELHSMRSTL | |
| peptide | ESIYKDMRQCPLLGCGDKSVISRLSQEAERKSDNGTRKGLSELDT | |
| | LFSRLEEYLHSRK | |
| IMP-109 | ATTTTTTTIKNTKPQCRPEDYATRLQDLRVTFHRVKPTLQREDDY | 388 |
| without signal | SVWLDGTVVKGCWGCSVMDWLLRRYLEIVFPAGDHVYPGLKTELH | |
| peptide | SMRSTLESIYKDMRQCPLLGCGDKSVISRLSQEAERKSDNGTRKG | |
| | LSELDTLFSRLEEYLHSRK | |
| IMP-110 | ATTTTTTMIKNTKPQCRPEDYATRLQDLRVTFHRVKPTLQREDDY | 389 |
| without signal | SVWLDGTVVKGCWGCSVMDWLLRRYLEIVFPAGDHVYPGLKTELH | |
| peptide | SMRSTLESIYKDMRQCPLLGCGDKSVISRLSQEAERKSDNGTRKG | |
| | LSELDTLFSRLEEYLHSRK | |
| IMP-111 | ATTTTTIKNTKPQCRPEDYATRLQDLRVTFHRVKPTLQREDDYSV | 390 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLR | |
| | ELDTLFSRLEEYLHSRK | |
| IMP-112 | AKPAATTTTTTTTIKNTKPQCRPEDYATRLQDLRVTFHRVKPTLQ | 391 |
| without signal | REDDYSVWLDGTVVKGCWGCSVMDWLLRRYLEIVFPAGDHVYPGL | |
| peptide | KTELHSMRSTLESIYKDMRQCPLLGCGDKSVISRLSQEAERKSDN | |
| | GTRKGLSELDTLFSRLEEYLHSRK | |
| IMP-113 | ATTTTTTIKNTKPQCRPEDYATRLQDLRVTFHRVKPTLQREDDYS | 392 |
| without signal | VWLDGTVVKGCWRCSVMDWLLRRYLEIVFPAGDHVYPGLKTELHS | |
| peptide | MRSTLESIYKDMRQCPLLGCGDKSVISRLSQEAERKSDNGTRKGL | |
| | SELDTLFSRLEEYLHSRK | |
| IMP-114 | ATTTTTIKNTKPQCRPEDYATRLQDLCVTFHRVKPTLQREDDYSV | 393 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-115 | ATTTTTTTTIKNTKPQCRPEDYATRLQDLRVTFHRVKPTLQREDD | 394 |
| without signal | YSVWLDGTVVKGCWGCSVMDWLLRRYLEIVFPAGDHVYPGLKTEL | |
| peptide | HSMRSTLESIYKDMRQCPLLGCGDKSVISRLSQEAERKSDNGTRK | |
| | GLSELDTLFSRLEEYLHSRK | |
| IMP-116 | ATTTTTTTIKNTKPQCRPEDYATRLQDLRVTFHRVKPTLQREDDY | 395 |
| without signal | SVWLDGTVVKGCWGCSVMDWLLRRYLEIVFPAGDHVYPGLKTELH | |
| peptide | SMRSTLESIYKDMRQCPLLGCGDKSVISRLSQEAERKSDNGTRKG | |
| | LSELDTLFIRLEEYLHSRK | |
| IMP-117 | ATTTTTIKNTKPQCRPEDYATRLQDLRVTFHRVKPTLQREDDYSV | 396 |
| without signal | WLDGTVVKGCWGRSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-118 | ATTTTTIKNTKPQCRPEDYATRLQDLRVTFHRVKPTLQREDDYSV | 397 |
| without signal | WLDGTVVKGCWGCSVMDWLLRRYLEIVFPAGDHVYPGLKTELHSM | |
| peptide | RSTLESICKDMRQRPLLGCGDKSVISRLSQEAERKSDNGTRKGLS | |
| | ELDTLFSRLEEYLHSRK | |
| IMP-119 | AKPAATTTTTTTTTIKNTKPQCRPEDYATRLQDFRVTFHRVKPTL | 398 |
| without signal | QREDDYSVWLDGTVVKGCWGCSVMDWLLRRYLEIVFPAGDHVYPG | |
| peptide | LKTELHSMRSTLESIYKDMRQCPLLGCGDKSVISRLSQEAERKSD | |
| | NGTRKGLSELDTLFSRLEEYLHSRK | |
| IMP-120 | ATIKNTKPQCRPEDYATRLQDLRVTFHRVKPTLPGHQREDDYSVW | 399 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-121 | CAIASAKKCDDVSFDYILKDLRSEFSKIKSFVQDNDQENMMLLSQ | 400 |
| without signal | SMLDKLISRIGCKSLSDMIKFYLNDVLPNAEKIEHMKNKITSIGE | |
| peptide | KLKSLKEKLISCDFLHCENHDEIKTVKTIFNKLKDKGIYKAMGEF | |
| | DIFINYLEKYIVKK | |
| IMP-122 | CAIASAKKCNDVSFDYILKDLRSEFSKIKSFVQDNDQENMMLLSQ | 401 |
| without signal | SMLDKLTSRIGCKSLSDMIKFYLNDVLPNAEKIEHMKNKITSIGE | |
| peptide | KLKSLKEKLISCDFLHCENHDEIKTVKTIFNKLKDKGIYKAMGEF | |
| | DIFINYLEKYIVKK | |
| IMP-123 | CVVASAKKCDDVSFDYILKDLRSEFSKIKSFVQDNDQENMMLLSQ | 402 |
| without signal | SMLDKLTSRIGCKSLSDMIKFYLNDVLPNAEKIEHMKNKITSIGE | |
| peptide | KLKSLKEKLISCDFLHCENHDEIKTVKTIFNKLKDKGIYKAMGEF | |
| | DIFINYLEKYIVKK | |
| IMP-124 | CVVAYAKKCDDVSFDYILKDLRSEFSKIKSFVQNNDQENMMLLSQ | 403 |
| without signal | SMLNKLTSCIGCKSLSDMIKFYLNDVLPNAEKIEQIKNIITSIGE | |
| peptide | KLKSLKEKLISCDFLHCENNDEIKTVKAIFNKLKDKGIYKAMGEF | |
| | DIFINYVEKYIVKT | |
| IMP-125 | CVVASAKKCDDVSFDYILKDLRSEFIKIKSFVQNNDQENMMLLSQ | 404 |
| without signal | SMLDKLTSCIGCKSLSDMIKFYLNDVLPNAEKIEQIKNIITSIGE | |
| peptide | KLKSLKEKLISCDFLHCENNDEIKTVKAIFNKLKDKGIYKAMGEF | |
| | DIFINYVEKYIVKT | |
| IMP-126 | KKCDDVSFDYILKDLRSEFSKIKSFVQNNDKENMMLLSQSMLDKL | 405 |
| without signal | TSCIGCKSLSDMIKFYLNDVLPNAEKIEHIKNKITSIGEKLKSLK | |
| peptide | EKLISCDFLHCENHDEIKAVKTIFNKLKDKGIYKAMGEFDIFINH | |
| | LEKYIVKK | |
| IMP-127 | CVVASAKKCDDVSFDYILKDLRSEFIKIKSFVQNNDQENMMLLSQ | 406 |
| without signal | SMLDKLISRIGCKSLSDMIKFYLNDVLPNAEKIEQIKNIITSIGE | |
| peptide | KLKSLKEKLISCDFLHCENNDEIKTVKAIFNKLKDKGIYKAMGEF | |
| | DIFINYVEKYIVKT | |
| IMP-128 | CTVASAKKCDDVSFDYILKDLRSEFSKIKSFVQNNDKENMMLLSQ | 407 |
| without signal | SMLDKLISCIGCKSLSDMIKFYLNDVLPNAEKIEHIKNKITSIGE | |
| peptide | KLKSLKEKLISCDFLHCENHDEIKAVKTIFNKLKDKGIYKAMGEF | |
| | DIFINHLEKYIVKK | |
| IMP-129 | ATTTTTIKNTKPQCRPEDYATRLQDLRVTFHRVKPTLDDYSVWLD | 408 |
| without signal | GTVVKGCWGCSVMDWLLRRYLEIVFPAGDHVYPGLKTELHSMRST | |
| peptide | LESIYKDMRQCPLLGCGDKAVISRLSQEAERKSDNGTRKGLSELD | |
| | TLFSRLEEYLHSRK | |
| IMP-130 | CTVASAKKCDDVSFDYILKDLRSEFSKIKSFVQNNDKENMMLLSQ | 409 |
| without signal | SMLDKLTRCIGCKSLSDMIKFYLNDVLPNAEKIEHIKNKITSIGE | |
| peptide | KLKSLKERLISCDFLHCENHDEIKAVKTIFNELKDKGIYKAMGEF | |
| | DIFINHLEKYIVKK | |
| IMP-131 | TDQCDNFPQMLRDLRDAFSRVKTFFQTKDEVDNLLLKESLLEDFK | 410 |
| without signal | GYLGCQALSEMIQFYLEEVMPQAENQDPEAKDHVNSLGENLKTLR | |
| peptide | LRLRRCHRFLPCENKSKAVEQIKMPLISCRKKEFTKP | |
| IMP-132 | KGRDSKPSPACDPMHGALAGIFKELRITYRSVRETLQTKDTVYYV | 411 |
| without signal | SLFHEQLLQEMLSPVGCRVTNELMQHYLDGVLPRAFHCGYDNTTL | |
| peptide | NALHFLSSSLSTLYQHMLKCPALACTGQTPAWTQFLDTEHKLDPW | |
| | KGTVKATAEMDLLLNYLETFLLQS | |
| IMP-133 | ATTTIKNTKPQCRPEDYATRLQDLRVTFHRVKPTLQREDDYSVWL | 412 |
| without signal | DGTVVKGCWGCSVMDWLLRRYLEIVFPAGDHVYPGLKTELHSMRS | |
| peptide | TLESIYKDMRQCEAERKSDNGTRKGLSELDTLFSRLEEYLHSRK | |
| IMP-134 | AHDHEHKVPPACDPVHGNLAGIFKELRTIYTSIREGLQKKDTVYY | 413 |
| without signal | TSLFNDRVLQEMLSPMGCRVTNEIMEHYLDGVLPRASHLDYDNST | |
| peptide | LNGLHAFASSMQALYQHMLKCPALACTGKTPAWMYFLEVEHKLNP | |
| | WRGTAKAAAEADLLLNYLETFLLQF | |
| IMP-135 | ASKPPVDCDPIHGTLSRIIKEVRTGYGSIKQALQSKDTVYYVSLF | 414 |
| without signal | HENLLNEMLSPVGCRVTNELMQHYLDGVLPRAFQCGYDNTTLDGL | |
| peptide | HSLVSSLDALYKHMLKCPALACTGQTPAWTQFLETEHKLDPWKGT | |
| | IKATAEMDLLVNYLETFLAQS | |
| IMP-136 | ATTTTTIKNTKPQCRPEDYATRLQDLRVTFHRVKPTLQREDDYSV | 415 |
| without signal | WLDGTVVKGCWGCSVMDWLLRRYLEIVFPAGDHVYPGLKTELHSM | |
| peptide | RSTLESIYKDMRQCPLLGCGDKSVISR | |
| IMP-137 | ATTTIKNTKPQCRPEDYATRLQDLRVTFHRVKPTLQREDDYSVWL | 416 |
| without signal | DGTVVKGCWGCSVMDWLLRRYLEIVFPAGDHVYPGLKTELHSMRS | |
| peptide | TLESIYKDMRQCVSVSVAALSAQR | |
| IMP-138 | IIDTCYDDQERERTKSNSISSVTPEMCKGLKQLVSTKLKDARQKE | 417 |
| without signal | KSVRDYFTSRDNDLDFMLLQGVKETHKKTCGCYVLYLLLSFYGKT | |
| peptide | IRDTIQSNKHKNLNTELTNLAVSVLSLEDLLEACGITCNPKKDSL | |
| | LKRIEEYMKEHGDDAIYKVIGEIEFLFQAIEKHVY | |
| IMP-139 | NSIIDMCYDDQERERTKSNSISSITPDMCKGLKQLVATKLKDARQ | 418 |
| without signal | KEKLVNSYFTSRDNDLTYMLLQGVRETHKKPCGCYVLYLLLTFYR | |
| peptide | KTIKDIIQSKKHESINTELINLAVTVLSLEDLLEACGITCNPKKD | |
| | SLLKRIEGYTKEHGDDAIYKVIGEIDFLFQAIERHVY | |
| IMP-140 | IIDTCYDDQERERTKSNSISSVTPEMCKGLKQLVATKLKDARQKE | 419 |
| without signal | KLVNDYFTGRDNDLSYMLLQGVRETHKKPCGCYVLYLLLSFYRKT | |
| peptide | IRDTIQSNKHASINAELINLAVSVLSLEDLLDACGITCNPKKDSL | |
| | LKRIEEYMKEHGDDAIYKLIGEIEFLFQAIERHVYT | |
| IMP-141 | IIDTYDEDEDEDSIKLSSIGSITPEMCKNLKQLVASKLKDIRQKE | 420 |
| without signal | KSLRDYFTNLDDELDYMLLQGVGENHKKKCGCYILHLLLKFYSKT | |
| peptide | IRNTIQSEKHKNVNLELTNIAVSMLALEDLLEKCGITCNPKKDPL | |
| | LKRIEDYMKQHGDDGVNKAIAELEFLFQMIEKQVYI | |
| IMP-142 | AAQCRKGTITSRLKMLRTAFEKVREFYEDRDEEETALASTEHLHG | 421 |
| without signal | PESCSVIDELITHYTKCVIPAANEEEGADLLSLDTLQVALENVKG | |
| peptide | LLANCQEEFGCKPPFSMRDYKKQYRQLNKEKNAGMIKAMGELGML | |
| | FNGIEERVIGM | |
| IMP-143 | MYVQHGSDYCTTTVHADIASAISGMRAEYDSGLGHYFKSLVPHPD | 422 |
| without signal | NPYDTDDYKYMINNTNSYNCHALQSTINALLGMYGYVDIDEPHQL | |
| peptide | AMMKLATHTMQAAMLLNKCAKQLGCYHIPFDVETLHEAHPDDVMA | |
| | SLDTALNLMSMVTNEI | |
| IMP-144 | AAQCRKGTITSRLKMLRTAFEKVREFYEDSDEEETALASTEHLHG | 423 |
| without signal | PESCSVIDELITHYTKCVIPAANEEEGADLLSLDTLQVALENVKG | |
| peptide | LLANCQEEFGCKPPFSMRDYKKQYRQLNKEKNAGMIKAMGELGML | |
| | FNGIEERVIGM | |
| IMP-145 | ATIKCVGMSTLFNPELIQLRRLFGDGIKDFFQNKDEDLDNAFLNE | 424 |
| without signal | DVQRELASDCGCDHLMDMLSLYVNDTIPKGMKTEDAPSGLGQMGQ | |
| peptide | LMSSLYRKMDMCWSELGCSHNTRLTLQEYADKKGGWDNKALGESD | |
| | ILFDALELFFSKIK | |
| IMP-146 | MYVQHGSDYCTTTVRADIASAISGMRAEYNNGLGDYFKSLAPHPN | 425 |
| without signal | NPYDTDDYKYMINSTNSYNCHALQSTINALLGMYGYVDIDEPHQL | |
| peptide | AMMKLATHTMQTAMLLNKCAAQLGCYHIPFDVETLHEAHPNDVMA | |
| | SLDTALNLMSMVTNEI | |
| IMP-147 | MYARRSGDYCTTTVRADIASAISGMRAEYNSGLRDYFKSLVPHPD | 426 |
| without signal | NPYDTDDYKYMLNNTNSYNCHALQSTINALLGMYGYVDIDESHQL | |
| peptide | AMMKLATHTMQTAMMLNKCAAQLGCYHIPFDLETLHEAHPDDVMA | |
| | SLDTALNLMSMITNEI | |
| IMP-148 | MYVQHGSDYCTTTVRADIASAISGMRAEYNNGLGDYFKSLAPHPN | 427 |
| without signal | NPYDTDDYKYMINSTNSYNCHALQSTINALLGMYGYVDIDEPHQL | |
| peptide | AMMKLATHTMQTAMLLNKCAAQLGCYHIPFDVETLHEAHPDDVMA | |
| | SLDTALNLMSMVTNEI | |
| IMP-149 | NVHSGTEDNPCTNSKTVLNTLLNQIKQEYINNLLPYYKALIPKPV | 428 |
| without signal | DVFDDSYTYSIQSTDYNCYTIYETLNFLLGDVFPRATTDATVRLS | |
| peptide | LAKIATSSQQASMLMNLCKKELACGPAPFDMIKLYHDTKEYGADN | |
| | IMGTLDTPFQYFVIV | |
| IMP-150 | MYVQHGRGDYCTTTVRADIASAISGMRAEYDSGLGHYFKSLVPHP | 429 |
| without signal | DNPYDTDDYKYMINNINSYNCHALQSTINALLGMYGYVDIDEPHQ | |
| peptide | LAMMKLATHTMQTAMLLNKCAEQLGCYHIPFDVEILHEAHPDDVM | |
| | ASLDTALNLMSMVTNEI | |
| IMP-151 | APATTPKDSCVYLIGQTPQLLRQLRNAYQAIIGADGSGVDEDDMP | 430 |
| without signal | IYPSDVMNELASTSVACDAIKKVLIMNIGILPNVTAAYPDKKSEV | |
| peptide | DEIGDNLSRLHQNIVNCRDFLKCEDLPHWHQMAENYKEKPMQGFS | |
| | EMDFVFQSVEKFLVAKDVKNMKTKRKH | |
| IMP-152 | DDDPCTNVKTQLNTLFNQIKTEYDTNLKTYYQSIAPSAFDPFNNT | 431 |
| without signal | NYLYSVQGNDYKCYTIFETLSFLMGDVYPRATTNESVRLSLAKVA | |
| peptide | TSSTQGAMVMNLCRQQLGCGPPPFDAKTLYDDRAEYGADDIMATL | |
| | DTALAKFKLVLESENVV | |
| IMP-153 | DDDPCTNVKTQLNTLFNQIKTEYDTNLKTYYQSIAPSAFDPFNNT | 432 |
| without signal | NYLYSVQGNDYKCYTIFETLSFLMGDVYPRATTNESVRLSLAKVA | |
| peptide | TSSTQGAMVMNLCREQLGCGPPPFDAKTLYDDRAEYGADDIMATL | |
| | DTALAKFKLVLESENVV | |
| IMP-154 | RRRGDYCTTTVRADIASAISGMRAEYNSGLGDYFKSLVPHPDNPY | 433 |
| without signal | DTDDYKHMIDNANSYNCHALQSTINALLGMYGYVDIDEPHQLAMM | |
| peptide | KLATHTMQTAMLLNKCAAQLGCYHIPFDLETLREAPPADVMASLD | |
| | TALNLMSMITNEI | |
| IMP-155 | ASLSTHYNNYDLTRIATIDKDVCKRVAQHINDDFVNMRKLYETQL | 434 |
| without signal | KNYFQQLVPNPTDVFKDDSYMYMINGIDYNCHIIYETMRFLSGDV | |
| peptide | FPFATETEAELQYMWKMMLGVSQLSAYIGNCYQYFKCGPAPFDPQ | |
| | VLYHDRELFHADTVMAYLDTAFSHFTL | |
| IMP-156 | LNCGIEHNELNNIKNIFFKVRNVVQADDVDHNLRILTPALLNNIT | 435 |
| without signal | VSETCFFIYDMFELYLNDVFVKYINTALKLNILKSLSSVANNFLA | |
| peptide | IFNKVKKRRVKKNTVNVLEIKKLLLIDNNCKKLFSEIDIFLTWVM | |
| | AKI | |
| IMP-157 | LNCGIEHNELNNIKNIFFKVRNVVQADDVDHNLRILTPALLNNIT | 436 |
| without signal | VSETCFFIYDMFELYLNDVFVKYTNTALKLNILKSLSSVANNFLA | |
| peptide | IFNKVKKRRVKKNNVNVLEIKKLLLIDNNCKKLFSEIDIFLTWVM | |
| | AKI | |
| IMP-158 | APATTPKDSCVYLIGQTPQLLRQLRNAYQAIIGADGSGVDEDDMP | 437 |
| without signal | IYPSDVMNELASTSVACDAIKKVLIMNIGILPNVTAAYPDKKSEV | |
| peptide | DEIGDNLSRLHQNIVNCVSRTQHLCYD | |
| IMP-159 | DNKYDSESGDDCPTLPTSLPHMLHELRAAFSRVKTFFQMKDQLDN | 438 |
| without signal | MLLDGSLLEDFKGYLGCQALSEMIQFYLEEVMPQAENHSPDQDKN | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-160 | DNKYDSESGNDCPTLPTSLPHMLHELRAAFSRVKTFFQMKDQLDN | 439 |
| without signal | MLLDGSLLEDFKGYLGCQALSEMIQFYLEEVMPQAENHSTGQEKD | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-161 | DNKYDSESGNDCPTLPTSLPHMLHELRAAFSRVKTFFQMKDQLDN | 440 |
| without signal | MLLDGSLLEDFKGYLGCQALSEMIQFYLEEVMPQAENHSTDQEKD | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-162 | DNKYDSESGDDCPTLPTSLPHMLHELRAAFSRVKTFFQMKDQLDN | 441 |
| without signal | MLLDGSLLEDFKGYLGCQALSEMIQFYLEEVMPQAENHSTGQEKD | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-163 | DNRYDGQDGNDCPTLPTSLPHMLHELRAAFSRVKTFFQMKDQLDN | 442 |
| without signal | MLLDGSLLEDFKGYLGCQALSEMIQFYLEEVMPQAENHSPDQDKN | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-164 | ATTAIKNTKPQCRPEDYATRLQDLRVTFHRVKPTLQREDDYSVWL | 443 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DTLFSRLEEYLHSRK | |
| IMP-165 | ATTTTIKNTKPQCRPEDYATRLQDLRVTFDRVKPTLQREDDYSVW | 444 |
| without signal | LDGTVVKGCWGCSVMDWLLRRYLEIVFPAGDHVYPGLKTELHSMR | |
| peptide | STLESIYKDMRQCPLLGCGDKSVISRLSQEAEKKSDNGTRKGLSE | |
| | LDTLFSRLEEYLHSRK | |
| IMP-166 | ATTTTTIKNTKPQCRPEDYATRLQDLRVTFYRVKPTLQREDDYSV | 445 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-167 | ATTTTTIKNTKPQCRPEDYATRLQDLRVTFHRVKPTLDDYSVWLD | 446 |
| without signal | GTVVKGCWGCSVMDWLLRRYLEIVFPAGDHVYPGLKTELHSMRST | |
| peptide | LESIYKDMRQCPLLGCGDKSVISRLSQEAERKSDNGTRKGLSELD | |
| | TLFSRLEEYLHSRK | |
| IMP-168 | STKKCDDVSFDYILKDLRSEFSKIKSFVQDNDQENMMLLSQSMLD | 447 |
| without signal | KLTSRIGCKSLSDMIKFYLNDVLPNAEKIEHMKNKITSIGEKLKS | |
| peptide | LKEKLISCDFLHCENHDEIKTVKTIFNKLKDKGIYKAMGEFDIFI | |
| | NYLEKYIVKK | |
| IMP-169 | ATTTIKNTKPQCRPEDYATRLQDLRVTFHRVKPTLQREDDYSVWL | 448 |
| without signal | DGDHVYPGLKTELHSMRSTLESIYKDMRQCPLLGCGDKSVISRLS | |
| peptide | QEAERKSDNGTRKGLSELDTLFSRLEEYLHSRK | |
| IMP-170 | MIGTCYDEDEEIERLKSNSISSITPGMCRNLKHSVMIRLIDARQI | 449 |
| without signal | EASIRSYFTDGDNNLSFMLLQGIREISKKKCGCYILNLMLRFYIQ | |
| peptide | TIKHTILSNKHKDMNLELINLAVTILSLESLLEKCGVTCNPVKDP | |
| | LLTRIEEYTRKHGDNAIYKTIGELEFLFDAIEKFV | |
| IMP-171 | QCRKGTITIRLKMLRTAFEKVREFYEDRDEEETALASTEHLHGPE | 450 |
| without signal | SCSVIDELITHYTKCVIPAANEEEGADLRSLDTLQFALENVKGLL | |
| peptide | ANCQEEFGCKPPFSMRDYKKQYRQLNKEKNAGMIKAMGELGMLFN | |
| | GIEERVIGM | |
| IMP-172 | QCRKGTITIRLKMLRTAFEKVREFYEDRDEEETALASTEHLHGPE | 451 |
| without signal | SCSVIDELITHYTKCVIPAANEEEGADLRSLDTLQFALENVKGLL | |
| peptide | ANCQEEFGCKPPFSMRDYKKQYRQLNKEKNAGMIKAMGELGMLEN | |
| | GIEERVNGM | |
| IMP-173 | QCRKGTITIRLKMLRTAFEKVREFYEDRDEEETALASTEHLHGPE | 452 |
| without signal | SCSVIDELITHYTKCVIPAANEEEGADLLSLDTLQFALENVKGLL | |
| peptide | ANCQEEFGCKPPFSMRDYKKQYRQLNKEKNAGMIKAMGELGMLFN | |
| | GIEERVIGM | |
| IMP-174 | AAQCRKGTITSRLKMLRTAFEKVREFYEDRDEEETALASTEHLHG | 453 |
| without signal | PESCSVIDELITHHTKCVIPAANEEEGADLLSLDTLQVALENVKG | |
| peptide | LLANCQEEFGCKPPFSMRDYKKQYRQLNKEKNAGMIKAMGELGML | |
| | FNGIEERVIGM | |
| IMP-175 | ATTTIKNTKPQCRPEDYATRLQDLRVTFHRVKPTLVGHVGDHVYP | 454 |
| without signal | GLKTELHSMRSTLESIYKDMRQCEAERKSDNGTRKGLSELDTLFS | |
| peptide | RLEEYLHSRK | |

In some embodiments, the immunomodulatory protein or polypeptide specifically binds hIL-10Rα. In some embodiments, the immunomodulatory protein or polypeptide specifically binds hIL-10Rβ. In some embodiments, the immunomodulatory protein or polypeptide specifically binds both hIL-10Rα and hIL-10Rβ. In some embodiments, the immunomodulatory protein or polypeptide specifically binds both hIL-10Rα and hIL-10Rβ but binds hIL-10Rα with higher affinity than hIL-10Rβ.

In some embodiments, the immunomodulatory protein or polypeptide is a hIL-10R agonist. In some embodiments, the immunomodulatory protein or polypeptide is a hIL-10Rα agonist. In some embodiments, the immunomodulatory protein or polypeptide is a hIL-10Rβ agonist. In some embodiments, the immunomodulatory protein or polypeptide is a hIL-10Rα agonist and a hIL-10Rβ agonist.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 2. For example, the amino acid sequence of the immunomodulatory protein or polypeptide may comprise an amino acid sequence at least 85% identical to the amino acid sequence of a polypeptide set forth in Table 2. The amino acid sequence of the immunomodulatory protein or polypeptide may comprise an amino acid sequence at least 90% identical to the amino acid sequence of a polypeptide set forth in Table 2. The amino acid sequence of the immunomodulatory protein or polypeptide may comprise an amino acid sequence at least 95% identical to the amino acid sequence of a polypeptide set forth in Table 2. In some embodiments, the amino acid sequence of the immunomodulatory protein or polypeptide may preferably comprise an amino acid sequence 100% identical to the amino acid sequence of a polypeptide set forth in Table 2. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 2. For example, the amino acid sequence of the immunomodulatory protein or polypeptide may consist of an amino acid sequence at least 85% identical to the amino acid sequence of a polypeptide set forth in Table 2. The amino acid sequence of the immunomodulatory protein or polypeptide may consist of an amino acid sequence at least 90% identical to the amino acid sequence of a polypeptide set forth in Table 2. The amino acid sequence of the immunomodulatory protein or polypeptide may consist of an amino acid sequence at least 95% identical to the amino acid sequence of a polypeptide set forth in Table 2. In some embodiments, the amino acid sequence of the immunomodulatory protein or polypeptide may preferably consist of an amino acid sequence 100% identical to the amino acid sequence of a polypeptide set forth in Table 2.

In some embodiments, the amino acid sequence of the immunomodulatory protein or polypeptide comprises or consists of the amino acid sequence of a polypeptide set forth in Table 2, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of a polypeptide set forth in Table 2, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of a polypeptide set forth in Table 2, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of a polypeptide set forth in Table 2, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory protein or polypeptide comprises or consists of the amino acid sequence of a polypeptide set forth in Table 2, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of a polypeptide set forth in Table 2, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of a polypeptide set forth in Table 2, and further comprises or consists of about 1, 2, 3, 4, 5, 6, 7, 8, 9, or 10 amino acid substitutions. In some embodiments, the amino acid sequence of the immunomodulatory protein or polypeptide comprises or consists of the amino acid sequence of a polypeptide set forth in Table 2, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-454. For example, the amino acid sequence of the immunomodulatory protein or polypeptide may comprise an amino acid sequence at least 85% identical to the amino acid sequence of any one of SEQ ID NOS: 108-454. The amino acid sequence of the immunomodulatory protein or polypeptide may comprise an amino acid sequence at least 90% identical to the amino acid sequence of any one of SEQ ID NOS: 108-454. The amino acid sequence of the immunomodulatory protein or polypeptide may comprise an amino acid sequence at least 95% identical to the amino acid sequence of any one of SEQ ID NOS: 108-454. In some embodiments, the amino acid sequence of the immunomodulatory protein or polypeptide may preferably comprise an amino acid sequence 100% identical to the amino acid sequence of any one of SEQ ID NOS: 108-454. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-454. For example, the amino acid sequence of the immunomodulatory protein or polypeptide may consist of an amino acid sequence at least 85% identical to the amino acid sequence of any one of SEQ ID NOS: 108-454. The amino acid sequence of the immunomodulatory protein or polypeptide may consist of an amino acid sequence at least 90% identical to the amino acid sequence of any one of SEQ ID NOS: 108-454. The amino acid sequence of the immunomodulatory protein or polypeptide may consist of an amino acid sequence at least 95% identical to the amino acid sequence of any one of SEQ ID NOS: 108-454. In some embodiments, the amino acid sequence of the immunomodulatory protein or polypeptide may preferably consist of an amino acid sequence 100% identical to the amino acid sequence of any one of SEQ ID NOS: 108-454.

In embodiments, the amino acid sequence of the immunomodulatory protein or polypeptide comprises or consists of the amino acid sequence of any one of SEQ ID NOS: 108-454, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-454, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-454, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-454, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 108-454, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 108-454, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 108-454, and further comprises or consists of about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 108-454, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-114 or 116-125. For example, the amino acid sequence of the immunomodulatory protein or polypeptide may comprise an amino acid sequence at least 85% identical to the amino acid sequence of any one of SEQ ID NOS: 108-114 or 116-125. The amino acid sequence of the immunomodulatory protein or polypeptide may comprise an amino acid sequence at least 90% identical to the amino acid sequence of any one of SEQ ID NOS: 108-114 or 116-125. The amino acid sequence of the immunomodulatory protein or polypeptide may comprise an amino acid sequence at least 95% identical to the amino acid sequence of any one of SEQ ID NOS: 108-114 or 116-125. In some embodiments, the amino acid sequence of the immunomodulatory protein or polypeptide may preferably comprise an amino acid sequence 100% identical to the amino acid sequence of any one of SEQ ID NOS: 108-114 or 116-125. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-114 or 116-125. For example, the amino acid sequence of the immunomodulatory protein or polypeptide may consist of an amino acid sequence at least 85% identical to the amino acid sequence of any one of SEQ ID NOS: 108-114 or 116-125. The amino acid sequence of the immunomodulatory protein or polypeptide may consist of an amino acid sequence at least 90% identical to the amino acid sequence of any one of SEQ ID NOS: 108-114 or 116-125. The amino acid sequence of the immunomodulatory protein or polypeptide may consist of an amino acid sequence at least 95% identical to the amino acid sequence of any one of SEQ ID NOS: 108-114 or 116-125. In some embodiments, the amino acid sequence of the immunomodulatory protein or polypeptide may preferably consist of an amino acid sequence 100% identical to the amino acid sequence of any one of SEQ ID NOS: 108-114 or 116-125.

In embodiments, the amino acid sequence of the immunomodulatory protein or polypeptide comprises or consists of the amino acid sequence of any one of SEQ ID NOS: 108-114 or 116-125, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-114 or 116-125, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-114 or 116-125, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-114 or 116-125, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 108-114 or 116-125, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 108-114 or 116-125, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 108-114 or 116-125, and further comprises or consists of about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 108-114 or 116-125, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-118 or 127-290. For example, the amino acid sequence of the immunomodulatory protein or polypeptide may comprise an amino acid sequence at least 85% identical to the amino acid sequence of any one of SEQ ID NOS: 108-118 or 127-290. The amino acid sequence of the immunomodulatory protein or polypeptide may comprise an amino acid sequence at least 90% identical to the amino acid sequence of any one of SEQ ID NOS: 108-118 or 127-290. The amino acid sequence of the immunomodulatory protein or polypeptide may comprise an amino acid sequence at least 95% identical to the amino acid sequence of any one of SEQ ID NOS: 108-118 or 127-290. In some embodiments, the amino acid sequence of the immunomodulatory protein or polypeptide may preferably comprise an amino acid sequence 100% identical to the amino acid sequence of any one of SEQ ID NOS: 108-118 or 127-290. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-118 or 127-290. For example, the amino acid sequence of the immunomodulatory protein or polypeptide may consist of an amino acid sequence at least 85% identical to the amino acid sequence of any one of SEQ ID NOS: 108-118 or 127-290. The amino acid sequence of the immunomodulatory protein or polypeptide may consist of an amino acid sequence at least 90% identical to the amino acid sequence of any one of SEQ ID NOS: 108-118 or 127-290. The amino acid sequence of the immunomodulatory protein or polypeptide may consist of an amino acid sequence at least 95% identical to the amino acid sequence of any one of SEQ ID NOS: 108-118 or 127-290. In some embodiments, the amino acid sequence of the immunomodulatory protein or polypeptide may preferably consist of an amino acid sequence 100% identical to the amino acid sequence of any one of SEQ ID NOS: 108-118 or 127-290.

In embodiments, the amino acid sequence of the immunomodulatory protein or polypeptide comprises or consists of the amino acid sequence of any one of SEQ ID NOS: 108-118 or 127-290, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-118 or 127-290, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-118 or 127-290, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-118 or 127-290, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 108-118 or 127-290, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 108-118 or 127-290, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 108-118 or 127-290, and further comprises or consists of about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 108-118 or 127-290, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-114 or 116-118. For example, the amino acid sequence of the immunomodulatory protein or polypeptide may comprise an amino acid sequence at least 85% identical to the amino acid sequence of any one of SEQ ID NOS: 108-114 or 116-118. The amino acid sequence of the immunomodulatory protein or polypeptide may comprise an amino acid sequence at least 90% identical to the amino acid sequence of any one of SEQ ID NOS: 108-114 or 116-118. The amino acid sequence of the immunomodulatory protein or polypeptide may comprise an amino acid sequence at least 95% identical to the amino acid sequence of any one of SEQ ID NOS: 108-114 or 116-118. In some embodiments, the amino acid sequence of the immunomodulatory protein or polypeptide may preferably comprise an amino acid sequence 100% identical to the amino acid sequence of any one of SEQ ID NOS: 108-114 or 116-118. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-114 or 116-118. For example, the amino acid sequence of the immunomodulatory protein or polypeptide may consist of an amino acid sequence at least 85% identical to the amino acid sequence of any one of SEQ ID NOS: 108-114 or 116-118. The amino acid sequence of the immunomodulatory protein or polypeptide may consist of an amino acid sequence at least 90% identical to the amino acid sequence of any one of SEQ ID NOS: 108-114 or 116-118. The amino acid sequence of the immunomodulatory protein or polypeptide may consist of an amino acid sequence at least 95% identical to the amino acid sequence of any one of SEQ ID NOS: 108-114 or 116-118. In some embodiments, the amino acid sequence of the immunomodulatory protein or polypeptide may preferably consist of an amino acid sequence 100% identical to the amino acid sequence of any one of SEQ ID NOS: 108-114 or 116-118.

In embodiments, the amino acid sequence of the immunomodulatory protein or polypeptide comprises or consists of the amino acid sequence of any one of SEQ ID NOS: 108-114 or 116-118, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-114 or 116-118, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-114 or 116-118, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108-114 or 116-118, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 108-114 or 116-118, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 108-114 or 116-118, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 108-114 or 116-118, and further comprises or consists of about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 108-114 or 116-118, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 119-126 or 291-454. For example, the amino acid sequence of the immunomodulatory protein or polypeptide may comprise an amino acid sequence at least 85% identical to the amino acid sequence of any one of SEQ ID NOS: 119-126 or 291-454. The amino acid sequence of the immunomodulatory protein or polypeptide may comprise an amino acid sequence at least 90% identical to the amino acid sequence of any one of SEQ ID NOS: 119-126 or 291-454. The amino acid sequence of the immunomodulatory protein or polypeptide may comprise an amino acid sequence at least 95% identical to the amino acid sequence of any one of SEQ ID NOS: 119-126 or 291-454. In some embodiments, the amino acid sequence of the immunomodulatory protein or polypeptide may preferably comprise an amino acid sequence 100% identical to the amino acid sequence of any one of SEQ ID NOS: 119-126 or 291-454. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 119-126 or 291-454. For example, the amino acid sequence of the immunomodulatory protein or polypeptide may consist of an amino acid sequence at least 85% identical to the amino acid sequence of any one of SEQ ID NOS: 119-126 or 291-454. The amino acid sequence of the immunomodulatory protein or polypeptide may consist of an amino acid sequence at least 90% identical to the amino acid sequence of any one of SEQ ID NOS: 119-126 or 291-454. The amino acid sequence of the immunomodulatory protein or polypeptide may consist of an amino acid sequence at least 95% identical to the amino acid sequence of any one of SEQ ID NOS: 119-126 or 291-454. In some embodiments, the amino acid sequence of the immunomodulatory protein or polypeptide may preferably consist of an amino acid sequence 100% identical to the amino acid sequence of any one of SEQ ID NOS: 119-126 or 291-454.

In embodiments, the amino acid sequence of the immunomodulatory protein or polypeptide comprises or consists of the amino acid sequence of any one of SEQ ID NOS: 119-126 or 291-454, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 119-126 or 291-454, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 119-126 or 291-454, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 119-126 or 291-454, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 119-126 or 291-454, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 119-126 or 291-454, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 119-126 or 291-454, and further comprises or consists of about 1, 2, 3, 4, 5, 6, 7, 8, 9, or 10 amino acid substitutions. In some embodiments, the amino acid sequence of the polypeptide comprises or consists of the amino acid sequence of any one of SEQ ID NOS: 119-126 or 291-454, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 119-125. For example, the amino acid sequence of the immunomodulatory protein or polypeptide may comprise an amino acid sequence at least 85% identical to the amino acid sequence of any one of SEQ ID NOS: 119-125. The amino acid sequence of the immunomodulatory protein or polypeptide may comprise an amino acid sequence at least 90% identical to the amino acid sequence of any one of SEQ ID NOS: 119-125. The amino acid sequence of the immunomodulatory protein or polypeptide may comprise an amino acid sequence at least 95% identical to the amino acid sequence of any one of SEQ ID NOS: 119-125. In some embodiments, the amino acid sequence of the immunomodulatory protein or polypeptide may preferably comprise an amino acid sequence 100% identical to the amino acid sequence of any one of SEQ ID NOS: 119-125. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 119-125. For example, the amino acid sequence of the immunomodulatory protein or polypeptide may consist of an amino acid sequence at least 85% identical to the amino acid sequence of any one of SEQ ID NOS: 119-125. The amino acid sequence of the immunomodulatory protein or polypeptide may consist of an amino acid sequence at least 90% identical to the amino acid sequence of any one of SEQ ID NOS: 119-125. The amino acid sequence of the immunomodulatory protein or polypeptide may consist of an amino acid sequence at least 95% identical to the amino acid sequence of any one of SEQ ID NOS: 119-125. In some embodiments, the amino acid sequence of the immunomodulatory protein or polypeptide may preferably consist of an amino acid sequence 100% identical to the amino acid sequence of any one of SEQ ID NOS: 119-125.

In embodiments, the amino acid sequence of the immunomodulatory protein or polypeptide comprises or consists of the amino acid sequence of any one of SEQ ID NOS: 119-125, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 119-125, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 119-125, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 119-125, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 119-125, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 119-125, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 119-125, and further comprises or consists of about 1, 2, 3, 4, 5, 6, 7, 8, 9, or 10 amino acid substitutions. In some embodiments, the amino acid sequence of the polypeptide comprises or consists of the amino acid sequence of any one of SEQ ID NOS: 119-125, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 109-111, 113, or 115. For example, the amino acid sequence of the immunomodulatory protein or polypeptide may comprise an amino acid sequence at least 85% identical to the amino acid sequence of any one of SEQ ID NOS: 109-111, 113, or 115. The amino acid sequence of the immunomodulatory protein or polypeptide may comprise an amino acid sequence at least 90% identical to the amino acid sequence of any one of SEQ ID NOS: 109-111, 113, or 115. The amino acid sequence of the immunomodulatory protein or polypeptide may comprise an amino acid sequence at least 95% identical to the amino acid sequence of any one of SEQ ID NOS: 109-111, 113, or 115. In some embodiments, the amino acid sequence of the immunomodulatory protein or polypeptide may preferably comprise an amino acid sequence 100% identical to the amino acid sequence of any one of SEQ ID NOS: 109-111, 113, or 115. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 109-111, 113, or 115. For example, the amino acid sequence of the immunomodulatory protein or polypeptide may consist of an amino acid sequence at least 85% identical to the amino acid sequence of any one of SEQ ID NOS: 109-111, 113, or 115. The amino acid sequence of the immunomodulatory protein or polypeptide may consist of an amino acid sequence at least 90% identical to the amino acid sequence of any one of SEQ ID NOS: 109-111, 113, or 115. The amino acid sequence of the immunomodulatory protein or polypeptide may consist of an amino acid sequence at least 95% identical to the amino acid sequence of any one of SEQ ID NOS: 109-111, 113, or 115. In some embodiments, the amino acid sequence of the immunomodulatory protein or polypeptide may preferably consist of an amino acid sequence 100% identical to the amino acid sequence of any one of SEQ ID NOS: 109-111, 113, or 115.

In embodiments, the amino acid sequence of the immunomodulatory protein or polypeptide comprises or consists of the amino acid sequence of any one of SEQ ID NOS: 109-111, 113, or 115, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 109-111, 113, or 115, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 109-111, 113, or 115, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 109-111, 113, or 115, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 109-111, 113, or 115, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 109-111, 113, or 115, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 109-111, 113, or 115, and further comprises or consists of about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 109-111, 113, or 115, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 109-111, or 113. For example, the amino acid sequence of the immunomodulatory protein or polypeptide may comprise an amino acid sequence at least 85% identical to the amino acid sequence of any one of SEQ ID NOS: 109-111, or 113. The amino acid sequence of the immunomodulatory protein or polypeptide may comprise an amino acid sequence at least 90% identical to the amino acid sequence of any one of SEQ ID NOS: 109-111, or 113. The amino acid sequence of the immunomodulatory protein or polypeptide may comprise an amino acid sequence at least 95% identical to the amino acid sequence of any one of SEQ ID NOS: 109-111, or 113. In some embodiments, the amino acid sequence of the immunomodulatory protein or polypeptide may preferably comprise an amino acid sequence 100% identical to the amino acid sequence of any one of SEQ ID NOS: 109-111, or 113. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 109-111, or 113. For example, the amino acid sequence of the immunomodulatory protein or polypeptide may consist of an amino acid sequence at least 85% identical to the amino acid sequence of any one of SEQ ID NOS: 109-111, or 113. The amino acid sequence of the immunomodulatory protein or polypeptide may consist of an amino acid sequence at least 90% identical to the amino acid sequence of any one of SEQ ID NOS: 109-111, or 113. The amino acid sequence of the immunomodulatory protein or polypeptide may consist of an amino acid sequence at least 95% identical to the amino acid sequence of any one of SEQ ID NOS: 109-111, or 113. In some embodiments, the amino acid sequence of the immunomodulatory protein or polypeptide may preferably consist of an amino acid sequence 100% identical to the amino acid sequence of any one of SEQ ID NOS: 109-111, or 113.

In embodiments, the amino acid sequence of the immunomodulatory protein or polypeptide comprises or consists of the amino acid sequence of any one of SEQ ID NOS: 109-111, or 113, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 109-111, or 113, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 109-111, or 113, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 109-111, or 113, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 109-111, or 113, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 109-111, or 113, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 109-111, or 113, and further comprises or consists of about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 109-111, or 113, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108, 112, 114, or 116-118. For example, the amino acid sequence of the immunomodulatory protein or polypeptide may comprise an amino acid sequence at least 85% identical to the amino acid sequence of any one of SEQ ID NOS: 108, 112, 114, or 116-118. The amino acid sequence of the immunomodulatory protein or polypeptide may comprise an amino acid sequence at least 90% identical to the amino acid sequence of any one of SEQ ID NOS: 108, 112, 114, or 116-118. The amino acid sequence of the immunomodulatory protein or polypeptide may comprise an amino acid sequence at least 95% identical to the amino acid sequence of any one of SEQ ID NOS: 108, 112, 114, or 116-118. In some embodiments the amino acid sequence of the immunomodulatory protein or polypeptide may preferably comprise an amino acid sequence 100% identical to the amino acid sequence of any one of SEQ ID NOS: 108, 112, 114, or 116-118. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108, 112, 114, or 116-118. For example, the amino acid sequence of the immunomodulatory protein or polypeptide may consist of an amino acid sequence at least 85% identical to the amino acid sequence of any one of SEQ ID NOS: 108, 112, 114, or 116-118. The amino acid sequence of the immunomodulatory protein or polypeptide may consist of an amino acid sequence at least 90% identical to the amino acid sequence of any one of SEQ ID NOS: 108, 112, 114, or 116-118. The amino acid sequence of the immunomodulatory protein or polypeptide may consist of an amino acid sequence at least 95% identical to the amino acid sequence of any one of SEQ ID NOS: 108, 112, 114, or 116-118. In some embodiments, the amino acid sequence of the immunomodulatory protein or polypeptide may preferably consist of an amino acid sequence 100% identical to the amino acid sequence of any one of SEQ ID NOS: 108, 112, 114, or 116-118.

In embodiments, the amino acid sequence of the immunomodulatory protein or polypeptide comprises or consists of the amino acid sequence of any one of SEQ ID NOS: 108, 112, 114, or 116-118, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108, 112, 114, or 116-118, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108, 112, 114, or 116-118, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 108, 112, 114, or 116-118, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 108, 112, 114, or 116-118, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 108, 112, 114, or 116-118, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 108, 112, 114, or 116-118, and further comprises or consists of about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 108, 112, 114, or 116-118, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 120-122, 124, or 126. For example, the amino acid sequence of the immunomodulatory protein or polypeptide may comprise an amino acid sequence at least 85% identical to the amino acid sequence of any one of SEQ ID NOS: 120-122, 124, or 126. The amino acid sequence of the immunomodulatory protein or polypeptide may comprise an amino acid sequence at least 90% identical to the amino acid sequence of any one of SEQ ID NOS: 120-122, 124, or 126. The amino acid sequence of the immunomodulatory protein or polypeptide may comprise an amino acid sequence at least 95% identical to the amino acid sequence of any one of SEQ ID NOS: 120-122, 124, or 126. In some embodiments, the amino acid sequence of the immunomodulatory protein or polypeptide may preferably comprise an amino acid sequence 100% identical to the amino acid sequence of any one of SEQ ID NOS: 120-122, 124, or 126. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 120-122, 124, or 126. For example, the amino acid sequence of the immunomodulatory protein or polypeptide may consist of an amino acid sequence at least 85% identical to the amino acid sequence of any one of SEQ ID NOS: 120-122, 124, or 126. The amino acid sequence of the immunomodulatory protein or polypeptide may consist of an amino acid sequence at least 90% identical to the amino acid sequence of any one of SEQ ID NOS: 120-122, 124, or 126. The amino acid sequence of the immunomodulatory protein or polypeptide may consist of an amino acid sequence at least 95% identical to the amino acid sequence of any one of SEQ ID NOS: 120-122, 124, or 126. In some embodiments, the amino acid sequence of the immunomodulatory protein or polypeptide may preferably consist of an amino acid sequence 100% identical to the amino acid sequence of any one of SEQ ID NOS: 120-122, 124, or 126.

In embodiments, the amino acid sequence of the immunomodulatory protein or polypeptide comprises or consists of the amino acid sequence of any one of SEQ ID NOS: 120-122, 124, or 126, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 120-122, 124, or 126, and further comprises or consists of at least about 1, 2, 3, 4, 5, 6, 7, 8, 9, or 10 amino acid variations (e.g., substitutions, additions, deletions, etc.). In some embodiments, the amino acid sequence of immunomodulatory protein or polypeptide comprises or consists of the amino acid sequence of any one of SEQ ID NOS: 120-122, 124, or 126, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 120-122, 124, or 126, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 120-122, 124, or 126, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 120-122, 124, or 126, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 120-122, 124, or 126, and further comprises or consists of about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 120-122, 124, or 126, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 120-122, or 124. For example, the amino acid sequence of the immunomodulatory protein or polypeptide may comprise an amino acid sequence at least 85% identical to the amino acid sequence of any one of SEQ ID NOS: 120-122, or 124. The amino acid sequence of the immunomodulatory protein or polypeptide may comprise an amino acid sequence at least 90% identical to the amino acid sequence of any one of SEQ ID NOS: 120-122, or 124. The amino acid sequence of the immunomodulatory protein or polypeptide may comprise an amino acid sequence at least 95% identical to the amino acid sequence of any one of SEQ ID NOS: 120-122, or 124. In some embodiments, the amino acid sequence of the immunomodulatory protein or polypeptide may comprise an amino acid sequence 100% identical to the amino acid sequence of any one of SEQ ID NOS: 120-122, or 124. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 120-122, or 124. For example, the amino acid sequence of the immunomodulatory protein or polypeptide may consist of an amino acid sequence at least 85% identical to the amino acid sequence of any one of SEQ ID NOS: 120-122, or 124. The amino acid sequence of the immunomodulatory protein or polypeptide may consist of an amino acid sequence at least 90% identical to the amino acid sequence of any one of SEQ ID NOS: 120-122, or 124. The amino acid sequence of the immunomodulatory protein or polypeptide may consist of an amino acid sequence at least 95% identical to the amino acid sequence of any one of SEQ ID NOS: 120-122, or 124. In some embodiments, the amino acid sequence of the immunomodulatory protein or polypeptide may preferably consist of an amino acid sequence 100% identical to the amino acid sequence of any one of SEQ ID NOS: 120-122, or 124.

In embodiments, the amino acid sequence of the immunomodulatory protein or polypeptide comprises or consists of the amino acid sequence of any one of SEQ ID NOS: 120-122, or 124, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 120-122, or 124, and further comprises or consists of at least about 1, 2, 3, 4, 5, 6, 7, 8, 9, or 10 amino acid variations (e.g., substitutions, additions, deletions, etc.). In some embodiments, the amino acid sequence of immunomodulatory protein or polypeptide comprises or consists of the amino acid sequence of any one of SEQ ID NOS: 120-122, or 124, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 120-122, or 124, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 120-122, or 124, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 120-122, or 124, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 120-122, or 124, and further comprises or consists of about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 120-122, or 124, and further comprises or consists of no more than about 1, 2, 3, 4, 5, 6, 7, 8, 9, or 10 amino acid substitutions.

In some embodiments, the amino acid sequence of the immunomodulatory protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 119, 123 or 125. For example, the amino acid sequence of the immunomodulatory protein or polypeptide may comprise an amino acid sequence at least 85% identical to the amino acid sequence of any one of SEQ ID NOS: 119, 123 or 125. The amino acid sequence of the immunomodulatory protein or polypeptide may comprise an amino acid sequence at least 90% identical to the amino acid sequence of any one of SEQ ID NOS: 119, 123 or 125. The amino acid sequence of the immunomodulatory protein or polypeptide may comprise an amino acid sequence at least 95% identical to the amino acid sequence of any one of SEQ ID NOS: 119, 123 or 125. In some embodiments, the amino acid sequence of the immunomodulatory protein or polypeptide may preferably comprise an amino acid sequence 100% identical to the amino acid sequence of any one of SEQ ID NOS: 119, 123 or 125. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 119, 123 or 125. For example, the amino acid sequence of the immunomodulatory protein or polypeptide may consist of an amino acid sequence at least 85% identical to the amino acid sequence of any one of SEQ ID NOS: 119, 123 or 125. The amino acid sequence of the immunomodulatory protein or polypeptide may consist of an amino acid sequence at least 90% identical to the amino acid sequence of any one of SEQ ID NOS: 119, 123 or 125. The amino acid sequence of the immunomodulatory protein or polypeptide may consist of an amino acid sequence at least 95% identical to the amino acid sequence of any one of SEQ ID NOS: 119, 123 or 125. In some embodiments, the amino acid sequence of the immunomodulatory protein or polypeptide may preferably consist of an amino acid sequence 100% identical to the amino acid sequence of any one of SEQ ID NOS: 119, 123 or 125.

In embodiments, the amino acid sequence of the immunomodulatory protein or polypeptide comprises or consists of the amino acid sequence of any one of SEQ ID NOS: 119, 123 or 125, and further comprises 1 or more but less than 15% (less than 12%, less than 10%, less than 8%), amino acid variations (e.g., substitutions, additions, deletions, etc.). In some embodiments, the amino acid sequence of the immunomodulatory protein or polypeptide comprises or consists of the amino acid sequence of any one of SEQ ID NOS: 119, 123 or 125, and further comprises or consists of at least about 1, 2, 3, 4, 5, 6, 7, 8, 9, or 10 amino acid variations (e.g., substitutions, additions, deletions, etc.). In some embodiments, the amino acid sequence of immunomodulatory protein or polypeptide comprises or consists of the amino acid sequence of any one of SEQ ID NOS: 119, 123 or 125, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory protein or polypeptide comprises or consists of the amino acid sequence of any one of SEQ ID NOS: 119, 123 or 125, and further comprises or consists of no more than about 1, 2, 3, 4, 5, 6, 7, 8, 9, or 10 amino acid variations (e.g., substitutions, additions, deletions, etc.).

In embodiments, the amino acid sequence of the immunomodulatory protein or polypeptide comprises or consists of the amino acid sequence of any one of SEQ ID NOS: 119, 123 or 125, and further comprises 1 or more but less than 15% (less than 12%, less than 10%, less than 8%), amino acid substitutions. In some embodiments, the amino acid sequence of the immunomodulatory protein or polypeptide comprises or consists of the amino acid sequence of any one of SEQ ID NOS: 119, 123 or 125, and further comprises or consists of at least about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 119, 123 or 125, and further comprises or consists of about 1, 2, 3, 4, 5, 6, 7, 8, 9, or 10 amino acid substitutions. In some embodiments, the amino acid sequence of the immunomodulatory protein or polypeptide comprises or consists of the amino acid sequence of any one of SEQ ID NOS: 119, 123 or 125, and further comprises or consists of no more than about 1, 2, 3, 4, 5, 6, 7, 8, 9, or 10 amino acid substitutions.

In some embodiments, of the immunomodulatory proteins or polypeptides referred to herein, SEQ ID NOS: 108, 111-119 or 122-126 may be preferred. Advantageously, said immunomodulatory proteins or polypeptides may engage hIL-10R with a higher potency than hIL-10. Thus, in some embodiments, an immunomodulatory protein or polypeptide may comprise an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108, 111-119 or 122-126. For example, in some embodiments, the amino acid sequence of the immunomodulatory protein or polypeptide may comprise an amino acid sequence at least 85% identical to the amino acid sequence of any one of SEQ ID NOS: 108, 111-119 or 122-126. In some embodiments, the amino acid sequence of the immunomodulatory protein or polypeptide may comprise an amino acid sequence at least 90% identical to the amino acid sequence of any one of SEQ ID NOS: 108, 111-119 or 122-126. In some embodiments, the amino acid sequence of the immunomodulatory protein or polypeptide may comprise an amino acid sequence at least 95% identical to the amino acid sequence of any one of SEQ ID NOS: 108, 111-119 or 122-126. In some embodiments, the amino acid sequence of the immunomodulatory protein or polypeptide may preferably comprise an amino acid sequence 100% identical to the amino acid sequence of any one of SEQ ID NOS: 108, 111-119 or 122-126. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108, 111-119 or 122-126. For example, in some embodiments, the amino acid sequence of the immunomodulatory protein or polypeptide may consist of an amino acid sequence at least 85% identical to the amino acid sequence of any one of SEQ ID NOS: 108, 111-119 or 122-126. In some embodiments, the amino acid sequence of the immunomodulatory protein or polypeptide may consist of an amino acid sequence at least 90% identical to the amino acid sequence of any one of SEQ ID NOS: 108, 111-119 or 122-126. In some embodiments, the amino acid sequence of the immunomodulatory protein or polypeptide may consist of an amino acid sequence at least 95% identical to the amino acid sequence of any one of SEQ ID NOS: 108, 111-119 or 122-126. In some embodiments, the amino acid sequence of the immunomodulatory protein or polypeptide may preferably consist of an amino acid sequence 100% identical to the amino acid sequence of any one of SEQ ID NOS: 108, 111-119 or 122-126.

In some embodiments, the immunomodulatory proteins or polypeptides referred to herein, SEQ ID NOS: 112-115 or 123-126 may be more preferred. Advantageously, said immunomodulatory proteins or polypeptides may be particularly effective at suppressing the production of proinflammatory cytokines, e.g., one or more of IFN-γ, IL-1β, IL-6, IL-8, TNF-α, and IL-4. For example, said immunomodulatory proteins or polypeptides may suppress the production of proinflammatory cytokines more effectively than hIL-10. This is preferably in addition to engaging hIL-10R with a higher potency than hIL-10. Thus, in some embodiments, an immunomodulatory protein or polypeptide may comprise an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 112-115 or 123-126. For example, in some embodiments, the amino acid sequence of the immunomodulatory protein or polypeptide may comprise an amino acid sequence at least 85% identical to the amino acid sequence of any one of SEQ ID NOS: 112-115 or 123-126. In some embodiments, the amino acid sequence of the immunomodulatory protein or polypeptide may comprise an amino acid sequence at least 90% identical to the amino acid sequence of any one of SEQ ID NOS: 112-115 or 123-126. In some embodiments, the amino acid sequence of the immunomodulatory protein or polypeptide may comprise an amino acid sequence at least 95% identical to the amino acid sequence of any one of SEQ ID NOS: 112-115 or 123-126. In some embodiments, the amino acid sequence of the immunomodulatory protein or polypeptide may comprise an amino acid sequence 100% identical to the amino acid sequence of any one of SEQ ID NOS: 112-115 or 123-126. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 112-115 or 123-126. For example, in some embodiments, the amino acid sequence of the immunomodulatory protein or polypeptide may consist of an amino acid sequence at least 85% identical to the amino acid sequence of any one of SEQ ID NOS: 112-115 or 123-126. In some embodiments, the amino acid sequence of the immunomodulatory protein or polypeptide may consist of an amino acid sequence at least 90% identical to the amino acid sequence of any one of SEQ ID NOS: 112-115 or 123-126. In some embodiments, the amino acid sequence of the immunomodulatory protein or polypeptide may consist of an amino acid sequence at least 95% identical to the amino acid sequence of any one of SEQ ID NOS: 112-115 or 123-126. In some embodiments, the amino acid sequence of the immunomodulatory protein or polypeptide may consist of an amino acid sequence 100% identical to the amino acid sequence of any one of SEQ ID NOS: 112-115 or 123-126.

In some embodiments, of the immunomodulatory proteins or polypeptides referred to herein, SEQ ID NO: 114 or 125 may be preferred. Advantageously, said immunomodulatory proteins or polypeptides may engage hIL-10R with a higher potency than hIL-10. Thus, in some embodiments, an immunomodulatory protein or polypeptide may comprise an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 114 or 125. For example, in some embodiments, the amino acid sequence of the immunomodulatory protein or polypeptide may comprise an amino acid sequence at least 85% identical to the amino acid sequence of any one of SEQ ID NO: 114 or 125. In some embodiments, the amino acid sequence of the immunomodulatory protein or polypeptide may comprise an amino acid sequence at least 90% identical to the amino acid sequence of any one of SEQ ID NO: 114 or 125. In some embodiments, the amino acid sequence of the immunomodulatory protein or polypeptide may comprise an amino acid sequence at least 95% identical to the amino acid sequence of any one of SEQ ID NO: 114 or 125. In some embodiments, the amino acid sequence of the immunomodulatory protein or polypeptide may preferably comprise an amino acid sequence 100% identical to the amino acid sequence of any one of SEQ ID NO: 114 or 125. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 114 or 125. For example, in some embodiments, the amino acid sequence of the immunomodulatory protein or polypeptide may consist of an amino acid sequence at least 85% identical to the amino acid sequence of any one of SEQ ID NO: 114 or 125. In some embodiments, the amino acid sequence of the immunomodulatory protein or polypeptide may consist of an amino acid sequence at least 90% identical to the amino acid sequence of any one of SEQ ID NO: 114 or 125. In some embodiments, the amino acid sequence of the immunomodulatory protein or polypeptide may consist of an amino acid sequence at least 95% identical to the amino acid sequence of any one of SEQ ID NO: 114 or 125. In some embodiments, the amino acid sequence of the immunomodulatory protein or polypeptide may preferably consist of an amino acid sequence 100% identical to the amino acid sequence of any one of SEQ ID NO: 114 or 125.

In some embodiments, of the immunomodulatory proteins or polypeptides referred to herein, SEQ ID NO: 114 may be preferred. Advantageously, said immunomodulatory proteins or polypeptides may engage hIL-10R with a higher potency than hIL-10. Thus, in some embodiments, an immunomodulatory protein or polypeptide may comprise an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 114. For example, in some embodiments, the amino acid sequence of the immunomodulatory protein or polypeptide may comprise an amino acid sequence at least 85% identical to the amino acid sequence of any one of SEQ ID NO: 114. In some embodiments, the amino acid sequence of the immunomodulatory protein or polypeptide may comprise an amino acid sequence at least 90% identical to the amino acid sequence of any one of SEQ ID NO: 114. In some embodiments, the amino acid sequence of the immunomodulatory protein or polypeptide may comprise an amino acid sequence at least 95% identical to the amino acid sequence of any one of SEQ ID NO: 114. In some embodiments, the amino acid sequence of the immunomodulatory protein or polypeptide may preferably comprise an amino acid sequence 100% identical to the amino acid sequence of any one of SEQ ID NO: 114. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 114. For example, in some embodiments, the amino acid sequence of the immunomodulatory protein or polypeptide may consist of an amino acid sequence at least 85% identical to the amino acid sequence of any one of SEQ ID NO: 114. In some embodiments, the amino acid sequence of the immunomodulatory protein or polypeptide may consist of an amino acid sequence at least 90% identical to the amino acid sequence of any one of SEQ ID NO: 114. In some embodiments, the amino acid sequence of the immunomodulatory protein or polypeptide may consist of an amino acid sequence at least 95% identical to the amino acid sequence of any one of SEQ ID NO: 114. In some embodiments, the amino acid sequence of the immunomodulatory protein or polypeptide may preferably consist of an amino acid sequence 100% identical to the amino acid sequence of any one of SEQ ID NO: 114.

In some embodiments, of the immunomodulatory proteins or polypeptides referred to herein, SEQ ID NO: 125 may be preferred. Advantageously, said immunomodulatory proteins or polypeptides may engage hIL-10R with a higher potency than hIL-10. Thus, in some embodiments, an immunomodulatory protein or polypeptide may comprise an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 125. For example, in some embodiments, the amino acid sequence of the immunomodulatory protein or polypeptide may comprise an amino acid sequence at least 85% identical to the amino acid sequence of any one of SEQ ID NO: 125. In some embodiments, the amino acid sequence of the immunomodulatory protein or polypeptide may comprise an amino acid sequence at least 90% identical to the amino acid sequence of any one of SEQ ID NO: 125. In some embodiments, the amino acid sequence of the immunomodulatory protein or polypeptide may comprise an amino acid sequence at least 95% identical to the amino acid sequence of any one of SEQ ID NO: 125. In some embodiments, the amino acid sequence of the immunomodulatory protein or polypeptide may preferably comprise an amino acid sequence 100% identical to the amino acid sequence of any one of SEQ ID NO: 125. In some embodiments, the amino acid sequence of the immunomodulatory protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NO: 125. For example, in some embodiments, the amino acid sequence of the immunomodulatory protein or polypeptide may consist of an amino acid sequence at least 85% identical to the amino acid sequence of any one of SEQ ID NO: 125. In some embodiments, the amino acid sequence of the immunomodulatory protein or polypeptide may consist of an amino acid sequence at least 90% identical to the amino acid sequence of any one of SEQ ID NO: 125. In some embodiments, the amino acid sequence of the immunomodulatory protein or polypeptide may consist of an amino acid sequence at least 95% identical to the amino acid sequence of any one of SEQ ID NO: 125. In some embodiments, the amino acid sequence of the immunomodulatory protein or polypeptide may preferably consist of an amino acid sequence 100% identical to the amino acid sequence of any one of SEQ ID NO: 125.

### 5.2.1 Signal Peptides

In some embodiments, the immunomodulatory protein or polypeptide comprises a homologous or heterologous signal peptide operably connected to the N-terminus of said immunomodulatory protein or polypeptide. In some embodiments, the immunomodulatory protein or polypeptide comprises an amino acid sequence set forth in any one of SEQ ID NOS: 108-454 and comprises a homologous signal peptide operably connected to the N-terminus of said polypeptide. In some embodiments, the immunomodulatory protein or polypeptide comprises an amino acid sequence set forth in any one of SEQ ID NOS: 108-454 and comprises a heterologous signal peptide operably connected to the N-terminus of said polypeptide. In some embodiments, the immunomodulatory protein or polypeptide comprises an amino acid sequence set forth in any one of SEQ ID NOS: 119-126 or 291-454 and comprises a homologous signal peptide operably connected to the N-terminus of said polypeptide. In some embodiments, the immunomodulatory protein or polypeptide comprises an amino acid sequence set forth in any one of SEQ ID NOS: 119-126 or 291-454 and comprises a heterologous signal peptide operably connected to the N-terminus of said polypeptide. In some embodiments, the immunomodulatory protein or polypeptide comprises an amino acid sequence set forth in any one of SEQ ID NOS: 108-118 or 127-290 and comprises a homologous signal peptide operably connected to the N-terminus of said polypeptide. In some embodiments, the immunomodulatory protein or polypeptide comprises an amino acid sequence set forth in any one of SEQ ID NOS: 108-118 or 127-290 and comprises a heterologous signal peptide operably connected to the N-terminus of said polypeptide.

In some embodiments, the immunomodulatory protein or polypeptide comprises an amino acid sequence set forth in any one of SEQ ID NO: 114 or 125 and comprises a homologous signal peptide operably connected to the N-terminus of said polypeptide. In some embodiments, the immunomodulatory protein or polypeptide comprises an amino acid sequence set forth in any one of SEQ ID NO: 114 or 125 and comprises a heterologous signal peptide operably connected to the N-terminus of said polypeptide.

Commonly used signal peptides are known in the art, for example, the native signal peptide of human interleukin 2 (hIL-2), human oncostatin M (hOSM), human chymotrypsinogen (hCTRB1), human trypsinogen 2 (hTRY2), and human insulin (hINS). A person of ordinary skill can determine the appropriate signal peptide using standard methodology known in the art. The amino acid sequence of exemplary signal peptides is provided in Table 3.

TABLE 3
The amino acid sequence of exemplary
signal peptides

| | | SEQ ID |
|---|---|---|
| Description | Amino Acid Sequence | NO |
| hIL-2 | MYRMQLLSCIALSLALVINS | 7 |
| hOSM | MGVLLTQRTLLSLVLALLFPSMASM | 8 |
| hCTRB1 | MASLWLLSCFSLVGAAFG | 9 |
| hTRY2 | MNLLLILTFVAAAVA | 10 |
| hINS | MALWMRLLPLLALLALWGPDPAAA | 11 |

In some embodiments, the amino acid sequence of the signal peptide comprises or consists of the amino acid sequence of any one of the signal peptides set forth in Table 3. In some embodiments, the amino acid sequence of the signal peptide comprises or consists of the amino acid sequence of any one of the signal peptides set forth in Table 3, and further comprises 1 or more but less than 15% (less than 12%, less than 10%, less than 8%), amino acid variations (e.g., amino acid substitutions, deletions, or additions). In some embodiments, the amino acid sequence of the signal peptide comprises or consists of the amino acid sequence of any one of the signal peptides set forth in Table 3, comprising 1, 2, or 3 amino acid variations (e.g., substitutions, deletions, additions). In some embodiments, the amino acid sequence of the signal peptide comprises or consists of the amino acid sequence of any one of the signal peptides set forth in Table 3, and further comprises 1 or more but less than 15% (less than 12%, less than 10%, less than 8%), amino acid substitutions. In some embodiments, the amino acid sequence of the signal peptide comprises or consists of the amino acid sequence of any one of the signal peptides set forth in Table 3, comprising 1, 2, or 3 amino acid substitutions.

In some embodiments, the amino acid sequence of the signal peptide comprises or consists of the amino acid sequence of any one of SEQ ID NOS: 7-11. In some embodiments, the amino acid sequence of the signal peptide comprises or consists of the amino acid sequence of any one of SEQ ID NOS: 7-11, and further comprises 1 or more but less than 15% (less than 12%, less than 10%, less than 8%), amino acid variations (e.g., amino acid substitutions, deletions, or additions). In some embodiments, the amino acid sequence of the signal peptide comprises or consists of the amino acid sequence of any one of SEQ ID NOS: 7-11, comprising 1, 2, or 3 amino acid variations (e.g., substitutions, deletions, additions). In some embodiments, the amino acid sequence of the signal peptide comprises or consists of the amino acid sequence of any one of SEQ ID NOS: 7-11, and further comprises 1 or more but less than 15% (less than 12%, less than 10%, less than 8%), amino acid substitutions. In some embodiments, the amino acid sequence of the signal peptide comprises or consists of the amino acid sequence of any one of SEQ ID NOS: 7-11, comprising 1, 2, or 3 amino acid substitutions.

### 5.2.2 Potency & Affinity of Immunomodulatory Proteins

In some embodiments, the immunomodulatory protein or polypeptide (or a fusion or conjugate described herein (e.g., an immunomodulatory fusion protein or polypeptide described herein)) increases the level of STAT3 in cells expressing the hIL-10R on the surface relative to the level of STAT3 in the absence of the immunomodulatory protein or polypeptide (or the fusion or conjugate (e.g., the immunomodulatory fusion protein or polypeptide)) or relative to the level of STAT3 level in the presence of a suitable control (e.g., a reference hIL-10 protein or polypeptide (e.g., SEQ ID NO: 1 or 2) (or a reference fusion or conjugate (e.g., a reference immunomodulatory fusion protein or polypeptide (e.g., any one of SEQ ID NOS: 102-105))). In some embodiments, the immunomodulatory protein or polypeptide (or a fusion or conjugate described herein (e.g., an immunomodulatory fusion protein or polypeptide described herein)) increases the level of phosphorylated STAT3 in cells expressing the hIL-10R on the surface relative in the absence of the immunomodulatory protein or polypeptide (or the fusion or conjugate (e.g., the immunomodulatory fusion protein or polypeptide)) or relative to the level of phosphorylated STAT3 the presence of a suitable control (e.g., a reference hIL-10 protein or polypeptide (e.g., SEQ ID NO: 1 or 2)) (or a reference fusion or conjugate (e.g., a reference immunomodulatory fusion protein or polypeptide (e.g., any one of SEQ ID NOS: 102-105))).

In some embodiments, the immunomodulatory protein or polypeptide (or a fusion or conjugate described herein (e.g., an immunomodulatory fusion protein or polypeptide described herein)) increases the level of phosphorylated STAT3 in cells expressing the hIL-10R on the surface with an EC50 of less than about 500 μM, 400 μM, 300 μM, 200 μM, 100 μM, 50 μM, 40 μM, 30 μM, 20 μM, 10 μM, 9 μM, 8 μM, 7 μM, 6 μM, 5 μM, 4 μM, 3 μM, 2 μM, 1 μM, 0.9 μM, 0.8 μM, 0.7 μM, 0.6 μM, 0.5 μM, 0.4 μM, 0.3 μM, 0.2 μM, or 0.1 μM. In some embodiments, the immunomodulatory protein or polypeptide (or a fusion or conjugate described herein (e.g., an immunomodulatory fusion protein or polypeptide described herein)) preferably increases the level of phosphorylated STAT3 in cells expressing the hIL-10R on the surface with an EC50 of less than about 25 μM, such as less than about 10 μM. In some embodiments, the immunomodulatory protein or polypeptide (or a fusion or conjugate described herein (e.g., an immunomodulatory fusion protein or polypeptide described herein)) increases the level of phosphorylated STAT3 cells expressing the hIL-10R on the surface with an EC50 of from about 500 μM-0.1 μM, 400 μM-0.1 μM, 300 μM-0.1 μM, 200 μM-0.1 μM, 100 μM-0.1 μM, 50 μM-0.1 μM, 25 μM-0.1 μM, 10 μM-0.1 μM, 5 μM-0.1 μM, or 1 μM-0.1 μM, 500 μM-0.5, 400 μM-0.5, 300 μM-0.5, 200 μM-0.5, 100 μM-0.5, 50 μM-0.5, 25 μM-0.5, 10 μM-0.5, 5 μM-0.5, or 1 μM-0.5 μM. In some embodiments, the immunomodulatory protein or polypeptide (or a fusion or conjugate described herein (e.g., an immunomodulatory fusion protein or polypeptide described herein)) increases the level of phosphorylated STAT3 in cells expressing the hIL-10R on the surface with an EC50 of no greater than about 0.1 μM, 0.2 μM, 0.3 μM, 0.4 μM, 0.5 μM, 0.6 μM, 0.7 pm, 0.8 μM, 0.9 μM, 1.0 μM, 5 μM, 10 μM, 20 μM, 30 μM, 40 μM, 50 μM, 60 μM, 70 μM, 80 μM, 90 μM, 100 μM, 200 μM, 300 μM, 400 μM, or 500 μM.

In some embodiments, the immunomodulatory protein or polypeptide (or a fusion or conjugate described herein (e.g., an immunomodulatory fusion protein or polypeptide described herein)) increases the level of phosphorylated STAT3 in cells expressing the hIL-10R on the surface with an EC50 that is at least about 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 140-fold, or 150-fold higher than that of a reference hIL-10 protein or polypeptide (e.g., a reference hIL-10 protein or polypeptide comprising the amino acid sequence set forth in SEQ ID NO: 1 or 2) (or a reference fusion or conjugate (e.g., a reference immunomodulatory fusion protein or polypeptide (e.g., a reference immunomodulatory fusion protein or polypeptide comprising the amino acid sequence set forth in any one of SEQ ID NOS: 102-105))). In some embodiments, the immunomodulatory protein or polypeptide (or a fusion or conjugate described herein (e.g., an immunomodulatory fusion protein or polypeptide described herein)) increases the level of phosphorylated STAT3 in cells expressing the hIL-10R on the surface with an EC50 that is from about 4-150-fold, 10-150-fold, 20-150-fold, 30-150-fold, 40-150-fold, 50-150-fold, 60-150-fold, 70-150-fold, 80-150-fold, 90-150-fold, 100-150-fold, 110-150-fold, 120-150-fold, 130-150-fold, or 140-150-fold, higher than that of a reference hIL-10 protein or polypeptide (e.g., a reference hIL-10 protein or polypeptide comprising the amino acid sequence set forth in SEQ ID NO: 1 or 2) (or a reference fusion or conjugate (e.g., a reference immunomodulatory fusion protein or polypeptide (e.g., any one of SEQ ID NOS: 102-105))).

In some embodiments, the immunomodulatory protein or polypeptide (or a fusion or conjugate described herein (e.g., an immunomodulatory fusion protein or polypeptide described herein)) binds to a cell expressing the hIL-10R on the surface with an EC50 of less than about 500 μM, 400 μM, 300 μM, 200 μM, 100 μM, 50 μM, 40 μM, 30 μM, 20 μM, 10 μM, 9 μM, 8 μM, 7 μM, 6 μM, 5 μM, 4 μM, 3 μM, 2 μM, 1 μM, 0.9 μM, 0.8 μM, 0.7 μM, 0.6 μM, 0.5 μM, 0.4 μM, 0.3 μM, 0.2 μM, or 0.1 μM. In some embodiments, the immunomodulatory protein or polypeptide (or a fusion or conjugate described herein (e.g., an immunomodulatory fusion protein or polypeptide described herein)) preferably binds to a cell expressing the hIL-10R on the surface with an EC50 of less than about 25 μM, such as less than about 10 μM. In some embodiments, the immunomodulatory protein or polypeptide (or a fusion or conjugate described herein (e.g., an immunomodulatory fusion protein or polypeptide described herein)) binds to a cell expressing the hIL-10R on the surface with an EC50 of from about 500 μM-0.1 μM, 400 μM-0.1 μM, 300 μM-0.1 μM, 200 μM-0.1 μM, 100 μM-0.1 μM, 50 μM-0.1 μM, 25 μM-0.1 μM, 10 μM-0.1 μM, 5 μM-0.1 μM, or 1 μM-0.1 μM, 500 μM-0.5, 400 μM-0.5, 300 μM-0.5, 200 μM-0.5, 100 μM-0.5, 50 μM-0.5, 25 μM-0.5, 10 μM-0.5, 5 μM-0.5, or 1 μM-0.5 μM. In some embodiments, the immunomodulatory protein or polypeptide (or a fusion or conjugate described herein (e.g., an immunomodulatory fusion protein or polypeptide described herein)) binds to a cell expressing the hIL-10R on the surface with an EC50 of no greater than about 0.1 μM, 0.2 μM, 0.3 μM, 0.4 μM, 0.5 μM, 0.6 μM, 0.7 pm, 0.8 μM, 0.9 μM, 1.0 μM, 5 μM, 10 μM, 20 μM, 30 μM, 40 μM, 50 μM, 60 μM, 70 μM, 80 μM, 90 μM, 100 μM, 200 μM, 300 μM, 400 μM, or 500 μM.

In some embodiments, the immunomodulatory protein or polypeptide (or a fusion or conjugate described herein (e.g., an immunomodulatory fusion protein or polypeptide described herein)) binds to a cell expressing the hIL-10R on the surface with an EC50 that is at least about 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 140-fold, or 150-fold higher than that of a reference hIL-10 protein or polypeptide (e.g., a reference hIL-10 protein or polypeptide comprising the amino acid sequence set forth in SEQ ID NO: 1 or 2) (or a reference fusion or conjugate (e.g., a reference immunomodulatory fusion protein or polypeptide (e.g., a reference immunomodulatory fusion protein or polypeptide (e.g., any one of SEQ ID NOS: 102-105))). In some embodiments, the immunomodulatory protein or polypeptide (or a fusion or conjugate described herein (e.g., an immunomodulatory fusion protein or polypeptide described herein)) binds to a cell expressing the hIL-10R on the surface with an EC50 that is from about 10-150-fold, 20-150-fold, 30-150-fold, 40-150-fold, 50-150-fold, 60-150-fold, 70-150-fold, 80-150-fold, 90-150-fold, 100-150-fold, 110-150-fold, 120-150-fold, 130-150-fold, or 140-150-fold, higher than that of a reference hIL-10 protein or polypeptide (e.g., a reference hIL-10 protein or polypeptide comprising the amino acid sequence set forth in SEQ ID NO: 1 or 2) (or a reference fusion or conjugate (e.g., a reference immunomodulatory fusion protein or polypeptide (e.g., a reference immunomodulatory fusion protein or polypeptide (e.g., any one of SEQ ID NOS: 102-105))).

In some embodiments, the immunomodulatory protein or polypeptide (or a fusion or conjugate described herein (e.g., an immunomodulatory fusion protein or polypeptide described herein)) binds to a cell expressing hIL-10Rα on the surface with an EC50 of less than about 500 μM, 400 μM, 300 μM, 200 μM, 100 μM, 50 μM, 40 μM, 30 μM, 20 μM, 10 μM, 9 μM, 8 μM, 7 μM, 6 μM, 5 μM, 4 μM, 3 μM, 2 μM, 1 μM, 0.9 μM, 0.8 μM, 0.7 μM, 0.6 μM, 0.5 μM, 0.4 μM, 0.3 μM, 0.2 μM, or 0.1 μM. In some embodiments, the immunomodulatory protein or polypeptide (or a fusion or conjugate described herein (e.g., an immunomodulatory fusion protein or polypeptide described herein)) preferably binds to a cell expressing hIL-10Rα on the surface with an EC50 of less than about 25 pM, such as less than about 10 μM. In some embodiments, the immunomodulatory protein or polypeptide (or a fusion or conjugate described herein (e.g., an immunomodulatory fusion protein or polypeptide described herein)) binds to a cell expressing hIL-10Rα on the surface with an EC50 of from about 500 μM-0.1 μM, 400 μM-0.1 μM, 300 μM-0.1 μM, 200 μM-0.1 μM, 100 μM-0.1 μM, 50 μM-0.1 μM, 25 μM-0.1 μM, 10 μM-0.1 μM, 5 μM-0.1 μM, or 1 μM-0.1 μM, 500 μM-0.5, 400 μM-0.5, 300 μM-0.5, 200 μM-0.5, 100 μM-0.5, 50 μM-0.5, 25 μM-0.5, 10 μM-0.5, 5 μM-0.5, or 1 μM-0.5 μM. In some embodiments, the immunomodulatory protein or polypeptide (or a fusion or conjugate described herein (e.g., an immunomodulatory fusion protein or polypeptide described herein)) binds to a cell expressing hIL-10Rα on the surface with an EC50 of no greater than about 0.1 μM, 0.2 μM, 0.3 μM, 0.4 μM, 0.5 μM, 0.6 μM, 0.7 pm, 0.8 μM, 0.9 μM, 1.0 μM, 5 μM, 10 μM, 20 μM, 30 μM, 40 μM, 50 μM, 60 μM, 70 μM, 80 μM, 90 μM, 100 μM, 200 μM, 300 μM, 400 μM, or 500 μM.

In some embodiments, the immunomodulatory protein or polypeptide (or a fusion or conjugate described herein (e.g., an immunomodulatory fusion protein or polypeptide described herein)) binds to a cell expressing hIL-10Rα on the surface with an EC50 that is at least about 4-fold, 5-fold, 10-fold, 20-fold, 30-fold, 40-fold, 50-fold, 60-fold, 70-fold, 80-fold, 90-fold, 100-fold, 110-fold, 120-fold, 130-fold, 140-fold, or 150-fold higher than that of a reference hIL-10 protein or polypeptide (e.g., a reference hIL-10 protein or polypeptide comprising the amino acid sequence set forth in SEQ ID NO: 1 or 2) (or a reference fusion or conjugate (e.g., a reference immunomodulatory fusion protein or polypeptide (e.g., a reference immunomodulatory fusion protein or polypeptide (e.g., any one of SEQ ID NOS: 102-105))). In some embodiments, the immunomodulatory protein or polypeptide (or a fusion or conjugate described herein (e.g., an immunomodulatory fusion protein or polypeptide described herein)) binds to a cell expressing hIL-10Rα on the surface with an EC50 that is from about 10-150-fold, 20-150-fold, 30-150-fold, 40-150-fold, 50-150-fold, 60-150-fold, 70-150-fold, 80-150-fold, 90-150-fold, 100-150-fold, 110-150-fold, 120-150-fold, 130-150-fold, or 140-150-fold, higher than that of a reference hIL-10 protein or polypeptide (e.g., a reference hIL-10 protein or polypeptide comprising the amino acid sequence set forth in SEQ ID NO: 1 or 2) (or a reference fusion or conjugate described herein (e.g., a reference immunomodulatory fusion protein or polypeptide (e.g., a reference immunomodulatory fusion protein or polypeptide (e.g., any one of SEQ ID NOS: 102-105))).

Assays suitable to measure the EC50 of an immunomodulatory protein or polypeptide described herein are standard and known to the person of ordinary skill in the art. For example, the EC50 can be determined by constructing a dose-response curve and examining the effect of different concentrations of the immunomodulatory protein or polypeptide in inducing activity in a particular functional assay (e.g., STAT3 signaling, STAT3 phosphorylation, STAT3 inducible SEAP expression). An exemplary method of determining the EC50 of an immunomodulatory protein or polypeptide described herein (including immunomodulatory fusion proteins described herein) is utilization of the hIL-10 HEKBlue reporter cell line (InvivoGen #hkb-il10). The hIL-10 HEKBlue reporter cell line expresses the hIL-10Rα and hIL-10Rβ subunits, human STAT3, and a STAT3-inducible SEAP (secreted embryonic alkaline phosphatase) reporter. Thereby, binding of a protein to the hIL-10R triggers JAK1/STAT3 signaling and the subsequent production of SEAP, which can be quantified using standard methods known in the art. Additionally for example, the level of phosphorylated STAT3 can be assessed by contacting cells expressing the hIL-10R with one or more concentration of an immunomodulatory protein or polypeptide described herein, lysing the cells, and assessing the level of phosphorylated STAT3, e.g., by Western blot, FRET-based assay or chemiluminescent assay (e.g., AlphaLISA-based assay). The cells in the cell-based assay may be cells, such as HEK293 cells, which recombinantly express the hIL-10R and/or human STAT3; or cells that naturally express hIL-10R and human STAT3.

In some embodiments, the immunomodulatory protein or polypeptide (or a fusion or conjugate described herein (e.g., an immunomodulatory fusion protein or polypeptide described herein)) binds to the hIL-10R (e.g., hIL-10Rα) with higher affinity relative to that of a reference hIL-10 protein or polypeptide (e.g., a reference hIL-10 protein or polypeptide comprising the amino acid sequence set forth in SEQ ID NO: 1 or 2) (or a reference fusion or conjugate described herein (e.g., a reference immunomodulatory fusion protein or polypeptide (e.g., a reference immunomodulatory fusion protein or polypeptide (e.g., any one of SEQ ID NOS: 102-105))). Binding affinity can be measured by standard assays known in the art. For example, binding affinity can be measured by surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated by reference herein for all purposes). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules (e.g., proteins). The dissociation constant for the complex can be determined by monitoring changes in the refractive index with respect to time as buffer is passed over the chip.

Other suitable assays for measuring the binding of one protein to another (e.g., binding of a protein or polypeptide described herein to the hIL-10R) include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, sequencing and other methods for detection of binding of proteins.

### 5.3 Immunomodulatory Polypeptide/Protein Fusions & Conjugates

In some embodiments, the immunomodulatory protein or polypeptide (e.g., described herein) is operably connected to a heterologous moiety (e.g., a heterologous polypeptide) forming a fusion or conjugate protein or polypeptide, respectively. As such, further provided herein are, inter alia, fusion proteins comprising an immunomodulatory protein or polypeptide (e.g., described herein) and one or more heterologous proteins (or a functional fragment, functional variant, or domain thereof). Further provided herein are, inter alia, conjugates comprising an immunomodulatory protein or polypeptide (e.g., described herein) (or a nucleic acid molecule encoding an immunomodulatory protein or polypeptide (e.g., described herein) and one or more heterologous moieties.

Heterologous moieties include, but are not limited to, proteins, peptides, small molecules, nucleic acid molecules (e.g., DNA, RNA, DNA/RNA hybrid molecules), carbohydrates, lipids, and synthetic polymers (e.g., polymers of PEG). In some embodiments, the heterologous moiety is a detectable moiety (e.g., polypeptide or protein, e.g., a fluorescent polypeptide or protein).

In some embodiments, the heterologous moiety is a half-life extension moiety. Exemplary half-life extension moieties include, but are not limited to, an immunoglobulin (e.g., human Ig (hIg), murine Ig (mIg)), a fragment of an Ig (e.g., hIg, mIg), an Ig (e.g., hIg, mIg) constant region, a fragment of an Ig (e.g., hIg, mIg) constant region, an Ig (e.g., hIg, mIg) Fc region human transferrin, human serum albumin (HSA), an HSA binding protein or peptide, and polyethylene glycol (PEG) (and polymers thereof). In some embodiments, the heterologous polypeptide is a half-life extension polypeptide. Exemplary half-life extension polypeptides include, but are not limited to, an Ig, a fragment of an Ig, one or more Ig heavy chain constant region, a fragment of an Ig constant region, an Ig Fc region, a hIg, a fragment of a hIg, one or more hIg heavy chain constant region, a fragment of a hIg constant region, a hIg Fc region, a mIg, a fragment of a mIg, one or more mIg heavy chain constant region, a fragment of a mIg constant region, a mIg Fc region, human transferrin, human serum albumin (HSA), and an HSA binding protein or peptide. The immunomodulatory protein or polypeptide described herein fused or conjugated to a half-life extending moiety or a half-life extending moiety can be evaluated for their pharmacokinetic properties utilizing standard in vivo methods known in the art.

### 5.3.1 Ig Fusion Proteins & Polypeptides

### 5.3.1.1 Antibody Fusion Proteins & Polypeptides

In some embodiments, the heterologous polypeptide comprises an antibody. An antibody fusion can act to further target the immunomodulatory protein or polypeptide e.g., to a specified cell type expressing a specific cell surface protein. Exemplary antibodies include, full-length antibodies, scFv, Fab, single domain antibodies (e.g., VHH), scFv-Fc, Fab-Fc, and single domain antibody-Fc (e.g., VHH-Fc).

### 5.3.1.2 Ig Fusion Proteins & Polypeptides

In some embodiments, the heterologous polypeptide comprises one or more Ig heavy chain constant regions (e.g., a CH2 region, a CH3 region, a hinge region, an Fc region (e.g., in some embodiments, preferably an Fc region). In some embodiments, the Ig is an IgG. In some embodiments, the IgG is IgG₁, IgG₂, IgG₃, or IgG₄ (e.g., in some embodiments preferably an IgG₄).

In some embodiments, the heterologous polypeptide comprises or consists of an IgG CH2 region and an IgG CH3 region. In some embodiments, the heterologous polypeptide comprises or consists of a partial IgG hinge region, IgG CH2 region, and IgG CH3 region. In some embodiments, the heterologous polypeptide comprises or consists of an IgG hinge region, IgG CH2 region, and IgG CH3 region. In some embodiments, the heterologous polypeptide comprises or consists of an IgG₁ CH2 region and an IgG₁ CH3 region. In some embodiments, the heterologous polypeptide comprises or consists of a partial IgG₁ hinge region, IgG₁ CH2 region, and IgG₁ CH3 region. In some embodiments, the heterologous polypeptide comprises or consists of an IgG₁ hinge region, IgG₁ CH2 region, and IgG₁ CH3 region. In some embodiments, the heterologous polypeptide comprises or consists of an IgG₄ CH2 region and an IgG₄ CH3 region. In some embodiments, the heterologous polypeptide comprises or consists of a partial IgG₄ hinge region, IgG₄ CH2 region, and IgG₄ CH3 region. In some embodiments, the heterologous polypeptide comprises or consists of an IgG₄ hinge region, IgG₄ CH2 region, and IgG₄ CH3 region.

In some embodiments, the heterologous polypeptide comprises or consists of an Ig Fc region. In some embodiments, the Ig Fc region comprises or consists of at least a portion of a hinge region, a CH2 region, and a CH3 region. In some embodiments, the Ig Fc region comprises or consists of a hinge region, a CH2 region, and a CH3 region. In some embodiments, the Ig Fc region comprises or consists of at least a portion of an IgG hinge region, an IgG CH2 region, and an IgG CH3 region. In some embodiments, the Ig Fc region comprises or consists of an IgG hinge region, an IgG CH2 region, and an IgG CH3 region. In some embodiments, the Ig Fc region comprises or consists of at least a portion of an IgG₁ hinge region, an IgG₁ CH2 region, and an IgG₁ CH3 region. In some embodiments, the Ig Fc region comprises or consists of an IgG₁ hinge region, an IgG₁ CH2 region, and an IgG₁ CH3 region. In some embodiments, the Ig Fc region comprises or consists of at least a portion of an IgG₄ hinge region, an IgG₄ CH2 region, and an IgG₄ CH3 region. In some embodiments, the Ig Fc region comprises or consists of an IgG₄ hinge region, an IgG₄ CH2 region, and an IgG₄ CH3 region.

In some embodiments, the heterologous polypeptide comprises one or more hIg heavy chain constant regions (e.g., a CH2 region, a CH3 region, a hinge region, an Fc region). In some embodiments, the hIg is a human IgG (hIgG). In some embodiments, the hIgG is hIgG1, IgG₂, IgG₃, or IgG₄. In some embodiments, the hIgG is IgG₁ or IgG₄. In some embodiments, the hIgG is hIgG1. In some embodiments, the hIgG is hIgG4.

In some embodiments, the heterologous polypeptide comprises or consists of a hIgG CH2 region and a hIgG CH3 region. In some embodiments, the heterologous polypeptide comprises or consists of a partial hIgG hinge region, hIgG CH2 region, and hIgG CH3 region. In some embodiments, the heterologous polypeptide comprises or consists of a hIgG hinge region, hIgG CH2 region, and hIgG CH3 region. In some embodiments, the heterologous polypeptide comprises or consists of a hIgG1 CH2 region and a hIgG1 CH3 region. In some embodiments, the heterologous polypeptide comprises or consists of a partial hIgG1 hinge region, hIgG1 CH2 region, and hIgG1 CH3 region. In some embodiments, the heterologous polypeptide comprises or consists of a hIgG1 hinge region, hIgG1 CH2 region, and hIgG1 CH3 region. In some embodiments, the heterologous polypeptide comprises or consists of a hIgG4 CH2 region and a hIgG4 CH3 region. In some embodiments, the heterologous polypeptide comprises or consists of a partial hIgG4 hinge region, hIgG4 CH2 region, and hIgG4 CH3 region. In some embodiments, the heterologous polypeptide comprises or consists of a hIgG4 hinge region, hIgG4 CH2 region, and hIgG4 CH3 region.

In some embodiments, the heterologous polypeptide comprises or consists of a hIg Fc region. In some embodiments, the hIg Fc region comprises or consists of at least a portion of a hinge region, a CH2 region, and a CH3 region. In some embodiments, the hIg Fc region comprises or consists of a hinge region, a CH2 region, and a CH3 region. In some embodiments, the hIg Fc region comprises or consists of at least a portion of a hIgG hinge region, a hIgG CH2 region, and a hIgG CH3 region. In some embodiments, the hIg Fc region comprises or consists of a hIgG hinge region, a hIgG CH2 region, and a hIgG CH3 region. In some embodiments, the hIg Fc region comprises or consists of at least a portion of a hIgG1 hinge region, a hIgG1 CH2 region, and a hIgG1 CH3 region. In some embodiments, the hIg Fc region comprises or consists of a hIgG1 hinge region, a hIgG1 CH2 region, and a hIgG1 CH3 region. In some embodiments, the hIg Fc region comprises or consists of at least a portion of a hIgG4 hinge region, a hIgG4 CH2 region, and a hIgG4 CH3 region. In some embodiments, the hIg Fc region comprises or consists of a hIgG4 hinge region, a hIgG4 CH2 region, and a hIgG4 CH3 region.

The amino acid sequence of exemplary reference hIgG1 and hIgG4 heavy chain constant regions and hIg light chain constant regions, which can be incorporated in one or more of the embodiments described herein (e.g., fusion proteins and polypeptide), is provided in Table 4.

TABLE 4
The Amino Acid Sequence of Exemplary hIg heavy chain constant
region components and hIg light chain constant regions

| | | SEQ ID |
|---|---|---|
| Description | Amino Acid Sequence | NO |
| hIgG1 CH1 Region | ASTKGPSVFPLAPSSKSTSGGTAALGCLVKDYFPEPVTVSW | 531 |
| | NSGALTSGVHTFPAVLQSSGLYSLSSVVTVPSSSLGTQTYI | |
| | CNVNHKPSNTKVDKKV | |
| hIgG1 Hinge Region | EPKSCDKTHTCP | 12 |
| hIgG1 CH2 Region | PCPAPELLGGPSVFLFPPKPKDTLMISRTPEVTCVVVDVSH | 13 |
| | EDPEVKFNWYVDGVEVHNAKTKPREEQYNSTYRVVSVLIVL | |
| | HQDWLNGKEYKCKVSNKALPAPIEKTISKAK | |
| hIgG1 CH3 Region | GQPREPQVYTLPPSRDELTKNQVSLTCLVKGFYPSDIAVEW | 14 |
| With C-terminal | ESNGQPENNYKTTPPVLDSDGSFFLYSKLTVDKSRWQQGNV | |
| Lysine | FSCSVMHEALHNHYTQKSLSLSPGK | |
| hIgG1 CH3 Region | GQPREPQVYTLPPSRDELTKNQVSLTCLVKGFYPSDIAVEW | 15 |
| Without C-terminal | ESNGQPENNYKTTPPVLDSDGSFFLYSKLTVDKSRWQQGNV | |
| Lysine | FSCSVMHEALHNHYTQKSLSLSPG | |
| hIgG1 CH2 Region + | PCPAPELLGGPSVFLFPPKPKDILMISRTPEVTCVVVDVSH | 16 |
| CH3 Region | EDPEVKFNWYVDGVEVHNAKTKPREEQYNSTYRVVSVLTVL | |
| With C-terminal | HQDWLNGKEYKCKVSNKALPAPIEKTISKAKGQPREPQVYT | |
| Lysine | LPPSRDELTKNQVSLTCLVKGFYPSDIAVEWESNGQPENNY | |
| | KTTPPVLDSDGSFFLYSKLTVDKSRWQQGNVFSCSVMHEAL | |
| | HNHYTQKSLSLSPGK | |
| hIgG1 CH2 Region + | PCPAPELLGGPSVFLFPPKPKDTLMISRTPEVTCVVVDVSH | 17 |
| CH3 Region | EDPEVKFNWYVDGVEVHNAKIKPREEQYNSTYRVVSVLIVL | |
| Without C-terminal | HQDWLNGKEYKCKVSNKALPAPIEKTISKAKGQPREPQVYT | |
| Lysine | LPPSRDELTKNQVSLTCLVKGFYPSDIAVEWESNGQPENNY | |
| | KTTPPVLDSDGSFFLYSKLTVDKSRWQQGNVFSCSVMHEAL | |
| | HNHYTQKSLSLSPG | |
| hIgG1 Partial Hinge | TCPPCPAPELLGGPSVFLFPPKPKDILMISRTPEVTCVVVD | 18 |
| Region + CH2 Region + | VSHEDPEVKFNWYVDGVEVHNAKTKPREEQYNSTYRVVSVL | |
| CH3 Region | TVLHQDWLNGKEYKCKVSNKALPAPIEKTISKAKGQPREPQ | |
| With C-terminal | VYTLPPSRDELTKNQVSLTCLVKGFYPSDIAVEWESNGQPE | |
| Lysine | NNYKTTPPVLDSDGSFFLYSKLTVDKSRWQQGNVFSCSVMH | |
| | EALHNHYTQKSLSLSPGK | |
| hIgG1 Partial Hinge | TCPPCPAPELLGGPSVFLFPPKPKDTLMISRTPEVTCVVVD | 19 |
| Region + CH2 Region + | VSHEDPEVKFNWYVDGVEVHNAKIKPREEQYNSTYRVVSVL | |
| CH3 Region | TVLHQDWLNGKEYKCKVSNKALPAPIEKTISKAKGQPREPQ | |
| Without C-terminal | VYTLPPSRDELTKNQVSLTCLVKGFYPSDIAVEWESNGQPE | |
| Lysine | NNYKTTPPVLDSDGSFFLYSKLTVDKSRWQQGNVFSCSVMH | |
| | EALHNHYTQKSLSLSPG | |
| hIgG1 Hinge Region + | EPKSCDKTHTCPPCPAPELLGGPSVFLFPPKPKDTLMISRT | 20 |
| CH2 Region + CH3 | PEVTCVVVDVSHEDPEVKFNWYVDGVEVHNAKTKPREEQYN | |
| Region | STYRVVSVLIVLHQDWLNGKEYKCKVSNKALPAPIEKTISK | |
| With C-terminal | AKGQPREPQVYTLPPSRDELTKNQVSLICLVKGFYPSDIAV | |
| Lysine | EWESNGQPENNYKTTPPVLDSDGSFFLYSKLIVDKSRWQQG | |
| | NVFSCSVMHEALHNHYTQKSLSLSPGK | |
| hIgG1 Hinge Region + | EPKSCDKTHTCPPCPAPELLGGPSVFLFPPKPKDTLMISRT | 21 |
| CH2 Region + CH3 | PEVTCVVVDVSHEDPEVKFNWYVDGVEVHNAKIKPREEQYN | |
| Region | STYRVVSVLTVLHQDWLNGKEYKCKVSNKALPAPIEKTISK | |
| Without C-terminal | AKGQPREPQVYTLPPSRDELTKNQVSLTCLVKGFYPSDIAV | |
| Lysine | EWESNGQPENNYKTTPPVLDSDGSFFLYSKLTVDKSRWQQG | |
| | NVFSCSVMHEALHNHYTQKSLSLSPG | |
| hIgG1 CH1+ Hinge | ASTKGPSVFPLAPSSKSTSGGTAALGCLVKDYFPEPVTVSW | 532 |
| Region + CH2 Region + | NSGALTSGVHTFPAVLQSSGLYSLSSVVTVPSSSLGTQTYI | |
| CH3 Region | CNVNHKPSNTKVDKKVEPKSCDKTHTCPPCPAPELLGGPSV | |
| With C-terminal | FLFPPKPKDTLMISRTPEVTCVVVDVSHEDPEVKFNWYVDG | |
| Lysine | VEVHNAKTKPREEQYNSTYRVVSVLIVLHQDWLNGKEYKCK | |
| | VSNKALPAPIEKTISKAKGQPREPQVYTLPPSRDELIKNQV | |
| | SLTCLVKGFYPSDIAVEWESNGQPENNYKTTPPVLDSDGSF | |
| | FLYSKLTVDKSRWQQGNVFSCSVMHEALHNHYTQKSLSLSP | |
| | GK | |
| hIgG1 CH1 + Hinge | ASTKGPSVFPLAPSSKSTSGGTAALGCLVKDYFPEPVTVSW | 533 |
| Region + CH2 Region + | NSGALISGVHTFPAVLQSSGLYSLSSVVTVPSSSLGTQTYI | |
| CH3 Region | CNVNHKPSNTKVDKKVEPKSCDKTHTCPPCPAPELLGGPSV | |
| Without C-terminal | FLFPPKPKDTLMISRTPEVTCVVVDVSHEDPEVKENWYVDG | |
| Lysine | VEVHNAKTKPREEQYNSTYRVVSVLIVLHQDWLNGKEYKCK | |
| | VSNKALPAPIEKTISKAKGQPREPQVYTLPPSRDELTKNQV | |
| | SLTCLVKGFYPSDIAVEWESNGQPENNYKTTPPVLDSDGSF | |
| | FLYSKLTVDKSRWQQGNVFSCSVMHEALHNHYTQKSLSLSP | |
| | G | |
| hIgG4 CH1 Region | ASTKGPSVFPLAPCSRSTSESTAALGCLVKDYFPEPVTVSW | 534 |
| | NSGALTSGVHTFPAVLQSSGLYSLSSVVTVPSSSLGTKTYT | |
| | CNVDHKPSNTKVDKRV | |
| hIgG4 Hinge Region | ESKYGPPCPSCP | 22 |
| hIgG4 Hinge Region | AESKYGPPCPSCP | 23 |
| (Variant) | | |
| hIgG4 CH2 Region | APEFLGGPSVFLFPPKPKDTLMISRTPEVTCVVVDVSQEDP | 24 |
| | EVQFNWYVDGVEVHNAKTKPREEQFNSTYRVVSVLTVLHQD | |
| | WLNGKEYKCKVSNKGLPSSIEKTISKAK | |
| hIgG4 CH3 Region | GQPREPQVYTLPPSQEEMTKNQVSLTCLVKGFYPSDIAVEW | 25 |
| With C-terminal | ESNGQPENNYKTTPPVLDSDGSFFLYSRLTVDKSRWQEGNV | |
| Lysine | FSCSVMHEALHNHYTQKSLSLSLGK | |
| hIgG4 CH3 Region | GQPREPQVYTLPPSQEEMTKNQVSLTCLVKGFYPSDIAVEW | 26 |
| Without C-terminal | ESNGQPENNYKTTPPVLDSDGSFFLYSRLTVDKSRWQEGNV | |
| Lysine | FSCSVMHEALHNHYTQKSLSLSLG | |
| hIgG4 CH2 Region + | APEFLGGPSVFLFPPKPKDTLMISRTPEVTCVVVDVSQEDP | 27 |
| CH3 Region | EVQFNWYVDGVEVHNAKTKPREEQFNSTYRVVSVLTVLHQD | |
| With C-terminal | WLNGKEYKCKVSNKGLPSSIEKTISKAKGQPREPQVYTLPP | |
| Lysine | SQEEMTKNQVSLTCLVKGFYPSDIAVEWESNGQPENNYKTT | |
| | PPVLDSDGSFFLYSRLTVDKSRWQEGNVFSCSVMHEALHNH | |
| | YTQKSLSLSLGK | |
| hIgG4 CH2 Region + | APEFLGGPSVFLFPPKPKDTLMISRTPEVTCVVVDVSQEDP | 28 |
| CH3 Region | EVQFNWYVDGVEVHNAKTKPREEQFNSTYRVVSVLTVLHQD | |
| Without C-terminal | WLNGKEYKCKVSNKGLPSSIEKTISKAKGQPREPQVYTLPP | |
| Lysine | SQEEMTKNQVSLTCLVKGFYPSDIAVEWESNGQPENNYKTT | |
| | PPVLDSDGSFFLYSRLTVDKSRWQEGNVFSCSVMHEALHNH | |
| | YTQKSLSLSLG | |
| hIgG4 Partial Hinge | PCPSCPAPEFLGGPSVFLFPPKPKDTLMISRTPEVTCVVVD | 29 |
| Region + CH2 Region + | VSQEDPEVQFNWYVDGVEVHNAKTKPREEQFNSTYRVVSVL | |
| CH3 Region | TVLHQDWLNGKEYKCKVSNKGLPSSIEKTISKAKGQPREPQ | |
| With C-terminal | VYTLPPSQEEMTKNQVSLTCLVKGFYPSDIAVEWESNGQPE | |
| Lysine | NNYKTTPPVLDSDGSFFLYSRLTVDKSRWQEGNVFSCSVMH | |
| | EALHNHYTQKSLSLSLGK | |
| hIgG4 Partial Hinge | PCPSCPAPEFLGGPSVFLFPPKPKDTLMISRTPEVTCVVVD | 30 |
| Region + CH2 Region + | VSQEDPEVQFNWYVDGVEVHNAKTKPREEQFNSTYRVVSVL | |
| CH3 Region | TVLHQDWLNGKEYKCKVSNKGLPSSIEKTISKAKGQPREPQ | |
| Without C-terminal | VYTLPPSQEEMTKNQVSLTCLVKGFYPSDIAVEWESNGQPE | |
| Lysine | NNYKTTPPVLDSDGSFFLYSRLTVDKSRWQEGNVFSCSVMH | |
| | EALHNHYTQKSLSLSLG | |
| hIgG4 Hinge Region + | ESKYGPPCPSCPAPEFLGGPSVFLFPPKPKDTLMISRTPEV | 31 |
| CH2 Region + CH3 | TCVVVDVSQEDPEVQFNWYVDGVEVHNAKTKPREEQFNSTY | |
| Region | RVVSVLTVLHQDWLNGKEYKCKVSNKGLPSSIEKTISKAKG | |
| With C-terminal | QPREPQVYTLPPSQEEMTKNQVSLTCLVKGFYPSDIAVEWE | |
| Lysine | SNGQPENNYKTTPPVLDSDGSFFLYSRLTVDKSRWQEGNVF | |
| | SCSVMHEALHNHYTQKSLSLSLGK | |
| hIgG4 Hinge Region + | ESKYGPPCPSCPAPEFLGGPSVFLFPPKPKDTLMISRTPEV | 32 |
| CH2 Region + CH3 | TCVVVDVSQEDPEVQFNWYVDGVEVHNAKTKPREEQFNSTY | |
| Region | RVVSVLTVLHQDWLNGKEYKCKVSNKGLPSSIEKTISKAKG | |
| Without C-terminal | QPREPQVYTLPPSQEEMTKNQVSLTCLVKGFYPSDIAVEWE | |
| Lysine | SNGQPENNYKTTPPVLDSDGSFFLYSRLTVDKSRWQEGNVF | |
| | SCSVMHEALHNHYTQKSLSLSLG | |
| hIgG4 Hinge Region + | AESKYGPPCPSCPAPEFLGGPSVFLFPPKPKDTLMISRTPE | 33 |
| CH2 Region + CH3 | VTCVVVDVSQEDPEVQFNWYVDGVEVHNAKTKPREEQFNST | |
| Region | YRVVSVLIVLHQDWLNGKEYKCKVSNKGLPSSIEKTISKAK | |
| (Variant) | GQPREPQVYTLPPSQEEMTKNQVSLICLVKGFYPSDIAVEW | |
| With C-terminal | ESNGQPENNYKTTPPVLDSDGSFFLYSRLTVDKSRWQEGNV | |
| Lysine | FSCSVMHEALHNHYTQKSLSLSLGK | |
| hIgG4 Hinge Region + | AESKYGPPCPSCPAPEFLGGPSVFLFPPKPKDILMISRTPE | 34 |
| CH2 Region + CH3 | VTCVVVDVSQEDPEVQFNWYVDGVEVHNAKTKPREEQFNST | |
| Region | YRVVSVLTVLHQDWLNGKEYKCKVSNKGLPSSIEKTISKAK | |
| (Variant) | GQPREPQVYTLPPSQEEMTKNQVSLTCLVKGFYPSDIAVEW | |
| Without C-terminal | ESNGQPENNYKTTPPVLDSDGSFFLYSRLTVDKSRWQEGNV | |
| Lysine | FSCSVMHEALHNHYTQKSLSLSLG | |
| hIgG4 CH1 + Hinge | ASTKGPSVFPLAPCSRSTSESTAALGCLVKDYFPEPVTVSW | 535 |
| Region + CH2 Region + | NSGALTSGVHTFPAVLQSSGLYSLSSVVTVPSSSLGTKTYT | |
| CH3 Region | CNVDHKPSNTKVDKRVESKYGPPCPSCPAPEFLGGPSVFLF | |
| With C-terminal | PPKPKDTLMISRTPEVTCVVVDVSQEDPEVQFNWYVDGVEV | |
| Lysine | HNAKTKPREEQFNSTYRVVSVLTVLHQDWLNGKEYKCKVSN | |
| | KGLPSSIEKTISKAKGQPREPQVYTLPPSQEEMTKNQVSLT | |
| | CLVKGFYPSDIAVEWESNGQPENNYKTTPPVLDSDGSFFLY | |
| | SRLTVDKSRWQEGNVFSCSVMHEALHNHYTQKSLSLSLGK | |
| hIgG4 CH1 + Hinge | ASTKGPSVFPLAPCSRSTSESTAALGCLVKDYFPEPVTVSW | 536 |
| Region + CH2 Region + | NSGALISGVHIFPAVLQSSGLYSLSSVVTVPSSSLGTKTYT | |
| CH3 Region | CNVDHKPSNTKVDKRVESKYGPPCPSCPAPEFLGGPSVFLF | |
| Without C-terminal | PPKPKDTLMISRIPEVTCVVVDVSQEDPEVQFNWYVDGVEV | |
| Lysine | HNAKTKPREEQFNSTYRVVSVLIVLHQDWLNGKEYKCKVSN | |
| | KGLPSSIEKTISKAKGQPREPQVYTLPPSQEEMTKNQVSLT | |
| | CLVKGFYPSDIAVEWESNGQPENNYKTTPPVLDSDGSFFLY | |
| | SRLTVDKSRWQEGNVFSCSVMHEALHNHYTQKSLSLSLG | |
| Ig light chain kappa | RTVAAPSVFIFPPSDEQLKSGTASVVCLLNNFYPREAKVQW | 537 |
| constant region (κCL) | KVDNALQSGNSQESVTEQDSKDSTYSLSSTLILSKADYEKH | |
| | KVYACEVTHQGLSSPVTKSFNRGEC | |
| Ig light chain kappa | GQPKANPTVTLFPPSSEELQANKATLVCLISDFYPGAVIVA | 538 |
| constant region (λCL) | WKADGSPVKAGVETTKPSKQSNNKYAASSYLSLTPEQWKSH | |
| | RSYSCQVTHEGSTVEKTVAPTECS | |

In some embodiments, the amino acid sequence of the heterologous polypeptide comprises an amino acid sequence set forth in Table 4. In some embodiments, the amino acid sequence of the heterologous polypeptide consists of an amino acid sequence set forth in Table 4. In some embodiments, the amino acid sequence of the heterologous polypeptide comprises or consists of an amino acid sequence set forth in Table 4, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide comprises or consists of an amino acid sequence set forth in Table 4, comprising or consisting of at least about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises or consists of an amino acid sequence set forth in Table 4, comprising or consisting of about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises or consists of an amino acid sequence set forth in Table 4, comprising or consisting of about no more than 1, 2, 3, 4, 5, 6, 7, 8, 9, 10, or more amino acid variations (e.g., amino acid substitutions, deletions, or additions).

In some embodiments, the amino acid sequence of the heterologous polypeptide comprises or consists of an amino acid sequence set forth in Table 4, and further comprises 1 or more but less than 15% (less than 12%, less than 10%, less than 8%), amino acid substitutions. In some embodiments, the amino acid sequence of the heterologous polypeptide comprises or consists of an amino acid sequence set forth in Table 4, comprising or consisting of at least about 1, 2, 3, 4, 5, 6, 7, 8, 9, 10, or more amino acid substitutions. In some embodiments, the amino acid sequence of the heterologous polypeptide comprises or consists of an amino acid sequence set forth in Table 4, comprising or consisting of about 1, 2, 3, 4, 5, 6, 7, 8, 9, 10, or more amino acid substitutions. In some embodiments, the amino acid sequence of the heterologous polypeptide comprises or consists of an amino acid sequence set forth in Table 4, comprising or consisting of about no more than about 1, 2, 3, 4, 5, 6, 7, 8, 9, 10, or more amino acid substitutions.

In some embodiments, the amino acid sequence of the heterologous polypeptide comprises the amino acid sequence of any one of SEQ ID NOS: 16-21 or 27-34. In some embodiments, the amino acid sequence of the heterologous polypeptide consists of the amino acid sequence of any one of SEQ ID NOS: 16-21 or 27-34. In some embodiments, the amino acid sequence of the heterologous polypeptide comprises or consists of the amino acid sequence of any one of SEQ ID NOS: 16-21 or 27-34, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide comprises or consists of the amino acid sequence of any one of SEQ ID NOS: 16-21 or 27-34, comprising or consisting at least about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises or consists of the amino acid sequence of any one of SEQ ID NOS: 16-21 or 27-34, comprising or consisting about 1, 2, 3, 4, 5, 6, 7, 8, 9, or 10 amino acid variations (e.g., amino acid substitutions, deletions, or additions). In some embodiments, the amino acid sequence of the heterologous polypeptide comprises or consists of the amino acid sequence of any one of SEQ ID NOS: 16-21 or 27-34, comprising or consisting of no more than about 1, 2, 3, 4, 5, 6, 7, 8, 9, or 10 amino acid variations (e.g., amino acid substitutions, deletions, or additions).

In some embodiments, the amino acid sequence of the heterologous polypeptide comprises or consists of the amino acid sequence of any one of SEQ ID NOS: 16-21 or 27-34, and further comprises 1 or more but less than 15% (less than 12%, less than 10%, less than 8%), amino acid substitutions. In some embodiments, the amino acid sequence of the heterologous polypeptide comprises or consists of the amino acid sequence of any one of SEQ ID NOS: 16-21 or 27-34. In some embodiments, the amino acid sequence of the heterologous polypeptide comprises or consists of the amino acid sequence of any one of SEQ ID NOS: 16-21 or 27-34, comprising or consisting at least about 1, 2, 3, 4, 5, 6, 7, 8, 9, 10, or more amino acid substitutions. In some embodiments, the amino acid sequence of the heterologous polypeptide comprises or consists of the amino acid sequence of any one of SEQ ID NOS: 16-21 or 27-34, comprising or consisting about 1, 2, 3, 4, 5, 6, 7, 8, 9, or 10 amino acid substitutions. In some embodiments, the amino acid sequence of the heterologous polypeptide comprises or consists of the amino acid sequence of any one of SEQ ID NOS: 16-21 or 27-34, comprising or consisting of no more than about 1, 2, 3, 4, 5, 6, 7, 8, 9, or 10 amino acid substitutions.

In some embodiments, wherein the heterologous polypeptide comprises a CH3 region (e.g., comprises an Fc region; a hinge region, CH2 region, and CH3 region, etc.), the CH3 region lacks the C-terminal lysine (e.g., residue 232 of SEQ ID NO: 20, numbering according to SEQ ID NO: 20; or e.g., residue 229 of SEQ ID NO: 31, numbering according to SEQ ID NO: 31). In some embodiments, the CH3 region further lacks the C-terminal glycine (e.g., residue 231 of SEQ ID NO: 20, numbering according to SEQ ID NO: 20; or e.g., residue 228 of SEQ ID NO: 31, numbering according to SEQ ID NO: 31).

In some embodiments, the heterologous polypeptide comprises one or more mIg heavy chain constant regions (e.g., a CH2 region, a CH3 region, a hinge region, an Fc region). In some embodiments, the mIg is mIgG (mIgG). In some embodiments, the mIgG is mIgG1, mIgG2a, mIgG2c, mIgG2b, or mIgG3. In some embodiments, the mIgG is mIgG1 or mIgG2a. In some embodiments, the mIgG is mIgG1. In some embodiments, the mIgG is mIgG2a.

In some embodiments, the heterologous polypeptide comprises or consists of a mIgG CH2 region and a mIgG CH3 region. In some embodiments, the heterologous polypeptide comprises or consists of a partial mIgG hinge region, mIgG CH2 region, and mIgG CH3 region. In some embodiments, the heterologous polypeptide comprises or consists of a mIgG hinge region, mIgG CH2 region, and mIgG CH3 region. In some embodiments, the heterologous polypeptide comprises or consists of a mIgG1 CH2 region and a mIgG1 CH3 region. In some embodiments, the heterologous polypeptide comprises or consists of a partial mIgG1 hinge region, mIgG1 CH2 region, and mIgG1 CH3 region. In some embodiments, the heterologous polypeptide comprises or consists of a mIgG1 hinge region, mIgG1 CH2 region, and mIgG1 CH3 region. In some embodiments, the heterologous polypeptide comprises or consists of a mIgG2a CH2 region and a mIgG2a CH3 region. In some embodiments, the heterologous polypeptide comprises or consists of a partial mIgG2a hinge region, mIg2a CH2 region, and mIgG2a CH3 region. In some embodiments, the heterologous polypeptide comprises or consists of a mIgG2a hinge region, mIgG2a CH2 region, and mIgG2a CH3 region.

In some embodiments, the heterologous polypeptide comprises or consists of a mIg Fc region. In some embodiments, the mIg Fc region comprises or consists of at least a portion of a hinge region, a CH2 region, and a CH3 region. In some embodiments, the mIg Fc region comprises or consists of a hinge region, a CH2 region, and a CH3 region. In some embodiments, the mIg Fc region comprises or consists of at least a portion of a mIgG hinge region, a mIgG CH2 region, and a mIgG CH3 region. In some embodiments, the mIg Fc region comprises or consists of a mIgG hinge region, a mIgG CH2 region, and a mIgG CH3 region. In some embodiments, the mIg Fc region comprises or consists of at least a portion of a mIgG1 hinge region, a mIgG1 CH2 region, and a mIgG1 CH3 region. In some embodiments, the mIg Fc region comprises or consists of a mIgG1 hinge region, a mIgG1 CH2 region, and a mIgG1 CH3 region. In some embodiments, the mIg Fc region comprises or consists of at least a portion of a mIgG2a hinge region, a mIgG2a CH2 region, and a mIgG2a CH3 region. In some embodiments, the mIg Fc region comprises or consists of a mIgG2a hinge region, a mIgG2a CH2 region, and a mIgG2a CH3 region.

The amino acid sequence of exemplary reference mIgG1 and mIgG2a heavy chain constant regions, which can be incorporated in one or more of the embodiments described herein (e.g., fusion proteins and polypeptide), is provided in Table 10.

TABLE 10
The Amino Acid Sequence of Exemplary mIg heavy chain constant
region components

| | | SEQ |
|---|---|---|
| Description | Amino Acid Sequence | ID NO |
| mIgG1 CH1 Region | AKTTPPSVYPLAPGSAAQTNSMVTLGCLVKGYFPEPVTVTWNSG | 539 |
| | SLSSGVHTFPAVLQSDLYTLSSSVTVPSSPRPSETVTCNVAHPA | |
| | SSTKVDKKI | |
| mIgG1 Hinge Region | VPRDCGCKPCICT | 455 |
| mIgG1 CH2 Region | VPEVSSVFIFPPKPKDVLIITLIPKVTCVVVAISKDDPEVQFSW | 456 |
| | FVDDVEVHTAQTQPREEQFNSTFRSVSELPIMHQDWLNGKEFKC | |
| | RVNSAAFPAPIEKTISKTK | |
| mIgG1 CH3 Region | GRPKAPQVYTIPPPKEQMAKDKVSLTCMITDFFPEDITVEWQWN | 457 |
| With C-terminal | GQPAENYKNTQPIMNINGSYFVYSKLNVQKSNWEAGNTFTCSVL | |
| Lysine | HEGLHNHHTEKSLSHSPGK | |
| mIgG1 CH3 Region | GRPKAPQVYTIPPPKEQMAKDKVSLTCMITDFFPEDITVEWQWN | 458 |
| Without C-terminal | GQPAENYKNTQPIMNINGSYFVYSKLNVQKSNWEAGNTFTCSVL | |
| Lysine | HEGLHNHHTEKSLSHSPG | |
| mIgG1 CH2 Region + | VPEVSSVFIFPPKPKDVLTITLTPKVTCVVVAISKDDPEVQFSW | 459 |
| CH3 Region | FVDDVEVHTAQTQPREEQFNSTFRSVSELPIMHQDWLNGKEFKC | |
| With C-terminal | RVNSAAFPAPIEKTISKTKGRPKAPQVYTIPPPKEQMAKDKVSL | |
| Lysine | TCMITDFFPEDITVEWQWNGQPAENYKNTQPIMNINGSYFVYSK | |
| | LNVQKSNWEAGNTFTCSVLHEGLHNHHTEKSLSHSPGK | |
| mIgG1 CH2 Region + | VPEVSSVFIFPPKPKDVLTITLTPKVTCVVVAISKDDPEVQFSW | 460 |
| CH3 Region | FVDDVEVHTAQTQPREEQFNSTFRSVSELPIMHQDWLNGKEFKC | |
| Without C-terminal | RVNSAAFPAPIEKTISKTKGRPKAPQVYTIPPPKEQMAKDKVSL | |
| Lysine | TCMITDFFPEDITVEWQWNGQPAENYKNTQPIMNINGSYFVYSK | |
| | LNVQKSNWEAGNTFTCSVLHEGLHNHHTEKSLSHSPG | |
| mIgG1 Hinge Region + | VPRDCGCKPCICTVPEVSSVFIFPPKPKDVLTITLTPKVTCVVV | 461 |
| CH2 Region + CH3 | AISKDDPEVQFSWFVDDVEVHTAQTQPREEQFNSTFRSVSELPI | |
| Region | MHQDWLNGKEFKCRVNSAAFPAPIEKTISKTKGRPKAPQVYTIP | |
| With C-terminal | PPKEQMAKDKVSLTCMITDFFPEDITVEWQWNGQPAENYKNTQP | |
| Lysine | IMNINGSYFVYSKLNVQKSNWEAGNTFTCSVLHEGLHNHHTEKS | |
| | LSHSPGK | |
| mIgG1 Hinge Region + | VPRDCGCKPCICTVPEVSSVFIFPPKPKDVLTITLTPKVTCVVV | 462 |
| CH2 Region + CH3 | AISKDDPEVQFSWFVDDVEVHTAQTQPREEQFNSTERSVSELPI | |
| Region | MHQDWLNGKEFKCRVNSAAFPAPIEKTISKTKGRPKAPQVYTIP | |
| Without C-terminal | PPKEQMAKDKVSLTCMITDFFPEDITVEWQWNGQPAENYKNTQP | |
| Lysine | IMNINGSYFVYSKLNVQKSNWEAGNTFTCSVLHEGLHNHHTEKS | |
| | LSHSPGK | |
| mIgG2a Hinge Region | EPRGPTIKPCPPCKCP | 463 |
| mIgG2a CH2 Region | APNAAGGPSVFIFLLKIKDVLMISLSPIVTCVVVDVSEDDPDVQ | 464 |
| | ISWFVNNVEVHTAQTQTHREDYNSTLRVVSALPIQHQDWMSGKE | |
| | FKCKVNNKDLPAPIERTISKPKGSVRAPQVYVLPPPEEEMTKKQ | |
| | VT | |
| mIgG2a CH3 Region | LTCMVTDFMPEDIYVEWTNNGKTELNYKNTEPVLDSDGSYFMYS | 465 |
| With C-terminal | KLRVEKKNWVERNSYSCSVVHEGLHNHHTTKSFSRTPGK | |
| Lysine | | |
| mIgG2a CH3 Region | LTCMVTDFMPEDIYVEWINNGKTELNYKNTEPVLDSDGSYFMYS | 466 |
| Without C-terminal | KLRVEKKNWVERNSYSCSVVHEGLHNHHTTKSFSRTPG | |
| Lysine | | |
| mIgG2a CH2 Region + | APNAAGGPSVFIFLLKIKDVLMISLSPIVTCVVVDVSEDDPDVQ | 467 |
| CH3 Region | ISWFVNNVEVHTAQTQTHREDYNSTLRVVSALPIQHQDWMSGKE | |
| With C-terminal | FKCKVNNKDLPAPIERTISKPKGSVRAPQVYVLPPPEEEMTKKQ | |
| Lysine | VILTCMVTDFMPEDIYVEWTNNGKTELNYKNTEPVLDSDGSYFM | |
| | YSKLRVEKKNWVERNSYSCSVVHEGLHNHHTTKSFSRTPGK | |
| mIgG2a CH2 Region + | APNAAGGPSVFIFLLKIKDVLMISLSPIVTCVVVDVSEDDPDVQ | 468 |
| CH3 Region | ISWFVNNVEVHTAQTQTHREDYNSTLRVVSALPIQHQDWMSGKE | |
| Without C-terminal | FKCKVNNKDLPAPIERTISKPKGSVRAPQVYVLPPPEEEMTKKQ | |
| Lysine | VTLTCMVTDFMPEDIYVEWTNNGKTELNYKNTEPVLDSDGSYFM | |
| | YSKLRVEKKNWVERNSYSCSVVHEGLHNHHTTKSFSRTPG | |
| mIgG2a Hinge Region + | EPRGPTIKPCPPCKCPAPNAAGGPSVFIFLLKIKDVLMISLSPI | 469 |
| CH2 Region + CH3 | VTCVVVDVSEDDPDVQISWFVNNVEVHTAQTQTHREDYNSTLRV | |
| Region | VSALPIQHQDWMSGKEFKCKVNNKDLPAPIERTISKPKGSVRAP | |
| With C-terminal | QVYVLPPPEEEMTKKQVTLTCMVTDEMPEDIYVEWINNGKTELN | |
| Lysine | YKNTEPVLDSDGSYFMYSKLRVEKKNWVERNSYSCSVVHEGLHN | |
| | HHTTKSFSRTPGK | |
| mIgG2a Hinge Region + | EPRGPTIKPCPPCKCPAPNAAGGPSVFIFLLKIKDVLMISLSPI | 470 |
| CH2 Region + CH3 | VTCVVVDVSEDDPDVQISWFVNNVEVHTAQTQTHREDYNSTLRV | |
| Region | VSALPIQHQDWMSGKEFKCKVNNKDLPAPIERTISKPKGSVRAP | |
| Without C-terminal | QVYVLPPPEEEMTKKQVTLTCMVTDFMPEDIYVEWINNGKTELN | |
| Lysine | YKNTEPVLDSDGSYFMYSKLRVEKKNWVERNSYSCSVVHEGLHN | |
| | HHTTKSFSRTPG | |
| mIgG2a CH1 Region + | AKTTPPSVYPLAPGSAAQTNSMVTLGCLVKGYFPEPVTVTWNSG | 540 |
| Hinge Region + CH2 | SLSSGVHIFPAVLQSDLYTLSSSVTVPSSPRPSETVTCNVAHPA | |
| Region + CH3 Region | SSTKVDKKIEPRGPTIKPCPPCKCPAPNAAGGPSVFIFLLKIKD | |
| With C-terminal | VLMISLSPIVICVVVDVSEDDPDVQISWFVNNVEVHTAQTQTHR | |
| Lysine | EDYNSTLRVVSALPIQHQDWMSGKEFKCKVNNKDLPAPIERTIS | |
| | KPKGSVRAPQVYVLPPPEEEMTKKQVTLTCMVTDFMPEDIYVEW | |
| | TNNGKTELNYKNTEPVLDSDGSYFMYSKLRVEKKNWVERNSYSC | |
| | SVVHEGLHNHHTTKSFSRTPGK | |
| mIgG2a CH1 Region + | AKTTPPSVYPLAPGSAAQINSMVILGCLVKGYFPEPVTVTWNSG | 541 |
| Hinge Region + CH2 | SLSSGVHIFPAVLQSDLYTLSSSVTVPSSPRPSETVTCNVAHPA | |
| Region + CH3 Region | SSTKVDKKIEPRGPTIKPCPPCKCPAPNAAGGPSVFIFLLKIKD | |
| Without C-terminal | VLMISLSPIVTCVVVDVSEDDPDVQISWFVNNVEVHTAQTQTHR | |
| Lysine | EDYNSTLRVVSALPIQHQDWMSGKEFKCKVNNKDLPAPIERTIS | |
| | KPKGSVRAPQVYVLPPPEEEMTKKQVTLTCMVTDEMPEDIYVEW | |
| | TNNGKTELNYKNTEPVLDSDGSYFMYSKLRVEKKNWVERNSYSC | |
| | SVVHEGLHNHHTTKSFSRTPG | |
| Ig light chain kappa | RADAAPTVSIFPPSSEQLTSGGASVVCFLNNFYPKDINVKWKID | 542 |
| constant region (κCL) | GSERQNGVLNSWTDQDSKDSTYSMSSTLILIKDEYERHNSYTCE | |
| | ATHKISTSPIVKSENRNEC | |
| Ig light chain kappa | QPKSSPSVTLFPPSSEELETNKATLVCTITDFYPGVVTVDWKVD | 543 |
| constant region (λCL) | GTPVTQGMETTQPSKQSNNKYMASSYLILTARAWERHSSYSCQV | |
| | THEGHTVEKSLSRADCS | |

In some embodiments, the amino acid sequence of the heterologous polypeptide comprises an amino acid sequence set forth in Table 10. In some embodiments, the amino acid sequence of the heterologous polypeptide consists of an amino acid sequence set forth in Table 10. In some embodiments, the amino acid sequence of the heterologous polypeptide comprises or consists of an amino acid sequence set forth in Table 10, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide comprises or consists of an amino acid sequence set forth in Table 10, comprising or consisting of at least about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises or consists of an amino acid sequence set forth in Table 10, comprising or consisting of about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises or consists of an amino acid sequence set forth in Table 10, comprising or consisting of about no more than 1, 2, 3, 4, 5, 6, 7, 8, 9, 10, or more amino acid variations (e.g., amino acid substitutions, deletions, or additions).

In some embodiments, the amino acid sequence of the heterologous polypeptide comprises or consists of an amino acid sequence set forth in Table 10, and further comprises 1 or more but less than 15% (less than 12%, less than 10%, less than 8%), amino acid substitutions. In some embodiments, the amino acid sequence of the heterologous polypeptide comprises or consists of an amino acid sequence set forth in Table 10, comprising or consisting of at least about 1, 2, 3, 4, 5, 6, 7, 8, 9, 10, or more amino acid substitutions. In some embodiments, the amino acid sequence of the heterologous polypeptide comprises or consists of an amino acid sequence set forth in Table 10, comprising or consisting of about 1, 2, 3, 4, 5, 6, 7, 8, 9, 10, or more amino acid substitutions. In some embodiments, the amino acid sequence of the heterologous polypeptide comprises or consists of an amino acid sequence set forth in Table 10, comprising or consisting of about no more than about 1, 2, 3, 4, 5, 6, 7, 8, 9, 10, or more amino acid substitutions.

In some embodiments, the amino acid sequence of the heterologous polypeptide comprises the amino acid sequence of any one of SEQ ID NOS: 459-462 or 467-470. In some embodiments, the amino acid sequence of the heterologous polypeptide consists of the amino acid sequence of any one of SEQ ID NOS: 459-462 or 467-470. In some embodiments, the amino acid sequence of the heterologous polypeptide comprises or consists of the amino acid sequence of any one of SEQ ID NOS: 459-462 or 467-470, and further comprises 1 or more but less than 15% (less than 12%, less than 10%, less than 8%), amino acid variations (e.g., amino acid substitutions, deletions, or additions). In some embodiments, the amino acid sequence of the heterologous polypeptide comprises or consists of the amino acid sequence of any one of SEQ ID NOS: 459-462 or 467-470, comprising or consisting at least about 1, 2, 3, 4, 5, 6, 7, 8, 9, 10, or more amino acid variations (e.g., amino acid substitutions, deletions, or additions). In some embodiments, the amino acid sequence of the heterologous polypeptide comprises or consists of the amino acid sequence of any one of SEQ ID NOS: 459-462 or 467-470, comprising or consisting about 1, 2, 3, 4, 5, 6, 7, 8, 9, or 10 amino acid variations (e.g., amino acid substitutions, deletions, or additions). In some embodiments, the amino acid sequence of the heterologous polypeptide comprises or consists of the amino acid sequence of any one of SEQ ID NOS: 459-462 or 467-470, comprising or consisting of no more than about 1, 2, 3, 4, 5, 6, 7, 8, 9, or 10 amino acid variations (e.g., amino acid substitutions, deletions, or additions).

In some embodiments, the amino acid sequence of the heterologous polypeptide comprises or consists of the amino acid sequence of any one of SEQ ID NOS: 459-462 or 467-470, and further comprises 1 or more but less than 15% (less than 12%, less than 10%, less than 8%), amino acid substitutions. In some embodiments, the amino acid sequence of the heterologous polypeptide comprises or consists of the amino acid sequence of any one of SEQ ID NOS: 459-462 or 467-470. In some embodiments, the amino acid sequence of the heterologous polypeptide comprises or consists of the amino acid sequence of any one of SEQ ID NOS: 459-462 or 467-470, comprising or consisting at least about 1, 2, 3, 4, 5, 6, 7, 8, 9, 10, or more amino acid substitutions. In some embodiments, the amino acid sequence of the heterologous polypeptide comprises or consists of the amino acid sequence of any one of SEQ ID NOS: 459-462 or 467-470, comprising or consisting about 1, 2, 3, 4, 5, 6, 7, 8, 9, or 10 amino acid substitutions. In some embodiments, the amino acid sequence of the heterologous polypeptide comprises or consists of the amino acid sequence of any one of SEQ ID NOS: 459-462 or 467-470, comprising or consisting of no more than about 1, 2, 3, 4, 5, 6, 7, 8, 9, or 10 amino acid substitutions.

In some embodiments, wherein the heterologous polypeptide comprises a CH3 region (e.g., comprises an Fc region; a hinge region, CH2 region, and CH3 region, etc.), the CH3 region lacks the C-terminal lysine (e.g., residue 227 of SEQ ID NO: 461, numbering according to SEQ ID NO: 461; or e.g., residue 223 of SEQ ID NO: 469, numbering according to SEQ ID NO: 469). In some embodiments, the CH3 region further lacks the C-terminal glycine (e.g., residue 226 of SEQ ID NO: 461, numbering according to SEQ ID NO: 461; or e.g., residue 222 of SEQ ID NO: 469, numbering according to SEQ ID NO:469). 5.3.1.3 Ig Effector Function

In some embodiments, the Ig (e.g., hIg, mIg) Fc region of a fusion protein or polypeptide described herein exhibits a decrease in one or more Fc effector function relative to a reference (e.g., wild type) Ig (e.g., hIg, mIg) Fc region. Exemplary Ig (e.g., hIg, mIg) Fc effector functions include, but are not limited to, antibody dependent cellular cytotoxicity (ADCC), antibody dependent cellular phagocytosis (ADCP), complement dependent cytotoxicity (CDC), and binding affinity to one or more human Fc receptor (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))).

Standard in vitro and/or in vivo assays known in the art can be conducted to evaluate Fe effector function, including, any one or more of ADCC, CDC, ADCP, Fc receptor (e.g., Fcγ receptor) binding affinity, and C1q binding affinity.

For example, ADCC activity can be assessed utilizing standard (radioactive and non-radioactive) methods known in the art (see, e.g., WO2006/082515, WO2012/130831), the entire contents of each of which is incorporated by reference herein for all purposes). For example, ADCC activity can be assessed using a chromium-5 (⁵¹Cr) assay. Briefly, ⁵¹Cr is pre-loaded into target cells expressing CD20, NK cells are added to the culture, and radioactivity in the cell culture supernatant is assessed (indicative of lysis of the target cells by the NK cells). Similar non-radioactive assays can also be utilized that employ a similar method, but the target cells are pre-loaded with fluorescent dyes, such as calcein-AM, CFSE, BCECF, or lanthanide flurophore (Europium). See, e.g., Parekh, Bhavin S et al. “Development and validation of an antibody-dependent cell-mediated cytotoxicity-reporter gene assay.” mAbs vol. 4, 3 (2012): 310-8. Doi:10.4161/mabs.19873, the entire contents of which is incorporated by reference herein for all purposes. Exemplary commercially available non-radioactive assays include, for example, ACTI™ non-radioactive cytotoxicity assay for flow cytometry (Cell Technology, Inc. Mountain View, Calif.; and CytoTox 96® non-radioactive cytotoxicity assay (Promega, Madison, Wis.). Additional non-limiting examples of in vitro assays that can be used to assess ADCC activity of a fusion protein described herein include those described in U.S. Pat. Nos. 5,500,362; 5,821,337; Hellstrom, I., et al., Proc. Nat'l Acad. Sci. USA 83 (1986) 7059-7063; Hellstrom, I., et al., Proc. Nat'l Acad. Sci. USA 82 (1985) 1499-1502; and Bruggemann, M., et al., J. Exp. Med. 166 (1987) 1351-1361, the entire contents of each of which is incorporated by reference herein. Alternatively, or additionally, ADCC activity of a fusion protein described herein may be assessed in vivo, e.g., in an animal model such as that disclosed in Clynes, et al., Proc. Nat'l Acad. Sci. USA 95 (1998) 652-656, the entire contents of which is incorporated by reference herein for all purposes.

C1q binding assays can be utilized to assess the ability of a hIg fusion protein or polypeptide described herein to bind C1q (or bind with less affinity than a reference fusion protein) and hence lack (or have decreased) CDC activity. The binding of a hIg fusion protein or polypeptide described herein to C1q can be determined by a variety of in vitro assays (e.g., biochemical or immunological based assays) known in the art for determining Fc-C1q interactions, including e.g., equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4^{th} Ed., Lippincott-Raven, Philadelphia (1999), the entire contents of which is incorporated by reference herein. For example, see, e.g., C1q and C3c binding ELISAs described in WO2006/029879 and WO2005/100402, the entire contents of each of which is incorporated by reference herein for all purposes. Additional CDC activity assays include those described in e.g., Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743), the entire contents of each of which is incorporated by reference herein for all purposes.

ADCP activity can be measured by in vitro or in vivo methods known in the art and also commercially available assays (see, e.g., van de Donk N W, Moreau P, Plesner T, et al. “Clinical efficacy and management of monoclonal antibodies targeting CD38 and SLAMF7 in multiple myeloma,” Blood, 127(6):681-695 (2016), the entire contents of each of which is incorporated by reference herein for all purposes). For example, a primary cell based ADCP assay can be used in which fresh human peripheral blood mononuclear cells (PBMCs) are isolated, monocytes isolated and differentiated in culture to macrophages using standard procedures. The macrophages are fluorescently labeled added to cultures containing fluorescently labeled target cells expressing CD20 and a fusion protein described herein. Phagocytosis events can be analyzed using FACS screening and/or microscopy. A modified reporter version of the above described assay can also be used that employs an engineered cell line that stably expresses FcγRIIa (CD32a) as the effector cell line (e.g., an engineered T cell line, e.g., THP-1), removing the requirement for primary cells. Exemplary ADCP assays are described in e.g., Ackerman, M. E. et al. A robust, high-throughput assay to determine the phagocytic activity of clinical antibody samples. J. Immunol. Methods 366, 8-19 (2011); and Mcandrew, E. G. et al. Determining the phagocytic activity of clinical antibody samples. J. Vis. Exp. 3588 (2011). Doi:10.3791/3588; the entire contents of each of which is incorporated by reference herein.

Binding of a hIg fusion protein or polypeptide described herein to an Ig (e.g., hIg, mIg) Fc receptor can be determined by a variety of in vitro assays (e.g., biochemical or immunological based assays) known in the art for determining Fc-Fc receptor interactions, i.e., specific binding of an Fc region to an Fc receptor. Common assays include equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4″ Ed., Lippincott-Raven, Philadelphia (1999), the entire contents of which is incorporated by reference herein for all purposes.

In some embodiments, the Ig (e.g., hIg, mIg) Fc region of a fusion protein or polypeptide described herein is varied (e.g., comprises one or more variation (e.g., one or more amino acid substitution, deletion, addition, etc.)) (referred to herein as a “variant Ig (e.g., hIg, mIg) Fc fusion polypeptide or protein”). In some embodiments, the one or more variation (e.g., the one or more amino acid substitution, deletion, addition, etc.)) decreases or abolishes one or more Fc effector function, relative to a reference Ig (e.g., hIg, mIg) Fc that does not comprise the modification (e.g., the one or more variation (e.g., the one or more amino acid substitution, deletion, addition, etc.)).

In some embodiments, the variant Ig (e.g., hIg, mIg) Fc fusion protein or polypeptide exhibits no detectable or decreased ADCC compared to a reference fusion protein or polypeptide that does not comprise the Ig (e.g., hIg, mIg) Fc modification (e.g., the one or more variation (e.g., one or more amino acid substitution, deletion, or addition)). In some embodiments, the variant Ig (e.g., hIg, mIg) Fc fusion protein or polypeptide exhibits no detectable or decreased CDC compared to a reference fusion protein or polypeptide that does not comprise the Ig (e.g., hIg, mIg) Fc modification (e.g., the one or more variation (e.g., one or more amino acid substitution, deletion, or addition)). In some embodiments, the variant Ig (e.g., hIg, mIg) Fc fusion protein or polypeptide exhibits no detectable or decreased ADCP compared to a reference fusion protein or polypeptide that does not comprise the Ig (e.g., hIg, mIg) Fc modification (e.g., the one or more variation (e.g., one or more amino acid substitution, deletion, or addition)). In some embodiments, the variant Ig (e.g., hIg, mIg) Fc fusion protein or polypeptide exhibits decreased or no binding affinity to one or more Fc receptor (e.g., human Fc receptor) (e.g., an Fcγ receptor (e.g., FcγRI, FcγRIIa, FcγRIIc, FcγRIIIa, and/or FcγRIIIb (e.g., FcγRI, FcγIIa, and/or FcγIIIa))) compared to a reference fusion protein or polypeptide that does not comprise the Ig (e.g., hIg, mIg) Fc modification (e.g., the one or more variation (e.g., one or more amino acid substitution, deletion, or addition)). In some embodiments, the variant Ig (e.g., hIg, mIg) Fc fusion protein or polypeptide exhibits decreased or no binding affinity to FcγRI, FcγIIa, and/or FcγIIIa compared to a reference fusion protein or polypeptide that does not comprise the hIg Fc modification (e.g., the one or more variation (e.g., one or more amino acid substitution, deletion, or addition)). In some embodiments, the variant Ig (e.g., hIg, mIg) Fc fusion protein or polypeptide exhibits decreased or no binding affinity to FcγRI compared to a reference fusion protein or polypeptide that does not comprise the Ig (e.g., hIg, mIg) Fc modification (e.g., the one or more variation (e.g., one or more amino acid substitution, deletion, or addition)). In some embodiments, the variant Ig (e.g., hIg, mIg) Fc fusion protein or polypeptide exhibits decreased or no binding affinity to FcγIIa compared to a reference fusion protein or polypeptide that does not comprise the Ig (e.g., hIg, mIg) Fc modification (e.g., the one or more variation (e.g., one or more amino acid substitution, deletion, or addition)). In some embodiments, the variant Ig (e.g., hIg, mIg) Fc fusion protein or polypeptide exhibits decreased or no binding affinity to FcγIIIa compared to a reference fusion protein or polypeptide that does not comprise the Ig (e.g., hIg, mIg) Fc modification (e.g., the one or more variation (e.g., one or more amino acid substitution, deletion, or addition)). In some embodiments, the variant Ig (e.g., hIg, mIg) Fc fusion protein or polypeptide exhibits decreased or no binding affinity to C1q compared to a reference fusion protein or polypeptide that does not comprise the Ig (e.g., hIg, mIg) Fc modification (e.g., the one or more variation (e.g., one or more amino acid substitution, deletion, or addition)).

Amino acid substitutions that decrease or abolish one or more Ig (e.g., hIg, mIg) Fc effector function are known in the art. See for example, Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of which is incorporated by reference herein for all purposes, see more particularly for example, e.g., Table 3 of Saunders.

In some embodiments, the variant Ig Fc fusion protein or polypeptide comprises a hIg Fc region comprising one or more amino acid variation. In some embodiments, the variant hIg Fc fusion protein or polypeptide comprises a hIg4 Fc region comprising one or more amino acid variation. In some embodiments, the hIgG4 Fc region comprises an amino acid substitution at amino acid positions S228, F234, and/or L235, EU numbering according to Kabat. In some embodiments, the hIgG4 Fc region comprises the following amino acid substitutions S228P, F234A, and/or L235A, EU numbering according to Kabat. In some embodiments, the hIgG4 Fc region comprises the following amino acid substitutions S228P, F234A, and/or L235E, EU numbering according to Kabat. In some embodiments, the hIgG4 Fc comprises the following amino acid substitutions S228P and/or L235E, EU numbering according to Kabat.

In some embodiments, the variant hIg Fc fusion protein or polypeptide comprises a hIgG1 Fc region comprising one or more amino acid variations. In some embodiments, the hIgG1 Fc region comprises an amino acid substitution at amino acid positions L234, L235, and/or P329, EU numbering according to Kabat. In some embodiments, the hIgG1 Fc region comprises the following amino acid substitutions L234A and/or L235A, EU numbering according to Kabat. In some embodiments, the hIgG1 Fc region comprises the following amino acid substitutions L234A, L235A, and P329G, EU numbering according to Kabat. In some embodiments, the hIgG1 Fc region comprises the following amino acid substitutions L234A, L235A, and P329A, EU numbering according to Kabat.

The amino acid sequence of exemplary variant hIg Fc regions that are known in the art to exhibit a decrease in one more effector function is provided in Table 5.

TABLE 5
The amino acid sequence of exemplary variant hlg Fc Regions

| | | SEQ ID |
|---|---|---|
| Description | Amino Acid Sequence | NO |
| hIgG1 CH2 Region + | PCPAPEAAGGPSVFLFPPKPKDTLMISRTPEVTCVVVDVSHE | 35 |
| CH3 Region | DPEVKFNWYVDGVEVHNAKTKPREEQYNSTYRVVSVLIVLHQ | |
| L234A/L235A | DWLNGKEYKCKVSNKALPAPIEKTISKAKGQPREPQVYTLPP | |
| With C-terminal Lysine | SRDELTKNQVSLICLVKGFYPSDIAVEWESNGQPENNYKITP | |
| | PVLDSDGSFFLYSKLTVDKSRWQQGNVFSCSVMHEALHNHYT | |
| | QKSLSLSPGK | |
| hIgG1 CH2 Region + | PCPAPEAAGGPSVFLFPPKPKDTLMISRTPEVTCVVVDVSHE | 36 |
| CH3 Region | DPEVKFNWYVDGVEVHNAKIKPREEQYNSTYRVVSVLIVLHQ | |
| L234A/L235A | DWLNGKEYKCKVSNKALPAPIEKTISKAKGQPREPQVYTLPP | |
| Without C-terminal | SRDELTKNQVSLTCLVKGFYPSDIAVEWESNGQPENNYKITP | |
| Lysine | PVLDSDGSFFLYSKLTVDKSRWQQGNVFSCSVMHEALHNHYT | |
| | QKSLSLSPG | |
| hIgG1 Partial Hinge | TCPPCPAPEAAGGPSVFLFPPKPKDTLMISRTPEVTCVVVDV | 37 |
| Region + CH2 Region + | SHEDPEVKFNWYVDGVEVHNAKTKPREEQYNSTYRVVSVLTV | |
| CH3 Region | LHQDWLNGKEYKCKVSNKALPAPIEKTISKAKGQPREPQVYT | |
| L234A/L235A | LPPSRDELTKNQVSLTCLVKGFYPSDIAVEWESNGQPENNYK | |
| With C-terminal Lysine | TTPPVLDSDGSFFLYSKLTVDKSRWQQGNVFSCSVMHEALHN | |
| | HYTQKSLSLSPGK | |
| hIgG1 Partial Hinge | TCPPCPAPEAAGGPSVFLFPPKPKDTLMISRTPEVTCVVVDV | 38 |
| Region + CH2 Region + | SHEDPEVKFNWYVDGVEVHNAKTKPREEQYNSTYRVVSVLIV | |
| CH3 Region | LHQDWLNGKEYKCKVSNKALPAPIEKTISKAKGQPREPQVYT | |
| L234A/L235A | LPPSRDELTKNQVSLTCLVKGFYPSDIAVEWESNGQPENNYK | |
| Without C-terminal | TTPPVLDSDGSFFLYSKLTVDKSRWQQGNVFSCSVMHEALHN | |
| Lysine | HYTQKSLSLSPG | |
| hIgG1 Hinge Region + | EPKSCDKTHTCPPCPAPEAAGGPSVFLFPPKPKDTLMISRTP | 39 |
| CH2 Region + CH3 | EVTCVVVDVSHEDPEVKFNWYVDGVEVHNAKTKPREEQYNST | |
| Region | YRVVSVLTVLHQDWLNGKEYKCKVSNKALPAPIEKTISKAKG | |
| L234A/L235A | QPREPQVYTLPPSRDELTKNQVSLTCLVKGFYPSDIAVEWES | |
| With C-terminal Lysine | NGQPENNYKITPPVLDSDGSFFLYSKLTVDKSRWQQGNVFSC | |
| | SVMHEALHNHYTQKSLSLSPGK | |
| hIgG1 Hinge Region + | EPKSCDKTHTCPPCPAPEAAGGPSVFLFPPKPKDILMISRTP | 40 |
| CH2 Region + CH3 | EVTCVVVDVSHEDPEVKFNWYVDGVEVHNAKTKPREEQYNST | |
| Region | YRVVSVLTVLHQDWLNGKEYKCKVSNKALPAPIEKTISKAKG | |
| L234A/L235A | QPREPQVYTLPPSRDELTKNQVSLTCLVKGFYPSDIAVEWES | |
| Without C-terminal | NGQPENNYKTTPPVLDSDGSFFLYSKLTVDKSRWQQGNVFSC | |
| Lysine | SVMHEALHNHYTQKSLSLSPG | |
| hIgG4 CH2 Region + | APEAAGGPSVFLFPPKPKDTLMISRTPEVTCVVVDVSQEDPE | 41 |
| CH3 Region | VQFNWYVDGVEVHNAKTKPREEQFNSTYRVVSVLTVLHQDWL | |
| S228P/F234A/L235A | NGKEYKCKVSNKGLPSSIEKTISKAKGQPREPQVYTLPPSQE | |
| With C-terminal Lysine | EMTKNQVSLTCLVKGFYPSDIAVEWESNGQPENNYKTTPPVL | |
| | DSDGSFFLYSRLTVDKSRWQEGNVFSCSVMHEALHNHYTQKS | |
| | LSLSLGK | |
| hIgG4 CH2 Region + | APEAAGGPSVFLFPPKPKDTLMISRTPEVTCVVVDVSQEDPE | 42 |
| CH3 Region | VQFNWYVDGVEVHNAKTKPREEQFNSTYRVVSVLTVLHQDWL | |
| S228P/F234A/L235A | NGKEYKCKVSNKGLPSSIEKTISKAKGQPREPQVYTLPPSQE | |
| Without C-terminal | EMTKNQVSLTCLVKGFYPSDIAVEWESNGQPENNYKTTPPVL | |
| Lysine | DSDGSFFLYSRLTVDKSRWQEGNVFSCSVMHEALHNHYTQKS | |
| | LSLSLG | |
| hIgG4 Partial Hinge | PCPSCPAPEAAGGPSVFLFPPKPKDTLMISRTPEVTCVVVDV | 43 |
| Region + CH2 Region + | SQEDPEVQFNWYVDGVEVHNAKTKPREEQFNSTYRVVSVLTV | |
| CH3 Region | LHQDWLNGKEYKCKVSNKGLPSSIEKTISKAKGQPREPQVYT | |
| S228P/F234A/L235A | LPPSQEEMTKNQVSLTCLVKGFYPSDIAVEWESNGQPENNYK | |
| With C-terminal Lysine | TTPPVLDSDGSFFLYSRLTVDKSRWQEGNVFSCSVMHEALHN | |
| | HYTQKSLSLSLGK | |
| hIgG4 Partial Hinge | PCPPCPAPEAAGGPSVFLFPPKPKDTLMISRTPEVTCVVVDV | 44 |
| Region + CH2 Region + | SQEDPEVQFNWYVDGVEVHNAKTKPREEQFNSTYRVVSVLIV | |
| CH3 Region | LHQDWLNGKEYKCKVSNKGLPSSIEKTISKAKGQPREPQVYT | |
| S228P/F234A/L235A | LPPSQEEMTKNQVSLTCLVKGFYPSDIAVEWESNGQPENNYK | |
| Without C-terminal | TTPPVLDSDGSFFLYSRLTVDKSRWQEGNVFSCSVMHEALHN | |
| Lysine | HYTQKSLSLSLG | |
| hIgG4 Hinge Region + | ESKYGPPCPPCPAPEAAGGPSVFLFPPKPKDTLMISRTPEVT | 45 |
| CH2 Region + CH3 | CVVVDVSQEDPEVQFNWYVDGVEVHNAKTKPREEQFNSTYRV | |
| Region | VSVLTVLHQDWLNGKEYKCKVSNKGLPSSIEKTISKAKGQPR | |
| S228P/F234A/L235A | EPQVYTLPPSQEEMTKNQVSLTCLVKGFYPSDIAVEWESNGQ | |
| With C-terminal Lysine | PENNYKTTPPVLDSDGSFFLYSRLTVDKSRWQEGNVFSCSVM | |
| | HEALHNHYTQKSLSLSLGK | |
| hIgG4 Hinge Region + | ESKYGPPCPPCPAPEAAGGPSVFLFPPKPKDTLMISRTPEVT | 46 |
| CH2 Region + CH3 | CVVVDVSQEDPEVQFNWYVDGVEVHNAKTKPREEQFNSTYRV | |
| Region | VSVLTVLHQDWLNGKEYKCKVSNKGLPSSIEKTISKAKGQPR | |
| S228P/F234A/L235A | EPQVYTLPPSQEEMTKNQVSLTCLVKGFYPSDIAVEWESNGQ | |
| Without C-terminal | PENNYKTTPPVLDSDGSFFLYSRLTVDKSRWQEGNVFSCSVM | |
| Lysine | HEALHNHYTQKSLSLSLG | |
| hIgG4 Hinge Region + | AESKYGPPCPPCPAPEAAGGPSVFLFPPKPKDTLMISRTPEV | 47 |
| CH2 Region + CH3 | TCVVVDVSQEDPEVQFNWYVDGVEVHNAKTKPREEQFNSTYR | |
| Region | VVSVLTVLHQDWLNGKEYKCKVSNKGLPSSIEKTISKAKGQP | |
| (Variant) | REPQVYTLPPSQEEMTKNQVSLTCLVKGFYPSDIAVEWESNG | |
| S228P/F234A/L235A | QPENNYKTTPPVLDSDGSFFLYSRLTVDKSRWQEGNVFSCSV | |
| With C-terminal Lysine | MHEALHNHYTQKSLSLSLGK | |
| hIgG4 Hinge Region + | AESKYGPPCPPCPAPEAAGGPSVFLFPPKPKDTLMISRTPEV | 48 |
| CH2 Region + CH3 | TCVVVDVSQEDPEVQFNWYVDGVEVHNAKTKPREEQFNSTYR | |
| Region | VVSVLIVLHQDWLNGKEYKCKVSNKGLPSSIEKTISKAKGQP | |
| (Variant) | REPQVYTLPPSQEEMTKNQVSLTCLVKGFYPSDIAVEWESNG | |
| S228P/F234A/L235A | QPENNYKTTPPVLDSDGSFFLYSRLTVDKSRWQEGNVFSCSV | |
| Without C-terminal | MHEALHNHYTQKSLSLSLG | |
| Lysine | | |

In some embodiments, the variant hIg Fe fusion protein or polypeptide comprises a hIg Fe region comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 5. For example, the variant hIg Fc fusion protein or polypeptide may comprise a hIg Fc region comprising an amino acid sequence at least 85% identical to the amino acid sequence of a polypeptide set forth in Table 5. The variant hIg Fc fusion protein or polypeptide may comprise a hIg Fc region comprising an amino acid sequence at least 90% identical to the amino acid sequence of a polypeptide set forth in Table 5. The variant hIg Fc fusion protein or polypeptide may comprise a hIg Fc region comprising an amino acid sequence at least 95% identical to the amino acid sequence of a polypeptide set forth in Table 5. In some embodiments, the variant hIg Fc fusion protein or polypeptide preferably may comprise a hIg Fc region comprising an amino acid sequence 100% identical to the amino acid sequence of a polypeptide set forth in Table 5.

In some embodiments, the amino acid sequence of the variant hIg Fc fusion protein or polypeptide comprises a hIg Fc region that comprises or consists of the amino acid sequence of a polypeptide set forth in Table 5, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein or polypeptide comprises a hIg Fc region that comprises or consists of the amino acid sequence of a polypeptide set forth in Table 5, and further comprises or consists of at least about 1, 2, 3, 4, 5, 6, 7, 8, 9, or 10 amino acid variations (e.g., substitutions, additions, deletions, etc.). In some embodiments, the amino acid sequence of the variant hIg Fc fusion protein or polypeptide comprises a hIg Fc region that comprises or consists of the amino acid sequence of a polypeptide set forth in Table 5, and further comprises or consists of about 1, 2, 3, 4, 5, 6, 7, 8, 9, or 10 amino acid variations (e.g., substitutions, additions, deletions, etc.). In some embodiments, the amino acid sequence of the variant hIg Fc fusion protein or polypeptide comprises a hIg Fc region that comprises or consists of the amino acid sequence of a polypeptide set forth in Table 5, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the variant hIg Fc fusion protein or polypeptide comprises a hIg Fc region that comprises or consists of the amino acid sequence of a polypeptide set forth in Table 5, and further comprises 1 or more but less than 15% (less than 12%, less than 10%, less than 8%), amino acid substitutions. In some embodiments, the amino acid sequence of the variant hIg Fc fusion protein or polypeptide comprises a hIg Fc region that comprises or consists of the amino acid sequence of a polypeptide set forth in Table 5, and further comprises or consists of at least about 1, 2, 3, 4, 5, 6, 7, 8, 9, or 10 amino acid substitutions. In some embodiments, the amino acid sequence of the variant hIg Fc fusion protein or polypeptide comprises a hIg Fc region that comprises or consists of the amino acid sequence of a polypeptide set forth in Table 5, and further comprises or consists of about 1, 2, 3, 4, 5, 6, 7, 8, 9, or 10 amino acid substitutions. In some embodiments, the amino acid sequence of the variant hIg Fc fusion protein or polypeptide comprises a hIg Fc region that comprises or consists of the amino acid sequence of a polypeptide set forth in Table 5, and further comprises or consists of no more than about 1, 2, 3, 4, 5, 6, 7, 8, 9, or 10 amino acid substitutions.

In some embodiments, the amino acid sequence of the variant hIg Fc fusion protein or polypeptide comprises a hIg Fc region that comprises an amino acid sequence at least 85%, 86%, 87%, 88%, 89%, 90%, 91%, 92%, 93%, 94%, 95%, 96%, 97%, 98%, or 100% identical to the amino acid sequence of any one of SEQ ID NOS: 35-48. For example, the amino acid sequence of the variant hIg Fc fusion protein or polypeptide may comprise a hIg Fc region that comprises an amino acid sequence at least 85% identical to the amino acid sequence of any one of SEQ ID NOS: 35-48. The amino acid sequence of the variant hIg Fc fusion protein or polypeptide may comprise a hIg Fc region that comprises an amino acid sequence at least 90% identical to the amino acid sequence of any one of SEQ ID NOS: 35-48. The amino acid sequence of the variant hIg Fc fusion protein or polypeptide may comprise a hIg Fc region that comprises an amino acid sequence at least 95% identical to the amino acid sequence of any one of SEQ ID NOS: 35-48. In some embodiments, the amino acid sequence of the variant hIg Fc fusion protein or polypeptide preferably may comprise a hIg Fc region that comprises an amino acid sequence 100% identical to the amino acid sequence of any one of SEQ ID NOS: 35-48. In some embodiments, the amino acid sequence of the variant hIg Fc fusion protein or polypeptide comprises a hIg Fc region that consists of an amino acid sequence at least 85%, 86%, 87%, 88%, 89%, 90%, 91%, 92%, 93%, 94%, 95%, 96%, 97%, 98%, or 100% identical to the amino acid sequence of any one of SEQ ID NOS: 35-48. For example, the amino acid sequence of the variant hIg Fc fusion protein or polypeptide may comprise a hIg Fc region that consists of an amino acid sequence at least 85% identical to the amino acid sequence of any one of SEQ ID NOS: 35-48. The amino acid sequence of the variant hIg Fc fusion protein or polypeptide may comprise a hIg Fc region that consists of an amino acid sequence at least 90% identical to the amino acid sequence of any one of SEQ ID NOS: 35-48. The amino acid sequence of the variant hIg Fc fusion protein or polypeptide may comprise a hIg Fc region that consists of an amino acid sequence at least 95% identical to the amino acid sequence of any one of SEQ ID NOS: 35-48. In some embodiments, the amino acid sequence of the variant hIg Fc fusion protein or polypeptide preferably may comprise a hIg Fc region that consists of an amino acid sequence 100% identical to the amino acid sequence of any one of SEQ ID NOS: 35-48.

In some embodiments, the amino acid sequence of the variant hIg Fc fusion protein or polypeptide comprises a hIg Fc region that comprises or consists of the amino acid sequence of any one of SEQ ID NOS: 35-48, and further comprises 1 or more but less than 15% (less than 12%, less than 10%, less than 8%), amino acid variations (e.g., amino acid substitutions, deletions, or additions). In some embodiments, the amino acid sequence of the variant hIg Fc fusion protein or polypeptide comprises a hIg Fc region that comprises or consists of the amino acid sequence of any one of SEQ ID NOS: 35-48, and further comprises or consists of at least about 1, 2, 3, 4, 5, 6, 7, 8, 9, or 10 amino acid variations (e.g., substitutions, additions, deletions, etc.). In some embodiments, the amino acid sequence of the variant hIg Fc fusion protein or polypeptide comprises a hIg Fc region that comprises or consists of the amino acid sequence of any one of SEQ ID NOS: 35-48, and further comprises or consists of about 1, 2, 3, 4, 5, 6, 7, 8, 9, or 10 amino acid variations (e.g., substitutions, additions, deletions, etc.). In some embodiments, the amino acid sequence of the variant hIg Fc fusion protein or polypeptide comprises a hIg Fc region that comprises or consists of the amino acid sequence of any one of SEQ ID NOS: 35-48, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the variant hIg Fc fusion protein or polypeptide comprises a hIg Fc region that comprises or consists of the amino acid sequence of any one of SEQ ID NOS: 35-48, and further comprises 1 or more but less than 15% (less than 12%, less than 10%, less than 8%), amino acid substitutions. In some embodiments, the amino acid sequence of the variant hIg Fc fusion protein or polypeptide comprises a hIg Fc region that comprises or consists of the amino acid sequence of any one of SEQ ID NOS: 35-48, and further comprises or consists of at least about 1, 2, 3, 4, 5, 6, 7, 8, 9, or 10 amino acid substitutions. In some embodiments, the amino acid sequence of the variant hIg Fc fusion protein or polypeptide comprises a hIg Fc region that comprises or consists of the amino acid sequence of any one of SEQ ID NOS: 35-48, and further comprises or consists of about 1, 2, 3, 4, 5, 6, 7, 8, 9, or 10 amino acid substitutions. In some embodiments, the amino acid sequence of the variant hIg Fc fusion protein or polypeptide comprises a hIg Fc region that comprises or consists of the amino acid sequence of any one of SEQ ID NOS: 35-48, and further comprises or consists of no more than about 1, 2, 3, 4, 5, 6, 7, 8, 9, or 10 amino acid substitutions.

In some embodiments, the variant mIg Fc fusion protein or polypeptide comprises a mIgG2a Fc region comprising one or more amino acid variations. In some embodiments, the mIgG2a Fc region comprises an amino acid substitution at amino acid positions L234, L235, and/or P329, EU numbering according to Kabat. In some embodiments, the mIgG2a Fc region comprises the following amino acid substitutions L234P and/or L235P, EU numbering according to Kabat. In some embodiments, the mIgG2a Fc region comprises the following amino acid substitutions L234P, L235P, and P329G, EU numbering according to Kabat. In some embodiments, the mIgG2a Fc region comprises the following amino acid substitutions L234P, L235P, and P329A, EU numbering according to Kabat.

In some embodiments, the variant mIg Fc fusion protein or polypeptide comprises a mIgG2a Fc region comprising one or more amino acid variations. In some embodiments, the mIgG2a Fc region comprises an amino acid substitution at amino acid positions L234, L235, and/or P329, EU numbering according to Kabat. In some embodiments, the mIgG2a Fc region comprises the following amino acid substitutions L234A and/or L235A, EU numbering according to Kabat. In some embodiments, the mIgG2a Fc region comprises the following amino acid substitutions L234A, L235A, and P329G, EU numbering according to Kabat. In some embodiments, the mIgG2a Fc region comprises the following amino acid substitutions L234A, L235A, and P329A, EU numbering according to Kabat.

The amino acid sequence of exemplary variant hIg Fc regions that are known in the art to exhibit a decrease in one more effector function is provided in Table 11.

TABLE 11
The amino acid sequence of exemplary variant mlg Fc Regions

| | | SEQ |
|---|---|---|
| Description | Amino Acid Sequence | ID NO |
| mIgG2a CH2 Region + | APNAAGGPSVFIFPPKIKDVLMISLSPIVTCVVVDVSEDDPDV | 471 |
| CH3 Region | QISWFVNNVEVHTAQTQTHREDYNSTLRVVSALPIQHQDWMSG | |
| L234P/L235P | KEFKCKVNNKDLPAPIERTISKPKGSVRAPQVYVLPPPEEEMT | |
| With C-terminal Lysine | KKQVTLTCMVIDFMPEDIYVEWINNGKTELNYKNTEPVLDSDG | |
| | SYFMYSKLRVEKKNWVERNSYSCSVVHEGLHNHHTTKSFSRTP | |
| | GK | |
| mIgG2a CH2 Region + | APNAAGGPSVFIFPPKIKDVLMISLSPIVTCVVVDVSEDDPDV | 472 |
| CH3 Region | QISWFVNNVEVHTAQTQTHREDYNSTLRVVSALPIQHQDWMSG | |
| L234P/L235P | KEFKCKVNNKDLPAPIERTISKPKGSVRAPQVYVLPPPEEEMT | |
| Without C-terminal | KKQVTLTCMVIDFMPEDIYVEWINNGKTELNYKNTEPVLDSDG | |
| Lysine | SYFMYSKLRVEKKNWVERNSYSCSVVHEGLHNHHTTKSFSRTP | |
| | G | |
| mIgG2a Hinge Region + | EPRGPTIKPCPPCKCPAPNAAGGPSVFIFPPKIKDVLMISLSP | 473 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234P/L235P /P329G | RAPQVYVLPPPEEEMTKKQVTLTCMVTDFMPEDIYVEWINNGK | |
| With C-terminal Lysine | TELNYKNIEPVLDSDGSYFMYSKLRVEKKNWVERNSYSCSVVH | |
| | EGLHNHHTTKSFSRTPGK | |
| mIgG2a Hinge Region + | EPRGPTIKPCPPCKCPAPNAAGGPSVFIFPPKIKDVLMISLSP | 474 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234P/L235P /P329G | RAPQVYVLPPPEEEMTKKQVTLTCMVTDFMPEDIYVEWINNGK | |
| Without C-terminal | TELNYKNTEPVLDSDGSYFMYSKLRVEKKNWVERNSYSCSVVH | |
| Lysine | EGLHNHHTTKSFSRTPG | |
| mIgG2a CH2 Region + | APNAAGGPSVFIFAAKIKDVLMISLSPIVTCVVVDVSEDDPDV | 475 |
| CH3 Region | QISWFVNNVEVHTAQTQTHREDYNSTLRVVSALPIQHQDWMSG | |
| L234A/L235A | KEFKCKVNNKDLPAPIERTISKPKGSVRAPQVYVLPPPEEEMT | |
| With C-terminal Lysine | KKQVTLTCMVTDFMPEDIYVEWTNNGKTELNYKNTEPVLDSDG | |
| | SYFMYSKLRVEKKNWVERNSYSCSVVHEGLHNHHTTKSFSRTP | |
| | GK | |
| mIgG2a CH2 Region + | APNAAGGPSVFIFAAKIKDVLMISLSPIVTCVVVDVSEDDPDV | 476 |
| CH3 Region | QISWFVNNVEVHTAQTQTHREDYNSTLRVVSALPIQHQDWMSG | |
| L234A/L235A | KEFKCKVNNKDLPAPIERTISKPKGSVRAPQVYVLPPPEEEMT | |
| Without C-terminal | KKQVTLTCMVTDFMPEDIYVEWTNNGKTELNYKNTEPVLDSDG | |
| Lysine | SYFMYSKLRVEKKNWVERNSYSCSVVHEGLHNHHTTKSFSRIP | |
| | G | |
| mIgG2a Hinge Region + | EPRGPTIKPCAACKCPAPNAAGGPSVFIFPPKIKDVLMISLSP | 477 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234A/L235A/P329G | RAPQVYVLPPPEEEMTKKQVTLTCMVTDEMPEDIYVEWINNGK | |
| With C-terminal Lysine | TELNYKNTEPVLDSDGSYFMYSKLRVEKKNWVERNSYSCSVVH | |
| | EGLHNHHTTKSFSRTPGK | |
| mIgG2a Hinge Region + | EPRGPTIKPCAACKCPAPNAAGGPSVFIFPPKIKDVLMISLSP | 478 |
| CH2 Region + CH3 | IVTCVVVDVSEDDPDVQISWFVNNVEVHTAQTQTHREDYNSTL | |
| Region | RVVSALPIQHQDWMSGKEFKCKVNNKDLGAPIERTISKPKGSV | |
| L234A/L235A/P329G | RAPQVYVLPPPEEEMTKKQVTLTCMVTDEMPEDIYVEWINNGK | |
| Without C-terminal | TELNYKNTEPVLDSDGSYFMYSKLRVEKKNWVERNSYSCSVVH | |
| Lysine | EGLHNHHTTKSFSRTPG | |

In some embodiments, the variant mIg Fe fusion protein or polypeptide comprises a mIg Fe region comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 11. For example, the variant mIg Fc fusion protein or polypeptide may comprise a mIg Fc region comprising an amino acid sequence at least 85% identical to the amino acid sequence of a polypeptide set forth in Table 11. The variant mIg Fc fusion protein or polypeptide may comprise a mIg Fc region comprising an amino acid sequence at least 90% identical to the amino acid sequence of a polypeptide set forth in Table 11. The variant mIg Fc fusion protein or polypeptide may comprise a mIg Fc region comprising an amino acid sequence at least 95% identical to the amino acid sequence of a polypeptide set forth in Table 11. In some embodiments, the variant mIg Fc fusion protein or polypeptide preferably may comprise a mIg Fc region comprising an amino acid sequence 100% identical to the amino acid sequence of a polypeptide set forth in Table 11.

In some embodiments, the amino acid sequence of the variant mIg Fc fusion protein or polypeptide comprises a mIg Fc region that comprises or consists of the amino acid sequence of a polypeptide set forth in Table 11, and further comprises 1 or more but less than 15% (less than 12%, less than 10%, less than 8%), amino acid variations (e.g., amino acid substitutions, deletions, or additions). In some embodiments, the amino acid sequence of the variant mIg Fc fusion protein or polypeptide comprises a mIg Fc region that comprises or consists of the amino acid sequence of a polypeptide set forth in Table 11, and further comprises or consists of at least about 1, 2, 3, 4, 5, 6, 7, 8, 9, or 10 amino acid variations (e.g., substitutions, additions, deletions, etc.). In some embodiments, the amino acid sequence of the variant mIg Fc fusion protein or polypeptide comprises a mIg Fc region that comprises or consists of the amino acid sequence of a polypeptide set forth in Table 11, and further comprises or consists of about 1, 2, 3, 4, 5, 6, 7, 8, 9, or 10 amino acid variations (e.g., substitutions, additions, deletions, etc.). In some embodiments, the amino acid sequence of the variant mIg Fc fusion protein or polypeptide comprises a mIg Fc region that comprises or consists of the amino acid sequence of a polypeptide set forth in Table 11, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the variant mIg Fc fusion protein or polypeptide comprises a mIg Fc region that comprises or consists of the amino acid sequence of a polypeptide set forth in Table 11, and further comprises 1 or more but less than 15% (less than 12%, less than 10%, less than 8%), amino acid substitutions. In some embodiments, the amino acid sequence of the variant mIg Fc fusion protein or polypeptide comprises a mIg Fc region that comprises or consists of the amino acid sequence of a polypeptide set forth in Table 11, and further comprises or consists of at least about 1, 2, 3, 4, 5, 6, 7, 8, 9, or 10 amino acid substitutions. In some embodiments, the amino acid sequence of the variant mIg Fc fusion protein or polypeptide comprises a mIg Fc region that comprises or consists of the amino acid sequence of a polypeptide set forth in Table 11, and further comprises or consists of about 1, 2, 3, 4, 5, 6, 7, 8, 9, or 10 amino acid substitutions. In some embodiments, the amino acid sequence of the variant mIg Fc fusion protein or polypeptide comprises a mIg Fc region that comprises or consists of the amino acid sequence of a polypeptide set forth in Table 11, and further comprises or consists of no more than about 1, 2, 3, 4, 5, 6, 7, 8, 9, or 10 amino acid substitutions.

In some embodiments, the amino acid sequence of the variant mIg Fc fusion protein or polypeptide comprises a mIg Fc region that comprises an amino acid sequence at least 85%, 86%, 87%, 88%, 89%, 90%, 91%, 92%, 93%, 94%, 95%, 96%, 97%, 98%, or 100% identical to the amino acid sequence of any one of SEQ ID NOS: 471-478. For example, the amino acid sequence of the variant mIg Fc fusion protein or polypeptide may comprise a mIg Fc region that comprises an amino acid sequence at least 85% identical to the amino acid sequence of any one of SEQ ID NOS: 471-478. The amino acid sequence of the variant mIg Fc fusion protein or polypeptide may comprise a mIg Fc region that comprises an amino acid sequence at least 90% identical to the amino acid sequence of any one of SEQ ID NOS: 471-478. The amino acid sequence of the variant mIg Fc fusion protein or polypeptide may comprise a mIg Fc region that comprises an amino acid sequence at least 95% identical to the amino acid sequence of any one of SEQ ID NOS: 471-478. In some embodiments, the amino acid sequence of the variant mIg Fc fusion protein or polypeptide preferably may comprise a mIg Fc region that comprises an amino acid sequence 100% identical to the amino acid sequence of any one of SEQ ID NOS: 471-478. In some embodiments, the amino acid sequence of the variant mIg Fc fusion protein or polypeptide comprises a mIg Fc region that consists of an amino acid sequence at least 85%, 86%, 87%, 88%, 89%, 90%, 91%, 92%, 93%, 94%, 95%, 96%, 97%, 98%, or 100% identical to the amino acid sequence of any one of SEQ ID NOS: 471-478. For example, the amino acid sequence of the variant mIg Fc fusion protein or polypeptide may comprise a mIg Fc region that consists of an amino acid sequence at least 85% identical to the amino acid sequence of any one of SEQ ID NOS: 471-478. The amino acid sequence of the variant mIg Fc fusion protein or polypeptide may comprise a hIg Fc region that consists of an amino acid sequence at least 90% identical to the amino acid sequence of any one of SEQ ID NOS: 471-478. The amino acid sequence of the variant mIg Fc fusion protein or polypeptide may comprise a mIg Fc region that consists of an amino acid sequence at least 95% identical to the amino acid sequence of any one of SEQ ID NOS: 471-478. In some embodiments, the amino acid sequence of the variant mIg Fc fusion protein or polypeptide preferably may comprise a mIg Fc region that consists of an amino acid sequence 100% identical to the amino acid sequence of any one of SEQ ID NOS: 471-478.

In some embodiments, the amino acid sequence of the variant mIg Fc fusion protein or polypeptide comprises a mIg Fc region that comprises or consists of the amino acid sequence of any one of SEQ ID NOS: 471-478, and further comprises 1 or more but less than 15% (less than 12%, less than 10%, less than 8%), amino acid variations (e.g., amino acid substitutions, deletions, or additions). In some embodiments, the amino acid sequence of the variant mIg Fc fusion protein or polypeptide comprises a mIg Fc region that comprises or consists of the amino acid sequence of any one of SEQ ID NOS: 471-478, and further comprises or consists of at least about 1, 2, 3, 4, 5, 6, 7, 8, 9, or 10 amino acid variations (e.g., substitutions, additions, deletions, etc.). In some embodiments, the amino acid sequence of the variant mIg Fc fusion protein or polypeptide comprises a mIg Fc region that comprises or consists of the amino acid sequence of any one of SEQ ID NOS: 471-478, and further comprises or consists of about 1, 2, 3, 4, 5, 6, 7, 8, 9, or 10 amino acid variations (e.g., substitutions, additions, deletions, etc.). In some embodiments, the amino acid sequence of the variant mIg Fc fusion protein or polypeptide comprises a mIg Fc region that comprises or consists of the amino acid sequence of any one of SEQ ID NOS: 471-478, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the variant mIg Fc fusion protein or polypeptide comprises a mIg Fc region that comprises or consists of the amino acid sequence of any one of SEQ ID NOS: 471-478, and further comprises 1 or more but less than 15% (less than 12%, less than 10%, less than 8%), amino acid substitutions. In some embodiments, the amino acid sequence of the variant mIg Fc fusion protein or polypeptide comprises a mIg Fc region that comprises or consists of the amino acid sequence of any one of SEQ ID NOS: 471-478, and further comprises or consists of at least about 1, 2, 3, 4, 5, 6, 7, 8, 9, or 10 amino acid substitutions. In some embodiments, the amino acid sequence of the variant mIg Fc fusion protein or polypeptide comprises a mIg Fc region that comprises or consists of the amino acid sequence of any one of SEQ ID NOS: 471-478, and further comprises or consists of about 1, 2, 3, 4, 5, 6, 7, 8, 9, or 10 amino acid substitutions. In some embodiments, the amino acid sequence of the variant mIg Fc fusion protein or polypeptide comprises a mIg Fc region that comprises or consists of the amino acid sequence of any one of SEQ ID NOS: 471-478, and further comprises or consists of no more than about 1, 2, 3, 4, 5, 6, 7, 8, 9, or 10 amino acid substitutions.

### 5.3.2 Linkers

As described herein, the heterologous moiety (e.g., heterologous polypeptide) can be directly operably connected or indirectly operably connected to the immunomodulatory protein or polypeptide (e.g., described herein). In some embodiments, the heterologous polypeptide is directly operably connected to the immunomodulatory protein or polypeptide (e.g., described herein) via a peptide bond. In some embodiment, the heterologous polypeptide is indirectly operably connected to the immunomodulatory protein or polypeptide (e.g., described herein) via a peptide linker.

In some embodiments, the peptide linker is one or any combination of a cleavable linker, a non-cleavable linker, a flexible linker, a rigid linker, a helical linker, and/or a non-helical linker.

In some embodiments, the peptide linker comprises from or from about 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, or 5-10 amino acid residues. In some embodiments, the peptide linker comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the linker comprises or consists of no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues.

In some embodiments, the amino acid sequence of the peptide linker comprises or consists of glycine, serine, or both glycine and serine amino acid residues. In some embodiments, the amino acid sequence of the peptide linker comprises or consists of glycine, serine, and proline amino acid residues.

The amino acid sequence of exemplary peptide linkers, which can be incorporated in one or more of the embodiments described herein (e.g., fusion proteins and polypeptide), is set provided in Table 6.

TABLE 6
The Amino Acid Sequence of Exemplary Peptide Linker

| | | SEQ ID |
|---|---|---|
| Description | Amino Acid Sequence | NO |
| A | GGGGGGGS | 49 |
| B | GGGGGGGGGGGGGGS | 50 |
| C | GGGGGGGSGGGGGGGSGGGGGGGS | 51 |
| D | GGGGS | 52 |
| E | GGGGSGGGGS | 53 |
| F | GGGGSGGGGSGGGGS | 54 |
| G | GGGS | 55 |
| H | GGGSGGGS | 56 |
| I | GGGSGGGSGGGS | 57 |

In some embodiments, the amino acid sequence of the peptide linker comprises the amino acid sequence of any one of the linkers set forth in Table 6. In some embodiments, the amino acid sequence of the peptide linker consists of the amino acid sequence of any one of the linkers set forth in Table 6. In some embodiments, the amino acid sequence of the peptide linker comprises or consists of the amino acid sequence of any one of the linkers set forth in Table 6, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of the linkers set forth in Table 6, comprising 1, 2, or 3 amino acid variations (e.g., substitutions, deletions, additions). In some embodiments, the amino acid sequence of the peptide linker comprises or consists of the amino acid sequence of any one of the linkers set forth in Table 6, and further comprises 1 or more but less than 15% (less than 12%, less than 10%, less than 8%), amino acid substitutions. In some embodiments, the amino acid sequence of the peptide linker comprises or consists of the amino acid sequence of any one of the linkers set forth in Table 6, comprising 1, 2, or 3 amino acid substitutions.

In some embodiments, the amino acid sequence of the peptide linker comprises the amino acid sequence of any one of SEQ ID NOS: 49-57. In some embodiments, the amino acid sequence of the peptide linker consists of the amino acid sequence of any one of SEQ ID NOS: 49-57. In some embodiments, the amino acid sequence of the peptide linker comprises or consists of the amino acid sequence of any one of SEQ ID NOS: 49-57, and further comprises 1 or more but less than 15% (less than 12%, less than 10%, less than 8%), amino acid variations (e.g., amino acid substitutions, deletions, or additions). In some embodiments, the amino acid sequence of the peptide linker comprises or consists of the amino acid sequence of any one of SEQ ID NOS: 49-57, comprising 1, 2, or 3 amino acid variations (e.g., substitutions, deletions, additions). In some embodiments, the amino acid sequence of the peptide linker comprises or consists of the amino acid sequence of any one of SEQ ID NOS: 49-57, and further comprises 1 or more but less than 15% (less than 12%, less than 10%, less than 8%), amino acid substitutions. In some embodiments, the amino acid sequence of the peptide linker comprises or consists of the amino acid sequence of any one of SEQ ID NOS: 49-57, comprising 1, 2, or 3 amino acid substitutions.

In some embodiments, the amino acid sequence of the peptide linker comprises the amino acid sequence of SEQ ID NO: 51, and further comprises 1 or more but less than 15% (less than 12%, less than 10%, less than 8%), amino acid substitutions. In some embodiments, the amino acid sequence of the peptide linker comprises the amino acid sequence of SEQ ID NO: 51. In some embodiments, the amino acid sequence of the peptide linker comprises the amino acid sequence of any one of SEQ ID NO: 51, comprising 1, 2, or 3 amino acid variations (e.g., substitutions, additions, deletions). In some embodiments, the amino acid sequence of the peptide linker comprises the amino acid sequence of any one of SEQ ID NO: 51, comprising 1, 2, or 3 amino acid substitutions. In some embodiments, the amino acid sequence of the peptide linker consists of the amino acid sequence of SEQ ID NO: 51, and further comprises 1 or more but less than 15% (less than 12%, less than 10%, less than 8%), amino acid substitutions. In some embodiments, the amino acid sequence of the peptide linker consists of the amino acid sequence of SEQ ID NO: 51. In some embodiments, the amino acid sequence of the peptide linker consists of the amino acid sequence of any one of SEQ ID NO: 51, comprising 1, 2, or 3 amino acid variations (e.g., substitutions, additions, deletions). In some embodiments, the amino acid sequence of the peptide linker consists of the amino acid sequence of any one of SEQ ID NO: 51, comprising 1, 2, or 3 amino acid substitutions. 5.3.3 Orientation

The heterologous moiety (e.g., heterologous polypeptide) and the immunomodulatory protein or polypeptide (e.g., described herein) can be arranged in any configuration or order as long as the immunomodulatory protein or polypeptide (e.g., described herein) maintains the ability to mediate its function (e.g., bind to the hIL-10R) and in the embodiments wherein the heterologous moiety (e.g., heterologous polypeptide) has a specific function, the heterologous moiety (e.g., heterologous polypeptide) can mediate its function.

In some embodiments, the heterologous moiety is a heterologous polypeptide (e.g., an Ig (e.g., hIg, mIg) Fc region (e.g., an Ig (e.g., hIg, mIg) Fc region described herein)) forming a fusion polypeptide or protein. In some embodiments, the fusion polypeptide or protein comprises from N- to C-terminus: an immunomodulatory polypeptide (e.g., described herein) and a heterologous polypeptide (e.g., an Ig (e.g., hIg, mIg) Fc region (e.g., an Ig (e.g., hIg, mIg) Fc region described herein)). In some embodiments, the fusion polypeptide or protein comprises from N- to C-terminus: an immunomodulatory polypeptide (e.g., described herein), a peptide linker (e.g., described herein), and a heterologous polypeptide (e.g., an Ig (e.g., hIg, mIg) Fc region (e.g., an Ig (e.g., hIg, mIg) Fc region described herein)). In this specific orientation, the N-terminus of the immunomodulatory polypeptide (e.g., described herein) is operably connected to the C-terminus of the heterologous polypeptide (e.g., an Ig (e.g., hIg, mIg) Fc region (e.g., an Ig (e.g., hIg, mIg) Fc region described herein)) either directly or indirectly through the peptide linker (e.g., described herein).

In some embodiments, the fusion polypeptide comprises from N- to C-terminus: a heterologous polypeptide (e.g., an Ig (e.g., hIg, mIg) Fc region (e.g., an Ig (e.g., hIg, mIg) Fc region described herein)) and an immunomodulatory polypeptide (e.g., described herein). In some embodiments, the fusion polypeptide comprises from N- to C-terminus: a heterologous polypeptide (e.g., an Ig (e.g., hIg, mIg) Fc region (e.g., an Ig (e.g., hIg, mIg) Fc region described herein)), a peptide linker (e.g., described herein), and an immunomodulatory polypeptide (e.g., described herein). In this specific orientation, the C-terminus of the immunomodulatory polypeptide (e.g., described herein) is operably connected to the N-terminus of the heterologous polypeptide (e.g., an Ig (e.g., hIg, mIg) Fc region (e.g., an Ig (e.g., hIg, mIg) Fc region described herein)) either directly or indirectly through the peptide linker (e.g., described herein).

### 5.3.4 Multimeric Fusion Proteins

In one aspect, provided herein are multimeric (e.g., dimeric) proteins comprising at least two polypeptide or protein fusions or conjugates described herein (e.g., Ig (e.g., hIg, mIg) Fc fusion proteins or polypeptides described herein). In some embodiments, the protein is dimeric. In some embodiments, the protein is homodimeric. In some embodiments, the protein is heterodimeric. In some embodiments, the at least two polypeptide or protein fusions herein (e.g., Ig (e.g., hIg, mIg) Fc fusion proteins or polypeptides described herein) or conjugates associate via covalent or non-covalent interactions. In some embodiments, the at least two polypeptide or protein fusions herein (e.g., Ig (e.g., hIg, mIg) Fc fusion proteins or polypeptides described herein) or conjugates associate via at least one covalent interaction. In some embodiments, the at least two polypeptide or protein fusions (e.g., Ig (e.g., hIg, mIg) Fc fusion proteins or polypeptides) or conjugates associate via one or more disulfide bond. In some embodiments, the at least two polypeptide or protein fusions (e.g., Ig (e.g., hIg, mIg) Fc fusion proteins or polypeptides) or conjugates associate via 1, 2, 3, 4, or more disulfide bonds.

In some embodiments, the protein is dimeric comprising a first polypeptide or protein fusion (e.g., a hIg Fc fusion protein or polypeptide) or conjugate described herein and a second polypeptide or protein fusion (e.g., an Ig (e.g., hIg, mIg) Fc fusion protein or polypeptide) or conjugate described herein, wherein the first polypeptide comprises an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second polypeptide. For example, the first polypeptide may comprise an amino acid sequence at least about 85% identical to the amino acid sequence of the second polypeptide. For example, the first polypeptide may comprise an amino acid sequence at least about 90% identical to the amino acid sequence of the second polypeptide. For example, the first polypeptide may comprise an amino acid sequence at least about 95% identical to the amino acid sequence of the second polypeptide. In some embodiments, the first polypeptide preferably may comprise an amino acid sequence 100% identical to the amino acid sequence of the second polypeptide.

In some embodiments, the protein is dimeric comprising a first Ig (e.g., hIg, mIg) Fc fusion protein or polypeptide and a second Ig (e.g., hIg, mIg) Fc fusion protein or polypeptide. In some embodiments, the dimeric protein is homodimeric. In some embodiments, the dimeric protein is heterodimeric. In some embodiments, the first Ig (e.g., hIg, mIg) Fc fusion protein or polypeptide comprises an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second Ig (e.g., hIg, mIg) Fc fusion protein or polypeptide.

An exemplary dimeric Ig (e.g., hIg, mIg) Fc fusion protein includes, for example, a protein comprising (i) a first Ig (e.g., hIg, mIg) Fc fusion protein or polypeptide comprising from N- to C-terminus: a first Ig (e.g., hIg, mIg) Fc region (e.g., described herein), a first peptide linker (e.g., described herein), and a first immunomodulatory polypeptide (e.g., described herein); and (ii) a second Ig (e.g., hIg, mIg) Fc fusion protein or polypeptide comprising from N- to C-terminus: a second Ig (e.g., hIg, mIg) Fc region (e.g., described herein), a second peptide linker (e.g., described herein), and a second immunomodulatory polypeptide (e.g., described herein). In some embodiments, the amino acid sequence of the first Ig (e.g., hIg, mIg) Fc fusion protein or polypeptide is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second Ig (e.g., hIg, mIg) Fc fusion protein or polypeptide. In this specific embodiment, the N-terminus of the immunomodulatory polypeptide (e.g., described herein) is operably connected to the C-terminus of the Ig (e.g., hIg, mIg) Fc region through the peptide linker (e.g., described herein).

Another exemplary dimeric Ig (e.g., hIg, mIg) Fc fusion protein includes, for example, a protein comprising (i) a first Ig (e.g., hIg, mIg) Fc fusion protein or polypeptide comprising from N- to C-terminus: a first immunomodulatory polypeptide (e.g., described herein), a first peptide linker (e.g., described herein), and a first Ig (e.g., hIg, mIg) Fc region (e.g., described herein); and (ii) a second immunomodulatory polypeptide (e.g., described herein), a second peptide linker (e.g., described herein), and a second Ig (e.g., hIg, mIg) Fc region (e.g., described herein). In some embodiments, the amino acid sequence of the first Ig (e.g., hIg, mIg) Fc fusion protein or polypeptide is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second Ig (e.g., hIg, mIg) Fc fusion protein or polypeptide. In this specific embodiment, the C-terminus of the immunomodulatory polypeptide (e.g., described herein) is operably connected to the N-terminus of the Ig (e.g., hIg, mIg) Fc region either directly or indirectly through the peptide linker (e.g., described herein).

### 5.3.5 Exemplary Ig Fusion Proteins & Polypeptides

The amino acid sequence of exemplary immunomodulatory fusion polypeptides & proteins (IFPs) described herein is provided in Table 7. Each of the IFPs 1-11 comprising the amino acid sequence of any one of SEQ ID NOS: 58-68 or 80-90 comprises from N- to C-terminus the hIL-2 signal sequence (hIL-2ss), an effector function reduced hIgG4 Fc region, a peptide linker, and an immunomodulatory protein identified herein (IMPs 1-11) (e.g., see Table 2, SEQ ID NOS: 108-126). Each of the IFPs 1-11 comprising the amino acid sequence ofany one of SEQ ID NOS: 69-79 or 91-101 comprises from N- to C-terminus an effector function reduced hIgG4 Fc region, a peptide linker, and an immunomodulatory protein identified herein (IMPs 1-11) (e.g., see Table 2, SEQ ID NOS: 108-126). m2a-IFP-7 comprises an effector function reduced mIgG2a Fc region and m1-IFP-7 comprises an mIgG Fc region. The fusion polypeptides and proteins provided in Table 7 are exemplary only, and not intended to be limiting. Similar fusion proteins can be made utilizing the additional IMPs listed in Table 2, e.g., any one of IMPs 12-175.

TABLE 7
The Amino Acid Sequence of Exemplary Ig Fusion Proteins & Polypeptides

| | | SEQ |
|---|---|---|
| Description | Amino Acid Sequence | ID NO |
| IFP-1 | MYRMQLLSCIALSLALVINSAESKYGPPCPPCPAPEAAGGPSVFLFPPKP | 58 |
| with hIL-2 | KDTLMISRTPEVTCVVVDVSQEDPEVQFNWYVDGVEVHNAKTKPREEQFN | |
| signal peptide | STYRVVSVLIVLHQDWLNGKEYKCKVSNKGLPSSIEKTISKAKGQPREPQ | |
| | VYTLPPSQEEMTKNQVSLTCLVKGFYPSDIAVEWESNGQPENNYKITPPV | |
| | LDSDGSFFLYSRLTVDKSRWQEGNVFSCSVMHEALHNHYTQKSLSLSLGG | |
| | GGGGGGSGGGGSGGGGSMGKRAFVVSVAMALLGIYVITNTVNARHCMFGD | |
| | SLRNSPDMKNMLQDLRGGYSGSGIKRTFQGKDTLDSMLLTQSLLDDEKGY | |
| | LGCQALSEMIQFYLEEVMPQAENHGPTDSVKQLGEKLHTLNQKFGECPRW | |
| | FPCYYNTTPAVENVKSVFSKLQERGVYKAMSEFDIFINYIETYTTMK | |
| IFP-2 | MYRMQLLSCIALSLALVINSAESKYGPPCPPCPAPEAAGGPSVFLFPPKP | 59 |
| with signal | KDTLMISRTPEVTCVVVDVSQEDPEVQFNWYVDGVEVHNAKIKPREEQFN | |
| peptide | STYRVVSVLIVLHQDWLNGKEYKCKVSNKGLPSSIEKTISKAKGQPREPQ | |
| | VYTLPPSQEEMTKNQVSLTCLVKGFYPSDIAVEWESNGQPENNYKITPPV | |
| | LDSDGSFFLYSRLTVDKSRWQEGNVFSCSVMHEALHNHYTQKSLSLSLGG | |
| | GGGGGGSGGGGSGGGGSMARRLTVASCGSVSLLAAFAAVLLIGCQLESGE | |
| | ALPLGSRSADSRSVDGQRVPAPQNNYPGLLRDLRLGYEGFKQKVTDSHPD | |
| | ETLLGSSRLAGDLKGPLRCQALSEMIQFLLQVVLPDAENSRQDLRSQFST | |
| | LGDRITGLRQQLRRDPTVFPCESRSDGVSDLRSAYTRLGSTGAEKVLSEF | |
| | DIFINYIEAYVTSV | |
| IFP-3 | MYRMQLLSCIALSLALVINSAESKYGPPCPPCPAPEAAGGPSVFLFPPKP | 60 |
| with hIL-2 | KDTLMISRTPEVTCVVVDVSQEDPEVQFNWYVDGVEVHNAKTKPREEQFN | |
| signal peptide | STYRVVSVLIVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSMSNNKILVCAVIILTYTLYTDAYCVEYAESDED | |
| | RQQCSSSSNFPASLPHMLRELRAAFGKVKTFFQMKDQLNSMLLTQSLLDD | |
| | FKGYLGCQALSEMIQFYLEEVMPQAENHGPDIKEHVNSLGEKLKTLRLRL | |
| | RRCHRFLPCENKSKAVEQVKRVFNMLQERGVYKAMSEFDIFINYIESYMT | |
| | TKM | |
| IFP-4 | MYRMQLLSCIALSLALVINSAESKYGPPCPPCPAPEAAGGPSVFLFPPKP | 61 |
| with hIL-2 | KDTLMISRTPEVTCVVVDVSQEDPEVQFNWYVDGVEVHNAKTKPREEQFN | |
| signal peptide | STYRVVSVLIVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSMANVVYVVLVISIMMANIHVSKTYCTSCSHHQC | |
| | TEDENQKQDCEDANHSLPHMLRELRAAFGKVKTFFQMKDQLHSLLLTQSL | |
| | LDDFKGYLGCQALSEMIQFYLEEVMPQAENHGPEEHDNSLSEHGPDVKEH | |
| | VNSLGEKLKTLRLRLRRCHRFLPCENKSKAVEKVKRVFSELQERGVYKAM | |
| | SEFDIFINYIETYMIT | |
| IFP-5 | MYRMQLLSCIALSLALVINSAESKYGPPCPPCPAPEAAGGPSVFLFPPKP | 62 |
| with hIL-2 | KDTLMISRTPEVTCVVVDVSQEDPEVQFNWYVDGVEVHNAKTKPREEQFN | |
| signal peptide | STYRVVSVLIVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSMQGLQLLRGLLCCGVFAAASSRSPKNKPSIDCN | |
| | PQTGDFVNMLKSMRQDYSRIRDTLHDRDKLHSSLLTGALLDEMMGYSGCR | |
| | TTLLLMEHYLDTWYPAAYRHHLYDNQTLVVVDRMGSTLVALLKAMVQCPM | |
| | LACGAPSPAMDKMLQQEAKMKKYTGVYKGISETDLLLGYLELYMMKFKR | |
| IFP-6 | MYRMQLLSCIALSLALVINSAESKYGPPCPPCPAPEAAGGPSVFLFPPKP | 63 |
| with hIL-2 | KDTLMISRTPEVTCVVVDVSQEDPEVQFNWYVDGVEVHNAKIKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSMRRRRSFGIVVSGAIRTLLMVAVVAVSVRGHEH | |
| | KVPPACDPVHGNLAGIFKELRAIYASIREALQKKDTVYYTSLENDRVLQE | |
| | MLSPMGCRVTNELMEHYLDGVLPRAAHFDYDNSTLNGLHAFTSSMQALYQ | |
| | HMLKCPALACTGKTPAWMYFLEVEHKLNPWRGTAKAAAEADLLLNYLETF | |
| | LLQF | |
| IFP-7 | MYRMQLLSCIALSLALVINSAESKYGPPCPPCPAPEAAGGPSVFLFPPKP | 64 |
| with hIL-2 | KDTLMISRTPEVTCVVVDVSQEDPEVQFNWYVDGVEVHNAKIKPREEQFN | |
| signal peptide | STYRVVSVLIVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSMGSRRLSRCSFATAVCLVAIVAAVAAKGRDSKP | |
| | SPACDPMHGALAGIFKELRITYRSVREALQTKDTVYYVSLFHEQLLQEML | |
| | SPVGCRVTNELMQHYLDGVLPRAFHCGYDNAILNALHALSSSLSTLYQHM | |
| | LKCPALACTGQTPAWTQFLDTEHKLDPWKGTVKATAEMDLLLNYLETFLL | |
| | QS | |
| m2a-IFP-7 | MYRMQLLSCIALSLALVINSEPRGPTIKPCPPCKCPAPNAAGGPSVFIFP | 479 |
| with hIL-2 | PKIKDVLMISLSPIVTCVVVDVSEDDPDVQISWFVNNVEVHTAQTQTHRE | |
| signal peptide | DYNSTLRVVSALPIQHQDWMSGKEFKCKVNNKDLGAPIERTISKPKGSVR | |
| | APQVYVLPPPEEEMTKKQVTLTCMVTDFMPEDIYVEWTNNGKTELNYKNT | |
| | EPVLDSDGSYFMYSKLRVEKKNWVERNSYSCSVVHEGLHNHHTTKSFSRT | |
| | PGKGGGGGGGGSGGGGSGGGGSMGSRRLSRCSFATAVCLVAIVAAVAAKG | |
| | RDSKPSPACDPMHGALAGIFKELRTTYRSVREALQTKDTVYYVSLFHEQL | |
| | LQEMLSPVGCRVTNELMQHYLDGVLPRAFHCGYDNATLNALHALSSSLST | |
| | LYQHMLKCPALACTGQTPAWTQFLDTEHKLDPWKGTVKATAEMDLLLNYL | |
| | ETFLLQS | |
| m1-IFP-7 | MYRMQLLSCIALSLALVINSVPRDCGCKPCICTVPEVSSVFIFPPKPKDV | 480 |
| with hIL-2 | LTITLTPKVTCVVVAISKDDPEVQFSWFVDDVEVHTAQTQPREEQFNSTF | |
| signal peptide | RSVSELPIMHQDWLNGKEFKCRVNSAAFPAPIEKTISKTKGRPKAPQVYT | |
| | IPPPKEQMAKDKVSLTCMITDFFPEDITVEWQWNGQPAENYKNTQPIMDT | |
| | DGSYFVYSKLNVQKSNWEAGNTFTCSVLHEGLHNHHTEKSLSHSPGGGGG | |
| | GGSGGGGSGGGGSMGSRRLSRCSFATAVCLVAIVAAVAAKGRDSKPSPAC | |
| | DPMHGALAGIFKELRTTYRSVREALQTKDTVYYVSLFHEQLLQEMLSPVG | |
| | CRVTNELMQHYLDGVLPRAFHCGYDNATLNALHALSSSLSTLYQHMLKCP | |
| | ALACTGQTPAWTQFLDTEHKLDPWKGTVKATAEMDLLLNYLETFLLQS | |
| IFP-8 | MYRMQLLSCIALSLALVINSAESKYGPPCPPCPAPEAAGGPSVFLFPPKP | 65 |
| with hIL-2 | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSMLSVMVSSSLVLIVFFLGASEEAKPATTTIKNT | |
| | KPQCRPEDYATRLQDLRVTFHRVKPTLQREDDYSVWLDGTVVKGCWGCSV | |
| | MDWLLRRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IFP-9 | MYRMQLLSCIALSLALVINSAESKYGPPCPPCPAPEAAGGPSVFLFPPKP | 66 |
| with hIL-2 | KDTLMISRTPEVTCVVVDVSQEDPEVQFNWYVDGVEVHNAKTKPREEQFN | |
| signal peptide | STYRVVSVLIVLHQDWLNGKEYKCKVSNKGLPSSIEKTISKAKGQPREPQ | |
| | VYTLPPSQEEMTKNQVSLTCLVKGFYPSDIAVEWESNGQPENNYKITPPV | |
| | LDSDGSFFLYSRLTVDKSRWQEGNVFSCSVMHEALHNHYTQKSLSLSLGG | |
| | GGGGGGSGGGGSGGGGSMALAHQLPVWIFSIWILYFTLPLSEERVLPLRG | |
| | NCKLLLQDTVIPNLLYSMRSIFQDIKPYFQGKDSLNNLLLSGQLLEDLQS | |
| | PIGCDALSEMIQFYLEEVMPQAEIHHPKHKNSVMQLGETLHTLISQLQEC | |
| | TALFPCKHKSLGAQKIKEEVSKLGQYGIIKAVAEFDIFINYMESYFGVK | |
| IFP-10 | MYRMQLLSCIALSLALVINSAESKYGPPCPPCPAPEAAGGPSVFLFPPKP | 67 |
| with hIL-2 | KDTLMISRTPEVTCVVVDVSQEDPEVQFNWYVDGVEVHNAKIKPREEQEN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSMRRRRRSFGIIVAGAIGTLLMMAVVVLSAHDHE | |
| | HKEVPPACDPVHGNLAGIFKELRATYASIREGLQKKDTVYYTSLENDRVL | |
| | HEMLSPMGCRVTNELMEHYLDGVLPRASHLDYDNSTLNGLHVFASSMQAL | |
| | YQHMLKCPALACTGKTPAWMYFLEVEHKLNPWRGTAKAAAEADLLLNYLE | |
| | TFLLQF | |
| IFP-11 | MYRMQLLSCIALSLALVINSAESKYGPPCPPCPAPEAAGGPSVFLFPPKP | 68 |
| with hIL-2 | KDTLMISRTPEVTCVVVDVSQEDPEVQFNWYVDGVEVHNAKTKPREEQFN | |
| signal peptide | STYRVVSVLIVLHQDWLNGKEYKCKVSNKGLPSSIEKTISKAKGQPREPQ | |
| | VYTLPPSQEEMTKNQVSLTCLVKGFYPSDIAVEWESNGQPENNYKITPPV | |
| | LDSDGSFFLYSRLTVDKSRWQEGNVFSCSVMHEALHNHYTQKSLSLSLGG | |
| | GGGGGGSGGGGSGGGGSMRRRRSFGIVVAGAIGTLLMMAVVVFSAHEHKE | |
| | VPPACDPVHGNLAGIFKELRATYASIREGLQKKDTVYYTSLENDRVLQEM | |
| | LSPMGCRVTNELMEHYLDGVLPRALHLDYDNSILNGLHAFASSMQALYQH | |
| | MLKCPALACTGKTPAWMYFLEVEHKLNPWRGTAKAAAEADLLLNYLETFL | |
| | LQF | |
| IFP-1 | AESKYGPPCPPCPAPEAAGGPSVFLFPPKPKDTLMISRTPEVTCVVVDVS | 69 |
| without hIL-2 | QEDPEVQFNWYVDGVEVHNAKTKPREEQFNSTYRVVSVLIVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSMGK | |
| | RAFVVSVAMALLGIYVITNTVNARHCMFGDSLRNSPDMKNMLQDLRGGYS | |
| | GSGIKRTFQGKDTLDSMLLTQSLLDDFKGYLGCQALSEMIQFYLEEVMPQ | |
| | AENHGPTDSVKQLGEKLHTLNQKFGECPRWFPCYYNTTPAVENVKSVFSK | |
| | LQERGVYKAMSEFDIFINYIETYTTMK | |
| IFP-2 | AESKYGPPCPPCPAPEAAGGPSVFLFPPKPKDTLMISRTPEVTCVVVDVS | 70 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSMAR | |
| | RLTVASCGSVSLLAAFAAVLLIGCQLESGEALPLGSRSADSRSVDGQRVP | |
| | APQNNYPGLLRDLRLGYEGFKQKVTDSHPDETLLGSSRLAGDLKGPLRCQ | |
| | ALSEMIQFLLQVVLPDAENSRQDLRSQFSTLGDRITGLRQQLRRDPTVFP | |
| | CESRSDGVSDLRSAYTRLGSTGAEKVLSEFDIFINYIEAYVTSV | |
| IFP-3 | AESKYGPPCPPCPAPEAAGGPSVFLFPPKPKDTLMISRTPEVTCVVVDVS | 71 |
| without hIL-2 | QEDPEVQFNWYVDGVEVHNAKTKPREEQFNSTYRVVSVLIVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSMSN | |
| | NKILVCAVIILTYTLYTDAYCVEYAESDEDRQQCSSSSNFPASLPHMLRE | |
| | LRAAFGKVKTFFQMKDQLNSMLLTQSLLDDFKGYLGCQALSEMIQFYLEE | |
| | VMPQAENHGPDIKEHVNSLGEKLKTLRLRLRRCHRFLPCENKSKAVEQVK | |
| | RVFNMLQERGVYKAMSEFDIFINYIESYMTTKM | |
| IFP-4 | AESKYGPPCPPCPAPEAAGGPSVFLFPPKPKDTLMISRTPEVTCVVVDVS | 72 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSMAN | |
| | VVYVVLVISIMMANIHVSKTYCTSCSHHQCTEDENQKQDCEDANHSLPHM | |
| | LRELRAAFGKVKTFFQMKDQLHSLLLTQSLLDDFKGYLGCQALSEMIQFY | |
| | LEEVMPQAENHGPEEHDNSLSEHGPDVKEHVNSLGEKLKTLRLRLRRCHR | |
| | FLPCENKSKAVEKVKRVFSELQERGVYKAMSEFDIFINYIETYMTT | |
| IFP-5 | AESKYGPPCPPCPAPEAAGGPSVFLFPPKPKDTLMISRTPEVTCVVVDVS | 73 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSMQG | |
| | LQLLRGLLCCGVFAAASSRSPKNKPSIDCNPQTGDFVNMLKSMRQDYSRI | |
| | RDTLHDRDKLHSSLLIGALLDEMMGYSGCRITLLLMEHYLDTWYPAAYRH | |
| | HLYDNQTLVVVDRMGSTLVALLKAMVQCPMLACGAPSPAMDKMLQQEAKM | |
| | KKYTGVYKGISETDLLLGYLELYMMKFKR | |
| IFP-6 | AESKYGPPCPPCPAPEAAGGPSVFLFPPKPKDTLMISRTPEVTCVVVDVS | 74 |
| without hIL-2 | QEDPEVQFNWYVDGVEVHNAKTKPREEQFNSTYRVVSVLIVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSMRR | |
| | RRSFGIVVSGAIRTLLMVAVVAVSVRGHEHKVPPACDPVHGNLAGIFKEL | |
| | RAIYASIREALQKKDTVYYTSLFNDRVLQEMLSPMGCRVTNELMEHYLDG | |
| | VLPRAAHFDYDNSTLNGLHAFTSSMQALYQHMLKCPALACTGKTPAWMYF | |
| | LEVEHKLNPWRGTAKAAAEADLLLNYLETFLLQF | |
| IFP-7 | AESKYGPPCPPCPAPEAAGGPSVFLFPPKPKDTLMISRTPEVTCVVVDVS | 75 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSMGS | |
| | RRLSRCSFATAVCLVAIVAAVAAKGRDSKPSPACDPMHGALAGIFKELRT | |
| | TYRSVREALQTKDTVYYVSLFHEQLLQEMLSPVGCRVTNELMQHYLDGVL | |
| | PRAFHCGYDNATLNALHALSSSLSTLYQHMLKCPALACTGQTPAWTQFLD | |
| | TEHKLDPWKGTVKATAEMDLLLNYLETFLLQS | |
| m2a-IFP-7 | EPRGPTIKPCPPCKCPAPNAAGGPSVFIFPPKIKDVLMISLSPIVTCVVV | 481 |
| without hIL-2 | DVSEDDPDVQISWFVNNVEVHTAQTQTHREDYNSTLRVVSALPIQHQDWM | |
| signal peptide | SGKEFKCKVNNKDLGAPIERTISKPKGSVRAPQVYVLPPPEEEMTKKQVT | |
| | LTCMVTDFMPEDIYVEWINNGKTELNYKNTEPVLDSDGSYFMYSKLRVEK | |
| | KNWVERNSYSCSVVHEGLHNHHTTKSFSRTPGKGGGGGGGGSGGGGSGGG | |
| | GSMGSRRLSRCSFATAVCLVAIVAAVAAKGRDSKPSPACDPMHGALAGIF | |
| | KELRTTYRSVREALQTKDTVYYVSLFHEQLLQEMLSPVGCRVTNELMQHY | |
| | LDGVLPRAFHCGYDNATLNALHALSSSLSTLYQHMLKCPALACTGQTPAW | |
| | TQFLDTEHKLDPWKGTVKATAEMDLLLNYLETFLLQS | |
| m1-IFP-7 | VPRDCGCKPCICTVPEVSSVFIFPPKPKDVLTITLTPKVTCVVVAISKDD | 482 |
| without hIL-2 | PEVQFSWFVDDVEVHTAQTQPREEQFNSTFRSVSELPIMHQDWLNGKEFK | |
| signal peptide | CRVNSAAFPAPIEKTISKTKGRPKAPQVYTIPPPKEQMAKDKVSLICMIT | |
| | DFFPEDITVEWQWNGQPAENYKNTQPIMDTDGSYFVYSKLNVQKSNWEAG | |
| | NTFTCSVLHEGLHNHHTEKSLSHSPGGGGGGGSGGGGSGGGGSMGSRRLS | |
| | RCSFATAVCLVAIVAAVAAKGRDSKPSPACDPMHGALAGIFKELRITYRS | |
| | VREALQTKDTVYYVSLFHEQLLQEMLSPVGCRVTNELMQHYLDGVLPRAF | |
| | HCGYDNATLNALHALSSSLSTLYQHMLKCPALACTGQTPAWTQFLDTEHK | |
| | LDPWKGTVKATAEMDLLLNYLETFLLQS | |
| IFP-8 | AESKYGPPCPPCPAPEAAGGPSVFLFPPKPKDTLMISRTPEVTCVVVDVS | 76 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSMLS | |
| | VMVSSSLVLIVFFLGASEEAKPATTTIKNTKPQCRPEDYATRLQDLRVTF | |
| | HRVKPTLQREDDYSVWLDGTVVKGCWGCSVMDWLLRRYLEIVFPAGDHVY | |
| | PGLKTELHSMRSTLESIYKDMRQCPLLGCGDKSVISRLSQEAERKSDNGT | |
| | RKGLSELDTLFSRLEEYLHSRK | |
| IFP-9 | AESKYGPPCPPCPAPEAAGGPSVFLFPPKPKDTLMISRTPEVTCVVVDVS | 77 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSMAL | |
| | AHQLPVWIFSIWILYFTLPLSEERVLPLRGNCKLLLQDTVIPNLLYSMRS | |
| | IFQDIKPYFQGKDSLNNLLLSGQLLEDLQSPIGCDALSEMIQFYLEEVMP | |
| | QAEIHHPKHKNSVMQLGETLHTLISQLQECTALFPCKHKSLGAQKIKEEV | |
| | SKLGQYGIIKAVAEFDIFINYMESYFGVK | |
| IFP-10 | AESKYGPPCPPCPAPEAAGGPSVFLFPPKPKDTLMISRIPEVTCVVVDVS | 78 |
| without hIL-2 | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSMRR | |
| | RRRSFGIIVAGAIGTLLMMAVVVLSAHDHEHKEVPPACDPVHGNLAGIFK | |
| | ELRATYASIREGLQKKDTVYYTSLFNDRVLHEMLSPMGCRVTNELMEHYL | |
| | DGVLPRASHLDYDNSTLNGLHVFASSMQALYQHMLKCPALACTGKTPAWM | |
| | YFLEVEHKLNPWRGTAKAAAEADLLLNYLETFLLQF | |
| IFP-11 | AESKYGPPCPPCPAPEAAGGPSVFLFPPKPKDTLMISRTPEVTCVVVDVS | 79 |
| without hIL-2 | QEDPEVQFNWYVDGVEVHNAKTKPREEQFNSTYRVVSVLIVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKITPPVLDSDGSFFLYSRLTVDKSRW | |
| | QEGNVFSCSVMHEALHNHYTQKSLSLSLGGGGGGGGSGGGGSGGGGSMRR | |
| | RRSFGIVVAGAIGTLLMMAVVVFSAHEHKEVPPACDPVHGNLAGIFKELR | |
| | ATYASIREGLQKKDTVYYTSLFNDRVLQEMLSPMGCRVTNELMEHYLDGV | |
| | LPRALHLDYDNSTLNGLHAFASSMQALYQHMLKCPALACTGKTPAWMYFL | |
| | EVEHKLNPWRGTAKAAAEADLLLNYLETFLLQF | |
| IFP-1 | MYRMQLLSCIALSLALVINSESKYGPPCPPCPAPEAAGGPSVFLFPPKPK | 80 |
| with hIL-2 | DTLMISRTPEVTCVVVDVSQEDPEVQFNWYVDGVEVHNAKTKPREEQENS | |
| signal peptide | TYRVVSVLIVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSMGKRAFVVSVAMALLGIYVITNTVNARHCMFGDS | |
| | LRNSPDMKNMLQDLRGGYSGSGIKRTFQGKDTLDSMLLTQSLLDDFKGYL | |
| | GCQALSEMIQFYLEEVMPQAENHGPTDSVKQLGEKLHTLNQKFGECPRWF | |
| | PCYYNTTPAVENVKSVFSKLQERGVYKAMSEFDIFINYIETYTTMK | |
| IFP-2 | MYRMQLLSCIALSLALVINSESKYGPPCPPCPAPEAAGGPSVFLFPPKPK | 81 |
| with hIL-2 | DTLMISRTPEVTCVVVDVSQEDPEVQFNWYVDGVEVHNAKIKPREEQFNS | |
| signal peptide | TYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKITPPVL | |
| | DSDGSFFLYSRLTVDKSRWQEGNVFSCSVMHEALHNHYTQKSLSLSLGGG | |
| | GGGGGSGGGGSGGGGSMARRLTVASCGSVSLLAAFAAVLLIGCQLESGEA | |
| | LPLGSRSADSRSVDGQRVPAPQNNYPGLLRDLRLGYEGFKQKVTDSHPDE | |
| | TLLGSSRLAGDLKGPLRCQALSEMIQFLLQVVLPDAENSRQDLRSQFSTL | |
| | GDRITGLRQQLRRDPTVFPCESRSDGVSDLRSAYTRLGSTGAEKVLSEFD | |
| | IFINYIEAYVISV | |
| IFP-3 | MYRMQLLSCIALSLALVINSESKYGPPCPPCPAPEAAGGPSVFLFPPKPK | 82 |
| with hIL-2 | DTLMISRTPEVTCVVVDVSQEDPEVQFNWYVDGVEVHNAKIKPREEQENS | |
| signal peptide | TYRVVSVLIVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSMSNNKILVCAVIILTYTLYTDAYCVEYAESDEDR | |
| | QQCSSSSNFPASLPHMLRELRAAFGKVKTFFQMKDQLNSMLLTQSLLDDF | |
| | KGYLGCQALSEMIQFYLEEVMPQAENHGPDIKEHVNSLGEKLKTLRLRLR | |
| | RCHRFLPCENKSKAVEQVKRVFNMLQERGVYKAMSEFDIFINYIESYMTT | |
| | KM | |
| IFP-4 | MYRMQLLSCIALSLALVINSESKYGPPCPPCPAPEAAGGPSVFLFPPKPK | 83 |
| with hIL-2 | DTLMISRTPEVTCVVVDVSQEDPEVQFNWYVDGVEVHNAKTKPREEQFNS | |
| signal peptide | TYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSMANVVYVVLVISIMMANIHVSKTYCTSCSHHQCT | |
| | EDENQKQDCEDANHSLPHMLRELRAAFGKVKTFFQMKDQLHSLLLTQSLL | |
| | DDFKGYLGCQALSEMIQFYLEEVMPQAENHGPEEHDNSLSEHGPDVKEHV | |
| | NSLGEKLKTLRLRLRRCHRFLPCENKSKAVEKVKRVFSELQERGVYKAMS | |
| | EFDIFINYIETYMIT | |
| IFP-5 | MYRMQLLSCIALSLALVTNSESKYGPPCPPCPAPEAAGGPSVFLFPPKPK | 84 |
| with hIL-2 | DTLMISRTPEVTCVVVDVSQEDPEVQFNWYVDGVEVHNAKTKPREEQFNS | |
| signal peptide | TYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSMQGLQLLRGLLCCGVFAAASSRSPKNKPSIDCNP | |
| | QTGDFVNMLKSMRQDYSRIRDTLHDRDKLHSSLLTGALLDEMMGYSGCRI | |
| | TLLLMEHYLDTWYPAAYRHHLYDNQTLVVVDRMGSTLVALLKAMVQCPML | |
| | ACGAPSPAMDKMLQQEAKMKKYTGVYKGISETDLLLGYLELYMMKFKR | |
| IFP-6 | MYRMQLLSCIALSLALVINSESKYGPPCPPCPAPEAAGGPSVFLFPPKPK | 85 |
| with hIL-2 | DTLMISRTPEVTCVVVDVSQEDPEVQFNWYVDGVEVHNAKTKPREEQFNS | |
| signal peptide | TYRVVSVLIVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSMRRRRSFGIVVSGAIRTLLMVAVVAVSVRGHEHK | |
| | VPPACDPVHGNLAGIFKELRAIYASIREALQKKDTVYYTSLFNDRVLQEM | |
| | LSPMGCRVTNELMEHYLDGVLPRAAHFDYDNSTLNGLHAFTSSMQALYQH | |
| | MLKCPALACTGKTPAWMYFLEVEHKLNPWRGTAKAAAEADLLLNYLETFL | |
| | LQF | |
| IFP-7 | MYRMQLLSCIALSLALVINSESKYGPPCPPCPAPEAAGGPSVFLFPPKPK | 86 |
| with hIL-2 | DTLMISRTPEVTCVVVDVSQEDPEVQFNWYVDGVEVHNAKTKPREEQENS | |
| signal peptide | TYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSMGSRRLSRCSFATAVCLVAIVAAVAAKGRDSKPS | |
| | PACDPMHGALAGIFKELRITYRSVREALQTKDTVYYVSLFHEQLLQEMLS | |
| | PVGCRVTNELMQHYLDGVLPRAFHCGYDNATLNALHALSSSLSTLYQHML | |
| | KCPALACTGQTPAWTQFLDTEHKLDPWKGTVKATAEMDLLLNYLETFLLQ | |
| | S | |
| IFP-8 | MYRMQLLSCIALSLALVINSESKYGPPCPPCPAPEAAGGPSVFLFPPKPK | 87 |
| with hIL-2 | DTLMISRTPEVTCVVVDVSQEDPEVQFNWYVDGVEVHNAKTKPREEQENS | |
| signal peptide | TYRVVSVLIVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSMLSVMVSSSLVLIVFFLGASEEAKPATTTIKNTK | |
| | PQCRPEDYATRLQDLRVTFHRVKPTLQREDDYSVWLDGTVVKGCWGCSVM | |
| | DWLLRRYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IFP-9 | MYRMQLLSCIALSLALVINSESKYGPPCPPCPAPEAAGGPSVFLFPPKPK | 88 |
| with hIL-2 | DTLMISRTPEVTCVVVDVSQEDPEVQFNWYVDGVEVHNAKTKPREEQFNS | |
| signal peptide | TYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSMALAHQLPVWIFSIWILYFTLPLSEERVLPLRGN | |
| | CKLLLQDTVIPNLLYSMRSIFQDIKPYFQGKDSLNNLLLSGQLLEDLQSP | |
| | IGCDALSEMIQFYLEEVMPQAEIHHPKHKNSVMQLGETLHTLISQLQECT | |
| | ALFPCKHKSLGAQKIKEEVSKLGQYGIIKAVAEFDIFINYMESYFGVK | |
| IFP-10 | MYRMQLLSCIALSLALVINSESKYGPPCPPCPAPEAAGGPSVFLFPPKPK | 89 |
| with hIL-2 | DTLMISRTPEVTCVVVDVSQEDPEVQFNWYVDGVEVHNAKTKPREEQFNS | |
| signal peptide | TYRVVSVLIVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSMRRRRRSFGIIVAGAIGTLLMMAVVVLSAHDHEH | |
| | KEVPPACDPVHGNLAGIFKELRATYASIREGLQKKDTVYYTSLFNDRVLH | |
| | EMLSPMGCRVTNELMEHYLDGVLPRASHLDYDNSTLNGLHVFASSMQALY | |
| | QHMLKCPALACTGKTPAWMYFLEVEHKLNPWRGTAKAAAEADLLLNYLET | |
| | FLLQF | |
| IFP-11 | MYRMQLLSCIALSLALVINSESKYGPPCPPCPAPEAAGGPSVFLFPPKPK | 90 |
| with hIL-2 | DTLMISRTPEVTCVVVDVSQEDPEVQFNWYVDGVEVHNAKTKPREEQFNS | |
| signal peptide | TYRVVSVLIVLHQDWLNGKEYKCKVSNKGLPSSIEKTISKAKGQPREPQV | |
| | YTLPPSQEEMTKNQVSLICLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSMRRRRSFGIVVAGAIGTLLMMAVVVFSAHEHKEV | |
| | PPACDPVHGNLAGIFKELRATYASIREGLQKKDTVYYTSLENDRVLQEML | |
| | SPMGCRVTNELMEHYLDGVLPRALHLDYDNSTLNGLHAFASSMQALYQHM | |
| | LKCPALACTGKTPAWMYFLEVEHKLNPWRGTAKAAAEADLLLNYLETFLL | |
| | QF | |
| IFP-1 | ESKYGPPCPPCPAPEAAGGPSVFLFPPKPKDTLMISRTPEVTCVVVDVSQ | 91 |
| without hIL-2 | EDPEVQFNWYVDGVEVHNAKTKPREEQFNSTYRVVSVLIVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSMGKR | |
| | AFVVSVAMALLGIYVITNTVNARHCMFGDSLRNSPDMKNMLQDLRGGYSG | |
| | SGIKRTFQGKDTLDSMLLTQSLLDDFKGYLGCQALSEMIQFYLEEVMPQA | |
| | ENHGPTDSVKQLGEKLHTLNQKFGECPRWFPCYYNTTPAVENVKSVFSKL | |
| | QERGVYKAMSEFDIFINYIETYTTMK | |
| IFP-2 | ESKYGPPCPPCPAPEAAGGPSVFLFPPKPKDTLMISRTPEVTCVVVDVSQ | 92 |
| without hIL-2 | EDPEVQFNWYVDGVEVHNAKTKPREEQFNSTYRVVSVLIVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSMARR | |
| | LTVASCGSVSLLAAFAAVLLIGCQLESGEALPLGSRSADSRSVDGQRVPA | |
| | PQNNYPGLLRDLRLGYEGFKQKVTDSHPDETLLGSSRLAGDLKGPLRCQA | |
| | LSEMIQFLLQVVLPDAENSRQDLRSQFSTLGDRITGLRQQLRRDPTVFPC | |
| | ESRSDGVSDLRSAYTRLGSTGAEKVLSEFDIFINYIEAYVTSV | |
| IFP-3 | ESKYGPPCPPCPAPEAAGGPSVFLFPPKPKDTLMISRTPEVTCVVVDVSQ | 93 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSMSNN | |
| | KILVCAVIILTYTLYTDAYCVEYAESDEDRQQCSSSSNFPASLPHMLREL | |
| | RAAFGKVKTFFQMKDQLNSMLLTQSLLDDFKGYLGCQALSEMIQFYLEEV | |
| | MPQAENHGPDIKEHVNSLGEKLKTLRLRLRRCHRFLPCENKSKAVEQVKR | |
| | VFNMLQERGVYKAMSEFDIFINYIESYMTTKM | |
| IFP-4 | ESKYGPPCPPCPAPEAAGGPSVFLFPPKPKDTLMISRTPEVTCVVVDVSQ | 94 |
| without hIL-2 | EDPEVQFNWYVDGVEVHNAKTKPREEQFNSTYRVVSVLIVLHQDWLNGKE | |
| signal peptide | YKCKVSNKGLPSSIEKTISKAKGQPREPQVYTLPPSQEEMIKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSMANV | |
| | VYVVLVISIMMANIHVSKTYCTSCSHHQCTEDENQKQDCEDANHSLPHML | |
| | RELRAAFGKVKTFFQMKDQLHSLLLTQSLLDDFKGYLGCQALSEMIQFYL | |
| | EEVMPQAENHGPEEHDNSLSEHGPDVKEHVNSLGEKLKTLRLRLRRCHRF | |
| | LPCENKSKAVEKVKRVFSELQERGVYKAMSEFDIFINYIETYMTT | |
| IFP-5 | ESKYGPPCPPCPAPEAAGGPSVFLFPPKPKDTLMISRTPEVTCVVVDVSQ | 95 |
| Without hIL-2 | EDPEVQFNWYVDGVEVHNAKTKPREEQFNSTYRVVSVLIVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSMQGL | |
| | QLLRGLLCCGVFAAASSRSPKNKPSIDCNPQTGDFVNMLKSMRQDYSRIR | |
| | DTLHDRDKLHSSLLTGALLDEMMGYSGCRTTLLLMEHYLDTWYPAAYRHH | |
| | LYDNQTLVVVDRMGSTLVALLKAMVQCPMLACGAPSPAMDKMLQQEAKMK | |
| | KYTGVYKGISETDLLLGYLELYMMKFKR | |
| IFP-6 | ESKYGPPCPPCPAPEAAGGPSVFLFPPKPKDILMISRTPEVTCVVVDVSQ | 96 |
| without hIL-2 | EDPEVQFNWYVDGVEVHNAKTKPREEQFNSTYRVVSVLIVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSMRRR | |
| | RSFGIVVSGAIRTLLMVAVVAVSVRGHEHKVPPACDPVHGNLAGIFKELR | |
| | AIYASIREALQKKDTVYYTSLFNDRVLQEMLSPMGCRVTNELMEHYLDGV | |
| | LPRAAHFDYDNSTLNGLHAFTSSMQALYQHMLKCPALACTGKTPAWMYFL | |
| | EVEHKLNPWRGTAKAAAEADLLLNYLETFLLQF | |
| IFP-7 | ESKYGPPCPPCPAPEAAGGPSVFLFPPKPKDTLMISRTPEVTCVVVDVSQ | 97 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSMGSR | |
| | RLSRCSFATAVCLVAIVAAVAAKGRDSKPSPACDPMHGALAGIFKELRTT | |
| | YRSVREALQTKDTVYYVSLFHEQLLQEMLSPVGCRVTNELMQHYLDGVLP | |
| | RAFHCGYDNATLNALHALSSSLSTLYQHMLKCPALACTGQTPAWTQFLDT | |
| | EHKLDPWKGTVKATAEMDLLLNYLETELLQS | |
| IFP-8 | ESKYGPPCPPCPAPEAAGGPSVFLFPPKPKDTLMISRTPEVTCVVVDVSQ | 98 |
| without hIL-2 | EDPEVQFNWYVDGVEVHNAKTKPREEQFNSTYRVVSVLIVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSMLSV | |
| | MVSSSLVLIVFFLGASEEAKPATTTIKNTKPQCRPEDYATRLQDLRVTFH | |
| | RVKPTLQREDDYSVWLDGTVVKGCWGCSVMDWLLRRYLEIVFPAGDHVYP | |
| | GLKTELHSMRSTLESIYKDMRQCPLLGCGDKSVISRLSQEAERKSDNGTR | |
| | KGLSELDTLFSRLEEYLHSRK | |
| IFP-9 | ESKYGPPCPPCPAPEAAGGPSVFLFPPKPKDTLMISRTPEVTCVVVDVSQ | 99 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKITPPVLDSDGSFFLYSRLTVDKSRWQ | |
| | EGNVFSCSVMHEALHNHYTQKSLSLSLGGGGGGGGSGGGGSGGGGSMALA | |
| | HQLPVWIFSIWILYFTLPLSEERVLPLRGNCKLLLQDTVIPNLLYSMRSI | |
| | FQDIKPYFQGKDSLNNLLLSGQLLEDLQSPIGCDALSEMIQFYLEEVMPQ | |
| | AEIHHPKHKNSVMQLGETLHTLISQLQECTALFPCKHKSLGAQKIKEEVS | |
| | KLGQYGIIKAVAEFDIFINYMESYFGVK | |
| IFP-10 | ESKYGPPCPPCPAPEAAGGPSVFLFPPKPKDTLMISRTPEVTCVVVDVSQ | 100 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKITPPVLDSDGSFFLYSRLTVDKSRWQ | |
| | EGNVFSCSVMHEALHNHYTQKSLSLSLGGGGGGGGSGGGGSGGGGSMRRR | |
| | RRSFGIIVAGAIGTLLMMAVVVLSAHDHEHKEVPPACDPVHGNLAGIFKE | |
| | LRATYASIREGLQKKDTVYYTSLFNDRVLHEMLSPMGCRVTNELMEHYLD | |
| | GVLPRASHLDYDNSTLNGLHVFASSMQALYQHMLKCPALACTGKTPAWMY | |
| | FLEVEHKLNPWRGTAKAAAEADLLLNYLETFLLQF | |
| IFP-11 | ESKYGPPCPPCPAPEAAGGPSVFLFPPKPKDTLMISRTPEVTCVVVDVSQ | 101 |
| without hIL-2 | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKITPPVLDSDGSFFLYSRLTVDKSRWQ | |
| | EGNVFSCSVMHEALHNHYTQKSLSLSLGGGGGGGGSGGGGSGGGGSMRRR | |
| | RSFGIVVAGAIGTLLMMAVVVFSAHEHKEVPPACDPVHGNLAGIFKELRA | |
| | TYASIREGLQKKDTVYYTSLFNDRVLQEMLSPMGCRVTNELMEHYLDGVL | |
| | PRALHLDYDNSTLNGLHAFASSMQALYQHMLKCPALACTGKTPAWMYFLE | |
| | VEHKLNPWRGTAKAAAEADLLLNYLETFLLQF | |
| IFP-1 | MYRMQLLSCIALSLALVINSAESKYGPPCPPCPAPEAAGGPSVFLFPPKP | 483 |
| with hIL-2 | KDTLMISRTPEVTCVVVDVSQEDPEVQFNWYVDGVEVHNAKTKPREEQFN | |
| signal peptide | STYRVVSVLIVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSRHCMFGDSLRNSPDMKNMLQDLRGGYSGSGIKR | |
| | TFQGKDTLDSMLLTQSLLDDFKGYLGCQALSEMIQFYLEEVMPQAENHGP | |
| | TDSVKQLGEKLHTLNQKFGECPRWFPCYYNTTPAVENVKSVFSKLQERGV | |
| | YKAMSEFDIFINYIETYTTMK | |
| IFP-2 | MYRMQLLSCIALSLALVINSAESKYGPPCPPCPAPEAAGGPSVFLFPPKP | 484 |
| with hIL2- | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSCQLESGEALPLGSRSADSRSVDGQRVPAPQNNY | |
| | PGLLRDLRLGYEGFKQKVTDSHPDETLLGSSRLAGDLKGPLRCQALSEMI | |
| | QFLLQVVLPDAENSRQDLRSQFSTLGDRITGLRQQLRRDPTVFPCESRSD | |
| | GVSDLRSAYTRLGSTGAEKVLSEFDIFINYIEAYVTSV | |
| IFP-3 | MYRMQLLSCIALSLALVINSAESKYGPPCPPCPAPEAAGGPSVFLFPPKP | 485 |
| with hIL2- | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSYCVEYAESDEDRQQCSSSSNFPASLPHMLRELR | |
| | AAFGKVKTFFQMKDQLNSMLLTQSLLDDFKGYLGCQALSEMIQFYLEEVM | |
| | PQAENHGPDIKEHVNSLGEKLKTLRLRLRRCHRFLPCENKSKAVEQVKRV | |
| | FNMLQERGVYKAMSEFDIFINYIESYMTTKM | |
| IFP-4 | MYRMQLLSCIALSLALVINSAESKYGPPCPPCPAPEAAGGPSVFLFPPKP | 486 |
| with hIL2- | KDTLMISRTPEVTCVVVDVSQEDPEVQFNWYVDGVEVHNAKTKPREEQEN | |
| signal peptide | STYRVVSVLIVLHQDWLNGKEYKCKVSNKGLPSSIEKTISKAKGQPREPQ | |
| | VYTLPPSQEEMTKNQVSLTCLVKGFYPSDIAVEWESNGQPENNYKITPPV | |
| | LDSDGSFFLYSRLTVDKSRWQEGNVFSCSVMHEALHNHYTQKSLSLSLGG | |
| | GGGGGGSGGGGSGGGGSYCTSCSHHQCTEDENQKQDCEDANHSLPHMLRE | |
| | LRAAFGKVKTFFQMKDQLHSLLLTQSLLDDFKGYLGCQALSEMIQFYLEE | |
| | VMPQAENHGPEEHDNSLSEHGPDVKEHVNSLGEKLKTLRLRLRRCHRFLP | |
| | CENKSKAVEKVKRVFSELQERGVYKAMSEFDIFINYIETYMTT | |
| IFP-5 | MYRMQLLSCIALSLALVINSAESKYGPPCPPCPAPEAAGGPSVFLFPPKP | 487 |
| with hIL2- | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKITPPV | |
| | LDSDGSFFLYSRLTVDKSRWQEGNVFSCSVMHEALHNHYTQKSLSLSLGG | |
| | GGGGGGSGGGGSGGGGSRSPKNKPSIDCNPQTGDFVNMLKSMRQDYSRIR | |
| | DTLHDRDKLHSSLLTGALLDEMMGYSGCRTTLLLMEHYLDTWYPAAYRHH | |
| | LYDNQTLVVVDRMGSTLVALLKAMVQCPMLACGAPSPAMDKMLQQEAKMK | |
| | KYTGVYKGISETDLLLGYLELYMMKFKR | |
| IFP-6 | MYRMQLLSCIALSLALVINSAESKYGPPCPPCPAPEAAGGPSVFLFPPKP | 488 |
| with hIL2- | KDTLMISRTPEVTCVVVDVSQEDPEVQFNWYVDGVEVHNAKTKPREEQEN | |
| signal peptide | STYRVVSVLIVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSHEHKVPPACDPVHGNLAGIFKELRAIYASIREA | |
| | LQKKDTVYYTSLFNDRVLQEMLSPMGCRVTNELMEHYLDGVLPRAAHFDY | |
| | DNSTLNGLHAFTSSMQALYQHMLKCPALACTGKTPAWMYFLEVEHKLNPW | |
| | RGTAKAAAEADLLLNYLETFLLQF | |
| IFP-7 | MYRMQLLSCIALSLALVINSAESKYGPPCPPCPAPEAAGGPSVFLFPPKP | 489 |
| with hIL2- | KDTLMISRTPEVTCVVVDVSQEDPEVQFNWYVDGVEVHNAKTKPREEQFN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKTTPPV | |
| | LDSDGSFFLYSRLTVDKSRWQEGNVFSCSVMHEALHNHYTQKSLSLSLGG | |
| | GGGGGGSGGGGSGGGGSKGRDSKPSPACDPMHGALAGIFKELRTTYRSVR | |
| | EALQTKDTVYYVSLFHEQLLQEMLSPVGCRVTNELMQHYLDGVLPRAFHC | |
| | GYDNATLNALHALSSSLSTLYQHMLKCPALACTGQTPAWTQFLDTEHKLD | |
| | PWKGTVKATAEMDLLLNYLETFLLQS | |
| m2a-IFP-7 | MYRMQLLSCIALSLALVINSEPRGPTIKPCPPCKCPAPNAAGGPSVFIFP | 490 |
| with hIL2- | PKIKDVLMISLSPIVTCVVVDVSEDDPDVQISWFVNNVEVHTAQTQTHRE | |
| signal peptide | DYNSTLRVVSALPIQHQDWMSGKEFKCKVNNKDLGAPIERTISKPKGSVR | |
| | APQVYVLPPPEEEMTKKQVTLTCMVTDFMPEDIYVEWTNNGKTELNYKNT | |
| | EPVLDSDGSYFMYSKLRVEKKNWVERNSYSCSVVHEGLHNHHTTKSFSRT | |
| | PGKGGGGGGGGSGGGGSGGGGSKGRDSKPSPACDPMHGALAGIFKELRTT | |
| | YRSVREALQTKDTVYYVSLFHEQLLQEMLSPVGCRVTNELMQHYLDGVLP | |
| | RAFHCGYDNATLNALHALSSSLSTLYQHMLKCPALACTGQTPAWTQFLDT | |
| | EHKLDPWKGTVKATAEMDLLLNYLETFLLQS | |
| m1-IFP-7 | MYRMQLLSCIALSLALVINSVPRDCGCKPCICTVPEVSSVFIFPPKPKDV | 491 |
| with hIL2- | LTITLTPKVTCVVVAISKDDPEVQFSWFVDDVEVHTAQTQPREEQFNSTF | |
| signal peptide | RSVSELPIMHQDWLNGKEFKCRVNSAAFPAPIEKTISKTKGRPKAPQVYT | |
| | IPPPKEQMAKDKVSLTCMITDFFPEDITVEWQWNGQPAENYKNTQPIMDT | |
| | DGSYFVYSKLNVQKSNWEAGNTFTCSVLHEGLHNHHTEKSLSHSPGGGGG | |
| | GGSGGGGSGGGGSKGRDSKPSPACDPMHGALAGIFKELRITYRSVREALQ | |
| | TKDTVYYVSLFHEQLLQEMLSPVGCRVTNELMQHYLDGVLPRAFHCGYDN | |
| | ATLNALHALSSSLSTLYQHMLKCPALACTGQTPAWTQFLDTEHKLDPWKG | |
| | TVKATAEMDLLLNYLETFLLQS | |
| IFP-8 | MYRMQLLSCIALSLALVINSAESKYGPPCPPCPAPEAAGGPSVFLFPPKP | 492 |
| with hIL2- | KDTLMISRTPEVTCVVVDVSQEDPEVQFNWYVDGVEVHNAKTKPREEQEN | |
| signal peptide | STYRVVSVLTVLHQDWLNGKEYKCKVSNKGLPSSIEKTISKAKGQPREPQ | |
| | VYTLPPSQEEMTKNQVSLTCLVKGFYPSDIAVEWESNGQPENNYKITPPV | |
| | LDSDGSFFLYSRLTVDKSRWQEGNVFSCSVMHEALHNHYTQKSLSLSLGG | |
| | GGGGGGSGGGGSGGGGSATTTIKNTKPQCRPEDYATRLQDLRVTFHRVKP | |
| | TLQREDDYSVWLDGTVVKGCWGCSVMDWLLRRYLEIVFPAGDHVYPGLKT | |
| | ELHSMRSTLESIYKDMRQCPLLGCGDKSVISRLSQEAERKSDNGIRKGLS | |
| | ELDTLFSRLEEYLHSRK | |
| IFP-1 | AESKYGPPCPPCPAPEAAGGPSVFLFPPKPKDTLMISRTPEVTCVVVDVS | 493 |
| without hIL2- | QEDPEVQFNWYVDGVEVHNAKTKPREEQFNSTYRVVSVLIVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSRHC | |
| | MFGDSLRNSPDMKNMLQDLRGGYSGSGIKRTFQGKDTLDSMLLTQSLLDD | |
| | FKGYLGCQALSEMIQFYLEEVMPQAENHGPTDSVKQLGEKLHTLNQKFGE | |
| | CPRWFPCYYNTTPAVENVKSVFSKLQERGVYKAMSEFDIFINYIETYTTM | |
| | K | |
| IFP-2 | AESKYGPPCPPCPAPEAAGGPSVFLFPPKPKDTLMISRTPEVTCVVVDVS | 494 |
| without hIL2- | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSCQL | |
| | ESGEALPLGSRSADSRSVDGQRVPAPQNNYPGLLRDLRLGYEGFKQKVTD | |
| | SHPDETLLGSSRLAGDLKGPLRCQALSEMIQFLLQVVLPDAENSRQDLRS | |
| | QFSTLGDRITGLRQQLRRDPTVFPCESRSDGVSDLRSAYTRLGSTGAEKV | |
| | LSEFDIFINYIEAYVTSV | |
| IFP-3 | AESKYGPPCPPCPAPEAAGGPSVFLFPPKPKDTLMISRTPEVTCVVVDVS | 495 |
| without hIL2- | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKITPPVLDSDGSFFLYSRLTVDKSRW | |
| | QEGNVFSCSVMHEALHNHYTQKSLSLSLGGGGGGGGSGGGGSGGGGSYCV | |
| | EYAESDEDRQQCSSSSNFPASLPHMLRELRAAFGKVKTFFQMKDQLNSML | |
| | LTQSLLDDFKGYLGCQALSEMIQFYLEEVMPQAENHGPDIKEHVNSLGEK | |
| | LKTLRLRLRRCHRFLPCENKSKAVEQVKRVENMLQERGVYKAMSEFDIFI | |
| | NYIESYMTTKM | |
| IFP-4 | AESKYGPPCPPCPAPEAAGGPSVFLFPPKPKDTLMISRTPEVTCVVVDVS | 496 |
| without hIL2- | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSYCT | |
| | SCSHHQCTEDENQKQDCEDANHSLPHMLRELRAAFGKVKTFFQMKDQLHS | |
| | LLLTQSLLDDFKGYLGCQALSEMIQFYLEEVMPQAENHGPEEHDNSLSEH | |
| | GPDVKEHVNSLGEKLKTLRLRLRRCHRFLPCENKSKAVEKVKRVFSELQE | |
| | RGVYKAMSEFDIFINYIETYMTT | |
| IFP-5 | AESKYGPPCPPCPAPEAAGGPSVFLFPPKPKDTLMISRTPEVTCVVVDVS | 497 |
| with hIL2- | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKITPPVLDSDGSFFLYSRLTVDKSRW | |
| | QEGNVFSCSVMHEALHNHYTQKSLSLSLGGGGGGGGSGGGGSGGGGSRSP | |
| | KNKPSIDCNPQTGDFVNMLKSMRQDYSRIRDILHDRDKLHSSLLTGALLD | |
| | EMMGYSGCRTTLLLMEHYLDTWYPAAYRHHLYDNQTLVVVDRMGSTLVAL | |
| | LKAMVQCPMLACGAPSPAMDKMLQQEAKMKKYTGVYKGISETDLLLGYLE | |
| | LYMMKFKR | |
| IFP-6 | AESKYGPPCPPCPAPEAAGGPSVFLFPPKPKDTLMISRTPEVTCVVVDVS | 498 |
| without hIL2- | QEDPEVQFNWYVDGVEVHNAKTKPREEQFNSTYRVVSVLTVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSHEH | |
| | KVPPACDPVHGNLAGIFKELRAIYASIREALQKKDTVYYTSLENDRVLQE | |
| | MLSPMGCRVTNELMEHYLDGVLPRAAHFDYDNSTLNGLHAFTSSMQALYQ | |
| | HMLKCPALACTGKTPAWMYFLEVEHKLNPWRGTAKAAAEADLLLNYLETF | |
| | LLQF | |
| IFP-7 | AESKYGPPCPPCPAPEAAGGPSVFLFPPKPKDTLMISRTPEVTCVVVDVS | 499 |
| without hIL2- | QEDPEVQFNWYVDGVEVHNAKTKPREEQFNSTYRVVSVLIVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGGGGGGGGSGGGGSGGGGSKGR | |
| | DSKPSPACDPMHGALAGIFKELRTTYRSVREALQTKDTVYYVSLFHEQLL | |
| | QEMLSPVGCRVTNELMQHYLDGVLPRAFHCGYDNATLNALHALSSSLSTL | |
| | YQHMLKCPALACTGQTPAWTQFLDTEHKLDPWKGTVKATAEMDLLLNYLE | |
| | TELLQS | |
| m2a-IFP-7 | EPRGPTIKPCPPCKCPAPNAAGGPSVFIFPPKIKDVLMISLSPIVTCVVV | 500 |
| without hIL2- | DVSEDDPDVQISWFVNNVEVHTAQTQTHREDYNSTLRVVSALPIQHQDWM | |
| signal peptide | SGKEFKCKVNNKDLGAPIERTISKPKGSVRAPQVYVLPPPEEEMTKKQVT | |
| | LTCMVTDFMPEDIYVEWTNNGKTELNYKNTEPVLDSDGSYFMYSKLRVEK | |
| | KNWVERNSYSCSVVHEGLHNHHTTKSFSRTPGKGGGGGGGGSGGGGSGGG | |
| | GSKGRDSKPSPACDPMHGALAGIFKELRITYRSVREALQTKDTVYYVSLF | |
| | HEQLLQEMLSPVGCRVTNELMQHYLDGVLPRAFHCGYDNATLNALHALSS | |
| | SLSTLYQHMLKCPALACTGQTPAWTQFLDTEHKLDPWKGTVKATAEMDLL | |
| | LNYLETELLQS | |
| m1-IFP-7 | VPRDCGCKPCICTVPEVSSVFIFPPKPKDVLTITLTPKVTCVVVAISKDD | 501 |
| without hIL2- | PEVQFSWFVDDVEVHTAQTQPREEQFNSTFRSVSELPIMHQDWLNGKEFK | |
| signal peptide | CRVNSAAFPAPIEKTISKTKGRPKAPQVYTIPPPKEQMAKDKVSLICMIT | |
| | DFFPEDITVEWQWNGQPAENYKNTQPIMDTDGSYFVYSKLNVQKSNWEAG | |
| | NTFTCSVLHEGLHNHHTEKSLSHSPGGGGGGGSGGGGSGGGGSKGRDSKP | |
| | SPACDPMHGALAGIFKELRTTYRSVREALQTKDTVYYVSLFHEQLLQEML | |
| | SPVGCRVTNELMQHYLDGVLPRAFHCGYDNAILNALHALSSSLSTLYQHM | |
| | LKCPALACTGQTPAWTQFLDTEHKLDPWKGTVKATAEMDLLLNYLETFLL | |
| | QS | |
| IFP-8 | AESKYGPPCPPCPAPEAAGGPSVFLFPPKPKDTLMISRTPEVTCVVVDVS | 502 |
| without hIL2- | QEDPEVQFNWYVDGVEVHNAKTKPREEQFNSTYRVVSVLIVLHQDWLNGK | |
| signal peptide | EYKCKVSNKGLPSSIEKTISKAKGQPREPQVYTLPPSQEEMTKNQVSLTC | |
| | LVKGFYPSDIAVEWESNGQPENNYKITPPVLDSDGSFFLYSRLTVDKSRW | |
| | QEGNVFSCSVMHEALHNHYTQKSLSLSLGGGGGGGGSGGGGSGGGGSATT | |
| | TIKNTKPQCRPEDYATRLQDLRVTFHRVKPTLQREDDYSVWLDGTVVKGC | |
| | WGCSVMDWLLRRYLEIVFPAGDHVYPGLKTELHSMRSTLESIYKDMRQCP | |
| | LLGCGDKSVISRLSQEAERKSDNGTRKGLSELDTLFSRLEEYLHSRK | |
| IFP-1 | MYRMQLLSCIALSLALVINSESKYGPPCPPCPAPEAAGGPSVFLFPPKPK | 503 |
| with hIL2- | DTLMISRTPEVTCVVVDVSQEDPEVQFNWYVDGVEVHNAKIKPREEQFNS | |
| signal peptide | TYRVVSVLIVLHQDWLNGKEYKCKVSNKGLPSSIEKTISKAKGQPREPQV | |
| | YTLPPSQEEMTKNQVSLICLVKGFYPSDIAVEWESNGQPENNYKITPPVL | |
| | DSDGSFFLYSRLTVDKSRWQEGNVFSCSVMHEALHNHYTQKSLSLSLGGG | |
| | GGGGGSGGGGSGGGGSRHCMFGDSLRNSPDMKNMLQDLRGGYSGSGIKRT | |
| | FQGKDTLDSMLLTQSLLDDFKGYLGCQALSEMIQFYLEEVMPQAENHGPT | |
| | DSVKQLGEKLHTLNQKFGECPRWFPCYYNTTPAVENVKSVFSKLQERGVY | |
| | KAMSEFDIFINYIETYTTMK | |
| IFP-2 | MYRMQLLSCIALSLALVINSESKYGPPCPPCPAPEAAGGPSVFLFPPKPK | 504 |
| with hIL2- | DTLMISRTPEVTCVVVDVSQEDPEVQFNWYVDGVEVHNAKTKPREEQENS | |
| signal peptide | TYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSCQLESGEALPLGSRSADSRSVDGQRVPAPQNNYP | |
| | GLLRDLRLGYEGFKQKVTDSHPDETLLGSSRLAGDLKGPLRCQALSEMIQ | |
| | FLLQVVLPDAENSRQDLRSQFSTLGDRITGLRQQLRRDPTVFPCESRSDG | |
| | VSDLRSAYTRLGSTGAEKVLSEFDIFINYIEAYVTSV | |
| IFP-3 | MYRMQLLSCIALSLALVINSESKYGPPCPPCPAPEAAGGPSVFLFPPKPK | 505 |
| with hIL2- | DTLMISRTPEVTCVVVDVSQEDPEVQFNWYVDGVEVHNAKTKPREEQFNS | |
| signal peptide | TYRVVSVLIVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSYCVEYAESDEDRQQCSSSSNFPASLPHMLRELRA | |
| | AFGKVKTFFQMKDQLNSMLLTQSLLDDFKGYLGCQALSEMIQFYLEEVMP | |
| | QAENHGPDIKEHVNSLGEKLKTLRLRLRRCHRFLPCENKSKAVEQVKRVF | |
| | NMLQERGVYKAMSEFDIFINYIESYMTTKM | |
| IFP-4 | MYRMQLLSCIALSLALVINSESKYGPPCPPCPAPEAAGGPSVFLFPPKPK | 506 |
| with hIL2- | DTLMISRTPEVTCVVVDVSQEDPEVQFNWYVDGVEVHNAKIKPREEQENS | |
| signal peptide | TYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKITPPVL | |
| | DSDGSFFLYSRLTVDKSRWQEGNVFSCSVMHEALHNHYTQKSLSLSLGGG | |
| | GGGGGSGGGGSGGGGSYCTSCSHHQCTEDENQKQDCEDANHSLPHMLREL | |
| | RAAFGKVKTFFQMKDQLHSLLLTQSLLDDFKGYLGCQALSEMIQFYLEEV | |
| | MPQAENHGPEEHDNSLSEHGPDVKEHVNSLGEKLKTLRLRLRRCHRFLPC | |
| | ENKSKAVEKVKRVFSELQERGVYKAMSEFDIFINYIETYMTT | |
| IFP-5 | MYRMQLLSCIALSLALVINSESKYGPPCPPCPAPEAAGGPSVFLFPPKPK | 507 |
| with hIL2- | DTLMISRTPEVTCVVVDVSQEDPEVQFNWYVDGVEVHNAKTKPREEQENS | |
| signal peptide | TYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSRSPKNKPSIDCNPQTGDFVNMLKSMRQDYSRIRD | |
| | TLHDRDKLHSSLLIGALLDEMMGYSGCRTTLLLMEHYLDTWYPAAYRHHL | |
| | YDNQTLVVVDRMGSTLVALLKAMVQCPMLACGAPSPAMDKMLQQEAKMKK | |
| | YTGVYKGISETDLLLGYLELYMMKFKR | |
| IFP-6 | MYRMQLLSCIALSLALVINSESKYGPPCPPCPAPEAAGGPSVFLFPPKPK | 508 |
| with hIL2- | DTLMISRTPEVTCVVVDVSQEDPEVQFNWYVDGVEVHNAKIKPREEQFNS | |
| signal peptide | TYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSHEHKVPPACDPVHGNLAGIFKELRAIYASIREAL | |
| | QKKDTVYYTSLFNDRVLQEMLSPMGCRVTNELMEHYLDGVLPRAAHFDYD | |
| | NSTLNGLHAFTSSMQALYQHMLKCPALACTGKTPAWMYFLEVEHKLNPWR | |
| | GTAKAAAEADLLLNYLETFLLQF | |
| IFP-7 | MYRMQLLSCIALSLALVINSESKYGPPCPPCPAPEAAGGPSVFLFPPKPK | 509 |
| with hIL2- | DTLMISRTPEVTCVVVDVSQEDPEVQFNWYVDGVEVHNAKTKPREEQFNS | |
| signal peptide | TYRVVSVLTVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSKGRDSKPSPACDPMHGALAGIFKELRTTYRSVRE | |
| | ALQTKDTVYYVSLFHEQLLQEMLSPVGCRVTNELMQHYLDGVLPRAFHCG | |
| | YDNATLNALHALSSSLSTLYQHMLKCPALACTGQTPAWTQFLDTEHKLDP | |
| | WKGTVKATAEMDLLLNYLETFLLQS | |
| IFP-8 | MYRMQLLSCIALSLALVINSESKYGPPCPPCPAPEAAGGPSVFLFPPKPK | 510 |
| with hIL2- | DTLMISRTPEVTCVVVDVSQEDPEVQFNWYVDGVEVHNAKTKPREEQFNS | |
| signal peptide | TYRVVSVLIVLHQDWLNGKEYKCKVSNKGLPSSIEKTISKAKGQPREPQV | |
| | YTLPPSQEEMTKNQVSLTCLVKGFYPSDIAVEWESNGQPENNYKTTPPVL | |
| | DSDGSFFLYSRLTVDKSRWQEGNVFSCSVMHEALHNHYTQKSLSLSLGGG | |
| | GGGGGSGGGGSGGGGSATTTIKNTKPQCRPEDYATRLQDLRVTFHRVKPT | |
| | LQREDDYSVWLDGTVVKGCWGCSVMDWLLRRYLEIVFPAGDHVYPGLKTE | |
| | LHSMRSTLESIYKDMRQCPLLGCGDKSVISRLSQEAERKSDNGTRKGLSE | |
| | LDTLFSRLEEYLHSRK | |
| IFP-1 | ESKYGPPCPPCPAPEAAGGPSVFLFPPKPKDTLMISRTPEVTCVVVDVSQ | 511 |
| without hIL2- | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSRHCM | |
| | FGDSLRNSPDMKNMLQDLRGGYSGSGIKRTFQGKDTLDSMLLTQSLLDDF | |
| | KGYLGCQALSEMIQFYLEEVMPQAENHGPTDSVKQLGEKLHTLNQKFGEC | |
| | PRWFPCYYNTTPAVENVKSVFSKLQERGVYKAMSEFDIFINYIETYTTMK | |
| IFP-2 | ESKYGPPCPPCPAPEAAGGPSVFLFPPKPKDTLMISRTPEVTCVVVDVSQ | 512 |
| without hIL2- | EDPEVQFNWYVDGVEVHNAKTKPREEQFNSTYRVVSVLIVLHQDWLNGKE | |
| signal peptide | YKCKVSNKGLPSSIEKTISKAKGQPREPQVYTLPPSQEEMTKNQVSLICL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSCQLE | |
| | SGEALPLGSRSADSRSVDGQRVPAPQNNYPGLLRDLRLGYEGFKQKVTDS | |
| | HPDETLLGSSRLAGDLKGPLRCQALSEMIQFLLQVVLPDAENSRQDLRSQ | |
| | FSTLGDRITGLRQQLRRDPTVFPCESRSDGVSDLRSAYTRLGSTGAEKVL | |
| | SEFDIFINYIEAYVTSV | |
| IFP-3 | ESKYGPPCPPCPAPEAAGGPSVFLFPPKPKDTLMISRTPEVTCVVVDVSQ | 513 |
| without hIL2- | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSYCVE | |
| | YAESDEDRQQCSSSSNFPASLPHMLRELRAAFGKVKTFFQMKDQLNSMLL | |
| | TQSLLDDFKGYLGCQALSEMIQFYLEEVMPQAENHGPDIKEHVNSLGEKL | |
| | KTLRLRLRRCHRFLPCENKSKAVEQVKRVFNMLQERGVYKAMSEFDIFIN | |
| | YIESYMTTKM | |
| IFP-4 | ESKYGPPCPPCPAPEAAGGPSVFLFPPKPKDILMISRTPEVTCVVVDVSQ | 514 |
| without hIL2- | EDPEVQFNWYVDGVEVHNAKTKPREEQFNSTYRVVSVLIVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSYCTS | |
| | CSHHQCTEDENQKQDCEDANHSLPHMLRELRAAFGKVKTFFQMKDQLHSL | |
| | LLTQSLLDDFKGYLGCQALSEMIQFYLEEVMPQAENHGPEEHDNSLSEHG | |
| | PDVKEHVNSLGEKLKTLRLRLRRCHRFLPCENKSKAVEKVKRVFSELQER | |
| | GVYKAMSEFDIFINYIETYMIT | |
| IFP-5 | ESKYGPPCPPCPAPEAAGGPSVFLFPPKPKDTLMISRIPEVTCVVVDVSQ | 515 |
| with hIL2- | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSRSPK | |
| | NKPSIDCNPQTGDFVNMLKSMRQDYSRIRDTLHDRDKLHSSLLTGALLDE | |
| | MMGYSGCRTTLLLMEHYLDTWYPAAYRHHLYDNQTLVVVDRMGSTLVALL | |
| | KAMVQCPMLACGAPSPAMDKMLQQEAKMKKYTGVYKGISETDLLLGYLEL | |
| | YMMKFKR | |
| IFP-6 | ESKYGPPCPPCPAPEAAGGPSVFLFPPKPKDTLMISRTPEVTCVVVDVSQ | 516 |
| without hIL2- | EDPEVQFNWYVDGVEVHNAKTKPREEQFNSTYRVVSVLTVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSHEHK | |
| | VPPACDPVHGNLAGIFKELRAIYASIREALQKKDTVYYTSLFNDRVLQEM | |
| | LSPMGCRVTNELMEHYLDGVLPRAAHFDYDNSTLNGLHAFTSSMQALYQH | |
| | MLKCPALACTGKTPAWMYFLEVEHKLNPWRGTAKAAAEADLLLNYLETFL | |
| | LQF | |
| IFP-7 | ESKYGPPCPPCPAPEAAGGPSVFLFPPKPKDILMISRIPEVTCVVVDVSQ | 517 |
| without hIL2- | EDPEVQFNWYVDGVEVHNAKTKPREEQFNSTYRVVSVLIVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKSRWQ | |
| | EGNVFSCSVMHEALHNHYTQKSLSLSLGGGGGGGGSGGGGSGGGGSKGRD | |
| | SKPSPACDPMHGALAGIFKELRTTYRSVREALQTKDTVYYVSLFHEQLLQ | |
| | EMLSPVGCRVTNELMQHYLDGVLPRAFHCGYDNATLNALHALSSSLSTLY | |
| | QHMLKCPALACTGQTPAWTQFLDTEHKLDPWKGTVKATAEMDLLLNYLET | |
| | FLLQS | |
| IFP-8 | ESKYGPPCPPCPAPEAAGGPSVFLFPPKPKDTLMISRTPEVTCVVVDVSQ | 518 |
| without hIL2- | EDPEVQFNWYVDGVEVHNAKTKPREEQFNSTYRVVSVLIVLHQDWLNGKE | |
| signal peptide | YKCKVSNKGLPSSIEKTISKAKGQPREPQVYTLPPSQEEMTKNQVSLTCL | |
| | VKGFYPSDIAVEWESNGQPENNYKITPPVLDSDGSFFLYSRLTVDKSRWQ | |
| | EGNVFSCSVMHEALHNHYTQKSLSLSLGGGGGGGGSGGGGSGGGGSATTT | |
| | IKNTKPQCRPEDYATRLQDLRVTFHRVKPTLQREDDYSVWLDGTVVKGCW | |
| | GCSVMDWLLRRYLEIVFPAGDHVYPGLKTELHSMRSTLESIYKDMRQCPL | |
| | LGCGDKSVISRLSQEAERKSDNGTRKGLSELDTLFSRLEEYLHSRK | |

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 7. For example, the amino acid sequence of the immunomodulatory fusion protein or polypeptide may comprise an amino acid sequence at least 85% identical to the amino acid sequence of a polypeptide set forth in Table 7. The amino acid sequence of the immunomodulatory fusion protein or polypeptide may comprise an amino acid sequence at least 90% identical to the amino acid sequence of a polypeptide set forth in Table 7. The amino acid sequence of the immunomodulatory fusion protein or polypeptide may comprise an amino acid sequence at least 95% identical to the amino acid sequence of a polypeptide set forth in Table 7. In some embodiments, the amino acid sequence of the immunomodulatory fusion protein or polypeptide preferably may comprise an amino acid sequence 100% identical to the amino acid sequence of a polypeptide set forth in Table 7. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 7. For example, the amino acid sequence of the immunomodulatory fusion protein or polypeptide may consist of an amino acid sequence at least 85% identical to the amino acid sequence of a polypeptide set forth in Table 7. The amino acid sequence of the immunomodulatory fusion protein or polypeptide may consist of an amino acid sequence at least 90% identical to the amino acid sequence of a polypeptide set forth in Table 7. The amino acid sequence of the immunomodulatory fusion protein or polypeptide may consist of an amino acid sequence at least 95% identical to the amino acid sequence of a polypeptide set forth in Table 7. In some embodiments, the amino acid sequence of the immunomodulatory fusion protein or polypeptide preferably may consist of an amino acid sequence 100% identical to the amino acid sequence of a polypeptide set forth in Table 7.

In some embodiments, the amino acid sequence of the immunomodulatory fusion protein or polypeptide comprises or consists of the amino acid sequence of a polypeptide set forth in Table 7, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of a polypeptide set forth in Table 7, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of a polypeptide set forth in Table 7, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of a polypeptide set forth in Table 7, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of a polypeptide set forth in Table 7, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of a polypeptide set forth in Table 7, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of a polypeptide set forth in Table 7, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of a polypeptide set forth in Table 7, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 58-101 or 479-518. For example, the amino acid sequence of the immunomodulatory fusion protein or polypeptide may comprise an amino acid sequence at least 85% identical to the amino acid sequence of any one of SEQ ID NOS: 58-101 or 479-518. The amino acid sequence of the immunomodulatory fusion protein or polypeptide may comprise an amino acid sequence at least 90% identical to the amino acid sequence of any one of SEQ ID NOS: 58-101 or 479-518. The amino acid sequence of the immunomodulatory fusion protein or polypeptide may comprise an amino acid sequence at least 95% identical to the amino acid sequence of any one of SEQ ID NOS: 58-101 or 479-518. In some embodiments, the amino acid sequence of the immunomodulatory fusion protein or polypeptide preferably may comprise an amino acid sequence 100% identical to the amino acid sequence of any one of SEQ ID NOS: 58-101 or 479-518. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 58-101 or 479-518. For example, the amino acid sequence of the immunomodulatory fusion protein or polypeptide may consist of an amino acid sequence at least 85% identical to the amino acid sequence of any one of SEQ ID NOS: 58-101 or 479-518. The amino acid sequence of the immunomodulatory fusion protein or polypeptide may consist of an amino acid sequence at least 90% identical to the amino acid sequence of any one of SEQ ID NOS: 58-101 or 479-518. The amino acid sequence of the immunomodulatory fusion protein or polypeptide may consist of an amino acid sequence at least 95% identical to the amino acid sequence of any one of SEQ ID NOS: 58-101 or 479-518. In some embodiments, the amino acid sequence of the immunomodulatory fusion protein or polypeptide preferably may consist of an amino acid sequence 100% identical to the amino acid sequence of any one of SEQ ID NOS: 58-101 or 479-518.

In some embodiments, the amino acid sequence of the immunomodulatory fusion protein or polypeptide comprises or consists of the amino acid sequence of any one of SEQ ID NOS: 58-101 or 479-518, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 58-101 or 479-518, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 58-101 or 479-518, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 58-101 or 479-518, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 58-101 or 479-518, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 58-101 or 479-518, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 58-101 or 479-518, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 58-101 or 479-518, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 58-68 or 479-480. For example, the amino acid sequence of the immunomodulatory fusion protein or polypeptide may comprise an amino acid sequence at least 85% identical to the amino acid sequence of any one of SEQ ID NOS: 58-68 or 479-480. The amino acid sequence of the immunomodulatory fusion protein or polypeptide may comprise an amino acid sequence at least 90% identical to the amino acid sequence of any one of SEQ ID NOS: 58-68 or 479-480. The amino acid sequence of the immunomodulatory fusion protein or polypeptide may comprise an amino acid sequence at least 95% identical to the amino acid sequence of any one of SEQ ID NOS: 58-68 or 479-480. In some embodiments, the amino acid sequence of the immunomodulatory fusion protein or polypeptide preferably may comprise an amino acid sequence 100% identical to the amino acid sequence of any one of SEQ ID NOS: 58-68 or 479-480. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 58-68 or 479-480. For example, the amino acid sequence of the immunomodulatory fusion protein or polypeptide may consist of an amino acid sequence at least 85% identical to the amino acid sequence of any one of SEQ ID NOS: 58-68 or 479-480. The amino acid sequence of the immunomodulatory fusion protein or polypeptide may consist of an amino acid sequence at least 90% identical to the amino acid sequence of any one of SEQ ID NOS: 58-68 or 479-480. The amino acid sequence of the immunomodulatory fusion protein or polypeptide may consist of an amino acid sequence at least 95% identical to the amino acid sequence of any one of SEQ ID NOS: 58-68 or 479-480. In some embodiments, the amino acid sequence of the immunomodulatory fusion protein or polypeptide preferably may consist of an amino acid sequence 100% identical to the amino acid sequence of any one of SEQ ID NOS: 58-68 or 479-480.

In some embodiments, the amino acid sequence of the immunomodulatory fusion protein or polypeptide comprises or consists of the amino acid sequence of any one of SEQ ID NOS: 58-68 or 479-480, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 58-68 or 479-480, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 58-68 or 479-480, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 58-68 or 479-480, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 58-68 or 479-480, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 58-68 or 479-480, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 58-68 or 479-480, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 58-68 or 479-480, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 69-79 or 481-482. For example, the amino acid sequence of the immunomodulatory fusion protein or polypeptide may comprise an amino acid sequence at least 85% identical to the amino acid sequence of any one of SEQ ID NOS: 69-79 or 481-482. The amino acid sequence of the immunomodulatory fusion protein or polypeptide may comprise an amino acid sequence at least 90% identical to the amino acid sequence of any one of SEQ ID NOS: 69-79 or 481-482. The amino acid sequence of the immunomodulatory fusion protein or polypeptide may comprise an amino acid sequence at least 95% identical to the amino acid sequence of any one of SEQ ID NOS: 69-79 or 481-482. In some embodiments, the amino acid sequence of the immunomodulatory fusion protein or polypeptide preferably may comprise an amino acid sequence 100% identical to the amino acid sequence of any one of SEQ ID NOS: 69-79 or 481-482. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 69-79 or 481-482. For example, the amino acid sequence of the immunomodulatory fusion protein or polypeptide may consist of an amino acid sequence at least 85% identical to the amino acid sequence of any one of SEQ ID NOS: 69-79 or 481-482. The amino acid sequence of the immunomodulatory fusion protein or polypeptide may consist of an amino acid sequence at least 90% identical to the amino acid sequence of any one of SEQ ID NOS: 69-79 or 481-482. The amino acid sequence of the immunomodulatory fusion protein or polypeptide may consist of an amino acid sequence at least 95% identical to the amino acid sequence of any one of SEQ ID NOS: 69-79 or 481-482. In some embodiments, the amino acid sequence of the immunomodulatory fusion protein or polypeptide preferably may consist of an amino acid sequence 100% identical to the amino acid sequence of any one of SEQ ID NOS: 69-79 or 481-482.

In some embodiments, the amino acid sequence of the immunomodulatory fusion protein or polypeptide comprises or consists of the amino acid sequence of any one of SEQ ID NOS: 69-79 or 481-482, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 69-79 or 481-482, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 69-79 or 481-482, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 69-79 or 481-482, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 69-79 or 481-482, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 69-79 or 481-482, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 69-79 or 481-482, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 69-79 or 481-482, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 80-90. For example, the amino acid sequence of the immunomodulatory fusion protein or polypeptide may comprise an amino acid sequence at least 85% identical to the amino acid sequence of any one of SEQ ID NOS: 80-90. The amino acid sequence of the immunomodulatory fusion protein or polypeptide may comprise an amino acid sequence at least 90% identical to the amino acid sequence of any one of SEQ ID NOS: 80-90. The amino acid sequence of the immunomodulatory fusion protein or polypeptide may comprise an amino acid sequence at least 95% identical to the amino acid sequence of any one of SEQ ID NOS: 80-90. In some embodiments, the amino acid sequence of the immunomodulatory fusion protein or polypeptide preferably may comprise an amino acid sequence 100% identical to the amino acid sequence of any one of SEQ ID NOS: 80-90. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 80-90. For example, the amino acid sequence of the immunomodulatory fusion protein or polypeptide may consist of an amino acid sequence at least 85% identical to the amino acid sequence of any one of SEQ ID NOS: 80-90. The amino acid sequence of the immunomodulatory fusion protein or polypeptide may consist of an amino acid sequence at least 90% identical to the amino acid sequence of any one of SEQ ID NOS: 80-90. The amino acid sequence of the immunomodulatory fusion protein or polypeptide may consist of an amino acid sequence at least 95% identical to the amino acid sequence of any one of SEQ ID NOS: 80-90. In some embodiments, the amino acid sequence of the immunomodulatory fusion protein or polypeptide preferably may consist of an amino acid sequence 100% identical to the amino acid sequence of any one of SEQ ID NOS: 80-90.

In some embodiments, the amino acid sequence of the immunomodulatory fusion protein or polypeptide comprises or consists of the amino acid sequence of any one of SEQ ID NOS: 80-90, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 80-90, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 80-90, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 80-90, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 80-90, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 80-90, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 80-90, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 80-90, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 91-101. For example, the amino acid sequence of the immunomodulatory fusion protein or polypeptide may comprise an amino acid sequence at least 85% identical to the amino acid sequence of any one of SEQ ID NOS: 91-101. The amino acid sequence of the immunomodulatory fusion protein or polypeptide may comprise an amino acid sequence at least 90% identical to the amino acid sequence of any one of SEQ ID NOS: 91-101. The amino acid sequence of the immunomodulatory fusion protein or polypeptide may comprise an amino acid sequence at least 95% identical to the amino acid sequence of any one of SEQ ID NOS: 91-101. In some embodiments, the amino acid sequence of the immunomodulatory fusion protein or polypeptide preferably may comprise an amino acid sequence 100% identical to the amino acid sequence of any one of SEQ ID NOS: 91-101. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 91-101. For example, the amino acid sequence of the immunomodulatory fusion protein or polypeptide may consist of an amino acid sequence at least 85% identical to the amino acid sequence of any one of SEQ ID NOS: 91-101. The amino acid sequence of the immunomodulatory fusion protein or polypeptide may consist of an amino acid sequence at least 90% identical to the amino acid sequence of any one of SEQ ID NOS: 91-101. The amino acid sequence of the immunomodulatory fusion protein or polypeptide may consist of an amino acid sequence at least 95% identical to the amino acid sequence of any one of SEQ ID NOS: 91-101. In some embodiments, the amino acid sequence of the immunomodulatory fusion protein or polypeptide preferably may consist of an amino acid sequence 100% identical to the amino acid sequence of any one of SEQ ID NOS: 91-101.

In some embodiments, the amino acid sequence of the immunomodulatory fusion protein or polypeptide comprises or consists of the amino acid sequence of any one of SEQ ID NOS: 91-101, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 91-101, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 91-101, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 91-101, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 91-101, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 91-101, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 91-101, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 91-101, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 483-518. For example, the amino acid sequence of the immunomodulatory fusion protein or polypeptide may comprise an amino acid sequence at least 85% identical to the amino acid sequence of any one of SEQ ID NOS: 483-518. The amino acid sequence of the immunomodulatory fusion protein or polypeptide may comprise an amino acid sequence at least 90% identical to the amino acid sequence of any one of SEQ ID NOS: 483-518. The amino acid sequence of the immunomodulatory fusion protein or polypeptide may comprise an amino acid sequence at least 95% identical to the amino acid sequence of any one of SEQ ID NOS: 483-518. In some embodiments, the amino acid sequence of the immunomodulatory fusion protein or polypeptide preferably may comprise an amino acid sequence 100% identical to the amino acid sequence of any one of SEQ ID NOS: 483-518. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 483-518. For example, the amino acid sequence of the immunomodulatory fusion protein or polypeptide may consist of an amino acid sequence at least 85% identical to the amino acid sequence of any one of SEQ ID NOS: 483-518. The amino acid sequence of the immunomodulatory fusion protein or polypeptide may consist of an amino acid sequence at least 90% identical to the amino acid sequence of any one of SEQ ID NOS: 483-518. The amino acid sequence of the immunomodulatory fusion protein or polypeptide may consist of an amino acid sequence at least 95% identical to the amino acid sequence of any one of SEQ ID NOS: 483-518. In some embodiments, the amino acid sequence of the immunomodulatory fusion protein or polypeptide preferably may consist of an amino acid sequence 100% identical to the amino acid sequence of any one of SEQ ID NOS: 483-518.

In some embodiments, the amino acid sequence of the immunomodulatory fusion protein or polypeptide comprises or consists of the amino acid sequence of any one of SEQ ID NOS: 483-518, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 483-518, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 483-518, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 483-518, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 483-518, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 483-518, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 483-518, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 483-518, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 483-492. For example, the amino acid sequence of the immunomodulatory fusion protein or polypeptide may comprise an amino acid sequence at least 85% identical to the amino acid sequence of any one of SEQ ID NOS: 483-492. The amino acid sequence of the immunomodulatory fusion protein or polypeptide may comprise an amino acid sequence at least 90% identical to the amino acid sequence of any one of SEQ ID NOS: 483-492. The amino acid sequence of the immunomodulatory fusion protein or polypeptide may comprise an amino acid sequence at least 95% identical to the amino acid sequence of any one of SEQ ID NOS: 483-492. In some embodiments, the amino acid sequence of the immunomodulatory fusion protein or polypeptide preferably may comprise an amino acid sequence 100% identical to the amino acid sequence of any one of SEQ ID NOS: 483-492. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 483-492. For example, the amino acid sequence of the immunomodulatory fusion protein or polypeptide may consist of an amino acid sequence at least 85% identical to the amino acid sequence of any one of SEQ ID NOS: 483-492. The amino acid sequence of the immunomodulatory fusion protein or polypeptide may consist of an amino acid sequence at least 90% identical to the amino acid sequence of any one of SEQ ID NOS: 483-492. The amino acid sequence of the immunomodulatory fusion protein or polypeptide may consist of an amino acid sequence at least 95% identical to the amino acid sequence of any one of SEQ ID NOS: 483-492. In some embodiments, the amino acid sequence of the immunomodulatory fusion protein or polypeptide preferably may consist of an amino acid sequence 100% identical to the amino acid sequence of any one of SEQ ID NOS: 483-492.

In some embodiments, the amino acid sequence of the immunomodulatory fusion protein or polypeptide comprises or consists of the amino acid sequence of any one of SEQ ID NOS: 483-492, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 483-492, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 483-492, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 483-492, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 483-492, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 483-492, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 483-492, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 483-492, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 493-510. For example, the amino acid sequence of the immunomodulatory fusion protein or polypeptide may comprise an amino acid sequence at least 85% identical to the amino acid sequence of any one of SEQ ID NOS: 493-510. The amino acid sequence of the immunomodulatory fusion protein or polypeptide may comprise an amino acid sequence at least 90% identical to the amino acid sequence of any one of SEQ ID NOS: 493-510. The amino acid sequence of the immunomodulatory fusion protein or polypeptide may comprise an amino acid sequence at least 95% identical to the amino acid sequence of any one of SEQ ID NOS: 493-510. In some embodiments, the amino acid sequence of the immunomodulatory fusion protein or polypeptide preferably may comprise an amino acid sequence 100% identical to the amino acid sequence of any one of SEQ ID NOS: 493-510. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 493-510. For example, the amino acid sequence of the immunomodulatory fusion protein or polypeptide may consist of an amino acid sequence at least 85% identical to the amino acid sequence of any one of SEQ ID NOS: 493-510. The amino acid sequence of the immunomodulatory fusion protein or polypeptide may consist of an amino acid sequence at least 90% identical to the amino acid sequence of any one of SEQ ID NOS: 493-510. The amino acid sequence of the immunomodulatory fusion protein or polypeptide may consist of an amino acid sequence at least 95% identical to the amino acid sequence of any one of SEQ ID NOS: 493-510. In some embodiments, the amino acid sequence of the immunomodulatory fusion protein or polypeptide preferably may consist of an amino acid sequence 100% identical to the amino acid sequence of any one of SEQ ID NOS: 493-510.

In some embodiments, the amino acid sequence of the immunomodulatory fusion protein or polypeptide comprises or consists of the amino acid sequence of any one of SEQ ID NOS: 493-510, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 493-510, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 493-510, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 493-510, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 493-510, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 493-510, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 493-510, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 493-510, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 511-518. For example, the amino acid sequence of the immunomodulatory fusion protein or polypeptide may comprise an amino acid sequence at least 85% identical to the amino acid sequence of any one of SEQ ID NOS: 511-518. The amino acid sequence of the immunomodulatory fusion protein or polypeptide may comprise an amino acid sequence at least 90% identical to the amino acid sequence of any one of SEQ ID NOS: 511-518. The amino acid sequence of the immunomodulatory fusion protein or polypeptide may comprise an amino acid sequence at least 95% identical to the amino acid sequence of any one of SEQ ID NOS: 511-518. In some embodiments, the amino acid sequence of the immunomodulatory fusion protein or polypeptide preferably may comprise an amino acid sequence 100% identical to the amino acid sequence of any one of SEQ ID NOS: 511-518. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 511-518. For example, the amino acid sequence of the immunomodulatory fusion protein or polypeptide may consist of an amino acid sequence at least 85% identical to the amino acid sequence of any one of SEQ ID NOS: 511-518. The amino acid sequence of the immunomodulatory fusion protein or polypeptide may consist of an amino acid sequence at least 90% identical to the amino acid sequence of any one of SEQ ID NOS: 511-518. The amino acid sequence of the immunomodulatory fusion protein or polypeptide may consist of an amino acid sequence at least 95% identical to the amino acid sequence of any one of SEQ ID NOS: 511-518. In some embodiments, the amino acid sequence of the immunomodulatory fusion protein or polypeptide may preferably consist of an amino acid sequence 100% identical to the amino acid sequence of any one of SEQ ID NOS: 511-518.

In some embodiments, the amino acid sequence of the immunomodulatory fusion protein or polypeptide comprises or consists of the amino acid sequence of any one of SEQ ID NOS: 511-518, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 511-518, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 511-518, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 511-518, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 511-518, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 511-518, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 511-518, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 511-518, and further comprises or consists of no more than about 1, 2, 3, 4, 5, 6, 7, 8, 9, or 10 amino acid substitutions.

Of the immunomodulatory fusion proteins or polypeptides referred to herein, in some embodiments, SEQ ID NOS: 58, 61-69, or 72-79 may be preferred. Advantageously, said immunomodulatory fusion proteins or polypeptides may engage hIL-10R with a higher potency than hIL-10. Thus, in some embodiments, an immunomodulatory fusion protein or polypeptide may comprise an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 58, 61-69, or 72-79. For example, the amino acid sequence of the immunomodulatory fusion protein or polypeptide may comprise an amino acid sequence at least 85% identical to the amino acid sequence of any one of SEQ ID NOS: 58, 61-69, or 72-79. The amino acid sequence of the immunomodulatory fusion protein or polypeptide may comprise an amino acid sequence at least 90% identical to the amino acid sequence of any one of SEQ ID NOS: 58, 61-69, or 72-79. The amino acid sequence of the immunomodulatory fusion protein or polypeptide may comprise an amino acid sequence at least 95% identical to the amino acid sequence of any one of SEQ ID NOS: 58, 61-69, or 72-79. In some embodiments, the amino acid sequence of the immunomodulatory fusion protein or polypeptide may preferably comprise an amino acid sequence 100% identical to the amino acid sequence of any one of SEQ ID NOS: 58, 61-69, or 72-79. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 58, 61-69, or 72-79. For example, the amino acid sequence of the immunomodulatory fusion protein or polypeptide may consist of an amino acid sequence at least 85% identical to the amino acid sequence of any one of SEQ ID NOS: 58, 61-69, or 72-79. The amino acid sequence of the immunomodulatory fusion protein or polypeptide may consist of an amino acid sequence at least 90% identical to the amino acid sequence of any one of SEQ ID NOS: 58, 61-69, or 72-79. The amino acid sequence of the immunomodulatory fusion protein or polypeptide may consist of an amino acid sequence at least 95% identical to the amino acid sequence of any one of SEQ ID NOS: 58, 61-69, or 72-79. In some embodiments, the amino acid sequence of the immunomodulatory fusion protein or polypeptide may preferably consist of an amino acid sequence 100% identical to the amino acid sequence of any one of SEQ ID NOS: 58, 61-69, or 72-79.

In some embodiments, of the immunomodulatory fusion proteins or polypeptides referred to herein, SEQ ID NOS: 62-65 or 73-76 may be preferred. Advantageously, said immunomodulatory fusion proteins or polypeptides may be particularly effective at suppressing the production of proinflammatory cytokines, e.g., one or more of IFN-γ, IL-1β, IL-6, IL-8, TNF-α, and IL-4. For example, said immunomodulatory fusion proteins or polypeptides may suppress the production of proinflammatory cytokines more effectively than hIL-10. In some embodiments this is preferably in addition to engaging hIL-10R with a higher potency than hIL-10. Thus, an immunomodulatory fusion protein or polypeptide may comprise an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 62-65 or 73-76. For example, the amino acid sequence of the immunomodulatory fusion protein or polypeptide may comprise an amino acid sequence at least 85% identical to the amino acid sequence of any one of SEQ ID NOS: 62-65 or 73-76. The amino acid sequence of the immunomodulatory fusion protein or polypeptide may comprise an amino acid sequence at least 90% identical to the amino acid sequence of any one of SEQ ID NOS: 62-65 or 73-76. The amino acid sequence of the immunomodulatory fusion protein or polypeptide may comprise an amino acid sequence at least 95% identical to the amino acid sequence of any one of SEQ ID NOS: 62-65 or 73-76. In some embodiments, the amino acid sequence of the immunomodulatory fusion protein or polypeptide may preferably comprise an amino acid sequence 100% identical to the amino acid sequence of any one of SEQ ID NOS: 62-65 or 73-76. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 62-65 or 73-76. For example, the amino acid sequence of the immunomodulatory fusion protein or polypeptide may consist of an amino acid sequence at least 85% identical to the amino acid sequence of any one of SEQ ID NOS: 62-65 or 73-76. The amino acid sequence of the immunomodulatory fusion protein or polypeptide may consist of an amino acid sequence at least 90% identical to the amino acid sequence of any one of SEQ ID NOS: 62-65 or 73-76. The amino acid sequence of the immunomodulatory fusion protein or polypeptide may consist of an amino acid sequence at least 95% identical to the amino acid sequence of any one of SEQ ID NOS: 62-65 or 73-76. In some embodiments, the amino acid sequence of the immunomodulatory fusion protein or polypeptide may preferably consist of an amino acid sequence 100% identical to the amino acid sequence of any one of SEQ ID NOS: 62-65 or 73-76.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 64, 75, 86, 97, 479, 480, 481, 482, 489, 490, 491, 499, 500, 501, 509, or 517. For example, the amino acid sequence of the immunomodulatory fusion protein or polypeptide may comprise an amino acid sequence at least 85% identical to the amino acid sequence of any one of SEQ ID NOS: 64, 75, 86, 97, 479, 480, 481, 482, 489, 490, 491, 499, 500, 501, 509, or 517. The amino acid sequence of the immunomodulatory fusion protein or polypeptide may comprise an amino acid sequence at least 90% identical to the amino acid sequence of any one of SEQ ID NOS: 64, 75, 86, 97, 479, 480, 481, 482, 489, 490, 491, 499, 500, 501, 509, or 517. The amino acid sequence of the immunomodulatory fusion protein or polypeptide may comprise an amino acid sequence at least 95% identical to the amino acid sequence of any one of SEQ ID NOS: 64, 75, 86, 97, 479, 480, 481, 482, 489, 490, 491, 499, 500, 501, 509, or 517. In some embodiments, the amino acid sequence of the immunomodulatory fusion protein or polypeptide preferably may comprise an amino acid sequence 100% identical to the amino acid sequence of any one of SEQ ID NOS: 64, 75, 86, 97, 479, 480, 481, 482, 489, 490, 491, 499, 500, 501, 509, or 517. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 64, 75, 86, 97, 479, 480, 481, 482, 489, 490, 491, 499, 500, 501, 509, or 517. For example, the amino acid sequence of the immunomodulatory fusion protein or polypeptide may consist of an amino acid sequence at least 85% identical to the amino acid sequence of any one of SEQ ID NOS: 64, 75, 86, 97, 479, 480, 481, 482, 489, 490, 491, 499, 500, 501, 509, or 517. The amino acid sequence of the immunomodulatory fusion protein or polypeptide may consist of an amino acid sequence at least 90% identical to the amino acid sequence of any one of SEQ ID NOS: 64, 75, 86, 97, 479, 480, 481, 482, 489, 490, 491, 499, 500, 501, 509, or 517. The amino acid sequence of the immunomodulatory fusion protein or polypeptide may consist of an amino acid sequence at least 95% identical to the amino acid sequence of any one of SEQ ID NOS: 64, 75, 86, 97, 479, 480, 481, 482, 489, 490, 491, 499, 500, 501, 509, or 517. In some embodiments, the amino acid sequence of the immunomodulatory fusion protein or polypeptide preferably may consist of an amino acid sequence 100% identical to the amino acid sequence of any one of SEQ ID NOS: 64, 75, 86, 97, 479, 480, 481, 482, 489, 490, 491, 499, 500, 501, 509, or 517.

In some embodiments, the amino acid sequence of the immunomodulatory fusion protein or polypeptide comprises or consists of the amino acid sequence of any one of SEQ ID NOS: 64, 75, 86, 97, 479, 480, 481, 482, 489, 490, 491, 499, 500, 501, 509, or 517, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 64, 75, 86, 97, 479, 480, 481, 482, 489, 490, 491, 499, 500, 501, 509, or 517, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 64, 75, 86, 97, 479, 480, 481, 482, 489, 490, 491, 499, 500, 501, 509, or 517, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 64, 75, 86, 97, 479, 480, 481, 482, 489, 490, 491, 499, 500, 501, 509, or 517, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 64, 75, 86, 97, 479, 480, 481, 482, 489, 490, 491, 499, 500, 501, 509, or 517, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 64, 75, 86, 97, 479, 480, 481, 482, 489, 490, 491, 499, 500, 501, 509, or 517, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 64, 75, 86, 97, 479, 480, 481, 482, 489, 490, 491, 499, 500, 501, 509, or 517, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 64, 75, 86, 97, 479, 480, 481, 482, 489, 490, 491, 499, 500, 501, 509, or 517, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 64, 75, 86, 97, 479, 480, 481, 482, 489, 490, 491, 499, 500, 501, 509, or 517. For example, the amino acid sequence of the immunomodulatory fusion protein or polypeptide may comprise an amino acid sequence at least 85% identical to the amino acid sequence of any one of SEQ ID NOS: 64, 75, 86, 97, 479, 480, 481, 482, 489, 490, 491, 499, 500, 501, 509, or 517. The amino acid sequence of the immunomodulatory fusion protein or polypeptide may comprise an amino acid sequence at least 90% identical to the amino acid sequence of any one of SEQ ID NOS: 64, 75, 86, 97, 479, 480, 481, 482, 489, 490, 491, 499, 500, 501, 509, or 517. The amino acid sequence of the immunomodulatory fusion protein or polypeptide may comprise an amino acid sequence at least 95% identical to the amino acid sequence of any one of SEQ ID NOS: 64, 75, 86, 97, 479, 480, 481, 482, 489, 490, 491, 499, 500, 501, 509, or 517. In some embodiments, the amino acid sequence of the immunomodulatory fusion protein or polypeptide preferably may comprise an amino acid sequence 100% identical to the amino acid sequence of any one of SEQ ID NOS: 64, 75, 86, 97, 479, 480, 481, 482, 489, 490, 491, 499, 500, 501, 509, or 517. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 64, 75, 86, 97, 479, 480, 481, 482, 489, 490, 491, 499, 500, 501, 509, or 517. For example, the amino acid sequence of the immunomodulatory fusion protein or polypeptide may consist of an amino acid sequence at least 85% identical to the amino acid sequence of any one of SEQ ID NOS: 64, 75, 86, 97, 479, 480, 481, 482, 489, 490, 491, 499, 500, 501, 509, or 517. The amino acid sequence of the immunomodulatory fusion protein or polypeptide may consist of an amino acid sequence at least 90% identical to the amino acid sequence of any one of SEQ ID NOS: 64, 75, 86, 97, 479, 480, 481, 482, 489, 490, 491, 499, 500, 501, 509, or 517. The amino acid sequence of the immunomodulatory fusion protein or polypeptide may consist of an amino acid sequence at least 95% identical to the amino acid sequence of any one of SEQ ID NOS: 64, 75, 86, 97, 479, 480, 481, 482, 489, 490, 491, 499, 500, 501, 509, or 517. In some embodiments, the amino acid sequence of the immunomodulatory fusion protein or polypeptide preferably may consist of an amino acid sequence 100% identical to the amino acid sequence of any one of SEQ ID NOS: 64, 75, 86, 97, 479, 480, 481, 482, 489, 490, 491, 499, 500, 501, 509, or 517.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 64, 75, 86, 97, 489, 499, 509, or 517. For example, the amino acid sequence of the immunomodulatory fusion protein or polypeptide may comprise an amino acid sequence at least 85% identical to the amino acid sequence of any one of SEQ ID NOS: 64, 75, 86, 97, 489, 499, 509, or 517. The amino acid sequence of the immunomodulatory fusion protein or polypeptide may comprise an amino acid sequence at least 90% identical to the amino acid sequence of any one of SEQ ID NOS: 64, 75, 86, 97, 489, 499, 509, or 517. The amino acid sequence of the immunomodulatory fusion protein or polypeptide may comprise an amino acid sequence at least 95% identical to the amino acid sequence of any one of SEQ ID NOS: 64, 75, 86, 97, 489, 499, 509, or 517. In some embodiments, the amino acid sequence of the immunomodulatory fusion protein or polypeptide preferably may comprise an amino acid sequence 100% identical to the amino acid sequence of any one of SEQ ID NOS: 64, 75, 86, 97, 489, 499, 509, or 517. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 64, 75, 86, 97, 489, 499, 509, or 517. For example, the amino acid sequence of the immunomodulatory fusion protein or polypeptide may consist of an amino acid sequence at least 85% identical to the amino acid sequence of any one of SEQ ID NOS: 64, 75, 86, 97, 489, 499, 509, or 517. The amino acid sequence of the immunomodulatory fusion protein or polypeptide may consist of an amino acid sequence at least 90% identical to the amino acid sequence of any one of SEQ ID NOS: 64, 75, 86, 97, 489, 499, 509, or 517. The amino acid sequence of the immunomodulatory fusion protein or polypeptide may consist of an amino acid sequence at least 95% identical to the amino acid sequence of any one of SEQ ID NOS: 64, 75, 86, 97, 489, 499, 509, or 517. In some embodiments, the amino acid sequence of the immunomodulatory fusion protein or polypeptide preferably may consist of an amino acid sequence 100% identical to the amino acid sequence of any one of SEQ ID NOS: 64, 75, 86, 97, 489, 499, 509, or 517.

In some embodiments, the amino acid sequence of the immunomodulatory fusion protein or polypeptide comprises or consists of the amino acid sequence of any one of SEQ ID NOS: 64, 75, 86, 97, 489, 499, 509, or 517, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 64, 75, 86, 97, 489, 499, 509, or 517, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 64, 75, 86, 97, 489, 499, 509, or 517, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 64, 75, 86, 97, 489, 499, 509, or 517, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 64, 75, 86, 97, 489, 499, 509, or 517, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 64, 75, 86, 97, 489, 499, 509, or 517, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 64, 75, 86, 97, 489, 499, 509, or 517, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 64, 75, 86, 97, 489, 499, 509, or 517, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 64, 75, 86, 97, 489, 499, 509, or 517. For example, the amino acid sequence of the immunomodulatory fusion protein or polypeptide may comprise an amino acid sequence at least 85% identical to the amino acid sequence of any one of SEQ ID NOS: 64, 75, 86, 97, 489, 499, 509, or 517. The amino acid sequence of the immunomodulatory fusion protein or polypeptide may comprise an amino acid sequence at least 90% identical to the amino acid sequence of any one of SEQ ID NOS: 64, 75, 86, 97, 489, 499, 509, or 517. The amino acid sequence of the immunomodulatory fusion protein or polypeptide may comprise an amino acid sequence at least 95% identical to the amino acid sequence of any one of SEQ ID NOS: 64, 75, 86, 97, 489, 499, 509, or 517. In some embodiments, the amino acid sequence of the immunomodulatory fusion protein or polypeptide preferably may comprise an amino acid sequence 100% identical to the amino acid sequence of any one of SEQ ID NOS: 64, 75, 86, 97, 489, 499, 509, or 517. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 64, 75, 86, 97, 489, 499, 509, or 517. For example, the amino acid sequence of the immunomodulatory fusion protein or polypeptide may consist of an amino acid sequence at least 85% identical to the amino acid sequence of any one of SEQ ID NOS: 64, 75, 86, 97, 489, 499, 509, or 517. The amino acid sequence of the immunomodulatory fusion protein or polypeptide may consist of an amino acid sequence at least 90% identical to the amino acid sequence of any one of SEQ ID NOS: 64, 75, 86, 97, 489, 499, 509, or 517. The amino acid sequence of the immunomodulatory fusion protein or polypeptide may consist of an amino acid sequence at least 95% identical to the amino acid sequence of any one of SEQ ID NOS: 64, 75, 86, 97, 489, 499, 509, or 517. In some embodiments, the amino acid sequence of the immunomodulatory fusion protein or polypeptide preferably may consist of an amino acid sequence 100% identical to the amino acid sequence of any one of SEQ ID NOS: 64, 75, 86, 97, 489, 499, 509, or 517.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 479, 480, 481, 482, 490, 491, 500, or 501. For example, the amino acid sequence of the immunomodulatory fusion protein or polypeptide may comprise an amino acid sequence at least 85% identical to the amino acid sequence of any one of SEQ ID NOS: 479, 480, 481, 482, 490, 491, 500, or 501. The amino acid sequence of the immunomodulatory fusion protein or polypeptide may comprise an amino acid sequence at least 90% identical to the amino acid sequence of any one of SEQ ID NOS: 479, 480, 481, 482, 490, 491, 500, or 501. The amino acid sequence of the immunomodulatory fusion protein or polypeptide may comprise an amino acid sequence at least 95% identical to the amino acid sequence of any one of SEQ ID NOS: 479, 480, 481, 482, 490, 491, 500, or 501. In some embodiments, the amino acid sequence of the immunomodulatory fusion protein or polypeptide preferably may comprise an amino acid sequence 100% identical to the amino acid sequence of any one of SEQ ID NOS: 479, 480, 481, 482, 490, 491, 500, or 501. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 479, 480, 481, 482, 490, 491, 500, or 501. For example, the amino acid sequence of the immunomodulatory fusion protein or polypeptide may consist of an amino acid sequence at least 85% identical to the amino acid sequence of any one of SEQ ID NOS: 479, 480, 481, 482, 490, 491, 500, or 501. The amino acid sequence of the immunomodulatory fusion protein or polypeptide may consist of an amino acid sequence at least 90% identical to the amino acid sequence of any one of SEQ ID NOS: 479, 480, 481, 482, 490, 491, 500, or 501. The amino acid sequence of the immunomodulatory fusion protein or polypeptide may consist of an amino acid sequence at least 95% identical to the amino acid sequence of any one of SEQ ID NOS: 479, 480, 481, 482, 490, 491, 500, or 501. In some embodiments, the amino acid sequence of the immunomodulatory fusion protein or polypeptide preferably may consist of an amino acid sequence 100% identical to the amino acid sequence of any one of SEQ ID NOS: 479, 480, 481, 482, 490, 491, 500, or 501.

In some embodiments, the amino acid sequence of the immunomodulatory fusion protein or polypeptide comprises or consists of the amino acid sequence of any one of SEQ ID NOS: 479, 480, 481, 482, 490, 491, 500, or 501, and further comprises 1 or more but less than 15% (less than 12%, less than 10%, less than 8%), amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 479, 480, 481, 482, 490, 491, 500, or 501, and further comprises or consists of at least about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 479, 480, 481, 482, 490, 491, 500, or 501, and further comprises or consists of about 1, 2, 3, 4, 5, 6, 7, 8, 9, or 10 amino acid variations (e.g., substitutions, additions, deletions, etc.). In some embodiments, the amino acid sequence of the immunomodulatory fusion protein or polypeptide comprises or consists of the amino acid sequence of any one of SEQ ID NOS: 479, 480, 481, 482, 490, 491, 500, or 501, and further comprises or consists of no more than about 1, 2, 3, 4, 5, 6, 7, 8, 9, or 10 amino acid variations (e.g., substitutions, additions, deletions, etc.).

In some embodiments, the amino acid sequence of the immunomodulatory fusion protein or polypeptide comprises or consists of the amino acid sequence of any one of SEQ ID NOS: 479, 480, 481, 482, 490, 491, 500, or 501, and further comprises 1 or more but less than 15% (less than 12%, less than 10%, less than 8%), amino acid substitutions. In some embodiments, the amino acid sequence of the immunomodulatory fusion protein or polypeptide comprises or consists of the amino acid sequence of any one of SEQ ID NOS: 479, 480, 481, 482, 490, 491, 500, or 501, and further comprises or consists of at least about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 479, 480, 481, 482, 490, 491, 500, or 501, and further comprises or consists of about 1, 2, 3, 4, 5, 6, 7, 8, 9, or 10 amino acid substitutions. In some embodiments, the amino acid sequence of the immunomodulatory fusion protein or polypeptide comprises or consists of the amino acid sequence of any one of SEQ ID NOS: 479, 480, 481, 482, 490, 491, 500, or 501, and further comprises or consists of no more than about 1, 2, 3, 4, 5, 6, 7, 8, 9, or 10 amino acid substitutions.

In some embodiments, the amino acid sequence of the immunomodulatory fusion protein or polypeptide comprises an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 479, 480, 481, 482, 490, 491, 500, or 501. For example, the amino acid sequence of the immunomodulatory fusion protein or polypeptide may comprise an amino acid sequence at least 85% identical to the amino acid sequence of any one of SEQ ID NOS: 479, 480, 481, 482, 490, 491, 500, or 501. The amino acid sequence of the immunomodulatory fusion protein or polypeptide may comprise an amino acid sequence at least 90% identical to the amino acid sequence of any one of SEQ ID NOS: 479, 480, 481, 482, 490, 491, 500, or 501. The amino acid sequence of the immunomodulatory fusion protein or polypeptide may comprise an amino acid sequence at least 95% identical to the amino acid sequence of any one of SEQ ID NOS: 479, 480, 481, 482, 490, 491, 500, or 501. In some embodiments, the amino acid sequence of the immunomodulatory fusion protein or polypeptide preferably may comprise an amino acid sequence 100% identical to the amino acid sequence of any one of SEQ ID NOS: 479, 480, 481, 482, 490, 491, 500, or 501. In some embodiments, the amino acid sequence of the immunomodulatory fusion protein or polypeptide consists of an amino acid sequence at least 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 479, 480, 481, 482, 490, 491, 500, or 501. For example, the amino acid sequence of the immunomodulatory fusion protein or polypeptide may consist of an amino acid sequence at least 85% identical to the amino acid sequence of any one of SEQ ID NOS: 479, 480, 481, 482, 490, 491, 500, or 501. The amino acid sequence of the immunomodulatory fusion protein or polypeptide may consist of an amino acid sequence at least 90% identical to the amino acid sequence of any one of SEQ ID NOS: 479, 480, 481, 482, 490, 491, 500, or 501. The amino acid sequence of the immunomodulatory fusion protein or polypeptide may consist of an amino acid sequence at least 95% identical to the amino acid sequence of any one of SEQ ID NOS: 479, 480, 481, 482, 490, 491, 500, or 501. In some embodiments, the amino acid sequence of the immunomodulatory fusion protein or polypeptide preferably may consist of an amino acid sequence 100% identical to the amino acid sequence of any one of SEQ ID NOS: 479, 480, 481, 482, 490, 491, 500, or 501.

In some embodiments, an immunomodulatory fusion protein or polypeptide described herein (e.g., any one of IFPs 1-11, see Table 7, e.g., any one of SEQ ID NOS: 58-101) is compared to a reference hIL-10 Fc fusion protein in the same format. The amino acid sequence of exemplary reference hIL-10-Fc fusion proteins is provided in Table 8. A person of ordinary skill in art can readily determine an appropriate reference fusion protein as needed.

TABLE 8
The Amino Acid Sequence of Exemplary Reference hIL-10 Fc and GFP-Fc Fusion
Proteins & Polypeptides

| | | SEQ |
|---|---|---|
| Description | Amino Acid Sequence | ID NO |
| Reference hIL-10 hIg Fc | MYRMQLLSCIALSLALVINSAESKYGPPCPPCPAPEAAGGPS | 102 |
| fusion protein | VFLFPPKPKDTLMISRTPEVTCVVVDVSQEDPEVQFNWYVDG | |
| (variant) | VEVHNAKTKPREEQFNSTYRVVSVLIVLHQDWLNGKEYKCKV | |
| (with signal sequence) | SNKGLPSSIEKTISKAKGQPREPQVYTLPPSQEEMTKNQVSL | |
| | TCLVKGFYPSDIAVEWESNGQPENNYKTTPPVLDSDGSFFLY | |
| | SRLTVDKSRWQEGNVFSCSVMHEALHNHYTQKSLSLSLGGGG | |
| | GGGGSGGGGSGGGGSMHSSALLCCLVLLTGVRASPGQGTQSE | |
| | NSCTHFPGNLPNMLRDLRDAFSRVKIFFQMKDQLDNLLLKES | |
| | LLEDFKGYLGCQALSEMIQFYLEEVMPQAENQDPDIKAHVNS | |
| | LGENLKTLRLRLRRCHRFLPCENKSKAVEQVKNAFNKLQEKG | |
| | IYKAMSEFDIFINYIEAYMTMKIRN | |
| Reference hIL-10 hIg Fc | AESKYGPPCPPCPAPEAAGGPSVFLFPPKPKDTLMISRTPEV | 103 |
| fusion protein | TCVVVDVSQEDPEVQFNWYVDGVEVHNAKTKPREEQFNSTYR | |
| (variant) | VVSVLTVLHQDWLNGKEYKCKVSNKGLPSSIEKTISKAKGQP | |
| (without signal sequence) | REPQVYTLPPSQEEMTKNQVSLTCLVKGFYPSDIAVEWESNG | |
| | QPENNYKTTPPVLDSDGSFFLYSRLTVDKSRWQEGNVFSCSV | |
| | MHEALHNHYTQKSLSLSLGGGGGGGGSGGGGSGGGGSMHSSA | |
| | LLCCLVLLTGVRASPGQGTQSENSCTHFPGNLPNMLRDLRDA | |
| | FSRVKTFFQMKDQLDNLLLKESLLEDFKGYLGCQALSEMIQF | |
| | YLEEVMPQAENQDPDIKAHVNSLGENLKTLRLRLRRCHRFLP | |
| | CENKSKAVEQVKNAFNKLQEKGIYKAMSEFDIFINYIEAYMT | |
| | MKIRN | |
| Reference hIL-10 hIg Fc | MYRMQLLSCIALSLALVINSESKYGPPCPPCPAPEAAGGPSV | 104 |
| fusion protein | FLFPPKPKDTLMISRTPEVTCVVVDVSQEDPEVQFNWYVDGV | |
| (with signal sequence) | EVHNAKTKPREEQFNSTYRVVSVLIVLHQDWLNGKEYKCKVS | |
| | NKGLPSSIEKTISKAKGQPREPQVYTLPPSQEEMTKNQVSLT | |
| | CLVKGFYPSDIAVEWESNGQPENNYKTTPPVLDSDGSFFLYS | |
| | RLTVDKSRWQEGNVFSCSVMHEALHNHYTQKSLSLSLGGGGG | |
| | GGGSGGGGSGGGGSMHSSALLCCLVLLTGVRASPGQGTQSEN | |
| | SCTHFPGNLPNMLRDLRDAFSRVKTFFQMKDQLDNLLLKESL | |
| | LEDFKGYLGCQALSEMIQFYLEEVMPQAENQDPDIKAHVNSL | |
| | GENLKTLRLRLRRCHRFLPCENKSKAVEQVKNAFNKLQEKGI | |
| | YKAMSEFDIFINYIEAYMTMKIRN | |
| Reference hIL-10 hIg Fc | ESKYGPPCPPCPAPEAAGGPSVFLFPPKPKDTLMISRTPEVT | 105 |
| fusion protein | CVVVDVSQEDPEVQFNWYVDGVEVHNAKTKPREEQFNSTYRV | |
| (without signal sequence) | VSVLTVLHQDWLNGKEYKCKVSNKGLPSSIEKTISKAKGQPR | |
| | EPQVYTLPPSQEEMTKNQVSLTCLVKGFYPSDIAVEWESNGQ | |
| | PENNYKTTPPVLDSDGSFFLYSRLTVDKSRWQEGNVFSCSVM | |
| | HEALHNHYTQKSLSLSLGGGGGGGGSGGGGSGGGGSMHSSAL | |
| | LCCLVLLTGVRASPGQGTQSENSCTHFPGNLPNMLRDLRDAF | |
| | SRVKTFFQMKDQLDNLLLKESLLEDFKGYLGCQALSEMIQFY | |
| | LEEVMPQAENQDPDIKAHVNSLGENLKILRLRLRRCHRFLPC | |
| | ENKSKAVEQVKNAFNKLQEKGIYKAMSEFDIFINYIEAYMTM | |
| | KIRN | |
| Reference GFP hIg Fc | MYRMQLLSCIALSLALVINSAESKYGPPCPPCPAPEAAGGPS | 106 |
| Fusion | VFLFPPKPKDTLMISRTPEVTCVVVDVSQEDPEVQFNWYVDG | |
| (with signal sequence) | VEVHNAKTKPREEQFNSTYRVVSVLTVLHQDWLNGKEYKCKV | |
| | SNKGLPSSIEKTISKAKGQPREPQVYTLPPSQEEMTKNQVSL | |
| | TCLVKGFYPSDIAVEWESNGQPENNYKTTPPVLDSDGSFFLY | |
| | SRLTVDKSRWQEGNVFSCSVMHEALHNHYTQKSLSLSLGGGG | |
| | GGGGSGGGGSGGGGSMRKGEELFTGVVPILVELDGDVNGHKF | |
| | SVSGEGEGDATNGKLILKFICTTGKLPVPWPTLVTTLTYGVQ | |
| | CFARYPDHMKQHDFFKSAMPEGYVQERTISFKDDGTYKTRAE | |
| | VKFEGDILVNRIELKGIDFKEDGNILGHKLEYNFNSHNVYIT | |
| | ADKQKNGIKANFKIRHNVEDGSVQLADHYQQNTPIGDGPVLL | |
| | PDNHYLSTQSALSKDPNEKRDHMVLLEFVTAAGITHGMDELY | |
| | KRPAANDENYAASV | |
| Reference GFP hIg Fc | AESKYGPPCPPCPAPEAAGGPSVFLFPPKPKDTLMISRTPEV | 107 |
| Fusion | TCVVVDVSQEDPEVQFNWYVDGVEVHNAKIKPREEQFNSTYR | |
| (without signal sequence) | VVSVLTVLHQDWLNGKEYKCKVSNKGLPSSIEKTISKAKGQP | |
| | REPQVYTLPPSQEEMTKNQVSLTCLVKGFYPSDIAVEWESNG | |
| | QPENNYKTTPPVLDSDGSFFLYSRLTVDKSRWQEGNVFSCSV | |
| | MHEALHNHYTQKSLSLSLGGGGGGGGSGGGGSGGGGSMRKGE | |
| | ELFTGVVPILVELDGDVNGHKFSVSGEGEGDATNGKLTLKFI | |
| | CTTGKLPVPWPTLVTTLTYGVQCFARYPDHMKQHDFFKSAMP | |
| | EGYVQERTISFKDDGTYKTRAEVKFEGDTLVNRIELKGIDEK | |
| | EDGNILGHKLEYNFNSHNVYITADKQKNGIKANFKIRHNVED | |
| | GSVQLADHYQQNTPIGDGPVLLPDNHYLSTQSALSKDPNEKR | |
| | DHMVLLEFVTAAGITHGMDELYKRPAANDENYAASV | |
| Reference hIL-10 mIgG1 | MYRMQLLSCIALSLALVINSVPRDCGCKPCICTVPEVSSVFI | 519 |
| Fc fusion protein | FPPKPKDVLTITLTPKVTCVVVAISKDDPEVQFSWFVDDVEV | |
| (with signal sequence) | HTAQTQPREEQFNSTFRSVSELPIMHQDWLNGKEFKCRVNSA | |
| | AFPAPIEKTISKTKGRPKAPQVYTIPPPKEQMAKDKVSLTCM | |
| | ITDFFPEDITVEWQWNGQPAENYKNTQPIMDTDGSYFVYSKL | |
| | NVQKSNWEAGNTFTCSVLHEGLHNHHTEKSLSHSPGGGGGGG | |
| | SGGGGSGGGGSSPGQGTQSENSCTHFPGNLPNMLRDLRDAFS | |
| | RVKTFFQMKDQLDNLLLKESLLEDFKGYLGCQALSEMIQFYL | |
| | EEVMPQAENQDPDIKAHVNSLGENLKTLRLRLRRCHRFLPCE | |
| | NKSKAVEQVKNAFNKLQEKGIYKAMSEFDIFINYIEAYMTMK | |
| | IRN | |
| Reference hIL-10 mIgG1 | VPRDCGCKPCICTVPEVSSVFIFPPKPKDVLTITLTPKVTCV | 520 |
| Fc fusion protein | VVAISKDDPEVQFSWFVDDVEVHTAQTQPREEQFNSTERSVS | |
| (without signal sequence) | ELPIMHQDWLNGKEFKCRVNSAAFPAPIEKTISKTKGRPKAP | |
| | QVYTIPPPKEQMAKDKVSLTCMITDFFPEDITVEWQWNGQPA | |
| | ENYKNTQPIMDTDGSYFVYSKLNVQKSNWEAGNTFTCSVLHE | |
| | GLHNHHTEKSLSHSPGGGGGGGSGGGGSGGGGSSPGQGTQSE | |
| | NSCTHFPGNLPNMLRDLRDAFSRVKIFFQMKDQLDNLLLKES | |
| | LLEDFKGYLGCQALSEMIQFYLEEVMPQAENQDPDIKAHVNS | |
| | LGENLKTLRLRLRRCHRFLPCENKSKAVEQVKNAFNKLQEKG | |
| | IYKAMSEFDIFINYIEAYMTMKIRN | |
| Reference hIL-10 | MYRMQLLSCIALSLALVINSEPRGPTIKPCLLCKCPAPNAAG | 521 |
| mIgG2a Fc fusion protein | GPSVFIFPPKIKDVLMISLSPIVTCVVVDVSEDDPDVQISWF | |
| (with signal sequence) | VNNVEVHTAQTQTHREDYNSTLRVVSALPIQHQDWMSGKEFK | |
| | CKVNNKDLPAPIERTISKPKGSVRAPQVYVLPPPEEEMTKKQ | |
| | VTLTCMVTDFMPEDIYVEWTNNGKTELNYKNTEPVLDSDGSY | |
| | FMYSKLRVEKKNWVERNSYSCSVVHEGLHNHHTTKSFSRTPG | |
| | KGGGGGGGSGGGGSGGGGSSPGQGTQSENSCTHFPGNLPNML | |
| | RDLRDAFSRVKTFFQMKDQLDNLLLKESLLEDFKGYLGCQAL | |
| | SEMIQFYLEEVMPQAENQDPDIKAHVNSLGENLKTLRLRLRR | |
| | CHRFLPCENKSKAVEQVKNAFNKLQEKGIYKAMSEFDIFINY | |
| | IEAYMTMKIRN | |
| Reference hIL-10 | EPRGPTIKPCLLCKCPAPNAAGGPSVFIFPPKIKDVLMISLS | 522 |
| mIgG2a Fc fusion protein | PIVTCVVVDVSEDDPDVQISWFVNNVEVHTAQTQTHREDYNS | |
| (without signal sequence) | TLRVVSALPIQHQDWMSGKEFKCKVNNKDLPAPIERTISKPK | |
| | GSVRAPQVYVLPPPEEEMTKKQVTLTCMVTDFMPEDIYVEWT | |
| | NNGKTELNYKNTEPVLDSDGSYFMYSKLRVEKKNWVERNSYS | |
| | CSVVHEGLHNHHTTKSFSRTPGKGGGGGGGSGGGGSGGGGSS | |
| | PGQGTQSENSCTHFPGNLPNMLRDLRDAFSRVKTFFQMKDQL | |
| | DNLLLKESLLEDFKGYLGCQALSEMIQFYLEEVMPQAENQDP | |
| | DIKAHVNSLGENLKTLRLRLRRCHRFLPCENKSKAVEQVKNA | |
| | FNKLQEKGIYKAMSEFDIFINYIEAYMTMKIRN | |
| Reference hIL-10 | MYRMQLLSCIALSLALVINSEPRGPTIKPCPPCKCPAPNAAG | 523 |
| mIgG2a Fc fusion protein | GPSVFIFPPKIKDVLMISLSPIVTCVVVDVSEDDPDVQISWF | |
| Variant Fc | VNNVEVHTAQTQTHREDYNSTLRVVSALPIQHQDWMSGKEFK | |
| (with signal sequence) | CKVNNKDLGAPIERTISKPKGSVRAPQVYVLPPPEEEMTKKQ | |
| | VTLTCMVTDFMPEDIYVEWTNNGKTELNYKNTEPVLDSDGSY | |
| | FMYSKLRVEKKNWVERNSYSCSVVHEGLHNHHTTKSFSRTPG | |
| | KGGGGGGGSGGGGSGGGGSSPGQGTQSENSCTHFPGNLPNML | |
| | RDLRDAFSRVKTFFQMKDQLDNLLLKESLLEDFKGYLGCQAL | |
| | SEMIQFYLEEVMPQAENQDPDIKAHVNSLGENLKILRLRLRR | |
| | CHRFLPCENKSKAVEQVKNAFNKLQEKGIYKAMSEFDIFINY | |
| | IEAYMTMKIRN | |
| Reference hIL-10 | EPRGPTIKPCPPCKCPAPNAAGGPSVFIFPPKIKDVLMISLS | 524 |
| mIgG2a Fc fusion protein | PIVTCVVVDVSEDDPDVQISWFVNNVEVHTAQTQTHREDYNS | |
| Variant Fc | TLRVVSALPIQHQDWMSGKEFKCKVNNKDLGAPIERTISKPK | |
| (without signal sequence) | GSVRAPQVYVLPPPEEEMTKKQVTLTCMVTDEMPEDIYVEWT | |
| | NNGKTELNYKNTEPVLDSDGSYFMYSKLRVEKKNWVERNSYS | |
| | CSVVHEGLHNHHTTKSFSRTPGKGGGGGGGSGGGGSGGGGSS | |
| | PGQGTQSENSCTHFPGNLPNMLRDLRDAFSRVKTFFQMKDQL | |
| | DNLLLKESLLEDFKGYLGCQALSEMIQFYLEEVMPQAENQDP | |
| | DIKAHVNSLGENLKTLRLRLRRCHRFLPCENKSKAVEQVKNA | |
| | FNKLQEKGIYKAMSEFDIFINYIEAYMTMKIRN | |
| Reference mIgG2a Fc | MYRMQLLSCIALSLALVINSEPRGPTIKPCPPCKCPAPNAAG | 525 |
| fusion protein | GPSVFIFPPKIKDVLMISLSPIVTCVVVDVSEDDPDVQISWF | |
| Variant Fc | VNNVEVHTAQTQTHREDYNSTLRVVSALPIQHQDWMSGKEFK | |
| (with signal sequence) | CKVNNKDLGAPIERTISKPKGSVRAPQVYVLPPPEEEMTKKQ | |
| | VTLTCMVTDFMPEDIYVEWINNGKTELNYKNTEPVLDSDGSY | |
| | FMYSKLRVEKKNWVERNSYSCSVVHEGLHNHHTTKSFSRTPG | |
| | K | |
| Reference mIgG2a Fc | EPRGPTIKPCPPCKCPAPNAAGGPSVFIFPPKIKDVLMISLS | 526 |
| fusion protein | PIVTCVVVDVSEDDPDVQISWFVNNVEVHTAQTQTHREDYNS | |
| Variant Fc | TLRVVSALPIQHQDWMSGKEFKCKVNNKDLGAPIERTISKPK | |
| (without signal sequence) | GSVRAPQVYVLPPPEEEMTKKQVTLICMVTDEMPEDIYVEWT | |
| | NNGKTELNYKNTEPVLDSDGSYFMYSKLRVEKKNWVERNSYS | |
| | CSVVHEGLHNHHTTKSFSRTPGK | |
| Reference mIgG1 Fc | MYRMQLLSCIALSLALVINSVPRDCGCKPCICTVPEVSSVFI | 527 |
| fusion protein | FPPKPKDVLTITLTPKVTCVVVAISKDDPEVQFSWFVDDVEV | |
| (with signal sequence) | HTAQTQPREEQFNSTFRSVSELPIMHQDWLNGKEFKCRVNSA | |
| | AFPAPIEKTISKTKGRPKAPQVYTIPPPKEQMAKDKVSLTCM | |
| | ITDFFPEDITVEWQWNGQPAENYKNIQPIMDTDGSYFVYSKL | |
| | NVQKSNWEAGNTFTCSVLHEGLHNHHTEKSLSHSP | |
| Reference mIgG1 Fc | VPRDCGCKPCICTVPEVSSVFIFPPKPKDVLTITLTPKVTCV | 528 |
| fusion protein | VVAISKDDPEVQFSWFVDDVEVHTAQTQPREEQFNSTERSVS | |
| (without signal sequence) | ELPIMHQDWLNGKEFKCRVNSAAFPAPIEKTISKTKGRPKAP | |
| | QVYTIPPPKEQMAKDKVSLICMITDFFPEDITVEWQWNGQPA | |
| | ENYKNTQPIMDTDGSYFVYSKLNVQKSNWEAGNIFTCSVLHE | |
| | GLHNHHTEKSLSHSP | |
| Reference hIgG4 Fc | MYRMQLLSCIALSLALVINSAESKYGPPCPPCPAPEAAGGPS | 529 |
| fusion protein | VFLFPPKPKDTLMISRTPEVICVVVDVSQEDPEVQFNWYVDG | |
| Variant Fc | VEVHNAKIKPREEQFNSTYRVVSVLIVLHQDWLNGKEYKCKV | |
| (with signal sequence) | SNKGLPSSIEKTISKAKGQPREPQVYTLPPSQEEMTKNQVSL | |
| | TCLVKGFYPSDIAVEWESNGQPENNYKITPPVLDSDGSFFLY | |
| | SRLTVDKSRWQEGNVFSCSVMHEALHNHYTQKSLSLSL | |
| Reference hIgG4 Fc | AESKYGPPCPPCPAPEAAGGPSVFLFPPKPKDTLMISRTPEV | 530 |
| fusion protein | TCVVVDVSQEDPEVQFNWYVDGVEVHNAKIKPREEQFNSTYR | |
| Variant Fc | VVSVLIVLHQDWLNGKEYKCKVSNKGLPSSIEKTISKAKGQP | |
| (without signal sequence) | REPQVYTLPPSQEEMTKNQVSLTCLVKGFYPSDIAVEWESNG | |
| | QPENNYKTTPPVLDSDGSFFLYSRLTVDKSRWQEGNVFSCSV | |
| | MHEALHNHYTQKSLSLSL | |

In some embodiments, the amino acid sequence of the reference hIL-10 Fc fusion protein comprises the amino acid sequence of a polypeptide set forth in Table 8. In some embodiments, the amino acid sequence of the reference hIL-10 Fc fusion protein comprises the amino acid sequence set forth in SEQ ID NO: 102. In some embodiments, the amino acid sequence of reference hIL-10 Fc fusion protein comprises the amino acid sequence set forth in SEQ ID NO: 103. In some embodiments, the amino acid sequence of reference hIL-10 Fc fusion protein comprises the amino acid sequence set forth in SEQ ID NO: 104. In some embodiments, the amino acid sequence of reference hIL-10 Fc fusion protein comprises the amino acid sequence set forth in SEQ ID NO: 105. In some embodiments, the amino acid sequence of reference hIL-10 Fc fusion protein comprises the amino acid sequence set forth in SEQ ID NO: 106. In some embodiments, the amino acid sequence of reference hIL-10 Fc fusion protein comprises the amino acid sequence set forth in SEQ ID NO: 107. In some embodiments, the amino acid sequence of reference hIL-10 Fc fusion protein comprises the amino acid sequence set forth in SEQ ID NO: 519. In some embodiments, the amino acid sequence of reference hIL-10 Fc fusion protein comprises the amino acid sequence set forth in SEQ ID NO: 520. In some embodiments, the amino acid sequence of reference hIL-10 Fc fusion protein comprises the amino acid sequence set forth in SEQ ID NO: 521. In some embodiments, the amino acid sequence of reference hIL-10 Fc fusion protein comprises the amino acid sequence set forth in SEQ ID NO: 522. In some embodiments, the amino acid sequence of reference hIL-10 Fc fusion protein comprises the amino acid sequence set forth in SEQ ID NO: 523. In some embodiments, the amino acid sequence of reference hIL-10 Fc fusion protein comprises the amino acid sequence set forth in SEQ ID NO: 524. In some embodiments, the amino acid sequence of reference hIL-10 Fc fusion protein comprises the amino acid sequence set forth in SEQ ID NO: 525. In some embodiments, the amino acid sequence of reference hIL-10 Fc fusion protein comprises the amino acid sequence set forth in SEQ ID NO: 526. In some embodiments, the amino acid sequence of reference hIL-10 Fc fusion protein comprises the amino acid sequence set forth in SEQ ID NO: 527. In some embodiments, the amino acid sequence of reference hIL-10 Fc fusion protein comprises the amino acid sequence set forth in SEQ ID NO: 528. In some embodiments, the amino acid sequence of reference hIL-10 Fc fusion protein comprises the amino acid sequence set forth in SEQ ID NO: 529. In some embodiments, the amino acid sequence of reference hIL-10 Fc fusion protein comprises the amino acid sequence set forth in SEQ ID NO: 530.

In some embodiments, the amino acid sequence of the reference hIL-10 Fc fusion protein consists of the amino acid sequence of a polypeptide set forth in Table 8. In some embodiments, the amino acid sequence of the reference hIL-10 Fc fusion protein consists of the amino acid sequence set forth in SEQ ID NO: 102. In some embodiments, the amino acid sequence of reference hIL-10 Fc fusion protein consists of the amino acid sequence set forth in SEQ ID NO: 103. In some embodiments, the amino acid sequence of reference hIL-10 Fc fusion protein consists of the amino acid sequence set forth in SEQ ID NO: 104. In some embodiments, the amino acid sequence of reference hIL-10 Fc fusion protein consists of the amino acid sequence set forth in SEQ ID NO: 105. In some embodiments, the amino acid sequence of reference hIL-10 Fc fusion protein consists of the amino acid sequence set forth in SEQ ID NO: 106. In some embodiments, the amino acid sequence of reference hIL-10 Fc fusion protein consists of the amino acid sequence set forth in SEQ ID NO: 107. In some embodiments, the amino acid sequence of reference hIL-10 Fc fusion protein consists of the amino acid sequence set forth in SEQ ID NO: 519. In some embodiments, the amino acid sequence of reference hIL-10 Fc fusion protein consists of the amino acid sequence set forth in SEQ ID NO: 520. In some embodiments, the amino acid sequence of reference hIL-10 Fc fusion protein consists of the amino acid sequence set forth in SEQ ID NO: 521. In some embodiments, the amino acid sequence of reference hIL-10 Fc fusion protein consists of the amino acid sequence set forth in SEQ ID NO: 522. In some embodiments, the amino acid sequence of reference hIL-10 Fc fusion protein consists of the amino acid sequence set forth in SEQ ID NO: 523. In some embodiments, the amino acid sequence of reference hIL-10 Fc fusion protein consists of the amino acid sequence set forth in SEQ ID NO: 524. In some embodiments, the amino acid sequence of reference hIL-10 Fc fusion protein consists of the amino acid sequence set forth in SEQ ID NO: 525. In some embodiments, the amino acid sequence of reference hIL-10 Fc fusion protein consists of the amino acid sequence set forth in SEQ ID NO: 526. In some embodiments, the amino acid sequence of reference hIL-10 Fc fusion protein consists of the amino acid sequence set forth in SEQ ID NO: 527. In some embodiments, the amino acid sequence of reference hIL-10 Fc fusion protein consists of the amino acid sequence set forth in SEQ ID NO: 528. In some embodiments, the amino acid sequence of reference hIL-10 Fc fusion protein consists of the amino acid sequence set forth in SEQ ID NO: 529. In some embodiments, the amino acid sequence of reference hIL-10 Fc fusion protein consists of the amino acid sequence set forth in SEQ ID NO: 530.

### 5.4 Immunogenic Peptides & Proteins

In one aspect, provided herein are immunogenic peptides or proteins comprising (a) at least a portion of an immunomodulatory protein or polypeptide described herein, or (b) an immunomodulatory protein or polypeptide described herein that comprises at least one amino acid variation (e.g., substitution, addition, deletion); and wherein the immunogenic peptide or protein does not specifically bind to the hIL-10R (e.g., hIL-10Rα) or binds to the hIL-10R (e.g., hIL-10Rα) with lower affinity relative to a reference immunomodulatory protein or polypeptide described herein.

In some embodiments, the immunogenic peptide or protein comprises or consists of at least about 10, 15, 20, 30, 40, 50, 60, 70, 80, 90, 100, 150, 200, or 250 amino acids. In some embodiments, the immunogenic peptide or protein comprises or consists of about 10, 15, 20, 30, 40, 50, 60, 70, 80, 90, 100, 150, 200, or 250 amino acids. In some embodiments, the immunogenic peptide or protein comprises or consists of no more than about 10, 15, 20, 30, 40, 50, 60, 70, 80, 90, 100, 150, 200, or 250 amino acids. In some embodiments, the immunogenic peptide or protein comprises or consists of from about 10-250, 10-200, 10-100, 10-90, 10-80, 10-70, 10-60, 10-50, 10-40, 10-30, 10-20, 10-250, 20-250, 30-250, 40-250, 50-250, 60-250, 70-250, 80-250, 90-250, 100-250, 10-200, 20-200, 30-200, 40-200, 50-200, 60-200, 70-200, 80-200, 90-200, or 100-200 amino acids. In some embodiments, the immunogenic peptide or protein comprises or consists of from about 10-200, 10-100, 10-90, 10-80, 10-70, 10-60, 10-50, 10-40, 10-30, or 10-20 amino acids.

In some embodiments, the immunogenic peptide or protein further comprises one or more heterologous peptide or protein element, or the nucleic acid molecules described herein that encodes at least one heterologous peptide or protein element. In some embodiments, the at least one heterologous peptide or protein element may impart an additional function to the immunogenic peptide or protein, e.g., to promote or improve secretion of the encoded immunogenic peptide or protein (e.g., a signal peptide (e.g., described herein), promote or improve anchoring of the encoded immunogenic peptide or protein described herein in the plasma membrane (e.g., via transmembrane elements), promote or improve formation of immunogen complexes (e.g., via multimerization domains or immunogen clustering elements), or promote or improve virus-like particle formation (VLP forming sequence).

Binding to and/or affinity for the hIL-10R (e.g., hIL-10Rα) can be assessed using standard methods known in the art and described herein, see, e.g., § 5.2.2.

### 5.4.1 Portions of Immunomodulatory Proteins

In some embodiments, the immunogenic peptide or protein comprises at least a portion of an immunomodulatory protein or polypeptide described herein. In some embodiments, the immunogenic peptide or protein comprises or consists of at least about 10, 15, 20, 30, 40, 50, 60, 70, 80, 90, 100, 150, or 200 contiguous amino acids of an immunomodulatory protein or polypeptide described herein. In some embodiments, the immunogenic peptide or protein comprises or consists of about 10, 15, 20, 30, 40, 50, 60, 70, 80, 90, 100, 150, or 200 contiguous amino acids of an immunomodulatory protein or polypeptide described herein. In some embodiments, the immunogenic peptide or protein comprises or consists of no more than about 10, 15, 20, 30, 40, 50, 60, 70, 80, 90, 100, 150, or 200 contiguous amino acids of an immunomodulatory protein or polypeptide described herein. In some embodiments, the immunogenic peptide or protein comprises or consists of from about 10-200, 10-100, 10-90, 10-80, 10-70, 10-60, 10-50, 10-40, 10-30, 10-20 contiguous amino acids of the immunomodulatory protein or polypeptide described herein.

In some embodiments, immunogenic peptide or protein comprises an amino acid sequence that is at least 80%, 85%, 90%, 91%, 92%, 93%, 94%, 95%, 96%, 97%, 98%, 99% or 100% identical to a portion of the amino acid sequence of an immunomodulatory protein or polypeptide described herein. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of at least about 10, 15, 20, 30, 40, 50, 60, 70, 80, 90, 100, 150, or 200 amino acids of the immunomodulatory protein or polypeptide described herein. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of about 10, 15, 20, 30, 40, 50, 60, 70, 80, 90, 100, 150, or 200 amino acids of the immunomodulatory protein or polypeptide described herein. In some embodiments, the immunogenic peptide or protein comprises an amino acid sequence that is at least 80%, 85%, 90%, 91%, 92%, 93%, 94%, 95%, 96%, 97%, 98%, 99% or 100% identical to a contiguous stretch of no more than about 10, 15, 20, 30, 40, 50, 60, 70, 80, 90, 100, 150, or 200 amino acids of the immunomodulatory protein or polypeptide described herein.

In some embodiments, the amino acid sequence of the immunogenic peptide or protein comprises 1 or more but less than 15% (less than 12%, less than 10%, less than 8%), amino acid variations (e.g., substitution, addition, deletion) relative to the amino acid sequence of an immunomodulatory protein or polypeptide described herein. In some embodiments, the amino acid sequence of the immunogenic peptide or protein comprises at least one amino acid variation (e.g., substitution, addition, deletion) relative to the amino acid sequence of an immunomodulatory protein or polypeptide described herein. In some embodiments, the amino acid sequence of the immunogenic peptide or protein comprises at least about 1, 2, 3, 4, 5, 6, 7, 8, 9, 10 or more amino acid variations (e.g., substitution, addition, deletion) relative to the amino acid sequence of an immunomodulatory protein or polypeptide described herein. In some embodiments, the amino acid sequence of the immunogenic peptide or protein comprises or consists of about 1, 2, 3, 4, 5, 6, 7, 8, 9, 10 or more amino acid variations (e.g., substitution, addition, deletion) relative to the amino acid sequence of an immunomodulatory protein or polypeptide described herein. In some embodiments, the amino acid sequence of the immunogenic peptide or protein comprises or consists of no more than about 1, 2, 3, 4, 5, 6, 7, 8, 9, 10 amino acid variations (e.g., substitution, addition, deletion) relative to the amino acid sequence of an immunomodulatory protein or polypeptide described herein.

In some embodiments, the amino acid sequence of the immunogenic peptide or protein comprises 1 or more but less than 15% (less than 12%, less than 10%, less than 8%), amino acid substitution relative to the amino acid sequence of an immunomodulatory protein or polypeptide described herein. In some embodiments, the amino acid sequence of the immunogenic peptide or protein comprises at least one amino acid substitution relative to the amino acid sequence of an immunomodulatory protein or polypeptide described herein. In some embodiments, the amino acid sequence of the immunogenic peptide or protein comprises at least about 1, 2, 3, 4, 5, 6, 7, 8, 9, 10 or more amino acid substitutions relative to the amino acid sequence of an immunomodulatory protein or polypeptide described herein. In some embodiments, the amino acid sequence of the immunogenic peptide or protein comprises or consists of about 1, 2, 3, 4, 5, 6, 7, 8, 9, 10 or more amino acid substitutions relative to the amino acid sequence of an immunomodulatory protein or polypeptide described herein. In some embodiments, the amino acid sequence of the immunogenic peptide or protein comprises or consists of no more than about 1, 2, 3, 4, 5, 6, 7, 8, 9, 10 amino acid substitutions relative to the amino acid sequence of an immunomodulatory protein or polypeptide described herein.

### 5.4.2 Variants of Immunomodulatory Proteins

In some embodiments, the amino acid sequence of the immunogenic peptide or protein comprises or consists of the amino acid sequence of an immunomodulatory protein or polypeptide described herein comprising or consisting of 1 or more but less than 15% (less than 12%, less than 10%, less than 8%), amino acid variations (e.g., substitution, addition, deletion) relative to the amino acid sequence of an immunomodulatory protein or polypeptide described herein. In some embodiments, the amino acid sequence of the immunogenic peptide or protein comprises or consists of the amino acid sequence of an immunomodulatory protein or polypeptide described herein comprising or consisting of at least one amino acid variation (e.g., substitution, addition, deletion). In some embodiments, the amino acid sequence of the immunogenic peptide or protein comprises or consists of the amino acid sequence of an immunomodulatory protein or polypeptide described herein comprising or consisting of at least one amino acid substitution or deletion. In some embodiments, the amino acid sequence of the immunogenic peptide or protein comprises or consists of the amino acid sequence of an immunomodulatory protein or polypeptide described herein comprising or consisting of at least one amino acid substitution. In some embodiments, the amino acid sequence of the immunogenic peptide or protein comprises or consists of the amino acid sequence of an immunomodulatory protein or polypeptide described herein comprising or consisting of at least about 1, 2, 3, 4, 5, 6, 7, 8, 9, 10 or more amino acid substitutions. In some embodiments, the amino acid sequence of the immunogenic peptide or protein comprises or consists of the amino acid sequence of an immunomodulatory protein or polypeptide described herein comprising or consisting of about 1, 2, 3, 4, 5, 6, 7, 8, 9, 10 or more amino acid substitutions. In some embodiments, the amino acid sequence of the immunogenic peptide or protein comprises or consists of the amino acid sequence of an immunomodulatory protein or polypeptide described herein comprising or consisting of no more than about 1, 2, 3, 4, 5, 6, 7, 8, 9, 10 amino acid substitutions.

In some embodiments, the immunogenic peptide or protein comprises an amino acid sequence that, other than the at least one amino acid variation (e.g., substitution, addition, deletion), is at least 80%, 85%, 90%, 91%, 92%, 93%, 94%, 95%, 96%, 97%, 98%, 99% or 100% identical to a contiguous stretch of at least 10, 15, 20, 30, 40, 50, 60, 70, 80, 90, 100, 150, or 200 amino acids of an immunomodulatory protein or polypeptide described herein. In some embodiments, immunogenic peptide or protein comprises an amino acid sequence that, other than the at least one amino acid substitution is at least 80%, 85%, 90%, 91%, 92%, 93%, 94%, 95%, 96%, 97%, 98%, 99% or 100% identical to the amino acid sequence of an immunomodulatory protein or polypeptide described herein.

### 5.4.3 Peptide and Protein-Based Vaccines

In some embodiments, an immunogenic peptide or protein described herein forms the basis for a pharmaceutical composition (e.g., a peptide or protein-based vaccine composition). Therefore, in one aspect, provided herein are pharmaceutical compositions (e.g., vaccine compositions) comprising an immunogenic peptide or protein described herein.

In some embodiments, the vaccine composition comprises a plurality of the immunogenic peptides or proteins (e.g., described herein). In some embodiments, the pharmaceutical composition (e.g., vaccine composition) comprises a plurality of substantially the same immunogenic peptide or protein (e.g., described herein). In some embodiments, the pharmaceutical composition (e.g., vaccine composition) comprises a plurality of different immunogenic peptide or protein (e.g., described herein). In some embodiments, the pharmaceutical composition (e.g., vaccine composition) comprises at least 1, 2, 3, 4, 5, 6, 7, 8, 9, or 10 different immunogenic peptides or proteins (e.g., described herein). In some embodiments, the pharmaceutical composition (e.g., vaccine composition) comprises or consists of 1, 2, 3, 4, 5, 6, 7, 8, 9, or 10 different immunogenic peptides or proteins (e.g., described herein). In some embodiments, the pharmaceutical composition (e.g., vaccine composition) comprises at least one (e.g., 2, 3, 4, 5, 6) immunogenic peptide or protein described herein and at least one immunogenic peptide or protein from a different virus.

### 5.4.4 Nucleic Acid-Based Vaccines

In some embodiments, a nucleic acid molecule encoding an immunogenic peptide or protein described herein (also referred to herein as an “immunogenic nucleic acid molecule”) forms the basis for a pharmaceutical composition (e.g., a vaccine composition (e.g., a nucleic acid-based vaccine)). In some embodiments, the nucleic acid molecule is RNA (e.g., mRNA or circular RNA) or DNA. In some embodiments, the nucleic acid molecule is mRNA. In some embodiments, the nucleic acid molecule is circular RNA (see, e.g., WO2019118919, the full contents of which are incorporated by reference herein for all purposes).

In some embodiments, the segment of the nucleic acid molecule encoding the immunogenic peptide or protein (e.g., described herein) comprises from about 30 to about 20000 nucleotides, about 50 to about 20000 nucleotides, about 500 to about 10000 nucleotides, about 1000 to about 10000 nucleotides, about 1000 to about 5000 nucleotides, or about 2000 to about 5000 nucleotides. In some embodiments, the segment of the nucleic acid molecule encoding the immunogenic peptide or protein (e.g., described herein) comprises at least 30 nucleotides, 50 nucleotides, 100 nucleotides, 200 nucleotides, 300 nucleotides, 400 nucleotides, 500 nucleotides, 1000 nucleotides, 2000 nucleotides, 3000 nucleotides, or 5000 nucleotides.

In some embodiments, the nucleic acid molecule is modified or varied (compared to a reference nucleic acid sequence), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide modification (see, e.g., description below), etc.

In some embodiments, the nucleic acid sequence is codon optimized, e.g., for expression in humans. Codon optimization, in some embodiments, may be used to match codon frequencies in target and host organisms to ensure proper folding; bias guanosine (G) and/or cytosine (C) content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified or varied to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

In some embodiments, the pharmaceutical composition comprises a plurality of substantially the same nucleic acid molecules encoding a plurality of immunogenic peptides or proteins (e.g., described herein). In some embodiments, the pharmaceutical composition comprises a plurality of different nucleic acid molecules encoding a plurality of different immunogenic peptides or proteins (e.g., described herein). In some embodiments, the pharmaceutical composition comprises at least 1, 2, 3, 4, 5, 6, 7, 8, 9, or 10 different nucleic acid molecules encoding at least 1, 2, 3, 4, 5, 6, 7, 8, 9, or 10 different immunogenic peptides or proteins (e.g., described herein). In some embodiments, the pharmaceutical composition comprises or consists of 1, 2, 3, 4, 5, 6, 7, 8, 9, or 10 different nucleic acid molecules encoding 1, 2, 3, 4, 5, 6, 7, 8, 9, or 10 different immunogenic peptides or proteins (e.g., described herein).

### 5.4.4.1 DNA Nucleic Acids

In some embodiments, the nucleic acid molecule is DNA. In some embodiments, the DNA is a linear coding DNA construct, contained within a plasmid, or contained within a viral vector. In some embodiments, the DNA is a linear coding DNA construct. In some embodiments, the DNA is contained within a plasmid. In some embodiments, the DNA is contained with a viral vector. A more detailed description of viral vectors for both RNA and DNA nucleic acids is provided in § 5.7.2.

The coding DNA may also comprise one or more heterologous nucleic acid elements to mediate expression of the coding region. These include, e.g., promoter(s), enhancer(s), polyadenylation signal(s), synthetic introns, transcriptional termination signals, polyadenylation sequences, and other transcription regulatory elements. A person of ordinary skill in the art is familiar with the transcriptional regulatory elements needed for expression of the coding DNA and can optimize the expression construct (e.g., linear DNA or a plasmid) accordingly.

In some embodiments, a promoter is operably linked to the respective coding nucleic acid sequence encoding the immunogenic peptide or protein. The person of ordinary skill in the art is aware of various promoters that can be employed, for example, a promoter from simian virus 40 (SV40), a mouse mammary tumor virus (MMTV) promoter, a human immunodeficiency virus (HIV) promoter, bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene, for example, from human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the entire contents of which is incorporated by reference herein for all purposes. Exemplary polyadenylation signals, include, but are not limited, to the bovine growth hormone (BGH) polyadenylation site, SV40 polyadenylation signals, and LTR polyadenylation signals.

In some embodiments, the DNA is contained within a plasmid. A person of ordinary skill in the art is aware of suitable plasmids for expression of the DNA of interest. For example, Suitable plasmid DNA may be generated to allow efficient production of the encoded immunogens in cell lines, e.g., in insect cell lines, for example using vectors as described in WO2009150222A2 and as defined in PCT claims 1 to 33, the disclosure relating to claim 1 to 33 of WO2009150222A2 the entire contents of which is incorporated by reference herein for all purposes.

### 5.4.4.2 RNA Nucleic Acids

In some embodiments, the nucleic acid molecule is RNA. In some embodiments, the RNA is selected from an mRNA, a self-replicating RNA, a circular RNA (e.g., a covalently closed RNA), a viral RNA, or a replicon RNA.

In some embodiments, the RNA is a mRNA. The basic components of an mRNA molecule typically include at least one coding region (herein a coding region encoding at least one immunogenic peptide or protein described herein), a 5′-untranslated region (UTR), a 3′-UTR, a 5′ cap, and a poly-A tail.

In some embodiments, the RNA (e.g., mRNA) comprises at least one heterologous UTR. The UTRs may harbor regulatory sequence elements that determine the RNA (e.g., mRNA) turnover, stability, localization, and/or expression of operably linked coding sequence(s). The heterologous UTRs may be derived from a naturally occurring genes or may be synthetically engineered. In some embodiments, the 5′-UTR comprises elements for controlling gene expression, e.g., ribosomal binding sites, miRNA binding sites. The 5′-UTR may be post-transcriptionally modified or varied, e.g., by enzymatic or post-transcriptional addition of a 5′cap structure. In some embodiments, the 3′-UTR comprises a polyadenylation signal. In some embodiments, the RNA (e.g., mRNA) comprises at least one coding region encoding the immunogenic peptide or protein described herein and 5′-UTR and/or a 3′-UTR. In some embodiments, the RNA (e.g., mRNA) comprises at least one coding sequence encoding an immunogenic peptide or protein described herein operably connected to at least one heterologous 5′-UTR and at least one 3′-UTR.

In some embodiments, the RNA (e.g., mRNA) comprises a poly(A) sequence. The poly(A) sequence may comprise from about 10 to 500 adenosine nucleotides (SEQ ID NO: 544), 10 to 200 adenosine nucleotides (SEQ ID NO: 545), 20 to 200 adenosine nucleotides (SEQ ID NO: 546), 30 to 200 adenosine nucleotides (SEQ ID NO: 547), 40 to 200 adenosine nucleotides (SEQ ID NO: 548), or 50 to 200 adenosine nucleotides (SEQ ID NO: 549). In some embodiments, poly(A) sequence comprises at least 10, 20, 30, 40, 50, 60, 70, 80, 90, 100, 200, 300, 400, or 500 adenosine nucleotides (SEQ ID NO: 550). In some embodiments, the RNA (e.g., mRNA) comprises a poly(A) sequence. The poly(A) sequence may comprise from about 10 to 500 adenosine nucleotides (SEQ ID NO: 544), 10 to 200 adenosine nucleotides (SEQ ID NO: 545), 20 to 200 adenosine nucleotides (SEQ ID NO: 546), 30 to 200 adenosine nucleotides (SEQ ID NO: 547), 40 to 200 adenosine nucleotides (SEQ ID NO: 548), or 50 to 200 adenosine nucleotides (SEQ ID NO: 549), wherein the 3′ terminal nucleotide of said nucleic acid molecule is an adenosine. In some embodiments, poly(A) sequence comprises at least 10, 20, 30, 40, 50, 60, 70, 80, 90, 100, 200, 300, 400, or 500 adenosine nucleotides (SEQ ID NO: 550), wherein the 3′ terminal nucleotide of said nucleic acid molecule is an adenosine.

In some embodiments, the RNA (e.g., mRNA) comprises a 5′-cap structure. In some embodiments, the 5′-cap structure stabilizes the RNA (e.g., mRNA), enhances expression of the encoded immunogen, and/or reduces the stimulation of the innate immune system (e.g., after administration to a subject).

Exemplary 5′-cap structures include, but are not limited to, cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g., phosphorothioate modified ARCA), inosine, N1-methyi-guanosine, 2′-fluoro-guanosine, 7-deaza-guanosine, 8-oxo-guanosine, 2-amino-guanosine, LNA-guanosine, and 2-azido-guanosine. In some embodiments, the 5′ cap structure comprises m7G, cap0, cap1, cap2, a modified capO, or a modified cap1 structure.

In some embodiments, the RNA (e.g., mRNA) comprises nucleotide analogues/modifications, e.g., backbone modifications, sugar modifications, and/or base modifications. A backbone modification in the context of the present disclosure is a modification, in which phosphates of the backbone of the nucleotides of the RNA (e.g., mRNA) are chemically modified. A sugar modification in the context of the present disclosure is a chemical modification of the sugar of the nucleotides of the RNA (e.g., mRNA). A base modification in the context of the present disclosure is a chemical modification of the base moiety of the nucleotides of the RNA (e.g., mRNA).

In some embodiments, the RNA (e.g., mRNA) comprises at least one modified nucleotide. Exemplary nucleotide analogues/modifications include, but are not limited to, 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thiocytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphatej-uridine, 5′-O-(1-thiophosphate)-pseudouridine, 6-aza-cytidine, 2-thio-cytidine, alpha-thio-cytidine, Pseudoiso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thioguanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thioadenosine, 8-azido-adenosine, and 7-deaza-adenosine.

In some embodiments, the RNA (e.g., mRNA) comprises pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and/or 2′-O-methyl uridine.

In some embodiments, the RNA (e.g., mRNA) comprises one or more pseudouridine (ψ), N 1-methylpseudouridine (m1ψ), 5-methylcytosine, and 5-methoxyuridine. In some embodiments, essentially all, e.g., essentially 100% of the uracil in the coding sequence of the RNA (e.g., mRNA) have a chemical modification, preferably a chemical modification is in the 5-position of the uracil. Incorporating modified nucleotides such as e.g., pseudouridine (ψ), N1-methylpseudouridine (m1ψ), 5-methylcytosine, and/or 5-methoxyuridine into the coding sequence may be advantageous as unwanted innate immune responses (upon administration of the coding RNA or the vaccine) may be adjusted or reduced (if required).

In one embodiment, the mRNA encoding an immunogen described herein comprises: (i) a 5′-cap structure; (ii) a 5′-UTR; (iii) N1-methyl-pseudouridine, cytosine, adenine, and guanine; (iv) a 3′-UTR; and (v) a poly-A region.

RNA (e.g., mRNA) described herein can be generated by e.g., in vitro transcription. In vitro transcription is a method well known to those of ordinary skill in the art for the production of RNA (e.g., mRNA). Generally, the RNA is obtained by DNA-dependent in vitro transcription of an appropriate DNA template, e.g., a linearized plasmid DNA template or a PCR-amplified DNA template. The promoter for controlling RNA in vitro transcription can be any promoter for any DNA-dependent RNA polymerase. Examples of DNA-dependent RNA polymerases include the 17, T3, SP6, or Syn5 RNA polymerases. In some instances, the DNA template is linearized with a suitable restriction enzyme before it is subjected to RNA in vitro transcription. Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); a DNA-dependent RNA polymerase capable of binding to the promoter sequence within the DNA template (e.g., T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCh, which supplies Mg2+ ions as a co-factor for the polymerase; a buffer (TRIS or HEPES) to maintain a suitable pH value, which can also contain antioxidants (e.g., DTT), and/or polyamines such as spermidine at optimal concentrations, e.g., a buffer system comprising TRIS-Citrate as disclosed in WO2017109161. The obtained RNA (e.g., mRNA) products can be purified according to methods known in the art. For example, using PureMessenger® (CureVac, Tubingen, Germany; RP-HPLC according to WO2008077592) and/or tangential flow filtration (as described in WO2016193206) and/or oligo d(T) purification (see WO2016180430); or using RP-HPLC, e.g., using Reversed-Phase High pressure liquid chromatography (RP-HPLC), the entire contents of each reference is incorporated by reference herein for all purposes.

### 5.5 Methods of Making Polypeptides & Proteins

Any protein or polypeptide described herein, e.g., including an immunomodulatory protein or polypeptide described herein (see, e.g., § 5.2), a fusion protein or polypeptide described herein (see, e.g., § 5.3), a conjugate described herein (see, e.g., § 5.3), an immunogenic peptide or protein described herein (see, e.g., § 5.4), or an antibody described herein (see, e.g., § 5.9), may be produced using standard methods known in the art. For example, each of the above may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the subject polypeptide (e.g., immunomodulatory polypeptide, fusion polypeptide, immunogenic peptide or protein, or antibody). Such general methods are common knowledge in the art. The expression vector typically contains an expression cassette that includes nucleic acid sequences capable of bringing about expression of the nucleic acid molecule encoding the protein or polypeptide of interest, such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein or polypeptide of interest are cultured under conditions conducive to expression of the nucleic acid molecule encoding the immunogenic peptide or protein. Culture media is available from various vendors, and a suitable medium can be routinely chosen for a host cell to express a protein or polypeptide of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein or polypeptide may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein or polypeptide may be concentrated, buffer exchanged by ultrafiltration, and the retentate from the ultrafiltration may be filtered through an appropriate filter, e.g., a 0.22 μm filter. See, e.g., Hacker, David (Ed.), Recombinant Protein Expression in Mammalian Cells: Methods and Protocols (Methods in Molecular Biology), Humana Press (2018); and McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes.

The proteins and polypeptide described herein (e.g., including immunomodulatory proteins and polypeptides, fusion proteins and polypeptides, immunogenic peptides and proteins, and antibodies described herein) may be produced synthetically. The proteins and polypeptides described herein (e.g., including the immunomodulatory proteins and polypeptides, fusion proteins and polypeptides, immunogenic peptides and proteins, and antibodies described herein) and particularly the immunogenic peptides and proteins described herein, may be produced by using an egg-based manufacturing method.

In some aspects and embodiments, the disclosure features methods of making a protein or polypeptide described herein (e.g., an immunomodulatory protein or polypeptide described herein, a fusion protein or polypeptide described herein, a conjugate described herein, an immunogenic peptide or protein described herein, or an antibody described herein). The method includes (a) recombinantly expressing the protein or polypeptide (e.g., the immunomodulatory protein or polypeptide described herein, fusion protein or polypeptide described herein, immunogenic peptide or protein described herein, or antibody described herein); (b) enriching, e.g., purifying, the protein or polypeptide (e.g., the immunomodulatory protein or polypeptide described herein, fusion protein or polypeptide described herein, immunogenic peptide or protein described herein, or antibody described herein); (c) evaluating the protein or polypeptide (e.g., the immunomodulatory protein or polypeptide described herein, fusion protein or polypeptide described herein, immunogenic peptide or protein described herein, or antibody described herein) for the presence of a process impurity or contaminant, and (d) formulating the protein or polypeptide (e.g., the immunomodulatory protein or polypeptide described herein, fusion protein or polypeptide described herein, immunogenic peptide or protein described herein, or antibody described herein) as a pharmaceutical composition if the protein or polypeptide (e.g., the immunomodulatory protein or polypeptide described herein, fusion protein or polypeptide described herein, immunogenic peptide or protein described herein, or antibody described herein) meets a threshold specification for the process impurity or contaminant. The process impurity or contaminant evaluated may be one or more of, e.g., a process-related impurity such as host cell proteins, host cell DNA, or a cell culture component (e.g., inducers, antibiotics, or media components); a product-related impurity (e.g., precursors, fragments, aggregates, degradation products); or contaminants, e.g., endotoxin, bacteria, viral contaminants.

### 5.6 Nucleic Acid Molecules

In one aspect, provided herein are polynucleotides (e.g., DNA, RNA) encoding any protein described herein (including, e.g., an immunomodulatory protein or polypeptide described herein (see, e.g., § 5.2), a fusion protein or polypeptide described herein (see, e.g., § 5.3), a conjugate described herein (see, e.g., § 5.3), an immunogenic peptide or protein described herein (see, e.g., § 5.4), an antibody described herein (see, e.g., § 5.9)). In some embodiments, the polynucleotide is a DNA polynucleotide or an RNA polynucleotide. In some embodiments, the polynucleotide is an mRNA polynucleotide.

In some embodiments, the polynucleotide is codon optimized. Codon optimization may be used to match codon frequencies in target and host organisms to ensure proper folding; bias guanosine (G) and/or cytosine content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the polynucleotide. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 5.7 Vectors

In one aspect, provided herein are vectors comprising a polynucleotide (e.g., DNA, RNA) described herein (e.g., a polynucleotide described in § 5.6) (e.g., an immunomodulatory protein or polypeptide described herein (see, e.g., § 5.2), a fusion protein or polypeptide described herein (see, e.g., § 5.3), a conjugate described herein (see, e.g., § 5.3), an immunogenic peptide or protein described herein (see, e.g., § 5.4), or an antibody described herein (see, e.g., § 5.9)). In some embodiments, the vector is a viral vector. In some embodiments, the vector is a non-viral vector (e.g., a plasmid, a minicircle).

### 5.7.1 Non-Viral Vectors

In some embodiments, the vector is a non-viral vector. In some embodiments, the vector is a minicircle. In some embodiments, the vector is a plasmid. A person of ordinary skill in the art is aware of suitable plasmids for expression of the DNA of interest. For example, plasmid DNA may be generated to allow efficient production of the encoded endonucleases in cell lines, e.g., in insect cell lines, for example using vectors as described in WO2009150222A2 and as defined in PCT claims 1 to 33, the disclosure relating to claim 1 to 33 of WO2009150222A2 the entire contents of which is incorporated by reference herein for all purposes.

5.7.2 Viral Vectors

In some embodiments, the nucleic acid molecules (e.g., DNA or RNA) encoding an immunogenic peptide or protein described herein are contained in a viral vector. Thus, also provided herein are viral vectors comprising the nucleic acid molecules encoding an immunogenic peptide or protein described herein. Such vectors can be easily manipulated by methods well known to the ordinary person of skill in the art. The vector used can be any vector that is suitable for cloning nucleic acids that can be used for transcription of the nucleic acid molecule of interest.

Viral vectors include both RNA and DNA based vectors. The vectors can be designed to meet a variety of specifications. For example, viral vectors can be engineered to be capable or incapable of replication in prokaryotic and/or eukaryotic cells. In some embodiments, the vector is replication deficient. In some embodiments, the vector is replication competent. Vectors can be engineered or selected that either will (or will not) integrate in whole or in part into the genome of host cells, resulting (or not (e.g., episomal expression)) in stable host cells comprising the desired nucleic acid in their genome.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors. In some embodiments, the viral vector is an adenovirus vector, adeno-associated virus vector, lentivirus vector, anellovector (as described, for example, in U.S. Pat. No. 11,446,344, the entire contents of which is incorporated by reference herein for all purposes).

In some embodiments, the vector is an adenoviral vector (e.g., human adenoviral vector, e.g., HAdV or AdHu). In some embodiments, the adenovirus vector has the E1 region deleted, rendering it replication-deficient in human cells. Other regions of the adenovirus such as E3 and E4 may also be deleted. Exemplary adenovirus vectors include, but are not limited to, those described in e.g., WO2005071093 or WQ2006048215, the entire contents of each of which is incorporated by reference herein for all purposes. In some embodiments, the adenovirus-based vector used is a simian adenovirus, thereby avoiding dampening of the immune response after vaccination by pre-existing antibodies to common human entities such as AdHu5. Exemplary, simian adenovirus vectors include AdCh63 (see, e.g., WO2005071093, the entire contents of which is incorporated by reference herein for all purposes) or AdCh68.

Viral vectors can be generated through the use of a packaging/producer cell line (e.g., a mammalian cell line) using standard methods known to the person of ordinary skill in the art. Generally, a nucleic acid construct (e.g., a plasmid) encoding the transgene (e.g., an immunogenic peptide or protein described herein) (along with additional elements e.g., a promoter, inverted terminal repeats (ITRs) flanking the transgene, a plasmid encoding e.g., viral replication and structural proteins, along with one or more helper plasmids a host cell (e.g., a host cell line) are transfected into a host cell line (i.e., the packing/producer cell line). In some instances, depending on the viral vector, a helper plasmid may also be needed that include helper genes from another virus (e.g., in the instance of adeno-associated viral vectors). Eukaryotic expression plasmids are commercially available from a variety of suppliers, for example the plasmid series: pcDNA™ pCR3.1™, pCMV™, pFRT™, pVAX1™, pCI™, Nanoplasmid™, and Pcaggs. The person of ordinary skill in the art is aware of numerous transfection methods and any suitable method of transfection may be employed (e.g., using a biochemical substance as carrier (e.g., lipofectamine), by mechanical means, or by electroporation). The cells are cultured under conditions suitable and for a sufficient time for plasmid expression. The viral particles may be purified from the cell culture medium using standard methods known to the person of ordinary skill in the art. For example, by centrifugation followed by e.g., chromatography or ultrafiltration.

### 5.8 Cells

In one aspect, provided herein are cells (e.g., host cells) comprising any one or more of an immunomodulatory protein or polypeptide described herein (see, e.g., § 5.2), a fusion protein or polypeptide described herein (see, e.g., § 5.3), a conjugate described herein (see, e.g., § 5.3), an immunogenic peptide or protein described herein (see, e.g., § 5.4), an antibody described herein (see, e.g., § 5.9), a polynucleotide described herein (see, e.g., § 5.6), a vector described herein (see, e.g., § 5.7), or a carrier described herein (see, e.g., § 5.10).

In some embodiments, the cell is a eukaryotic cell. In some embodiments, the cell is mammalian cell. In some embodiments, the cell is an animal cell. In some embodiments, the cell is a human cell. In some embodiments, the cell is in vitro. In some embodiments, the cell is in vivo. In some embodiments, the cell is ex vivo.

Standard methods known in the art can be utilized to deliver any one of the foregoing (e.g., immunomodulatory protein, fusion protein, vector, polynucleotide, carrier, etc.) into a cell (e.g., a host cell). Standard methods known in the art can be utilized to culture cells (e.g., host cells) in vitro or ex vivo.

### 5.9 Antibodies

In one aspect, provided herein are antibodies (and functional fragments and variants thereof) that specifically bind an immunomodulatory protein or polypeptide described herein and inhibit binding of the immunomodulatory protein or polypeptide to the hIL-10R (e.g., hIL-10Rα). The antibody can be for example, a full-length antibody, a Fab, a scFv, or a single domain antibody. In some embodiments, the antibody (e.g., an antibody that specifically binds an immunomodulatory protein or polypeptide described herein) is labeled with a tag (e.g., a fluorescent tag) to aid in detection. In some embodiments, the antibody is utilized in a therapeutic method, e.g., a method of preventing or treating a viral infection (see, e.g., § 5.13.8). In some embodiments, the antibody is utilized in a diagnostic method, e.g., a method of diagnosing a subject with a viral infection (see, e.g., § 5.13.9). In some embodiments, the antibody is contained in a kit described herein (see, e.g., § 5.14).

### 5.10 Carriers

In some embodiments, an immunomodulatory protein or polypeptide described herein (see, e.g., § 5.2), a fusion protein or polypeptide described herein (see, e.g., § 5.3), a conjugate described herein (see, e.g., § 5.3), an immunogenic peptide or protein described herein (see, e.g., § 5.4), an antibody described herein (see, e.g., § 5.9), a polynucleotide described herein (see, e.g., § 5.6), or a vector described herein (see, e.g., § 5.7), is formulated within a carrier.

As, such, the disclosure provides, inter alia, carriers comprising any one of more of an immunomodulatory protein or polypeptide described herein, a fusion polypeptide or protein described herein, a conjugate described herein, an immunogenic peptide or protein described herein, an antibody described herein, a polynucleotide described herein (e.g., a polynucleotide encoding an immunomodulatory protein or polypeptide described herein, a fusion polypeptide or protein described herein, an immunogenic peptide or protein described herein, an antibody described herein, etc.), or a vector described herein (e.g., a vector comprising a polynucleotide described herein).

Any of the foregoing (e.g., proteins, polynucleotides, vectors, etc.) can be encapsulated within a carrier, chemically conjugated to a carrier, associated with the carrier. In this context, the term “associated” refers to the essentially stable combination of any one of the foregoing, e.g., a protein, polynucleotide, vector, etc., with one or more molecules of a carrier (e.g., one or more lipids of a lipid-based carrier, e.g., an LNP, liposome, lipoplex, and/or nanoliposome) into larger complexes or assemblies without covalent binding. In this context, the term “encapsulation” refers to the incorporation of any one of the foregoing, e.g., a protein, a polynucleotide, a vector, etc.) into a carrier (e.g., a lipid-based carrier, e.g., an LNP, liposome, lipoplex, and/or nanoliposome) wherein the molecule (e.g., the protein, polynucleotide, vector, etc.) is entirely contained within the interior space of the carrier (e.g., the lipid-based carrier, e.g., the LNP, liposome, lipoplex, and/or nanoliposome).

Exemplary carriers include, but are not limited to, lipid-based carriers (e.g., lipid nanoparticles (LNPs), liposomes, lipoplexes, and nanoliposomes). In some embodiments, the carrier is a lipid-based carrier. In some embodiments, the carrier is an LNP. In some embodiments, the LNP comprises a cationic lipid, a neutral lipid, a cholesterol, and/or a PEG lipid. Lipid based carriers are further described below in § 5.10.1.

### 5.10.1 Lipid Based Carriers

In some embodiments, an immunomodulatory protein or polypeptide described herein (see, e.g., § 5.2), a fusion protein or polypeptide described herein (see, e.g., § 5.3), a conjugate described herein (see, e.g., § 5.3), an immunogenic peptide or protein described herein (see, e.g., § 5.4), an antibody described herein (see, e.g., § 5.9), a polynucleotide described herein (see, e.g., § 5.6), a vector described herein (see, e.g., § 5.7), a cell described herein (see, e.g., § 5.8); a vaccine composition (see, e.g., § 5.4), or a pharmaceutical composition described herein (see, e.g., § 5.12) is encapsulated or associated with one or more lipids (e.g., cationic lipids and/or neutral lipids), thereby forming lipid-based carriers such as lipid nanoparticles (LNPs), liposomes, lipoplexes, or nanoliposomes.

In some embodiments, any of the foregoing (e.g., proteins, polynucleotides, vectors, etc.) is encapsulated in one or more lipids (e.g., cationic lipids and/or neutral lipids), thereby forming lipid-based carriers such as lipid nanoparticles (LNPs), liposomes, lipoplexes, or nanoliposomes. In some embodiments, the molecule (e.g., the protein, polynucleotide, vector, etc.) is associated with one or more lipids (e.g., cationic lipids and/or neutral lipids), thereby forming lipid-based carriers such as lipid nanoparticles (LNPs), liposomes, lipoplexes, or nanoliposomes. In some embodiments, the molecule (e.g., the protein, polynucleotide, vector, etc.) is encapsulated in LNPs (e.g., as described herein). LNPs are described in further detail in § 5.10.1.1. The use of LNPs for mRNA delivery is further detailed in e.g., Hou X et al. Lipid nanoparticles for mRNA delivery. Nat Rev Mater. 2021; 6(12):1078-1094. doi: 10.1038/s41578-021-00358-0. Epub 2021 Aug. 10. PMID: 34394960; PMCID: PMC8353930, the entire contents of which are incorporated by reference herein for all purposes.

The molecules (e.g., the protein, polynucleotides, vectors, etc.) may be completely or partially located in the interior space of the LNPs, liposomes, lipoplexes, and/or nanoliposomes, within the lipid layer/membrane, or associated with the exterior surface of the lipid layer/membrane. One purpose of incorporating the molecule (e.g., the protein, polynucleotide, vector, etc.) into LNPs, liposomes, lipoplexes, and/or nanoliposomes is to protect the molecule (e.g., the protein, polynucleotide, vector, etc.) from an environment which may contain enzymes or chemicals or conditions that degrade the molecule (e.g., the protein, polynucleotide, vector, etc.) from molecules or conditions that cause the rapid excretion of the molecule (e.g., the protein, polynucleotide, vector, etc.). Moreover, incorporating the molecules (e.g., the proteins, polynucleotides, vectors, systems, etc.) into LNPs, liposomes, lipoplexes, and/or nanoliposomes may promote the uptake of the molecules (e.g., the proteins, polynucleotides, vectors, etc.), and hence, may enhance the therapeutic effect of the proteins or polynucleotides (e.g., RNA, e.g., mRNA). Accordingly, incorporating a molecule (e.g., protein, polynucleotide, vector, etc.), into LNPs, liposomes, lipoplexes, and/or nanoliposomes may be particularly suitable for a pharmaceutical composition described herein, e.g., for intramuscular and/or intradermal administration.

LNPs, liposomes, lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. In some embodiments, the LNPs, liposomes, lipoplexes, and/or nanoliposomes has a diameter from about 10 to 500 nm, 10 to 400 nm, 10 to 300 nm, 10 to 200 nm, 10 to 100 nm, or 10 to 50 nm. In some embodiments, the LNPs, liposomes, lipoplexes, and/or nanoliposomes has a diameter of at least about 10 nm, 20 nm, 30 nm, 40 nm, 50 nm, 60 nm, 70 nm, 80 nm, 90 nm, 100 nm, 150 nm, 200 nm, 250 nm, 300 nm, 350 nm, 400 nm, 450 nm, or 500 nm.

### 5.10.1.1 Lipid Nanoparticles (LNPs)

In some embodiments, an immunomodulatory protein or polypeptide described herein (see, e.g., § 5.2), a fusion protein or polypeptide described herein (see, e.g., § 5.3), a conjugate described herein (see, e.g., § 5.3), an immunogenic peptide or protein described herein (see, e.g., § 5.4), an antibody described herein (see, e.g., § 5.9), a polynucleotide described herein (e.g., an mRNA encoding a immunomodulatory protein or polypeptide described herein) (see, e.g., § 5.6), a vector described herein (see, e.g., § 5.7), a cell described herein (see, e.g., § 5.8); a vaccine composition (see, e.g., § 5.4), or a pharmaceutical composition described herein (see, e.g., § 5.12) is comprised in an LNP. In some embodiments, LNPs include one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; the entire contents of which is incorporated by reference herein for all purposes); one or more sterols (e.g., cholesterol). In some embodiments, an LNP preparation comprises a cationic lipid, a neutral lipid, a cholesterol, and a PEG lipid, and has a mean particle size of between 50-200 nm, e.g., between 80 nm and 160 nm.

Lipids that can be used in nanoparticle formations (e.g., LNPs) include, for example those described in Table 4 of WO2019217941, which is incorporated by reference—e.g., a lipid-containing nanoparticle can include one or more of the lipids in Table 4 of WO2019217941. LNPs can include additional elements, such as polymers, such as the polymers described in Table 5 of WO2019217941, the entire contents of which is incorporated by reference herein for all purposes.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (the entire contents of which is incorporated by reference herein for all purposes), and combinations of the foregoing.

In some embodiments, sterols that can be incorporated into lipid nanoparticles include one or more of cholesterol or cholesterol derivatives, such as those in WO2009/127060 or US2010/0130588, the entire contents of which is incorporated by reference herein for all purposes. Additional exemplary sterols include phytosterols, including those described in Eygeris et al. (2020), dx.doi.org/10.1021/acs.nanolett.0c01386, the entire contents of which is incorporated by reference herein for all purposes.

In some embodiments, the lipid particle includes an ionizable lipid, a non-cationic lipid, a conjugated lipid that inhibits aggregation of particles, and a sterol. The amounts of these components can be varied independently and to achieve desired properties. For example, In some embodiments, the lipid nanoparticle includes an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Non-limiting examples of lipid compounds that may be used (e.g., in combination with other lipid components) to form lipid nanoparticles for the delivery of agents described herein, e.g., proteins, polynucleotides, vectors, etc., includes,

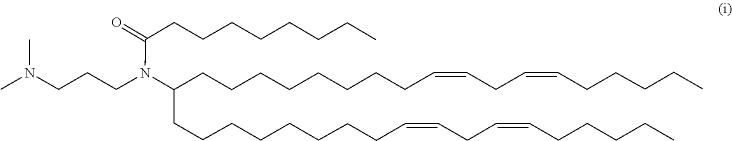 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP including Formula (i) is used to deliver an agent described herein (e.g., a protein, polynucleotide, vector, etc. described herein) to cells.

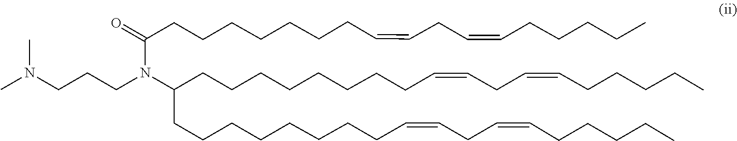 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP including Formula (ii) is used to deliver an agent described herein (e.g., a protein, polynucleotide, vector, etc. described herein) to cells.

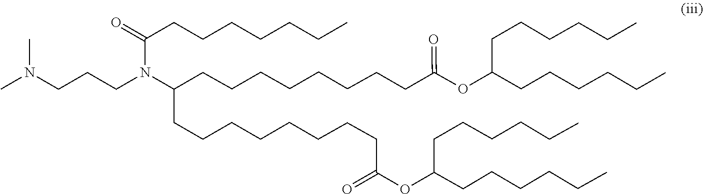 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP including Formula (iii) is used to deliver an agent described herein (e.g., a protein, polynucleotide, vector, etc. described herein) to cells.

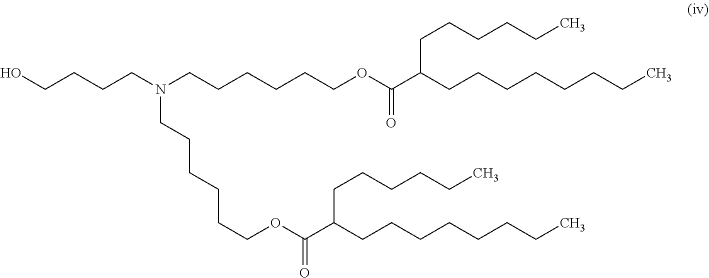 [SMILES: CCCCCCCCC(CCCCCC)C(=O)OCCCCCCN(CCCCO)CCCCCCOC(=O)C(CCCCCC)CCCCCCCC]

In some embodiments an LNP including Formula (iv) is used to deliver an agent described herein (e.g., a protein, polynucleotide, vector, etc. described herein) to cells.

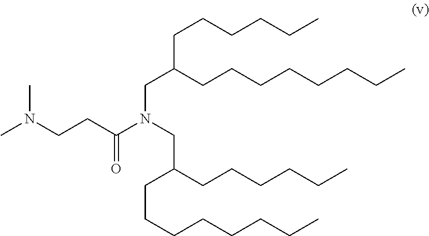 [SMILES: CCCCCCCCC(CCCCCC)CN(CC(CCCCCC)CCCCCCCC)C(=O)CCN(C)C]

In some embodiments an LNP including Formula (v) is used to deliver an agent described herein (e.g., a protein, polynucleotide, vector, etc. described herein) to cells.

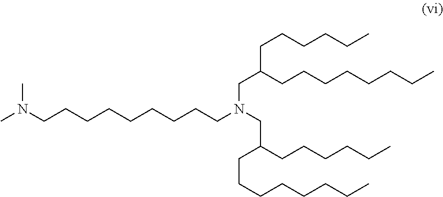 [SMILES: CCCCCCCCC(CCCCCC)CN(CCCCCCCCCN(C)C)CC(CCCCCC)CCCCCCCC]

In some embodiments an LNP including Formula (vi) is used to deliver an agent described herein (e.g., a protein, polynucleotide, vector, etc. described herein) to cells.

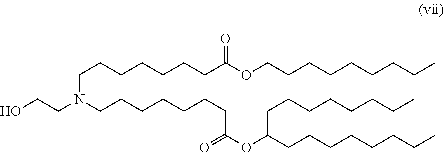 [SMILES: CCCCCCCCCOC(=O)CCCCCCCN(CCO)CCCCCCCC(=O)OC(CCCCCCCC)CCCCCCCC]

In some embodiments an LNP including Formula (vii) is used to deliver an agent described herein (e.g., a protein, polynucleotide, vector, etc. described herein) to cells.

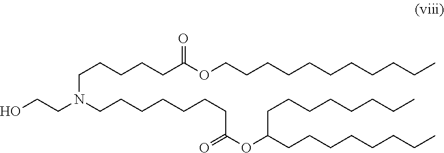 [SMILES: CCCCCCCCCCCOC(=O)CCCCCN(CCO)CCCCCCCC(=O)OC(CCCCCCCC)CCCCCCCC]

In some embodiments an LNP including Formula (viii) is used to deliver an agent described herein (e.g., a protein, polynucleotide, vector, etc. described herein) to cells.

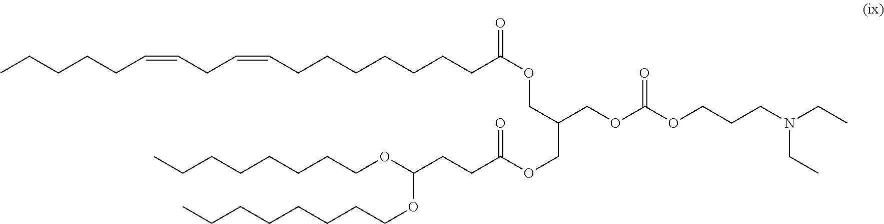 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP including Formula (ix) is used to deliver an agent described herein (e.g., a protein, polynucleotide, vector, etc. described herein) to cells.

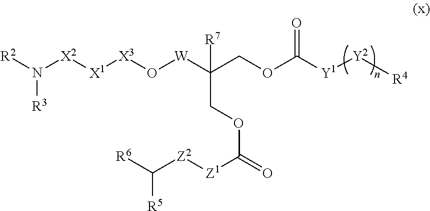

wherein
X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

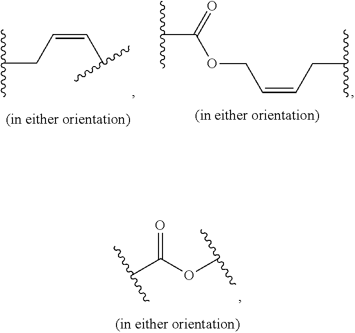 [SMILES: */C=C\C* ; *C/C=C\COC(*)=O ; *OC(*)=O]

n is 0 to 3, R⁴ is C1-15 alkyl, Z¹ is C1-6 alkylene or a direct bond,
Z² is

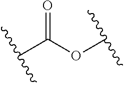 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

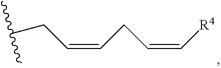

R⁴ is linear C5 alkyl, Z¹ is C2 alkylene, Z² is absent, W is methylene, and R⁷ is H, then R⁵ and R⁶ are not Cx alkoxy.

In some embodiments an LNP including Formula (x) is used to deliver an agent described herein (e.g., a protein, polynucleotide, vector, etc. described herein) to cells.

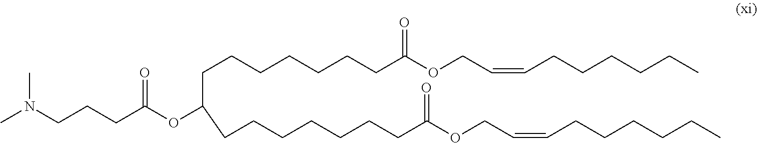 [SMILES: CCCCCC/C=C\COC(=O)CCCCCCCC(CCCCCCCC(=O)OC/C=C\CCCCCC)OC(=O)CCCN(C)C]

In some embodiments an LNP including Formula (xi) is used to deliver an agent described herein (e.g., a protein, polynucleotide, vector, etc. described herein) to cells.

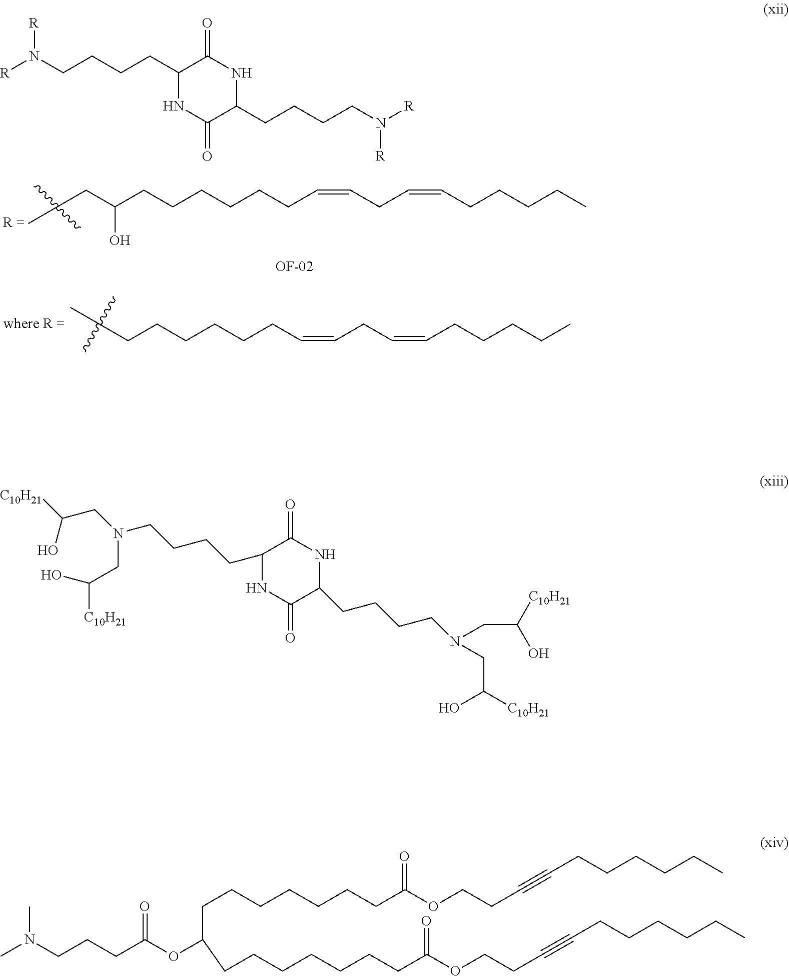

In some embodiments an LNP includes a compound of Formula (xiii) and a compound of Formula (xiv).

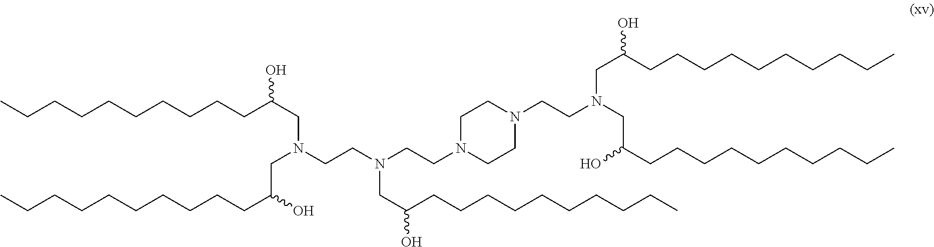 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP including Formula (xv) is used to deliver an agent described herein (e.g., a protein, polynucleotide, vector, etc. described herein) to cells.

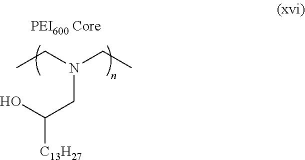

In some embodiments an LNP including a formulation of Formula (xvi) is used to deliver an agent described herein (e.g., a protein, polynucleotide, vector, etc. described herein) to cells.

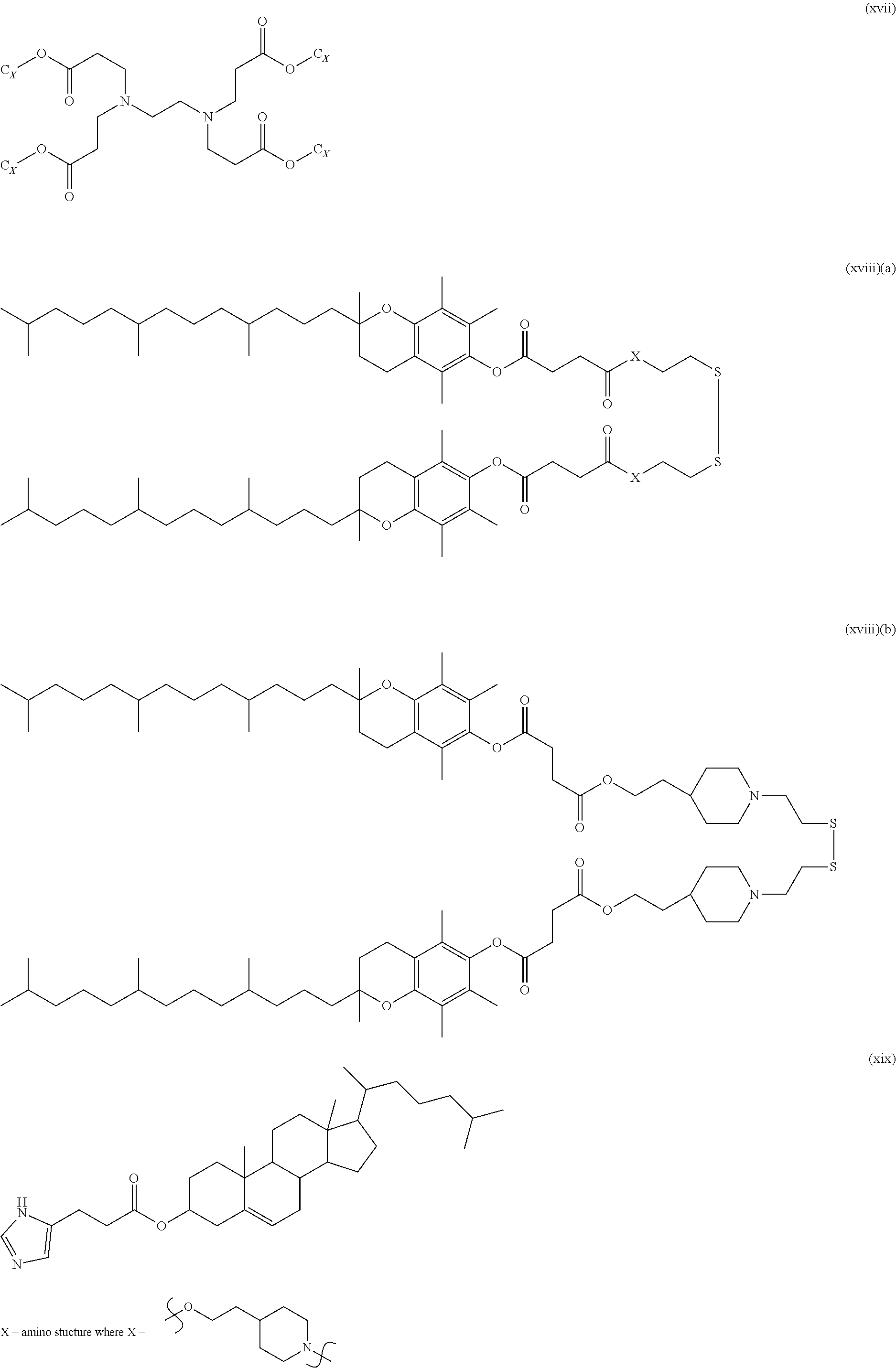

In some embodiments, a lipid compound used to form lipid nanoparticles for the delivery of agents described herein (e.g., proteins, polynucleotides, vectors, etc. described herein) is made by one of the following reactions:

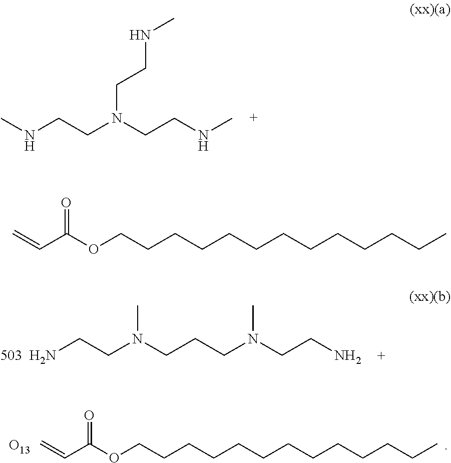

In some embodiments an LNP including Formula (xxi) is used to deliver an agent described herein (e.g., a protein, polynucleotide, vector, etc. described herein) to cells. In some embodiments the LNP of Formula (xxi) is an LNP described by WO2021113777 (e.g., a lipid of Formula (1) such as a lipid of Table 1 of WO2021113777, the entire contents of which is incorporated by reference herein for all purposes).

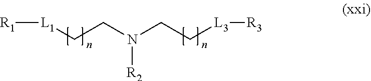

wherein
each n is independently an integer from 2-15; Li and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and
R₂ is selected from a group consisting of:

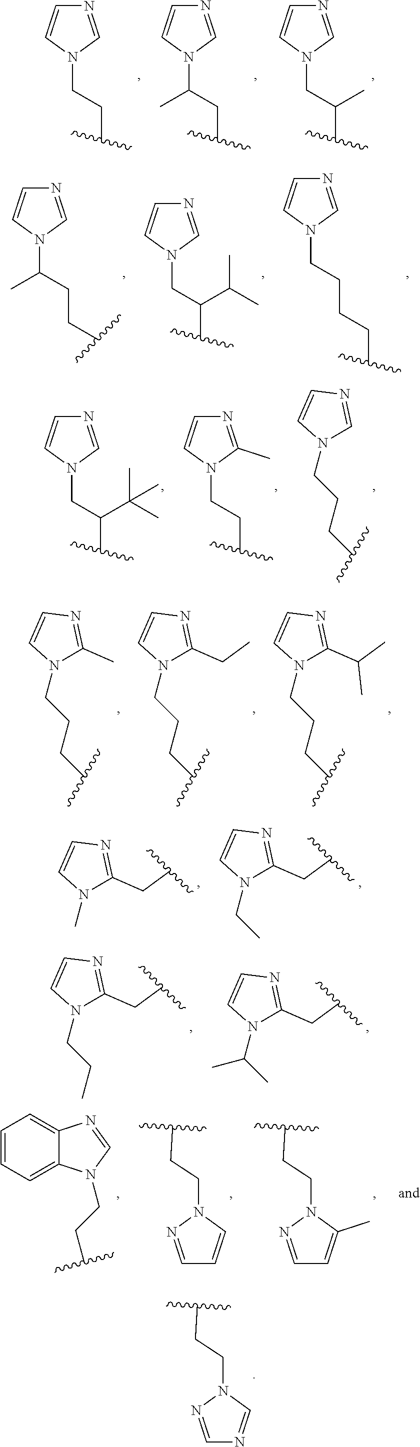

In some embodiments an LNP including Formula (xxii) is used to deliver a an agent described herein (e.g., a protein, polynucleotide, vector, etc. described herein) to cells. In some embodiments the LNP of Formula (xxii) is an LNP described by WO2021113777 (e.g., a lipid of Formula (2) such as a lipid of Table 2 of WO2021113777).

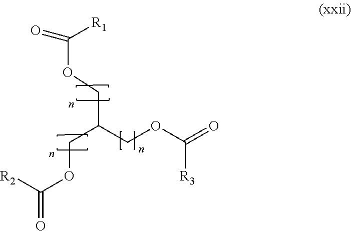

wherein
each n is independently an integer from 1-15;
R₁ and R₂ are each independently selected from a group consisting of:

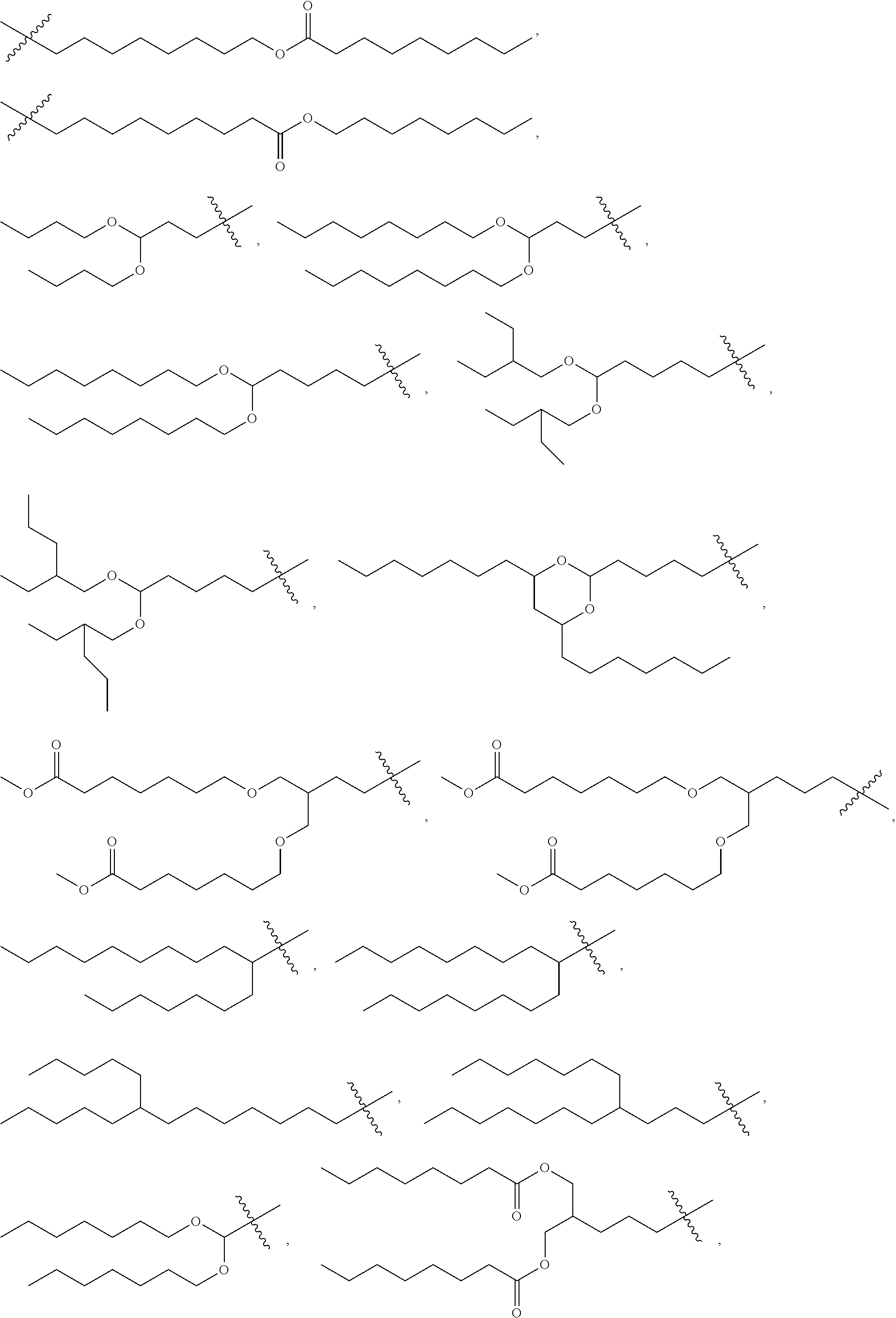

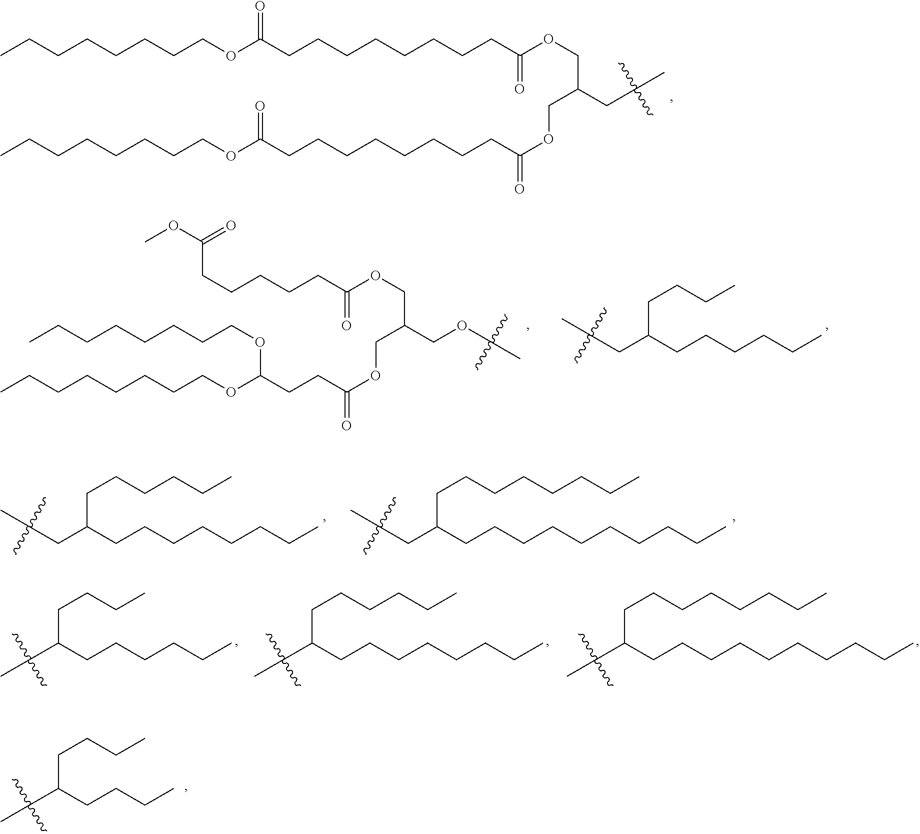

R₃ is selected from a group consisting of:

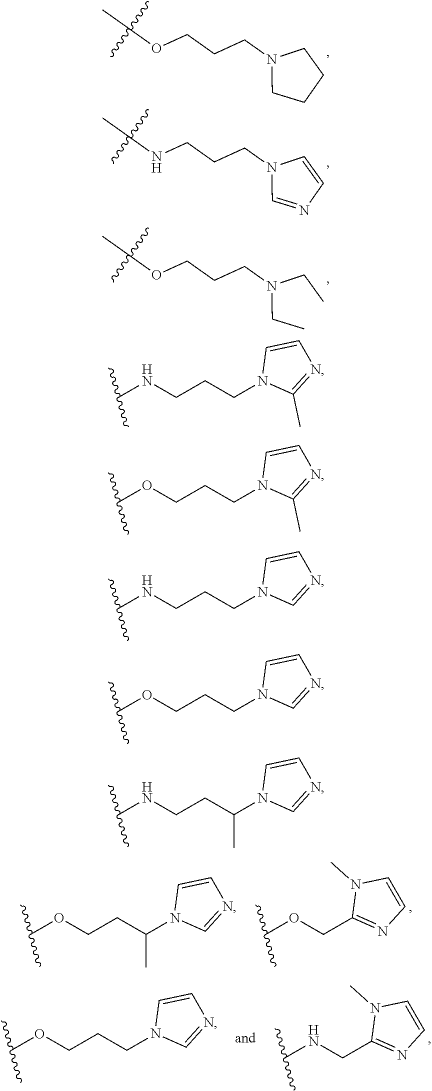

In some embodiments an LNP including Formula (xxiii) is used to deliver an agent described herein (e.g., a protein, polynucleotide, vector, etc. described herein) to cells. In some embodiments the LNP of Formula (xxiii) is an LNP described by WO2021113777 (e.g., a lipid of Formula (3) such as a lipid of Table 3 of WO2021113777).

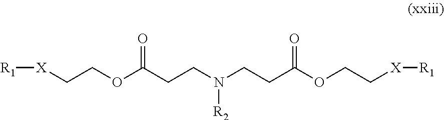

wherein
X is selected from —O—, —S—, or —OC(O)—*, wherein * indicates the attachment point to R₁;
R₁ is selected from a group consisting of:

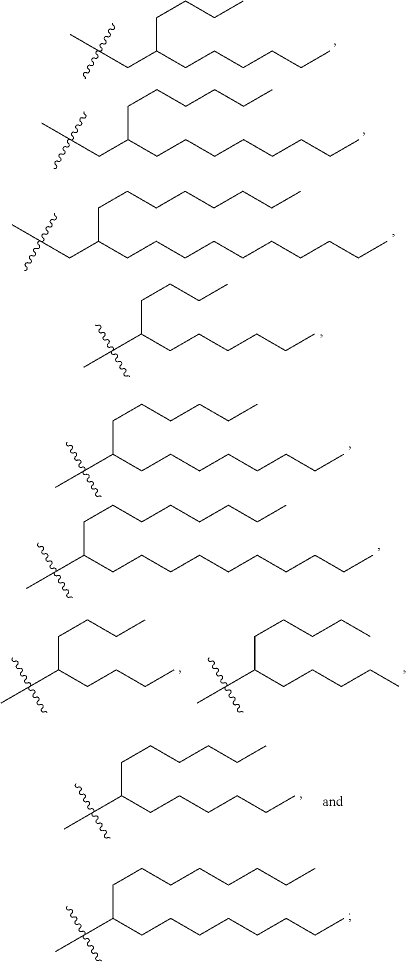

and R₂ is selected from a group consisting of:

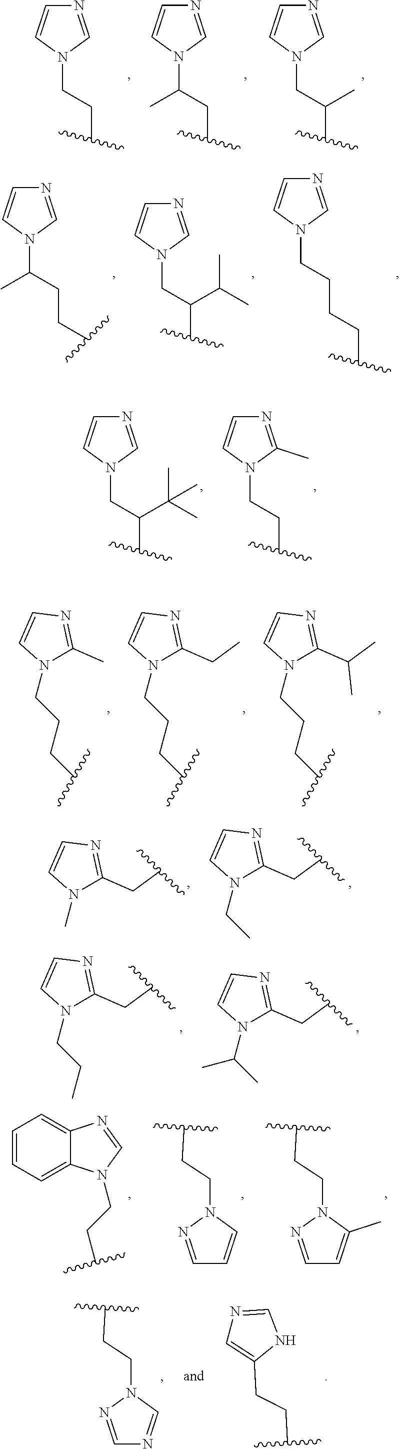

In some embodiments, agents described herein (e.g., proteins, polynucleotides, vectors, etc. described herein) are provided in an LNP that includes an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g., as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (the entire contents of which is incorporated by reference herein for all purposes).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle includes a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids. In some embodiments, the cationic lipid may be an ionizable cationic lipid. An exemplary cationic lipid as disclosed herein may have an effective pKa over 6.0. In some embodiments, a lipid nanoparticle may include a second cationic lipid having a different effective pKa (e.g., greater than the first effective pKa), than the first cationic lipid. A lipid nanoparticle may include between 40 and 60 mol percent of a cationic lipid, a neutral lipid, a steroid, a polymer conjugated lipid, and an agent described herein (e.g., a protein, polynucleotide, vector, etc. described herein), encapsulated within or associated with the lipid nanoparticle. In some embodiments, the nucleic acid is co-formulated with the cationic lipid. The nucleic acid may be adsorbed to the surface of an LNP, e.g., an LNP including a cationic lipid. In some embodiments, the nucleic acid may be encapsulated in an LNP, e.g., an LNP including a cationic lipid. In some embodiments, the lipid nanoparticle may include a targeting moiety, e.g., coated with a targeting agent. In some embodiments, the LNP formulation is biodegradable. In some embodiments, a lipid nanoparticle including one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an agent described herein (e.g., a protein, polynucleotide, vector, etc. described herein).

Exemplary ionizable lipids that can be used in lipid nanoparticle formulations include, without limitation, those listed in Table 1 of WO2019051289, the entire contents of which is incorporated by reference herein for all purposes. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; 111-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-O13 or 503-O13 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777. The entire contents of each reference is incorporated by reference herein for all purposes

In some embodiments, the ionizable lipid is MC3 (6Z,9Z,28Z,3 lZ)-heptatriaconta-6,9,28,3 1-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (the entire contents of which is incorporated by reference herein for all purposes). In some embodiments, the ionizable lipid is (13Z,16Z)-A,A-dimethyl-3-nonyldocosa-13, 16-dien-1-amine (Compound 32), e.g., as described in Example 11 of WO2019051289A9 (the entire contents of which is incorporated by reference herein for all purposes). In some embodiments, the ionizable lipid is Compound 6 or Compound 22, e.g., as described in Example 12 of WO2019051289A9 (the entire contents of which is incorporated by reference herein for all purposes).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in some embodiments, include, without limitation, those described in Kim et al. (2020) dx.doi.org/10.1021/acs.nanolett.0c01386, incorporated herein by reference. Such lipids include, In some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the entire contents of which is incorporated by reference herein for all purposes.

In some embodiments, the non-cationic lipid is oleic acid or a compound of Formula I, II, or IV of US2018/0028664, the entire contents of which is incorporated by reference herein for all purposes.

The non-cationic lipid can include, for example, 0-30% (mol) of the total lipid present in the lipid nanoparticle. In some embodiments, the non-cationic lipid content is 5-20% (mol) or 10-15% (mol) of the total lipid present in the lipid nanoparticle. In some embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not include any phospholipids.

In some embodiments, the lipid nanoparticle further include a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, the entire contents of each of which is incorporated by reference herein for all purposes.

In some embodiments, a component providing membrane integrity, such as a sterol, can include 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can include a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the entire contents of each of which is incorporated by reference herein for all purposes. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the entire contents of each of which is incorporated by reference herein for all purposes. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid includes PEG-DMG, 1,2-dimyristoyl-sn-glycero-3-phosphoethanolamine-N-[methoxy(polyethylene glycol)-2000]. In some embodiments, the PEG-lipid includes a structure selected from:

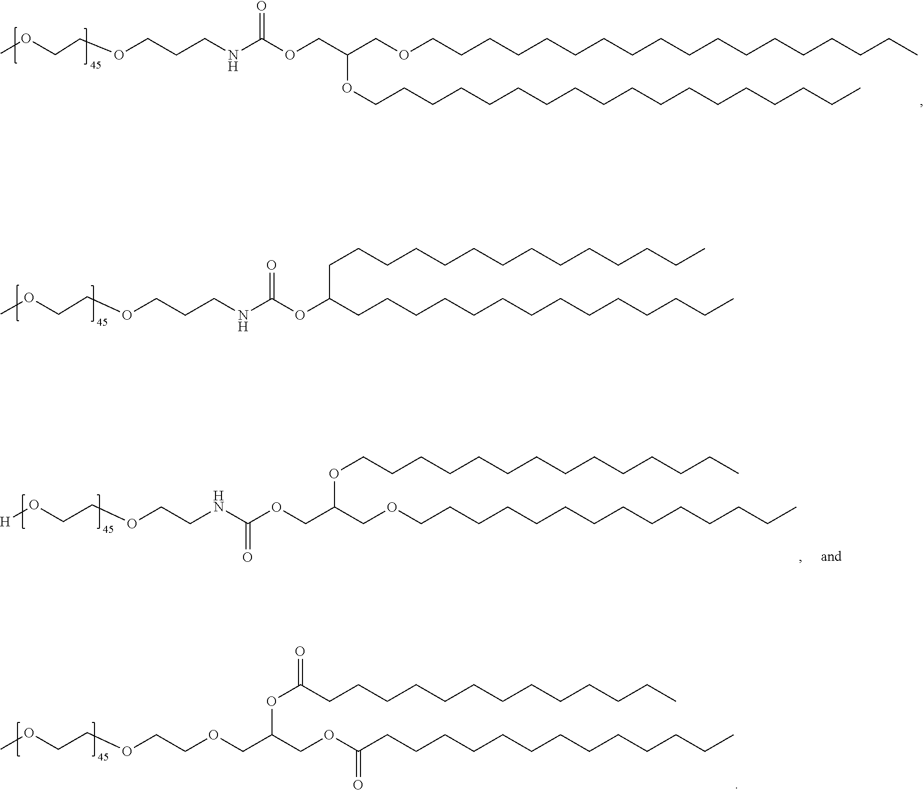

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the entire contents of which is incorporated by reference herein for all purposes.

In some embodiments, the PEG or the conjugated lipid can include 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can include 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition includes 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example including 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation includes ionizable lipid, phospholipid, cholesterol and a PEG-ylated lipid in a molar ratio of 50:10:38.5:1.5. In some other embodiments, the lipid particle formulation includes ionizable lipid, cholesterol and a PEG-ylated lipid in a molar ratio of 60:38.5:1.5.

In some embodiments, the lipid particle includes ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle includes ionizable lipid/non-cationic-lipid/sterol/conjugated lipid at a molar ratio of 50:10:38.5:1.5.

In some embodiments, the disclosure provides a lipid nanoparticle formulation including phospholipids, lecithin, phosphatidylcholine and phosphatidylethanolamine.

In some embodiments, one or more additional compounds can also be included. Those compounds can be administered separately, or the additional compounds can be included in the lipid nanoparticles described herein. In other words, the lipid nanoparticles can contain other compounds in addition to the agent (e.g., a protein, polynucleotide, vector, etc. described herein) or at least a second agent described herein (e.g., a protein, polynucleotide, vector, etc. described herein), different than the first. Without limitations, other additional compounds can be selected from the group consisting of small or large organic or inorganic molecules, monosaccharides, disaccharides, trisaccharides, oligosaccharides, polysaccharides, proteins, peptide analogs and derivatives thereof, peptidomimetics, nucleic acids, nucleic acid analogs and derivatives, an extract made from biological materials, or any combinations thereof.

In some embodiments, the LNPs include biodegradable, ionizable lipids. In some embodiments, the LNPs include (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, the entire contents of each of which is incorporated by reference herein for all purposes, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of an agent described herein (e.g., a protein, polynucleotide, vector, etc. described herein), describes the amount of an agent described herein (e.g., a protein, polynucleotide, vector, etc. described herein) that is encapsulated or otherwise associated with a LNP after preparation, relative to the initial amount provided. The encapsulation efficiency is desirably high (e.g., close to 100%). The encapsulation efficiency may be measured, for example, by comparing the amount of the agent (e.g., a protein, polynucleotide, vector, etc. described herein) in a solution containing the lipid nanoparticle before and after breaking up the lipid nanoparticle with one or more organic solvents or detergents. An anion exchange resin may be used to measure the amount of free agent (e.g., a protein, polynucleotide, vector, etc. described herein) in a solution. Fluorescence may be used to measure the amount of free agent (e.g., a protein, polynucleotide, vector, etc. described herein) in a solution. For the lipid nanoparticles described herein, the encapsulation efficiency of an agent (e.g., a protein, polynucleotide, vector, etc. described herein) may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

An LNP may optionally include one or more coatings. In some embodiments, a LNP may be formulated in a capsule, film, or table having a coating. A capsule, film, or tablet including a composition described herein may have any useful size, tensile strength, hardness, or density.

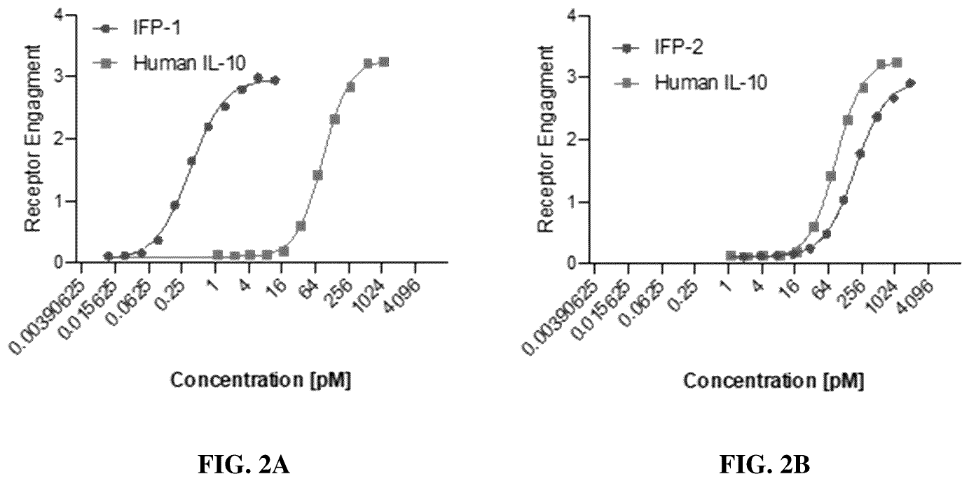

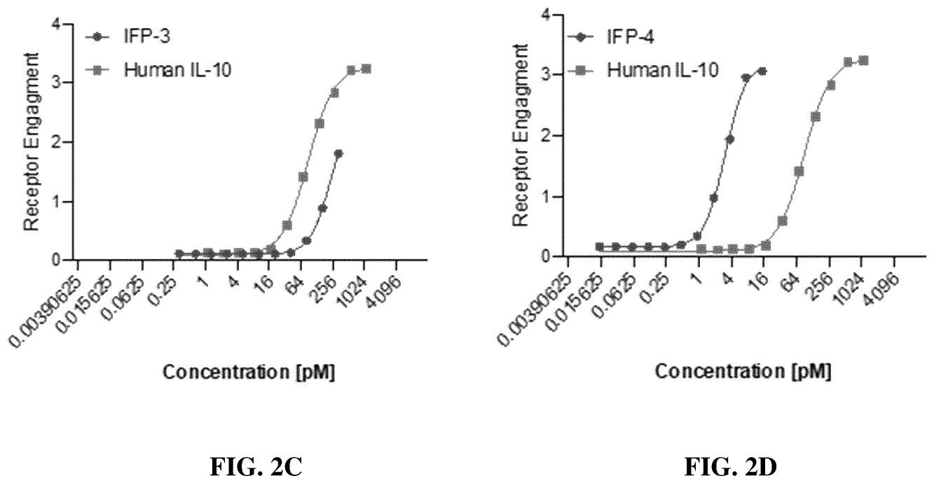

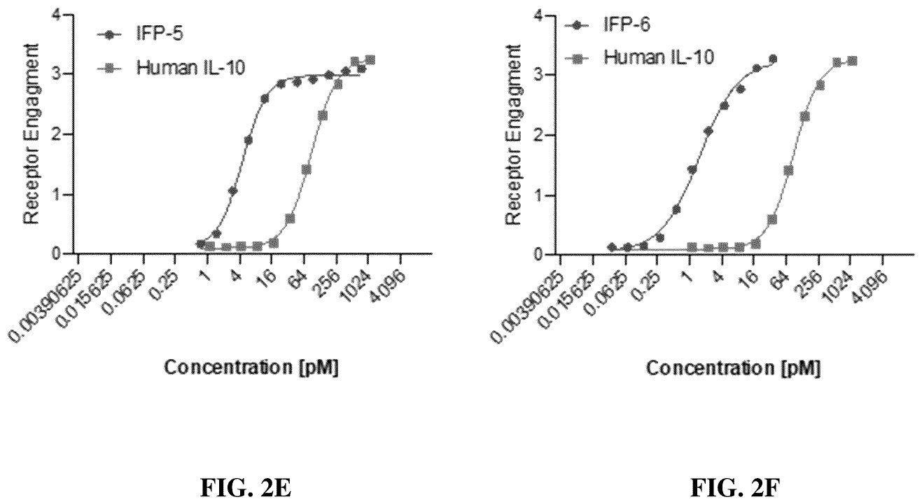

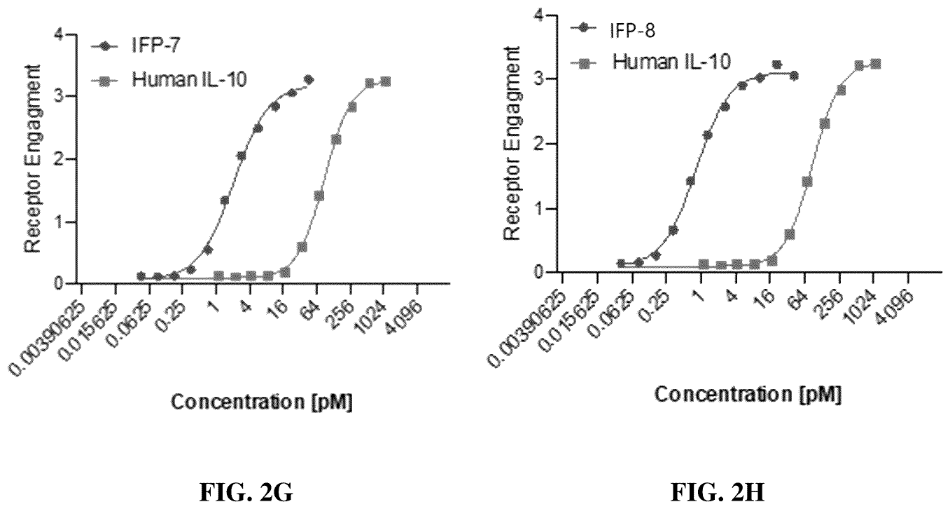

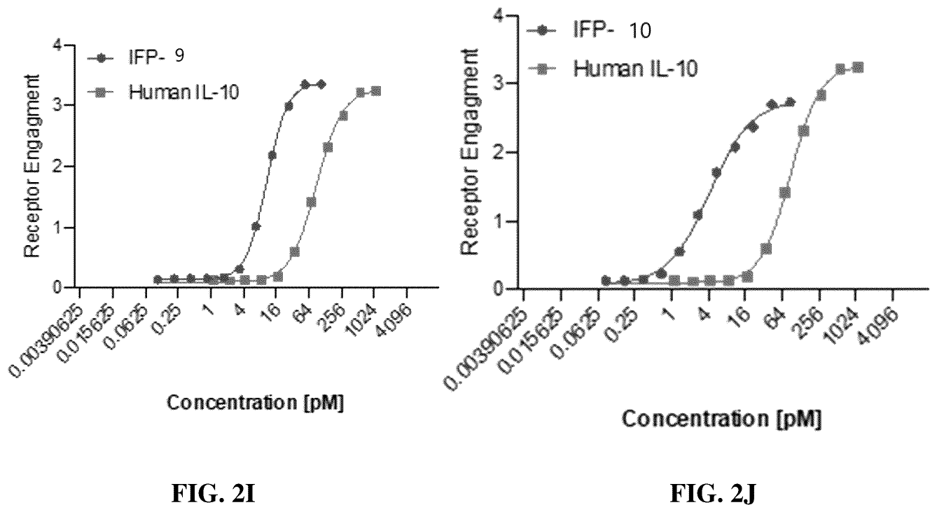

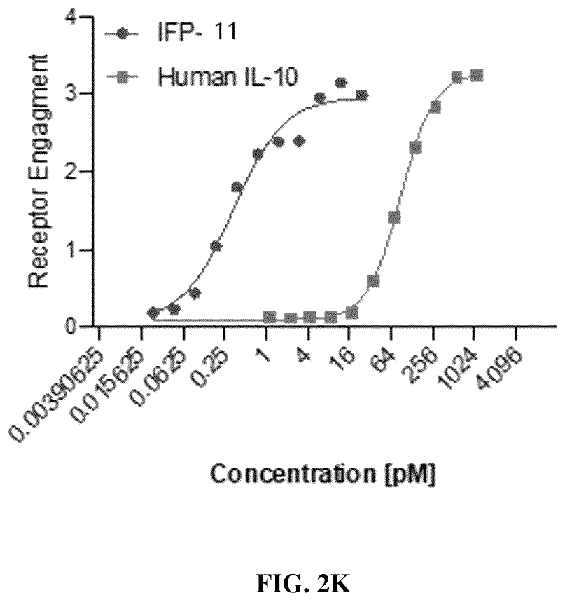

Additional exemplary lipids, formulations, methods, and characterization of LNPs are taught by WO2020/061457 and WO2021/113777, the entire contents of each of which is incorporated by reference herein for all purposes. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.), the entire contents of which is incorporated by reference herein for all purposes.

In some embodiments, in vitro or ex vivo cell lipofections are performed using Lipofectamine MessengerMax (Thermo Fisher) or TransIT-mRNA Transfection Reagent (Mirus Bio). In Certain some embodiments, LNPs are formulated using the GenVoy_ILM ionizable lipid mix (Precision NanoSystems). In Certain some embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), the entire contents of which is incorporated by reference herein for all purposes.

Additional specific LNP formulations useful for delivery of nucleic acids are described in U.S. Pat. Nos. 8,158,601 and 8,168,775, the entire contents of each of which is incorporated by reference herein for all purposes, which include formulations used in patisiran, sold under the name ONPATTRO. Exemplary dosing of LNPs may include about 0.1, 0.25, 0.3, 0.5, 1, 2, 3, 4, 5, 6, 8, 10, or 100 mg/kg (RNA). Exemplary dosing of AAV including a polyribonucleotide (e.g., a circular polyribonucleotide, a linear polyribonucleotide) may include an MOI of about 10¹¹, 10¹², 10¹³, and 10¹⁴ vg/kg.

### 5.11 Adjuvants

The pharmaceutical compositions described herein (e.g., including vaccine compositions) (e.g., pharmaceutical compositions and vaccine compositions comprising an immunogenic peptide or protein (e.g., described herein) or a polynucleotide (e.g., DNA, RNA (e.g., mRNA)) encoding the immunogenic peptide or protein (e.g., described herein)) may comprise one or more adjuvants or be co-administered with one or more adjuvants. Adjuvants are known in the art to further increase the immune response to an immunogen. General categories of adjuvants include, but are not limited to, inorganic adjuvants, small molecule adjuvants, oil in water emulsions, lipids or polymers, peptides or peptidoglycans, carbohydrates or polysaccharides, RNA-based adjuvants, DNA-based adjuvants, viral particles, bacterial adjuvants, inorganic nanoparticles, and multi-component adjuvants. Examples of adjuvants include, but are not limited to, aluminum salts such as aluminum hydroxide and/or aluminum phosphate; oil-emulsion compositions (or oil-in-water compositions), including squalene-water emulsions, such as MF59 (see, e.g., WO90/14837), MF59, ASO3, and Montanide; saponin formulations, such as for example QS21 and Immunostimulating Complexes (ISCOMS) (see, e.g., U.S. Pat. No. 5,057,540; WO90/03184, WO96/11711, WO2004/004762, WO2005/002620, the entire contents of each of which is incorporated by reference herein for all purposes); protamine or a protamine salt (e.g., protamine sulfate); calcium salt; bacterial or microbial derivatives, examples of which include monophosphoryl lipid A (MPL), 3-O-deacylated MPL (3 dMPL), CpG-motif containing polynucleotides, ADP-ribosylating bacterial toxins or mutants thereof, such as E. coli heat labile enterotoxin LT, cholera toxin CT, and the like; eukaryotic proteins (e.g., antibodies or fragments thereof (e.g., directed against the immunogen itself or CD1a, CD3, CD7, CD80) and ligands to receptors (e.g., CD40L, GMCSF, GCSF, etc.).

Exemplary RNA-based adjuvants include, but are not limited to, Poly IC, Poly IC:LC, hairpin RNAs, e.g., with a 5′PPP containing sequence, viral sequences, polyU containing sequences, dsRNA, natural or synthetic immunostimulatory RNA sequences, nucleic acids analogs, optionally cyclic GMP-AMP or a cyclic dinucleotide such as cyclic di-GMP, and immunostimulatory base analogs, e.g., C8-substituted or an N7,C8-disubstituted guanine ribonucleotide. Exemplary DNA-based adjuvants, include, but are not limited to, CpGs, dsDNA, or natural or synthetic immunostimulatory DNA sequences. Exemplary bacteria-based adjuvants include, but are not limited, to bacterial adjuvant is flagellin, LPS, or a bacterial toxin, e.g., enterotoxins, heat-labile toxins, and Cholera toxins. Exemplary carbohydrate or polysaccharide adjuvants include, but are not limited to, dextran (branched microbial polysaccharide), dextran-sulfate, Lentinan, zymosan, Betaglucan, Deltin, Mannan, and Chitin. Exemplary small molecule adjuvants, include, but are not limited to, imiquimod, resiquimod, and gardiquimod. Exemplary lipid or polymer adjuvants, include, but are not limited to, polymeric nanoparticles (e.g., PLGA, PLG, PLA, PGA, or PHB), liposomes (e.g., Virosomes and CAF01), LNPs or a component thereof, lipopolysaccharide (LPS) (e.g., monophosphoryl lipid A (MPLA) or glucopyranosyl Lipid A (GLA)), lipopeptides (e.g., Pam2 (Pam2CSK4) or Pam3 (Pam3CSK4)), and glycolipid (e.g., trehalose dimycolate). Exemplary peptides or peptidoglycan include, but are not limited to, N-acetyl-muramyl-L-alanyl-D-isoglutamine (MDP), flagellin-fusion protein, mannose-binding lectin (MBL), cytokines, and chemokine. Exemplary inorganic nanoparticle adjuvants, include, but are not limited to, gold nanorods, silica-based nanoparticles (e.g., mesoporous silica nanoparticles (MSN)). Exemplary multicomponent adjuvants include, but are not limited to, ASO1, ASO3, ASO4, Complete Freunds Adjuvant, and CAF01.

### 5.12 Pharmaceutical Compositions

In one aspect, provided herein are pharmaceutical compositions comprising any one or more of an immunomodulatory protein or polypeptide described herein (see, e.g., § 5.2), a fusion protein or polypeptide described herein (see, e.g., § 5.3), a conjugate described herein (see, e.g., § 5.3), an immunogenic peptide or protein described herein (see, e.g., § 5.4), an antibody described herein (see, e.g., § 5.9), a polynucleotide described herein (see, e.g., § 5.6), a vector described herein (see, e.g., § 5.7), a cell described herein (see, e.g., § 5.8), or a carrier described herein (see, e.g., § 5.10), and a pharmaceutically acceptable excipient (see, e.g., Remington's Pharmaceutical Sciences (1990) Mack Publishing Co., Easton, PA, the entire contents of which is incorporated by reference herein for all purposes).

Also provided herein are pharmaceutical compositions comprising an immunomodulatory protein or polypeptide described herein, a fusion polypeptide or protein described herein, a conjugate described herein, an immunogenic peptide or protein described herein, an antibody described herein, a polynucleotide described herein, a vector described herein, a host cell described herein, or a carrier described herein, wherein the pharmaceutical composition lacks a predetermined threshold amount or a detectable amount of a process impurity or contaminant, e.g., lacks a predetermined threshold amount or a detectable amount of a process-related impurity such as host cell proteins, host cell DNA, or a cell culture component (e.g., inducers, antibiotics, or media components); a product-related impurity (e.g., precursors, fragments, aggregates, degradation products); or a contaminant, e.g., endotoxin, bacteria, viral contaminant.

In one aspect, also provided herein are methods of making pharmaceutical compositions described herein comprising providing an immunomodulatory protein or polypeptide described herein, a fusion polypeptide or protein described herein, a conjugate described herein, an immunogenic peptide or protein described herein, an antibody described herein, a polynucleotide described herein, a vector described herein, a host cell described herein, or a carrier described herein, and formulating it into a pharmaceutically acceptable composition by the addition of one or more pharmaceutically acceptable excipient.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

A pharmaceutical composition may be formulated for any route of administration to a subject. The skilled person knows the various possibilities to administer a pharmaceutical composition described herein a in order to induce an immune response to the immunogen(s) in the pharmaceutical composition. Non-limiting embodiments include parenteral administration, such as intramuscular, intradermal, subcutaneous, transcutaneous, or mucosal administration, e.g., inhalation, intranasal, oral, and the like. In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; orsodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.13 Methods of Use

Provided herein are various methods of utilizing any one or more agent described herein, including e.g., an immunomodulatory protein or polypeptide described herein (see, e.g., § 5.2), a fusion protein or polypeptide described herein (see, e.g., § 5.3), a conjugate described herein (see, e.g., § 5.3), an immunogenic peptide or protein described herein (see, e.g., § 5.4), an antibody described herein (see, e.g., § 5.9), a polynucleotide described herein (see, e.g., § 5.6), a vector described herein (see, e.g., § 5.7), a cell described herein (see, e.g., § 5.8), a carrier described herein (see, e.g., § 5.10), a vaccine composition described herein (see, e.g., § 5.4), or a pharmaceutical composition described herein (see, e.g., § 5.12). Exemplary subjects include mammals, e.g., humans, non-human mammals, e.g., non-human primates. In some embodiments, the subject is a human.

In some embodiments, the subject exhibits a genetic mutation in the gene encoding IL-10 (e.g., hIL-10), IL-10Rα (e.g., hIL-10Rα), and/or IL-10Rβ (e.g., hIL-10Rβ). In some embodiments, the genetic mutation is associated with the development of a disease (e.g., the early development of a disease relative to a subject that does not exhibit the genetic mutation) (e.g., inflammatory bowel disease (e.g., Chron's disease, ulcerative colitis, see, e.g., § 5.13.3))).

The dosage of an immunomodulatory protein or polypeptide described herein, a fusion polypeptide or protein described herein, a conjugate described herein, an immunogenic peptide or protein described herein, an antibody described herein, a polynucleotide described herein, a vector described herein, a host cell described herein, a carrier described herein, a vaccine composition, or a pharmaceutical composition described herein to be administered to a subject in accordance with any of the methods described herein can be determined in accordance with standard techniques known to those of ordinary skill in the art, including the route of administration, the age and weight of the subject, and the type (if any) adjuvant is used.

### 5.13.1 Methods of Delivery

In one aspect, provided herein are methods of delivering (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) to a subject, the method comprising administering the immunomodulatory protein or polypeptide, the fusion polypeptide or protein, the conjugate, the immunogenic peptide or protein, the antibody, the polynucleotide, the vector, the host cell, the carrier, the vaccine composition, or the pharmaceutical composition to the subject, to thereby deliver the immunomodulatory protein or polypeptide, the fusion polypeptide or protein, the conjugate, the immunogenic peptide or protein, the antibody, the polynucleotide, the vector, the host cell, the carrier, the vaccine composition, or the pharmaceutical composition to the subject. In some embodiments, the immunomodulatory protein or polypeptide, the fusion polypeptide or protein, the conjugate, the immunogenic peptide or protein, the antibody, the polynucleotide, the vector, the host cell, the carrier, the vaccine composition, or the pharmaceutical composition is administered to the subject in an amount and for a time sufficient to deliver the immunomodulatory protein or polypeptide, the fusion polypeptide or protein, the conjugate, the immunogenic peptide or protein, the antibody, the polynucleotide, the vector, the host cell, the carrier, the vaccine composition, or the pharmaceutical composition to the subject.

### 5.13.2 Methods of Stimulating the IL-10 Pathway

In one aspect, provided herein are methods of stimulating the IL-10 pathway in a subject in need thereof, the method comprising administering to the subject (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12); to thereby stimulate the IL-10 pathway in the subject. In some embodiments, the immunomodulatory protein or polypeptide, the fusion polypeptide or protein, the conjugate, the immunogenic peptide or protein, the antibody, the polynucleotide, the vector, the host cell, the carrier, the vaccine composition, or the pharmaceutical composition is administered to the subject the in an amount and for a time sufficient to stimulate the IL-10 pathway in the subject.

Stimulation of the IL-10 pathway in a subject can be measured by methods known to those of skill in the art. For example, it is known that in vitro IL-10 inhibits endotoxin (e.g., Escherichia coli lipopolysaccharide (LPS) induced monocyte production of pro-inflammatory cytokines, such as IL-1β, TNFα, IL-6, and IL-8. As such, a cell-based ex vivo stimulation assay may be utilized to assess activation of the IL-10 pathway in a subject. For example, a whole blood sample can be obtained from the subject prior to and following administration of the immunomodulatory protein or polypeptide, the fusion polypeptide or protein, the polynucleotide, the vector, the host cell, the carrier, vaccine composition, or the pharmaceutical composition (e.g., samples can be obtained at multiple time points following administration). The cells in the whole blood sample can be cultured and stimulated with an endotoxin (e.g., Escherichia coli LPS) and the level of one or more pro-inflammatory cytokines known to be downregulated by the IL-10 pathway (e.g., TNFα, IL-1β) assessed. Cytokine expression can be assessed using methods know in the art, e.g., an enzyme-linked immunosorbent assay (ELISA), radioimmunoassay (RAI). See, e.g., Chernoff, A E et al. “A randomized, controlled trial of IL-10 in humans. Inhibition of inflammatory cytokine production and immune responses.” Journal of immunology (Baltimore, Md.: 1950) vol. 154, 10 (1995): 5492-9; and Huhn, R D et al. “Pharmacodynamics of subcutaneous recombinant human interleukin-10 in healthy volunteers.” Clinical pharmacology and therapeutics vol. 62, 2 (1997): 171-80. Doi:10.1016/S0009-9236(97)₉₀₀₆₅-5, the entire contents of each of which are incorporated by reference herein.

### 5.13.3 Methods of Treating or Preventing an IL-10 Responsive Disease

In one aspect, provided herein are methods of treating, ameliorating, or preventing an IL-10 responsive disease in a subject in need thereof, the method comprising administering to the subject (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12); to thereby treat, ameliorate, or prevent the IL-10 responsive disease. In some embodiments, the immunomodulatory protein or polypeptide, the fusion polypeptide or protein, the conjugate, the immunogenic peptide or protein, the antibody, the polynucleotide, the vector, the host cell, the carrier, the vaccine composition, or the pharmaceutical composition is administered to the subject the in an amount and for a time sufficient to treat, ameliorate, or prevent the IL-10 response disease in the subject.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in the treatment, amelioration, or prevention the IL-10 response disease in the subject.

In one aspect, provided herein are uses of (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for the treatment, amelioration, or prevention the IL-10 response disease in the subject.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in the manufacture of a medicament for the treatment, amelioration, or prevention the IL-10 response disease in the subject.

In some embodiments, the IL-10 responsive disease is a pro-inflammatory disease, an autoimmune disease, or a metabolic inflammatory disease. In some embodiments, the IL-10 responsive disease is an autoimmune disease, e.g., an inflammatory bowel disease (e.g., ulcerative colitis, Crohn's disease), rheumatoid arthritis, multiple sclerosis, psoriasis, or autoimmune hepatitis. In some embodiments, the IL-10 responsive disease is a pro-inflammatory disease, e.g., cytokine storm, arthritis (e.g., osteoarthritis), or graft-versus host disease (e.g., in the context of organ, tissue, or cell transplant). In some embodiments, the IL-10 responsive disease is an allergy, e.g., allergic asthma or a food allergy. In some embodiments, the IL-10 responsive disease is an infectious disease, e.g., a viral infection, e.g., hepatitis C. In some embodiments, the IL-10 responsive disease is a fibrotic disease, e.g., pulmonary fibrosis, liver fibrosis, pancreatitis, chronic kidney disease, cardiovascular fibrosis, or myocardial infarction. In some embodiments, the IL-10 responsive disease is an arterial disease, e.g., atherosclerosis. In some embodiments, the IL-10 responsive disease is a disease associated with pregnancy, e.g., pre-eclampsia or inflammation-driven fetal death. In some embodiments, the IL-10 responsive disease is a nervous system disease, e.g., a neurodegenerative disease (e.g., Parkinson's disease), neuropathic nerve pain, or peripheral nerve pain. In some embodiments, the IL-10 responsive disease is cancer. In some embodiments, the IL-10 responsive disease is metabolic inflammatory syndrome. In some embodiments, the IL-10 responsive disease is insulin resistance, atherosclerosis, or type 2 diabetes. In some embodiments, the subject has one or more of insulin resistance, atherosclerosis, or type 2 diabetes. In some embodiments, the metabolic inflammatory syndrome comprises one or more of insulin resistance, atherosclerosis, or type 2 diabetes. In some embodiments, the IL-10 responsive disease is a liver disease. In some embodiments, the liver disease is fatty liver, liver inflammation, nonalcoholic steatohepatitis (NASH), or nonalcoholic fatty liver disease (NAFLD).

### 5.13.4 Methods of Suppressing or Preventing an Immune Response

In one aspect, provided herein are methods of suppressing or preventing an immune response in a subject in need thereof, the method comprising administering to the subject (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12), to thereby suppress or prevent an immune response in the subject. In some embodiments, the immunomodulatory protein or polypeptide, the fusion polypeptide or protein, the conjugate, the immunogenic peptide or protein, the antibody, the polynucleotide, the vector, the host cell, the carrier, or the pharmaceutical composition is administered to the subject the in an amount and for a time sufficient to suppress or prevent an immune response in the subject.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in suppression or prevention of an immune response in the subject.

In one aspect, provided herein are uses of (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for the suppression or prevention of an immune response in the subject.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in the manufacture of a medicament for the suppression or prevention of an immune response in the subject.

In some embodiments, the level of one or more pro-inflammatory cytokine (e.g., IFNγ, TNFα, IL-17A) is suppressed (e.g., as measured by an assay described herein). In some embodiments, the level of one or more of IFNγ, TNFα, and/or IL-17A is suppressed (e.g., as measured by an assay described herein). In some embodiments, the level of immunoglobulin (e.g., IgA, IgG, IgE, IgM, IgD) is suppressed (e.g., as measured by an assay described herein). In some embodiments, the level of immunogen specific immunoglobulin (e.g., IgA, IgG, IgE, IgM, IgD) is suppressed (e.g., as measured by an assay described herein). In some embodiments, the level of IgE (e.g., immunogen specific IgE, e.g., allergen specific IgE) is suppressed (e.g., as measured by an assay described herein).

The level of a protein (e.g., a pro-inflammatory cytokine, an Ig (e.g., IgE)) can be measured utilizing standard methods known in the art. For example, standard in vitro protein (e.g., cytokine, Ig (e.g., IgE)) quantification assays (e.g., commercially available assays) can be utilized to determine the level of a given protein (e.g., a cytokine, Ig (e.g., IgE)) in a sample (e.g., blood, serum, cell, tissue) from a subject (e.g., a human subject). For example, multiplex and single analyte ELISAs (as described herein) can be utilized, see, e.g., Liu, C., Chu, D., Kalantar-Zadeh, K., George, J., Young, H. A., Liu, G., Cytokines: From Clinical Significance to Quantification. Adv. Sci. 2021, 8, 2004433. https://doi.org/10.1002/advs.202004433; and Liu BM, Martins TB, Peterson LK, Hill HR. Clinical significance of measuring serum cytokine levels as inflammatory biomarkers in adult and pediatric COVID-19 cases: A review. Cytokine. 2021; 142:155478. doi:10.1016/j.cyto.2021.155478, Ansotegui I J, Melioli G, Canonica G W, et al. IgE allergy diagnostics and other relevant tests in allergy, a World Allergy Organization position paper [published correction appears in World Allergy Organ J. 2021 Jun. 17; 14(7):100557]. World Allergy Organ J. 2020; 13(2):100080. Published 2020 Feb. 25. doi:10.1016/j.waojou.2019.100080; Liang Y, Ganley-Leal L M. A simple method for measuring human cell-bound IgE levels in whole blood. J Immunol Methods. 2009; 343(2):134-139. doi:10.1016/j.jim.2009.01.012; the entire contents of each of which is incorporated by reference herein for all purposes.

In some embodiments, the subject has an IL-10 responsive disease. In some embodiments, the IL-10 responsive disease is a pro-inflammatory disease, an autoimmune disease, or a metabolic inflammatory disease. In some embodiments, the IL-10 responsive disease is an autoimmune disease, e.g., an inflammatory bowel disease (e.g., ulcerative colitis, Crohn's disease), rheumatoid arthritis, multiple sclerosis, psoriasis, or autoimmune hepatitis. In some embodiments, the IL-10 responsive disease is a pro-inflammatory disease, e.g., cytokine storm, arthritis (e.g., osteoarthritis), or graft-versus host disease (e.g., in the context of organ, tissue, or cell transplant). In some embodiments, the IL-10 responsive disease is an allergy, e.g., allergic asthma or a food allergy (e.g., an allergy to any of: milk, eggs, fish, shellfish, tree nuts, peanuts, wheat, or soybeans). In some embodiments, the IL-10 responsive disease is an infectious disease, e.g., a viral infection, e.g., hepatitis C. In some embodiments, the IL-10 responsive disease is a fibrotic disease, e.g., pulmonary fibrosis, liver fibrosis, pancreatitis, chronic kidney disease, cardiovascular fibrosis, or myocardial infarction. In some embodiments, the IL-10 responsive disease is an arterial disease, e.g., atherosclerosis. In some embodiments, the IL-10 responsive disease is a disease associated with pregnancy, e.g., pre-eclampsia or inflammation-driven fetal death. In some embodiments, the IL-10 responsive disease is a nervous system disease, e.g., a neurodegenerative disease (e.g., Parkinson's disease), neuropathic nerve pain, or peripheral nerve pain. In some embodiments, the IL-10 responsive disease is cancer. In some embodiments, the IL-10 responsive disease is metabolic inflammatory syndrome. In some embodiments, the IL-10 responsive disease is insulin resistance, atherosclerosis, or type 2 diabetes. In some embodiments, the subject has one or more of insulin resistance, atherosclerosis, or type 2 diabetes. In some embodiments, the metabolic inflammatory syndrome comprises one or more of insulin resistance, atherosclerosis, or type 2 diabetes. In some embodiments, the subject has received an allogenic cell, tissue, or organ transplant. In some embodiments, the subject has graft versus host disease. In some embodiments, the graft versus host disease is acute or chronic. In some embodiments, the IL-10 responsive disease is a liver disease. In some embodiments, the liver disease is fatty liver, liver inflammation, nonalcoholic steatohepatitis (NASH), or nonalcoholic fatty liver disease (NAFLD).

In some embodiments, the IL-10 responsive disease is refractory to one or more immunosuppressive agent (e.g., the one or more steroid (e.g., corticosteroid). In some embodiments, the IL-10 responsive disease is refractory to the standard of care therapy. In some embodiments, the immunomodulatory protein or polypeptide, the fusion polypeptide or protein, the immunogenic peptide or protein, the antibody, the polynucleotide, the vector, the host cell, the carrier, or the pharmaceutical composition is administered in combination with one or more immunosuppressive agent (e.g., one or more steroid (e.g., corticosteroid)). In some embodiments, the immunosuppressive agent is a protein (e.g., an antibody, fusion protein) or a small molecule. In some embodiments, the immunosuppressive agent is a steroid (e.g., a corticosteroid). In some embodiments, prior to the administering of the immunomodulatory protein or polypeptide, the fusion polypeptide or protein, the immunogenic peptide or protein, the antibody, the polynucleotide, the vector, the host cell, the carrier, the vaccine composition, or the pharmaceutical composition to the subject, the subject has been administered one or more immunosuppressive agent (e.g., one or more steroid (e.g., corticosteroid)). In some embodiments, the immunosuppressive agent is a protein (e.g., an antibody, fusion protein) or a small molecule. In some embodiments, the immunosuppressive agent is a steroid (e.g., a corticosteroid).

### 5.13.5 Methods of Inducing or Enhancing Tolerance to an Immunogen

In one aspect, provided herein are methods of inducing or enhancing tolerance to an immunogen in a subject in need thereof, the method comprising administering to the subject (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12); to thereby induce or enhance tolerance to an immunogen in the subject. In some embodiments, the immunomodulatory protein or polypeptide, the fusion polypeptide or protein, the conjugate, the immunogenic peptide or protein, the antibody, the polynucleotide, the vector, the host cell, the carrier, or the pharmaceutical composition is administered to the subject the in an amount and for a time sufficient to induce or enhance tolerance to an immunogen in the subject.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in the induction or enhancement of tolerance to an immunogen in the subject.

In one aspect, provided herein are uses of (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for the induction or enhancement of tolerance to an immunogen in the subject.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in the manufacture of a medicament for the induction or enhancement of tolerance to an immunogen in the subject.

In some embodiments, the level of one or more pro-inflammatory cytokine (e.g., IFNγ, TNFα, IL-17A) is suppressed (e.g., as measured by an assay described herein). In some embodiments, the level of one or more of IFNγ, TNFα, and/or IL-17A is suppressed (e.g., as measured by an assay described herein). In some embodiments, the level of an immunoglobulin (e.g., IgA, IgG, IgE, IgM, IgD) is suppressed (e.g., as measured by an assay described herein). In some embodiments, the level of immunogen specific immunoglobulin (e.g., IgA, IgG, IgE, IgM, IgD) is suppressed (e.g., as measured by an assay described herein). In some embodiments, the level of IgE (e.g., immunogen specific IgE, e.g., allergen specific IgE) is suppressed (e.g., as measured by an assay described herein). The level of a pro-inflammatory cytokine or Ig (e.g., IgE) can be measured utilizing standard methods known in the art and described herein. See, e.g., § 5.13.4.

In some embodiments, the subject has an IL-10 responsive disease. In some embodiments, the IL-10 responsive disease is a pro-inflammatory disease, an autoimmune disease, or a metabolic inflammatory disease. In some embodiments, the IL-10 responsive disease is an autoimmune disease, e.g., an inflammatory bowel disease (e.g., ulcerative colitis, Crohn's disease), rheumatoid arthritis, multiple sclerosis, psoriasis, or autoimmune hepatitis. In some embodiments, the IL-10 responsive disease is a pro-inflammatory disease, e.g., cytokine storm, arthritis (e.g., osteoarthritis), or graft-versus host disease (e.g., in the context of organ, tissue, or cell transplant). In some embodiments, the IL-10 responsive disease is an allergy, e.g., allergic asthma or a food allergy. In some embodiments, the IL-10 responsive disease is an infectious disease, e.g., a viral infection, e.g., hepatitis C. In some embodiments, the IL-10 responsive disease is a fibrotic disease, e.g., pulmonary fibrosis, liver fibrosis, pancreatitis, chronic kidney disease, cardiovascular fibrosis, or myocardial infarction. In some embodiments, the IL-10 responsive disease is an arterial disease, e.g., atherosclerosis. In some embodiments, the IL-10 responsive disease is a disease associated with pregnancy, e.g., pre-eclampsia or inflammation-driven fetal death. In some embodiments, the IL-10 responsive disease is a nervous system disease, e.g., a neurodegenerative disease (e.g., Parkinson's disease), neuropathic nerve pain, or peripheral nerve pain. In some embodiments, the IL-10 responsive disease is cancer. In some embodiments, the IL-10 responsive disease is metabolic inflammatory syndrome. In some embodiments, the IL-10 responsive disease is insulin resistance, atherosclerosis, or type 2 diabetes. In some embodiments, the subject has one or more of insulin resistance, atherosclerosis, or type 2 diabetes. In some embodiments, the metabolic inflammatory syndrome comprises one or more of insulin resistance, atherosclerosis, or type 2 diabetes.

In some embodiments, the IL-10 responsive disease is refractory to one or more immunosuppressive agent (e.g., the one or more steroid (e.g., corticosteroid). In some embodiments, the IL-10 responsive disease is refractory to the standard of care therapy. In some embodiments, the immunomodulatory protein or polypeptide, the fusion polypeptide or protein, the immunogenic peptide or protein, the antibody, the polynucleotide, the vector, the host cell, the carrier, or the pharmaceutical composition is administered in combination with one or more immunosuppressive agent (e.g., one or more steroid (e.g., corticosteroid)). In some embodiments, the immunosuppressive agent is a protein (e.g., an antibody, fusion protein) or a small molecule. In some embodiments, the immunosuppressive agent is a steroid (e.g., a corticosteroid). In some embodiments, prior to the administering of the immunomodulatory protein or polypeptide, the fusion polypeptide or protein, the immunogenic peptide or protein, the antibody, the polynucleotide, the vector, the host cell, the carrier, or the pharmaceutical composition to the subject, the subject has been administered one or more immunosuppressive agent (e.g., one or more steroid (e.g., corticosteroid)). In some embodiments, the immunosuppressive agent is a protein (e.g., an antibody, fusion protein) or a small molecule. In some embodiments, the immunosuppressive agent is a steroid (e.g., a corticosteroid).

In some embodiments, the immunogen is an allergen. In some embodiments, the subject has been administered an allergen-specific therapy (e.g., as described herein (e.g., the subject has been administered at least 1 (e.g., at least 2, 3, 4, 5, 6, 7, 8, 9, 10, or more doses (e.g., escalating doses) of an allergen)). In some embodiments, the allergen-specific therapy induced a degree of tolerance to the immunogen in the subject. In some embodiments, the administration of the immunomodulatory protein or polypeptide, the fusion polypeptide or protein, the immunogenic peptide or protein, the antibody, the polynucleotide, the vector, the host cell, the carrier, or the pharmaceutical composition enhances the degree of tolerance induced by the immunogen-specific therapy.

In some embodiments, the subject has received an allogenic or autologous cell, tissue, or organ transplant. In some embodiments, the immunogen is part of an allogenic or autologous cell, tissue, or organ. In some embodiments, the subject has graft versus host disease. In some embodiments, the graft versus host disease is acute or chronic.

### 5.13.6 Methods of Reducing IgE Expression

In one aspect, provided herein are methods of reducing IgE expression in a subject in need thereof, the method comprising administering to the subject (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12); to thereby reduce IgE expression in the subject. In some embodiments, the immunomodulatory protein or polypeptide, the fusion polypeptide or protein, the conjugate, the immunogenic peptide or protein, the antibody, the polynucleotide, the vector, the host cell, the carrier, or the pharmaceutical composition is administered to the subject the in an amount and for a time sufficient to reduce IgE expression in the subject. The level of IgE can be measured utilizing standard methods known in the art and described herein. See, e.g., § 5.13.4.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in the reduction of IgE expression in the subject.

In one aspect, provided herein are uses of (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for the reduction of IgE expression in the subject.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in the manufacture of a medicament for the reduction of IgE expression in the subject.

In some embodiments, the subject has an IL-10 responsive disease. In some embodiments, the IL-10 responsive disease is a pro-inflammatory disease, an autoimmune disease, or a metabolic inflammatory disease. In some embodiments, the IL-10 responsive disease is an allergy, e.g., allergic asthma or a food allergy. In some embodiments, the immunogen the immunogen is an allergen. In some embodiments, the subject has been administered an allergen-specific therapy (e.g., as described herein (e.g., the subject has been administered at least 1 (e.g., at least 2, 3, 4, 5, 6, 7, 8, 9, 10, or more doses (e.g., escalating doses) of an allergen)). In some embodiments, the allergen-specific therapy induced a degree of tolerance to the immunogen in the subject. In some embodiments, the administration of the hIL-10R binding protein enhances the degree of tolerance induced by the immunogen-specific therapy.

In some embodiments, the IL-10 responsive disease is refractory to one or more immunosuppressive agent (e.g., the one or more steroid (e.g., corticosteroid). In some embodiments, the IL-10 responsive disease is refractory to the standard of care therapy. In some embodiments, the immunomodulatory protein or polypeptide, the fusion polypeptide or protein, the immunogenic peptide or protein, the antibody, the polynucleotide, the vector, the host cell, the carrier, or the pharmaceutical composition is administered in combination with one or more immunosuppressive agent (e.g., one or more steroid (e.g., corticosteroid)). In some embodiments, the immunosuppressive agent is a protein (e.g., an antibody, fusion protein) or a small molecule. In some embodiments, the immunosuppressive agent is a steroid (e.g., a corticosteroid). In some embodiments, prior to the administering of the immunomodulatory protein or polypeptide, the fusion polypeptide or protein, the immunogenic peptide or protein, the antibody, the polynucleotide, the vector, the host cell, the carrier, or the pharmaceutical composition to the subject, the subject has been administered one or more immunosuppressive agent (e.g., one or more steroid (e.g., corticosteroid)). In some embodiments, the immunosuppressive agent is a protein (e.g., an antibody, fusion protein) or a small molecule. In some embodiments, the immunosuppressive agent is a steroid (e.g., a corticosteroid).

### 5.13.7 Methods of Inducing or Enhancing an Immune Response

In one aspect, provided herein are vaccines and methods of inducing or enhancing an immune response in a subject in need thereof, comprising administering to the subject (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12); to thereby induce or enhance an immune response in the subject. In some embodiments, the immunomodulatory protein or polypeptide, the fusion polypeptide or protein, the conjugate, the immunogenic peptide or protein, the antibody, the polynucleotide, the vector, the host cell, the carrier, or the pharmaceutical composition is administered to the subject the in an amount and for a time sufficient to induce or enhance an immune response in the subject.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in the induction or enhancement of an immune response in the subject.

In one aspect, provided herein are uses of (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for the induction or enhancement of an immune response in the subject.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in the manufacture of a medicament for the induction or enhancement of an immune response in the subject.

In some embodiments, the method comprises administering the immunogenic peptide or protein described herein (or a nucleic acid molecule encoding the same) or a fusion, conjugate, vector, host cell, carrier, or pharmaceutical composition comprising the same. In some embodiments, wherein the immune response is immunogen specific.

Enhancing an immune response (e.g., an immunogen specific immune responses) includes e.g., increasing the duration of an immune response, increasing the magnitude of an immune response, and/or changing the nature of the immune response.

An immune response in a subject can be measured by common methods known to those of skill in the art. For example, serological assays can be employed to detect a humoral response by measuring titers of anti-immunogen (e.g., anti-immunomodulatory protein) IgG antibodies post administration. For example, an enzyme-linked immunosorbent assay (ELISA) is a standard laboratory test for detecting and quantifying antibodies well known to the person of skill in the art. Generally, blood is collected from a consenting subject, centrifuged, and the serum isolated according to standard techniques. The recombinant target immunogen (e.g., immunomodulatory polypeptide or protein) is immobilized in microplate wells. The microplate is blocked by through the incubation with an irrelevant immunogen (e.g., bovine serum albumin). The serum sample from the subject is prepared and added to the blocked wells to allow for binding of an immunogen specific antibodies to the immobilized immunogen. The bound antibodies are detected using a secondary tagged antibody that binds to the previously bound antibodies (e.g., anti-human IgG antibodies). See, e.g., Yannick G. et al. Humoral Responses and Serological Assays in SARS-CoV-2 Infections, Frontiers in Immunology, Vol 11 (2020) 10.3389/fimmu.2020.610688; Forgacs David et al., SARS-CoV-2 mRNA Vaccines Elicit Different Responses in Immunologically Naïve and Pre-Immune Humans; Front. Immunol., Vol 12 (27 Sep. 2021) https://doi.org/10.3389/fimmu.2021.728021, the entire contents of each of which is incorporated by reference herein for all purposes.

Cell based assays can also be utilized to detect a cell based immune response (e.g., T cell immune response). For example, immunogen specific T cells (e.g., CD4+ or CD8+ T cells) can be measured using an enzyme-linked immunospot (ELISpot), an intracellular cytokine staining (ICS) assay, or an activation induced marker assay (AIM). Each of these assays is commonly used to detect cell based (e.g., T cell) immune responses to vaccines and well known to the person of ordinary skill in the art. See, e.g., Bowyer, Georgina et al. “Activation-induced Markers Detect Vaccine-Specific CD4⁺ T Cell Responses Not Measured by Assays Conventionally Used in Clinical Trials.” Vaccines vol. 6, 3 50. 31 Jul. 2018, doi:10.3390/vaccines6030050, the entire contents of which is incorporated by reference herein for all purposes.

### 5.13.8 Methods of Preventing or Treating a Viral Infection

In one aspect, provided herein are methods of preventing, ameliorating, or treating a viral infection in a subject, comprising administering to the subject (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12); to thereby prevent, ameliorate, or treat the viral infection in the subject. In some embodiments, the immunomodulatory protein or polypeptide, the fusion polypeptide or protein, the conjugate, the immunogenic peptide or protein, the antibody, the polynucleotide, the vector, the host cell, the carrier, or the pharmaceutical composition is administered to the subject the in an amount and for a time sufficient prevent, ameliorate, or treat the viral infection in the subject.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in prevention, amelioration, or treatment of the viral infection in the subject.

In one aspect, provided herein are uses of (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for the in prevention, amelioration, or treatment of the viral infection in the subject.

In one aspect, provided herein is (i) an immunomodulatory protein or polypeptide described herein (e.g., described in § 5.2); (ii) a fusion polypeptide or protein described herein (e.g., described in § 5.3); (iii) a conjugate described herein (e.g., described in § 5.3); (iv) an immunogenic peptide or protein described herein (e.g., described in § 5.4; (v) an antibody described herein (e.g., described in § 5.5); (vi) a polynucleotide described herein (e.g., described in § 5.6); (vii) a vector described herein (e.g., described in § 5.7); (viii) a cell described herein (e.g., described in § 5.8); (ix) a carrier described herein (e.g., described in § 5.10); (x) a vaccine composition described herein (e.g., described in § 5.4); or (xi) a pharmaceutical composition described herein (e.g., described in § 5.12) for use in the manufacture of a medicament for the in prevention, amelioration, or treatment of the viral infection in the subject.

In some embodiments, the pharmaceutical composition is administered to the subject as a prophylactic treatment. In some embodiments, the pharmaceutical composition is administered as a treatment after the onset of at least one symptom of an infection or disease. The pharmaceutical compositions described herein may be administered as a prime and/or a boost in a homologous or heterologous prime-boost regimen.

In some embodiments, the pharmaceutical composition prevents infection with the virus, reduces the likelihood or severity of infection with the virus, reduces the likelihood of developing an established infection after challenge with the virus, prevents or delays onset one or more symptoms of a disease associated with the viral infection, reduces in frequency and/or severity one or more symptoms of the disease, and/or reduces the risk of hospitalization or death associated with the disease.

In one aspect, provided herein are methods of treating a viral infection in a subject, the method comprising (a) receiving testing results that determined the presence of an immunomodulatory protein described herein (or a fragment or variant thereof) or a polynucleotide encoding the immunomodulatory protein described herein (or a fragment or variant thereof) in a sample from the subject, (b) diagnosing the subject as having the viral infection, and (c) administering a therapeutic agent to treat the viral infection.

Exemplary viral infections include, but are not limited to, infections with a virus of family Herpesviridae (a herpes virus) (e.g., a betaherpesvirus (e.g., a cytomegalovirus, a muromegalovirus, a proboscivirus, a quwivirus, a roseolovirus); a gammaherpesvirus (e.g., a bossavirus, a lymphocryptovirus, a macavirus, a manticavirus, a patagivirus, a percavirus, a rhadinovirus); an alphaherpesvirus (e.g., an iltovirus a mardivirus, a scutavirus, a simplexvirus, a varicellovirus). In some embodiments, the virus is a human herpesviridae virus. in some embodiments, the virus is a cytomegalovirus (e.g., a human cytomegalovirus). In some embodiments, the virus is an Epstein-Barr virus (e.g., a human Epstein-Barr virus).

In some embodiments, the virus a virus of the family Poxviridae (a poxvirus) (e.g., chordopoxvirus (e.g., a parapoxvirus (e.g., an ORF virus), a pseudocowpox virus)). In some embodiments, the virus is a chordopoxvirus (e.g., a human chordopoxvirus). In some embodiments, the virus is a parapoxvirus (e.g., a human parapoxvirus) (e.g., an ORF virus (e.g., a human ORF virus)). In some embodiments, the virus is an ORF virus (e.g., a human ORF virus). In some embodiments, the virus is a pseudocowpox virus (e.g., a human pseudocowpox virus).

An appropriate therapeutic agent for treatment of the viral infection can be selected by a person of ordinary skill in art according to standard practices. For example, the antiviral agent may be an attachment inhibitor, post-attachment inhibitor, fusion inhibitor, entry inhibitor, uncoating inhibitor, protease inhibitor, polymerase inhibitor, nucleotide reverse transcriptase inhibitor, nucleoside reverse transcriptase inhibitor, non-nucleoside reverse transcriptase inhibitor, and/or integrase inhibitor. In some embodiments, the antiviral agent is a capsid inhibitor, a secretion inhibitor, a microRNA, an antisense RNA agent, an RNAi agent, or other agent designed to inhibit viral RNA. In some embodiments, the antiviral agent is a small molecule, a lipid, a polynucleotide, a peptide, or an antibody. In some embodiments, the antiviral agent is a small molecule antiviral agent. In some embodiments, the antiviral agent is a nucleoside analog, a peptide, or a nonribosomal peptide.

In some embodiments, the antiviral agent targets a DNA virus. In some embodiments, the antiviral agent targets an RNA virus. In some embodiments, the antiviral agent has broad spectrum activity against numerous types of viruses, e.g., and is capable of targeting both a DNA virus and an RNA virus.

The antiviral agent may function by targeting a specific viral function, such as inhibiting a viral nucleic acid polymerase, viral protease, viral integrase, or viral neuraminidase. In another embodiment, the antiviral agent functions by targeting a host cell function required for successful viral replication, such as viral entry into a host cell, nucleic acid synthesis, protein synthesis, viral capsid assembly, or viral exit from the host cell.

Exemplary antiviral agents include, but are not limited to, abacavir, acyclovir, amantadine, ampligen, amprenavir, umifenovir, atripia, alazanavir, biktarvy, baloxavir marboxil, bulevirtide, boceprevir, chloroquine, cidofovir, cobicistat, combivir, daclatasvir, darunavir, delavirdine, descovy, didanosine, docosanol, dolutegravir, doravirine, edoxudine, efavirenz, elvitegravir, emtricitabine, enfuvirtide, entecavir, etravirine, ensitrelvir, famciclovir, favipirvir, fomivirsen, fosamprenavir, foscamet, ganciclovir, hydroxychloroquine, ibacitabine, ibalizumab, idoxuridine, imiquimod, imunovir, ivermectin, indinavir, lamivudine, letermovir, lopinavir, loviride, maraviroc, methisazone, moroxydine, nelfinavir, nexavir, nitazoxanide, norvir, oseltamivir, penciclovir, peramvir, pleconaril, pieconaril, raltegravir, rilpivirine, ribavirin, remdesivir, ritonavir, saquinavir, sofosbuvir, taribavirin, telaprevir, tenofovir, telbivudine, trizivir, tipranavir, truvada, tromantadine, trifluridine, vidarabine, umifenovir, umifenovir, valaciclovir, vicriviroc, vidarabine, zalcitabine, zanamivir, and zicovidinr.

### 5.13.9 Diagnostic Methods

Further provided herein are diagnostic methods. Some of the methods described herein (e.g., diagnostic methods (see, e.g., § 5.13.9) and therapeutic methods (see, e.g., § 5.13.8)) utilize a sample from a subject. A suitable sample source, size, etc. can be determined by a person of ordinary skill in the art in accordance with use in the selected method. Exemplary subject samples include, but are not limited to, blood, plasma, cell, tissue, saliva sample, and nasal swab. Other samples include, but are not limited to, semen, sputum, mucous, sweat, urine, and feces. A diagnostic method herein may be an in vitro method.

In one aspect, provided herein are methods of determining the presence of a virus in a subject, the method comprising (a) obtaining a sample from a subject, and (b) determining the presence or absence of an immunomodulatory protein or polypeptide described herein (or a fragment or variant thereof) or a polynucleotide encoding the immunomodulatory protein or polypeptide described herein (or a fragment or variant thereof) in the sample.

In one aspect, provided herein are methods of diagnosing a viral infection in a subject, the method comprising (a) obtaining a sample from a subject, (b) determining the presence or absence of an immunomodulatory protein or polypeptide described herein (or a fragment or variant thereof) or a polynucleotide encoding the immunomodulatory protein or polypeptide described herein (or a fragment or variant thereof) in the sample, and (c) diagnosing the subject as having the viral infection if the immunomodulatory protein or polypeptide described herein (or a fragment or variant thereof) or the polynucleotide encoding the immunomodulatory protein or polypeptide described herein (or a fragment or variant thereof), is determined to be present in the sample in step (b).

In some embodiments, an antibody described herein (e.g., an antibody that specifically binds an immunomodulatory protein or polypeptide described herein) is utilized in any one of the diagnostic methods in order to determine the presence or absence of an immunomodulatory protein described herein (or a fragment or variant thereof). In some embodiments, the antibody (e.g., an antibody that specifically binds an immunomodulatory protein or polypeptide described herein) is labeled with a tag (e.g., a fluorescent tag) to aid in detection.

Exemplary viral infections include, but are not limited to, infections with a virus of family Herpesviridae (a herpes virus) (e.g., a betaherpesvirus (e.g., a cytomegalovirus, a muromegalovirus, a proboscivirus, a quwivirus, a roseolovirus); a gammaherpesvirus (e.g., a bossavirus, a lymphocryptovirus, a macavirus, a manticavirus, a patagivirus, a percavirus, a rhadinovirus); an alphaherpesvirus (e.g., an iltovirus a mardivirus, a scutavirus, a simplexvirus, a varicellovirus). In some embodiments, the virus is a human herpesviridae virus. in some embodiments, the virus is a cytomegalovirus (e.g., a human cytomegalovirus). In some embodiments, the virus is an Epstein-Barr virus (e.g., a human Epstein-Barr virus).

In some embodiments, the virus a virus of the family Poxviridae (a poxvirus) (e.g., chordopoxvirus (e.g., a parapoxvirus (e.g., an ORF virus), a pseudocowpox virus)). In some embodiments, the virus is a chordopoxvirus (e.g., a human chordopoxvirus). In some embodiments, the virus is a parapoxvirus (e.g., a human parapoxvirus) (e.g., an ORF virus (e.g., a human ORF virus)). In some embodiments, the virus is an ORF virus (e.g., a human ORF virus). In some embodiments, the virus is a pseudocowpox virus (e.g., a human pseudocowpox virus).

### 5.14 Kits

In a one aspect, provided herein are kits comprising an immunomodulatory protein or polypeptide described herein (see, e.g., § 5.2), a fusion protein or polypeptide described herein (see, e.g., § 5.3), a conjugate described herein (see, e.g., § 5.3), an immunogenic peptide or protein described herein (see, e.g., § 5.4), an antibody described herein (see, e.g., § 5.9), a polynucleotide described herein (see, e.g., § 5.6), a vector described herein (see, e.g., § 5.7), a cell described herein (see, e.g., § 5.8), a carrier described herein (see, e.g., § 5.10), a vaccine composition described herein (see, e.g., § 5.4), or a pharmaceutical composition described herein (see, e.g., § 5.12). In addition, the kit may comprise a liquid vehicle for solubilizing or diluting, and/or technical instructions. The technical instructions of the kit may contain information about administration and dosage and subject groups.

In some embodiments, the immunomodulatory protein or polypeptide described herein, the fusion polypeptide or protein described herein, the conjugate described herein, the immunogenic peptide or protein described herein, the antibody described herein, the polynucleotide described herein, the vector described herein, the host cell described herein, the carrier described herein, the vaccine composition described herein, or the pharmaceutical composition described herein is provided in a separate part of the kit, wherein the immunomodulatory protein or polypeptide described herein, the fusion polypeptide or protein described herein, the conjugate, the immunogenic peptide or protein described herein, the antibody described herein, the polynucleotide described herein, the vector described herein, the host cell described herein, the carrier described herein, the vaccine composition described herein, or the pharmaceutical composition described herein is optionally lyophilized, spray-dried, or spray-freeze dried. The kit may further contain as a part a vehicle (e.g., buffer solution) for solubilizing the dried or lyophilized immunomodulatory protein or polypeptide described herein, fusion polypeptide or protein described herein, the conjugate, immunogenic peptide or protein described herein, antibody described herein, polynucleotide described herein, vector described herein, host cell described herein, carrier described herein, the vaccine composition described herein, or pharmaceutical composition described herein.

In some embodiments, the kit comprises a single dose container. In some embodiments, the kit comprises a multi-dose container. In some embodiments, the kit comprises an administration device (e.g., an injector for intradermal injection or a syringe for intramuscular injection). In some embodiments, the kit comprises adjuvant in a separate container. The kit may further contain technical instructions for mixing the adjuvant prior to administration or for co-administration.

In some embodiments, the kit comprises an antibody described herein that specifically binds an immunomodulatory protein or polypeptide described herein. In some embodiments, the antibody (e.g., an antibody that specifically binds an immunomodulatory protein or polypeptide described herein) is labeled with a tag (e.g., a fluorescent tag) to aid in detection. In some embodiments, the comprises one or more reagent (e.g., a buffer) for a sample described herein. In some embodiments, the kit is for use in a method of determining the presence of a virus in a subject or a method of diagnosing a subject with a viral infection.

Any of the kits described herein may be used in any of the methods described herein (see, e.g., § 5.13).

### 6. EXAMPLES

### 6.1 Example 1. Immunomodulatory Protein Identification and Expression

Immunomodulatory Proteins 1-175 (IMPs 1-175) (SEQ ID NOS: 108-454) were identified by the inventors through a process of searching, screening, and analysis of viral genomes for proteins having the ability to, inter alia, e.g., bind hIL-10R.

Of the 175 IMPs identified, a set of 97 immunomodulatory fusion proteins (IFPs), each comprising one of IMPs 1-12, 21, 26-27, 31-32, 36, 38, 41, 49-50, 53, 55, 58, 62, 67-70, 72-73, 75-82, 84, 86-87, 89, 91, 93, 95, 97-100, 102-103, 106-109, 111-112, 115, 117-118, 120-127, 129-130, 132, 134-136, 138-158 (mature sequences are set forth in SEQ ID NOS: 108-127, 136, 141-142, 146-147, 151, 153, 156, 164-165, 168, 170, 173, 177, 182-185, 187-188, 190-197, 199, 201-202, 204, 206, 208, 210, 212-215, 217-218, 221-224, 226-227, 230, 232-233, 235-242, 244-245, 247, 249-251, 253-273, respectively), including IFPs 1-11 (SEQ ID NOS: 58-68), were generated. Each IFP comprised from N- to C-terminus the hIL-2 signal sequence, an effector function reduced hIgG4 Fc region, a peptide linker, and one IMP identified herein (i.e., one of IMPs 1-12, 21, 26-27, 31-32, 36, 38, 41, 49-50, 53, 55, 58, 62, 67-70, 72-73, 75-82, 84, 86-87, 89, 91, 93, 95, 97-100, 102-103, 106-109, 111-112, 115, 117-118, 120-127, 129-130, 132, 134-136, 138-158 (mature sequences are set forth in SEQ ID NOS: 108-127, 136, 141-142, 146-147, 151, 153, 156, 164-165, 168, 170, 173, 177, 182-185, 187-188, 190-197, 199, 201-202, 204, 206, 208, 210, 212-215, 217-218, 221-224, 226-227, 230, 232-233, 235-242, 244-245, 247, 249-251, 253-273, respectively), including e.g., one of IMPs 1-11 (see SEQ ID NOS: 108-118)). The IFPs were generated using standard methods known in the art. Briefly, a DNA polynucleotide encoding each of the fusion proteins was synthesized and inserted into an expression plasmid. Expi293 cells (Thermo Fisher #A14527) were transfected using the Expi293 expression kit (Thermo Fisher #A14635) according to the manufacturer's protocol. Briefly, Expi293 cells were grown in suspension at 37° C., 8% CO₂ in Expi293 growth medium (Thermo Fisher #A1435101). The cells were counted using a hemocytometer to ensure a density of 2.5-3 million cells per mL, and a viability above 95%, prior to transfection. Transfections were performed in 2.5 ml of cell containing medium (7.5-9 million cells per reaction). 1 μg/ml of plasmid DNA was pre-incubated with Opti-MEM for 5 minutes at room temperature (RT) and ExpiFectamine was pre-incubated with Opti-MEM for 5 minutes at RT. The plasmid mixture was subsequently mixed with the ExpiFectamine mixture and incubated for 10-20 minutes at RT. After incubation, the mixture was added to the Expi293 cells and incubated overnight. On day 1 post-transfection, ExpiFectamine Enhancer 1 and ExpiFectamine Enhancer 2 were added to the cell culture. On day 3 post-transfection, the supernatant was removed and maintained at −20° C., and the cells were discarded. The amino acid sequence of a subset of the generated IFPs, i.e., IFPs 1-11, is set forth in SEQ ID NOS: 58-68 and in Table 7. Likewise, a reference hIL-10 Fc fusion protein in the same format was generated. The amino acid sequence of the reference hIL-10 Fc fusion protein is set forth in SEQ ID NO: 102.

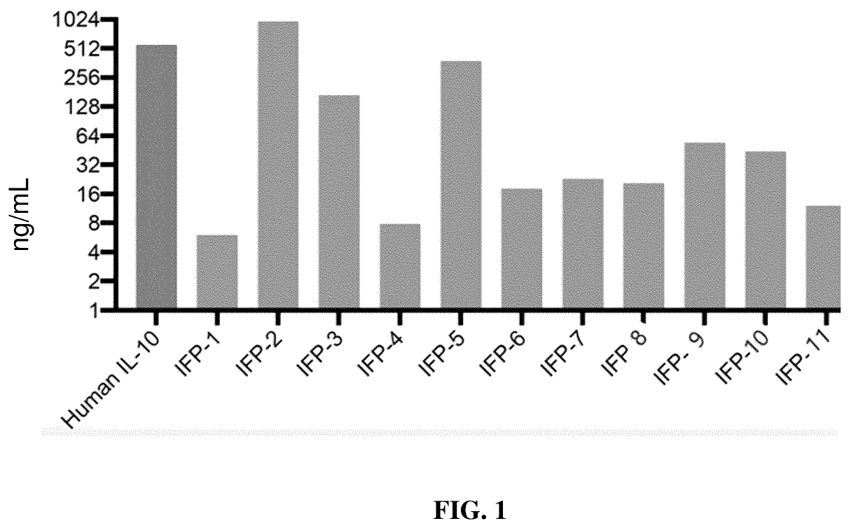

The concentration of each IFP in the supernatant was determined using a hIgG ELISA (Abcam #195215) according to the manufacturer's protocol. Briefly, the transfection supernatant was incubated with anti-IgG antibody for 40 minutes with shaking, subsequently washed twice in wash buffer PT. The supernatants were then incubated with TMB development solution for 5-20 minutes, and subsequently incubated with stop solution to intensify the signal. Colorimetric intensity was determined using a 96 well plate reader at 450 nm in order to quantify the level of IFP in each sample. Each of the 97 IFPs was produced at a concentration greater than or equal to 6 ng/mL. For example, FIG. 1 shows the expression level of IFPs 1-11 and the reference human IL-10.

### 6.2 Example 2. Sequence and Structure Based Similarity of Immunomodulatory Proteins

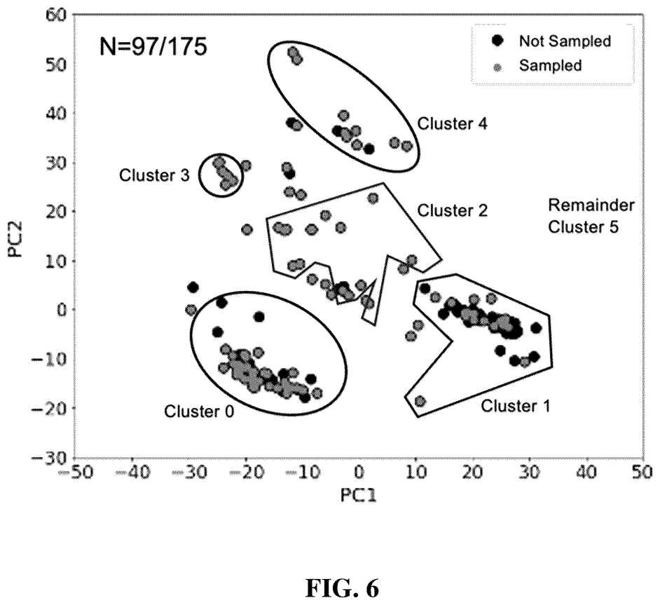

IMPs 1-175 (SEQ ID NOS: 108-454) were clustered by sequence and structural similarity using principal component analysis (PCA). As shown in FIG. 6, clusters of IMPs similar in sequence and structure are apparent. The 97 IMPs generated as described in Example 1 represent multiple species from each cluster. Cluster “0” included IMPs-8, 64, 65, 66, 67, 68, 69, 70, 71, 72, 73, 74, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 129, 133, 164, 165, 166, 167, 169, and 175. Cluster “1” included IMPs-1, 2, 3, 4, 9, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 131, 159, 160, 161, 162, and 163. Cluster “2” included IMPs-138, 139, 140, 141, 142, 144, 145, 151, 152, 153, 158, 170, 171, 172, 173, and 174. Cluster “4” for example, included IMPs-121, 122, 123, 124, 125, 126, 127, 128, 130, 156, 157, and 168. Cluster “5” for example, included IMPs-5, 6, 7, 10, 11, 75, 132, 134, 135, 136, 137, 149, and 155.

### 6.3 Example 3. Immunomodulatory Fusion Protein Engagement of the hIL-10R

The ability of the 97 IFPs produced in Example 1 to engage the hIL-10R was assessed using a cell-based reporter assay known in the art, namely the hIL-10 HEKBlue reporter cell line (InvivoGen #hkb-il10). The hIL-10 HEKBlue reporter cell line expresses the hIL-10Rα and hIL-10Rβ subunits, human STAT3, and a STAT3-inducible SEAP (secreted embryonic alkaline phosphatase) reporter. Thereby, binding of a protein to the hIL-10R triggers JAK1/STAT3 signaling and the subsequent production of SEAP, which can be quantified using standard methods known in the art. The assay was conducted in accordance with the manufacturer's instructions. Briefly, the hIL-10 HEKBlue reporter cell line (InvivoGen #hkb-il10), was incubated with a dose titration of an IFP (i.e., one of 97 IFPs generated) (8 to 24 doses) in DMEM medium with 10% heat-inactivated fetal bovine serum (FBS) at 37° C., 5% CO₂ for 20-24 hours. Subsequently, QuantiBlue substrate (InvivoGen #rep-qbs) was added to the samples and incubated for 1-3 hours. Colorimetric intensity was determined using a 96 well plate reader at 620-655 nm. As shown in FIGS. 2A-2K, IFPs-1-11 for example, engaged the hIL-10R, and IFPs 1, 4-7, and 8-11 did so with a higher potency than hIL-10.

The average EC50 value for the hIL-10R across all 97 IFPs tested was determined to be 22.43 μM. For example, the average EC50 range of a single (IFPs-3-4) or duplicate (IFPs-1-2, 5-7, and 8-11) determinations for each of IFPs 1-11 for the hIL-10R is set forth in Table 9. Also, determined and provided in Table 9 are the relative affinity of each IFP among the group of IFPs 1-11 based on the determined EC50s. “++++” indicates the highest potency among the group of IFPs with those of lower potency denoted as +++, ++ and + in order of decreasing potency. Further provided in Table 9 is the fold difference in EC50 for each IFP relative to the reference Fc-hIL-10 fusion protein.

TABLE 9
IL-10R Binding Characteristics of
Immunomodulatory Fusion Proteins

| | | Relative | ~Average EC50 |
|---|---|---|---|
| | | potency | Fold Difference |
| | ~Average EC50 | of each | Relative to |
| Description | Range (pM) | IFP | Reference Fc-hIL-10 |
| IFP-1 | <1 | ++++ | >100-fold higher |
| (SEQ ID NO: 58) | | | |
| IFP-2 | >200 | + | <1-fold higher |
| (SEQ ID NO: 59) | | | |
| IFP-3 | >200 | + | <1-fold higher |
| (SEQ ID NO: 60) | | | |
| IFP-4 | <5 | +++ | 25-50-fold higher |
| (SEQ ID NO: 61) | | | |
| IFP-5 | 5-20 | +++ | 5-20-fold higher |
| (SEQ ID NO: 62) | | | |
| IFP-6 | <5 | +++ | 25-50-fold higher |
| (SEQ ID NO: 63) | | | |
| IFP-7 | <5 | +++ | 25-50-fold higher |
| (SEQ ID NO: 64) | | | |
| IFP-8 | <5 | +++ | 50-100-fold higher |
| (SEQ ID NO: 65) | | | |
| IFP-9 | 5-20 | ++ | 5-20-fold higher |
| (SEQ ID NO: 66) | | | |
| IFP-10 | 5-20 | +++ | 5-20-fold higher |
| (SEQ ID NO: 67) | | | |
| IFP-11 | <1 | ++++ | >100-fold higher |
| (SEQ ID NO: 68) | | | |
| Reference hIL-10 | 50-100 | + | N/A |
| Fc fusion protein | | | |
| (SEQ ID NO: 102) | | | |

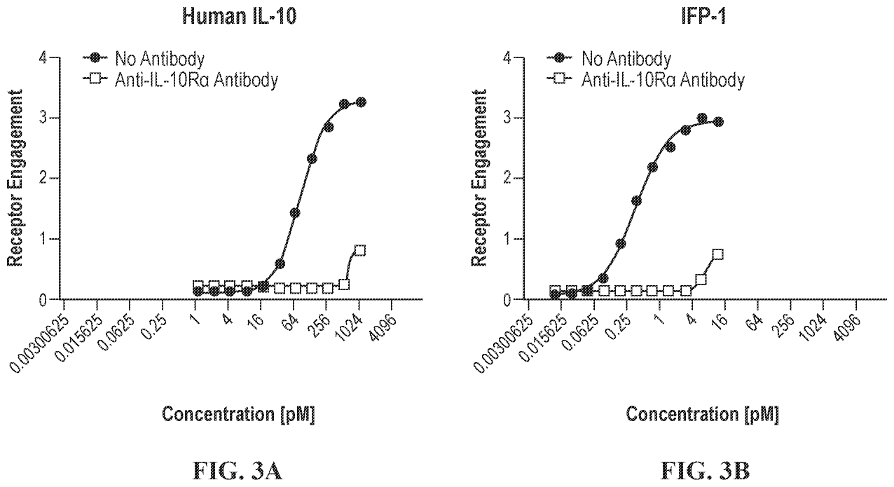

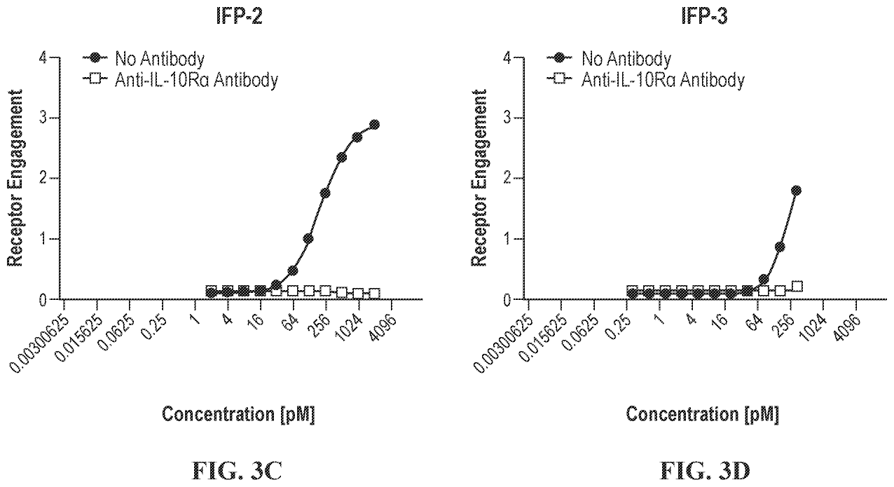

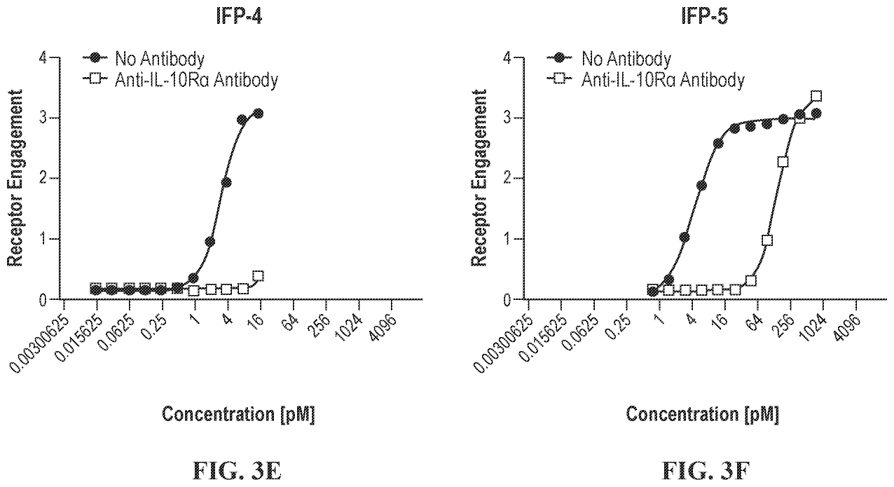

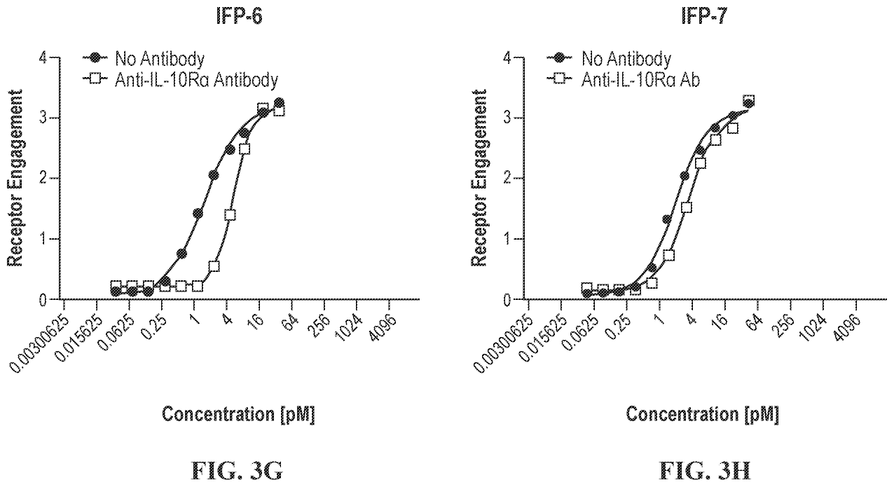

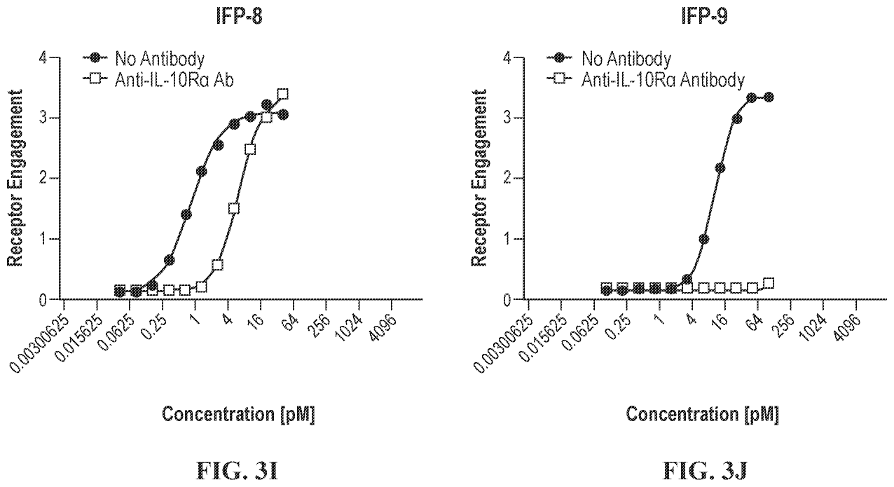

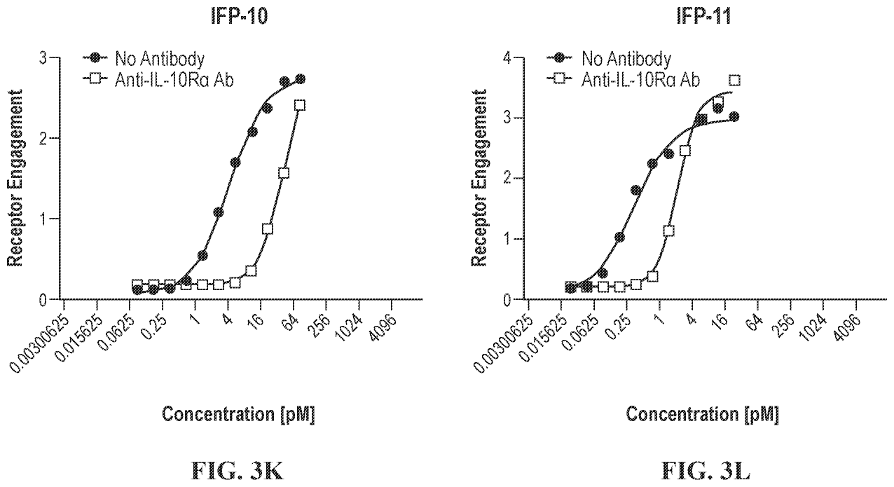

The ability of IFPs 1-11 to engage the hIL-10R in the presence of an anti-hIL-10Rα subunit monoclonal antibody that blocks binding of hIL-10 to the hIL-10Rα was also assessed using the hIL-10 HEKBlue reporter cell line (InvivoGen #hkb-il10) (as described above). The assay was conducted in accordance with the manufacturer's instructions. Briefly, the IL-10 HEKBlue reporter cell line was incubated with a dose titration (8 to 24 doses) of an IFP (i.e., one of IFPs 1-11), in the presence of an anti-hIL-10Rα subunit monoclonal antibody that blocks binding of hIL-10 to the hIL-10Rα (Invitrogen #16-7108-85), or with a constant dosage of an IFP (i.e., one of IFPs 1-11) in the presence of a dose titration of blocking antibody (8 to 24 doses). Cells were incubated in DMEM media with 10% heat inactivated FBS at 37° C., 5% CO₂ for 20-24 hours. QuantiBlue substrate was added to the samples and incubated for 1-3 hours. Colorimetric intensity was determined using a 96 well plate reader at 620-655 nm. As shown in the FIG. 3A-3L, IFPs-1, 5-7, 8, and 10-11 engaged the hIL-10R in the presence of the anti-hIL-10Rα antibody, in contrast to hIL-10.

### 6.4 Example 4. In Vitro Suppression of Pro-Inflammatory Cytokines by Immunomodulatory Fusion Proteins

IFPs 1-11 were assessed for their ability to suppress the production of pro-inflammatory cytokines from stimulated human peripheral blood mononuclear cells (hPBMCs) in vitro. Briefly, donor hPBMCs were thawed and plated in a 96 well plate at a concentration of 200,000 cells/well in HPLM media with 2% type AB human serum. The PBMCs were pretreated with the IFP (i.e., one of IFPs 1-11) and incubated for 20 minutes at 37° C., 5% CO₂ for 20 minutes. After the pre-treatment, the hPBMCs were stimulated with either (i) 10 ng/mL of LPS (Thermo Fisher #00-4976-03) or (ii) 100 ng/mL of anti-CD3 (BioLegend #300332) and anti-CD28 (BioLegend 302902) antibodies, and incubated at 37° C., 5% CO₂ for 24 hours. Subsequently, the cells were spun for 1 minute at 500 g and the was supernatant removed for multiplex ELISA analysis. The concentration of one or more of IFN-γ, IL-1β, IL-4, IL-6, IL-8, and TNFα in the hPBMC stimulated supernatant was determined using the V-Plex Proinflammatory Panel 2 human kit (Meso Scale Diagnostics #K15346D-2) per the manufacturer's protocol. Briefly, culture plates were washed 3 times, the hPBMC stimulated supernatant was added, and the culture plates were sealed and incubated at RT with shaking for 2 hours. The culture plates were washed 3 times, detection antibodies added, and subsequently sealed and incubated at RT for 2 hours. The culture plates were subsequently washed 3 times and Read Buffer T added. Analysis was performed using an MSD instrument and Discovery Workbench. An Fc-GFP fusion protein was also tested as a negative control. The amino acid sequence of the Fc-GFP control is set forth in SEQ ID NO: 106.

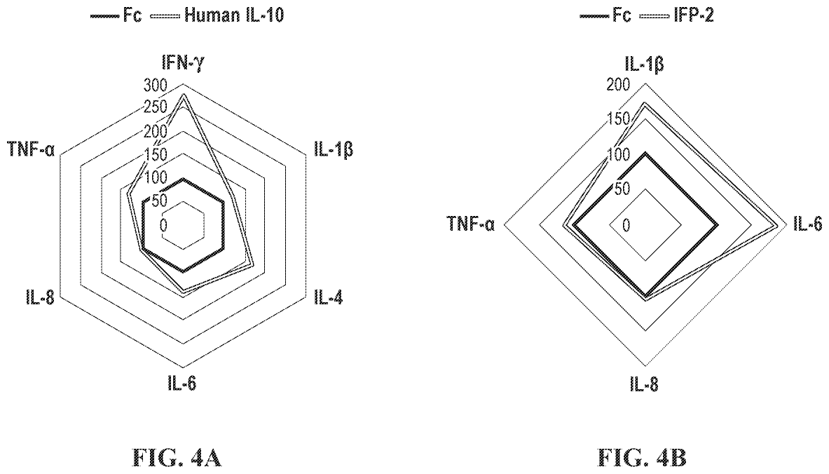

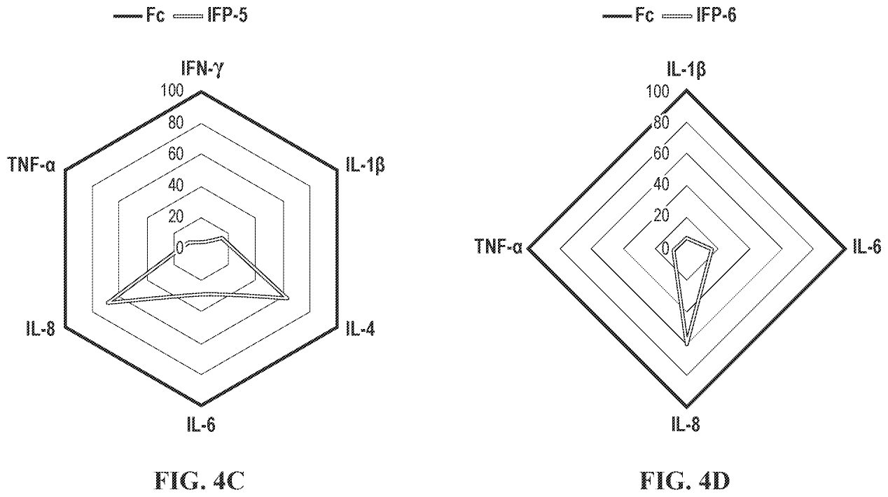

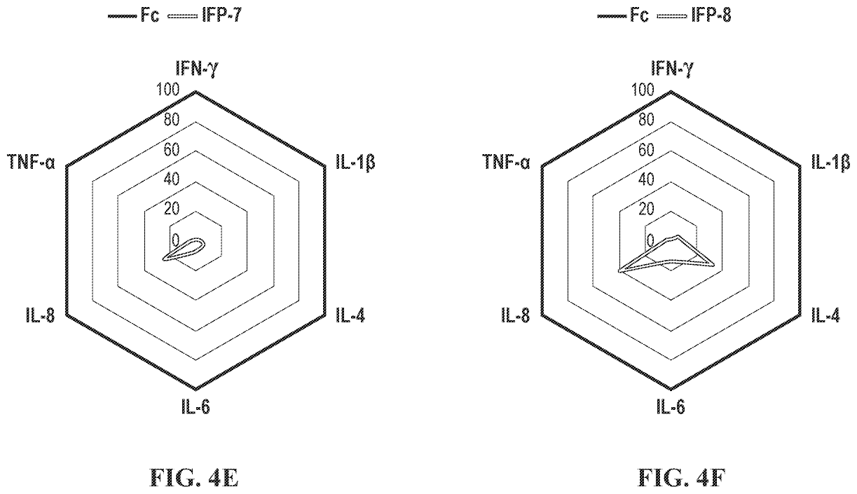

As shown in FIG. 4A-4F, IFPs-5-7 and 8 highly suppress the production of each of IFN-γ, IL-1β, IL-6, IL-8, TNF-α, and IL-4 (in the case of IFP-6, expression of only cytokines IL-1β, IL-6, IL-8, and TNF-α was determined). As shown in FIG. 4A, hIL-10 did not exhibit suppression of the indicated proinflammatory cytokines in the instant Example. This is likely due to the relative low potency of hIL-10 compared to IFPs-5-8 (see Table 9). Furthermore, IFPs-5-7 and 8 were shown to suppress the pro-inflammatory cytokine production to a much greater extent than hIL-10 (compare FIG. 4A (Fc-hIL-10) to FIGS. 4C-F (IFPs-5-8, respectively)).

### 6.5 Example 5. In Vitro Suppression of Pro-Inflammatory Cytokines by Immunomodulatory Fusion Proteins Alone and in Combination with Dexamethasone

IFP-7 was assessed for its ability to suppress the production of pro-inflammatory cytokines from stimulated human peripheral blood mononuclear cells (hPBMCs) in vitro both alone and in combination with the corticosteroid dexamethasone. Briefly, donor hPBMCs were thawed by standard methods known in the art and plated at a concentration of 200,000 cell/well in HPLM media with 2% type AB human serum in a 96 well plate. Cells were pre-treated with 100 nM dexamethasone plus IFP-7 (SEQ ID NO: 64) or a reference hIL-10 Fc fusion protein (SEQ ID NO: 102) and incubated at 37° C. 5% CO₂ for 20 minutes. After pre-treatment the cells were stimulated with 100 ng/mL of anti-CD3 (BioLegend #300332) and anti-CD28 (BioLegend 302902) antibodies and subsequently incubated 37° C. 5% CO₂ for 24 hours. After 24 hours, the cells were spun for 1 minute at 500 g and supernatant removed for multiplex ELISA analysis. The concentration of IFNγ, TNFα, and IL-17a from the supernatant was determined using the V-Plex Proinflammatory Panel 2 human kit (Meso Scale Diagnostics #K15346D-2) per the manufacturer's protocol. Briefly, the plate was washed 3 times, the supernatant was added, then the plate was sealed and incubated at room temperature while shaking for 2 hours. The plate was subsequently washed 3 times and detection antibodies were added. The plate was subsequently sealed and incubated at room temperature for 2 hours. The plate was washed 3 times and Read Buffer T was added. Analysis was performed using an MSD instrument and Discovery Workbench.

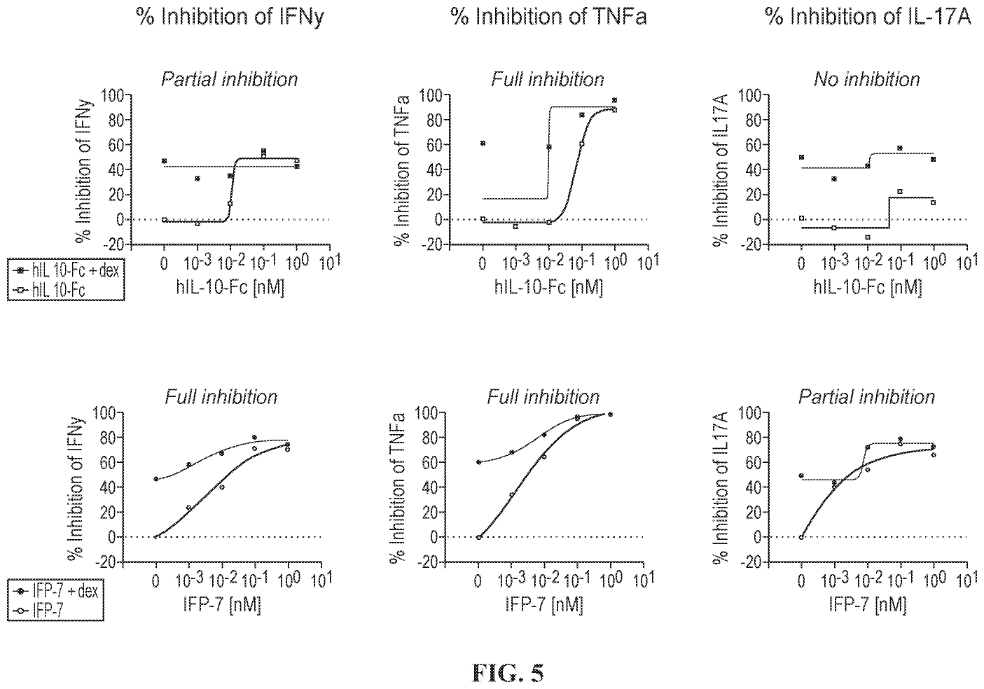

As shown in FIG. 5, IFP-7 fully inhibited TNFα production, almost fully inhibited IFNγ production, and partially inhibited IL-17a production. The addition of a maintenance dose of dexamethasone had no additional impact on the maximum level of inhibition for these 3 cytokines (FIG. 5). IFP-7 showed greater inhibition of pro-inflammatory cytokine expression compared to the reference hIL-10 Fc fusion protein, which only partially inhibited IFNγ production, fully inhibited TNFα production, and had minimal effects on IL-17a levels (FIG. 5). Additionally, IFP-7 was more potent than dexamethasone alone in suppressing IFNγ, TNFα, and IL-17a expression (data not shown).

### 6.6 Example 6. In Vivo Assessment of Human Immune Cell Engraftment and T Cell Phenotypes in Xenograft Mice Treated with Immunomodulatory Fusion Proteins in Combination with Dexamethasone

IFP-7 was assessed for its effect on the engraftment of human immune cells and T cell phenotypes in a xenograft mouse model. Briefly, 6-week-old male, NSG (NOD SCID gamma) mice were administered 100 μg of plasmid encoding IFP-7 (SEQ ID NO: 64), a reference hIL-10 Fc fusion protein (SEQ ID NO: 102), or a control human Fc region (SEQ ID NO: 530), in a 2 mL volume, by hydrodynamic delivery. The following day, the mice were given 100 Rads of irradiation, and subsequently inoculated with 10 million hPBMCs by tail vein intravenous administration. Mice were given intravenous doses of 100 μg dexamethasone (Sigma #D2915) in 200 uL of PBS, or a vehicle control, on days 4, 10, 14, and 20 post-hPBMC engraftment.

On day 14 post-hPBMC engraftment, peripheral blood and plasma were isolated by tail vein draw. For immunophenotyping, the red blood cells in the blood samples were lysed with ACK (ammonium-chloride-potassium) lysis buffer, fixed and permeabilized, then stained for the following markers: mCD45 (TFS #407-0451-82), hCD45 (TFS #78-9459-42), hCD20 (TFS #404-0209-42), hCD4 (TFS #69-0049-42), hCD62L (TFS #63-0629-42), hCD56 (TFS #64-0566-42), hCD27 (TFS #11-0271-82), hFOXP3 (TFS #12-4774-42), hCD45RA (TFS #61-0458-42), hCD3 (TFS #25-0037-42), hCD25 (TFS #51-0259-42), and hCD8 (TFS #56-0088-42). Samples were measured using an Attune NXT flow cytometer and analyzed using FlowJo. hCD45+ cell engraftment was measured as the ratio of hCD45+ cells against mCD45+ cells. CD4+ FOXP3− T cells were characterized as: mCD45−, hCD45+, hCD3+, hCD56−, hCD4+, hFOXP3−. The percentage of hCD4+ FOXP3− T cells that were naïve (hCD27+, hCD45Ra+), or effector (hCD27-, hCD45Ra−) was also calculated.

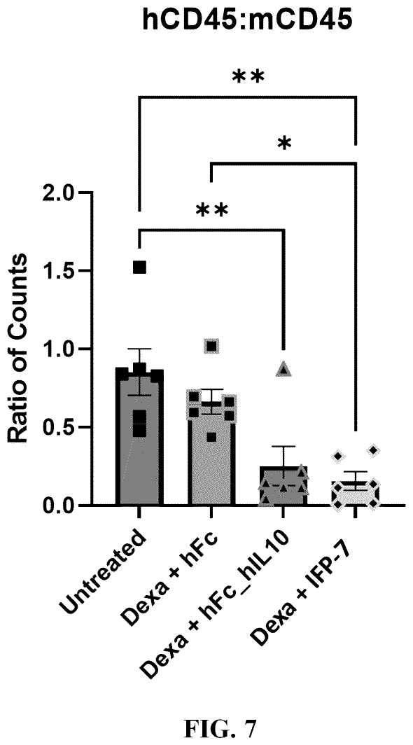

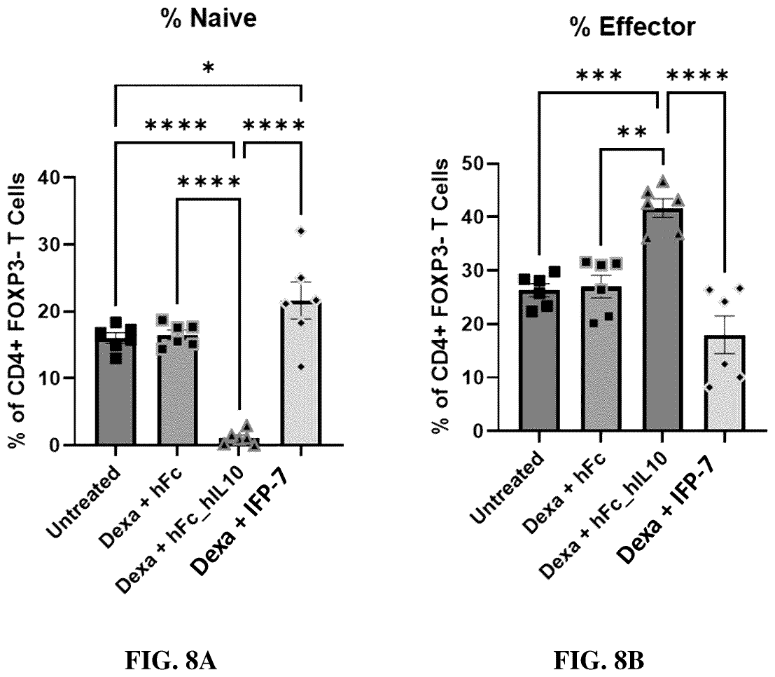

As shown in FIG. 7, treatment with dexamethasone and IFP-7 resulted in significantly decreased human immune cell (i.e., hCD45+ cells) engraftment versus the untreated and human Fc controls. Moreover, the percentage of naïve hCD4+ FOXP3− T cells was significantly higher in the group treated with dexamethasone and IFP-7 compared to the mice treated with dexamethasone and the reference hIL-10 Fc fusion protein or the untreated and dexamethasone plus human Fc controls (FIG. 8A); and the percentage of effector hCD4+ FOXP3− T cells was significantly lower in the group treated with dexamethasone and IFP-7 compared to the mice treated with dexamethasone and the reference hIL-10 Fc fusion protein or the untreated and dexamethasone plus human Fc controls (FIG. 8B).

### 6.7 Example 7. In Vivo Suppression of Pro-Inflammatory Cytokines by Immunomodulatory Fusion Proteins in Combination with Dexamethasone

IFP-7 was assessed for its ability to suppress pro-inflammatory cytokine expression in a xenograft mouse model. Briefly, 6-week-old male, NSG (NOD SCID gamma) mice were administered 100 μg of plasmid encoding IFP-7 (SEQ ID NO: 64), a reference hIL-10 Fc fusion protein (SEQ ID NO: 102), or a human Fc region (SEQ ID NO: 530), in a 2 mL volume, by hydrodynamic delivery. The following day, the mice were given 100 Rads of irradiation, and subsequently inoculated with 10 million hPBMCs by tail vein intravenous administration. Mice were given intravenous doses of 100 μg dexamethasone (Sigma #D2915) in 200 uL of PBS, or a vehicle control, on days 4, 10, 14, and 20 post-hPBMC engraftment.

On day 14 post-PBMC engraftment, peripheral blood and plasma were isolated by tail vein draw. The concentration of IFNγ in the plasma was characterized by MSD (Meso Scale Discovery). After washing the analysis plate 3 times with wash buffer, diluted samples and standards of known concentration were added to the plate and incubated for 2 hours. After washing the plate 3 times, samples were incubated with anti-IFNγ detection antibodies (Meso Scale Diagnostics #K15346D-2) for 2 hours before 3 final washes. Samples were read using a MESO QuickPlex SQ 120MM and data was analyzed using Discovery Workbench Desktop Analysis Software.

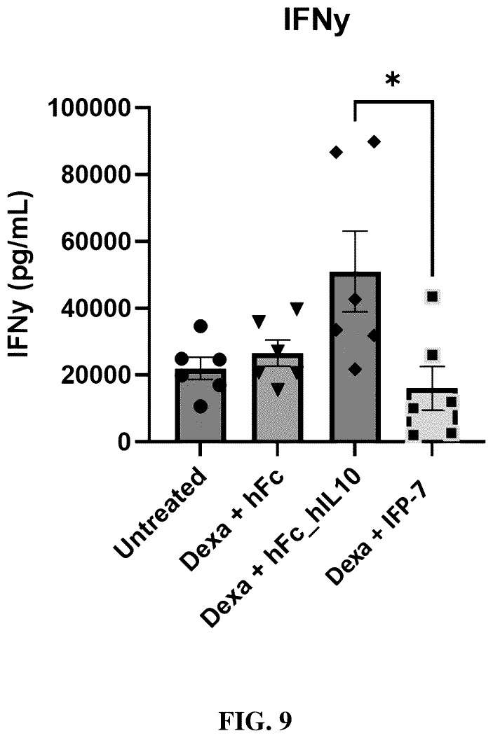

As shown in FIG. 9, treatment with dexamethasone and IFP-7 resulted in significantly lower IFNγ expression compared to the mice treated with dexamethasone and the reference hIL-10 Fc fusion protein, or the two controls (human Fc control and untreated control).

### 6.8 Example 8. In Vivo Assessment of Immunomodulatory Fusion Proteins in Combination with Dexamethasone in a Preclinical Mouse Model of GVHD

IFP-7 was assessed for its effect on the body weight and survival of mice in a preclinical xenograft mouse model of graft versus host disease (GVHD). Briefly, 6-week-old male, NSG (NOD SCID gamma) mice were administered 100 μg of plasmid encoding IFP-7 (SEQ ID NO: 64), a reference hIL-10 Fc fusion protein SEQ ID NO: 102), or a human Fc region (SEQ ID NO: 530), in a 2 mL volume, by hydrodynamic delivery. The following day, the mice were given 100 Rads of irradiation, and subsequently inoculated with 10 million hPBMCs by tail vein intravenous administration. Mice were given intravenous doses of 100 μg dexamethasone (Sigma #D2915) in 200 uL of PBS, or a vehicle control, on days 4, 10, 14, and 20 post-hPBMC engraftment.

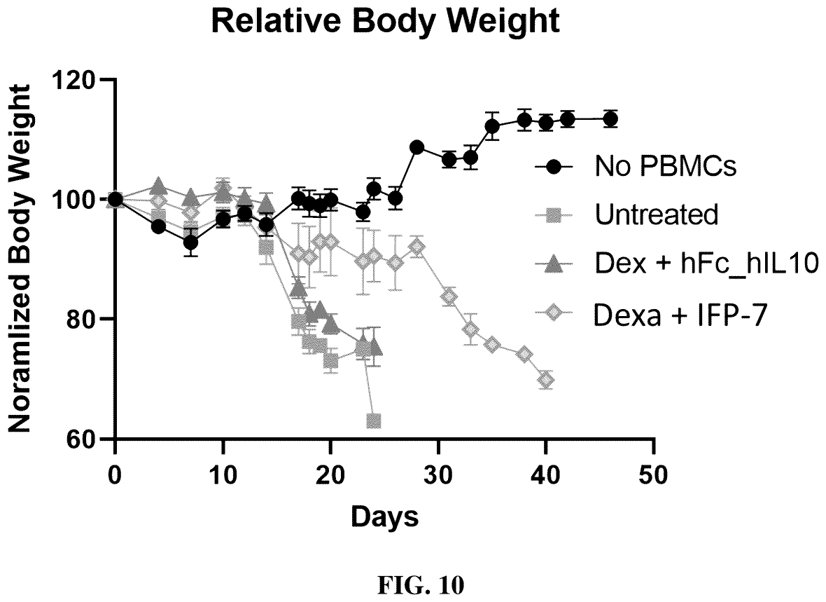

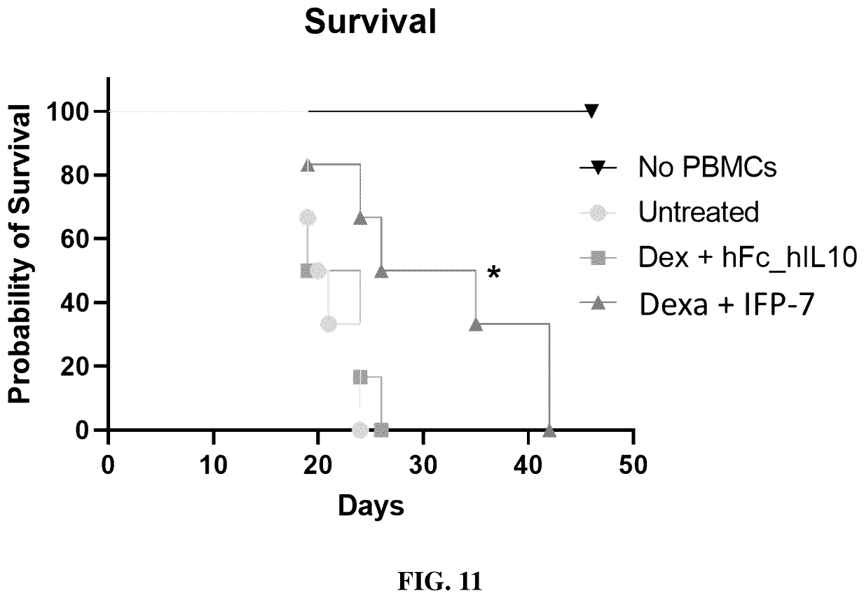

The health of the mice across all control and treatment groups was monitored over time through body weight measurements 3 times per week and overall survival. As shown in FIG. 10, mice treated with dexamethasone and IFP-7 had normal body weight for a significantly longer time period than the mice treated with dexamethasone and the reference hIL-10 Fc fusion protein or the untreated controls. The mice treated with dexamethasone and IFP-7 also survived well past the mice treated with dexamethasone and the reference hIL-10 Fc fusion protein or the untreated control (FIG. 11).

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. An isolated polypeptide or protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-454, wherein the polypeptide or protein specifically binds the human IL-10 Receptor (hIL-10R).

2.-22. (canceled)

23. The isolated polypeptide or protein of claim 1 comprising an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 119-126 or 291-454, wherein the polypeptide or protein specifically binds the hIL-10R.

24-28. (canceled)

29. The isolated polypeptide or protein of claim 1 comprising an amino acid sequence at least about 85%, 86%, 87%, 88%, 89%, 90%, 91%, 92%, 93%, 94%, 95%, 96%, 97%, 98%, 99%, or 100% identical to the amino acid sequence of any one of SEQ ID NOS: 108-118 or 127-290, wherein the polypeptide or protein specifically binds the hIL-10R.

30-45. (canceled)

46. A fusion polypeptide or protein comprising the polypeptide or protein of claim 1 operably connected to a heterologous polypeptide or protein.

47.-113. (canceled)

114. A conjugate comprising the polypeptide or protein of claim 1 operably connected to a heterologous moiety.

115. An immunogenic peptide or protein comprising
(a) at least a portion of the polypeptide or protein of claim 1; and/or
(b) the polypeptide or protein of claim 1 comprising at least one amino acid variation, and
wherein the immunogenic peptide or protein does not specifically bind the hIL-10R or binds the hIL-10R with lower affinity relative to a reference polypeptide or protein of claim 1.

116.-121. (canceled)

122. An isolated antibody that specifically binds to a polypeptide or protein of claim 1.

123. A polynucleotide encoding the polypeptide or protein of claim 1.

124.-130. (canceled)

131. An expression vector comprising the polynucleotide of claim 123.

132. (canceled)

133. A cell comprising the polypeptide or protein of claim 1.

134. A carrier comprising the polypeptide or protein of claim 1.

135.-136. (canceled)

137. A vaccine composition comprising the immunogenic peptide or protein of claim 115 or a nucleic acid molecule encoding the same (or a vector encoding the nucleic acid molecule)).

138. A pharmaceutical composition comprising the polypeptide or protein of claim 1; and a pharmaceutically acceptable excipient.

139. A kit comprising the polypeptide or protein of claim 1; and optionally instructions for using any one or more of the foregoing.

140. A method of delivering a polypeptide or protein to a subject the method comprising administering to the subject the polypeptide or protein of claim 1, to thereby deliver the polypeptide or protein to the subject.

141. (canceled)

142. A method of stimulating the IL-10 pathway in a subject in need thereof, the method comprising administering to the subject the polypeptide or protein of claim 1, to thereby stimulate the IL-10 pathway in the subject.

143.-144. (canceled)

145. A method of treating, preventing, or ameliorating an IL-10 responsive disease in a subject in need thereof, the method comprising administering to the subject the polypeptide or protein of claim 1, to thereby treat, prevent, or ameliorate the IL-10 responsive disease.

146.-150. (canceled)

151. A method of suppressing or preventing an immune response in a subject in need thereof, the method comprising administering to the subject the polypeptide or protein of claim 1, to thereby suppress or prevent an immune response in the subject.

152.-153. (canceled)

154. A method of inducing tolerance to an immunogen in a subject in need thereof, the method comprising administering to the subject the polypeptide or protein of claim 1, to thereby induce tolerance to an immunogen in the subject.

155.-156. (canceled)

157. A method of suppressing IgE expression in a subject in need thereof, the method comprising administering to the subject the polypeptide or protein of claim 1, to thereby suppress IgE expression in the subject.

158.-174. (canceled)

175. A method of inducing an immune response in a subject in need thereof, the method comprising administering to the subject the polypeptide or protein of claim 1, to thereby induce an immune response in the subject.

176.-177. (canceled)

178. A method of preventing, treating, or ameliorating a viral infection in a subject in need thereof, the method comprising administering to the subject the polypeptide or protein of claim 1, to thereby prevent, treat, or ameliorate the viral infection in the subject.

179.-180. (canceled)

181. A method of determining the presence of a virus in a subject, the method comprising
(a) obtaining a sample from the subject or providing a sample that has been obtained from the subject, and
(b) determining the presence or absence of the polypeptide or protein of claim 1 (or a fragment or variant thereof) or a polynucleotide encoding the polypeptide or protein of claim 1 (or the fragment or variant thereof) in the sample.

182. A method of diagnosing a viral infection in a subject, the method comprising
(a) obtaining a sample from a subject or providing a sample that has been obtained from a subject,
(b) determining the presence or absence of the polypeptide or protein of claim 1 (or a fragment or variant thereof) or a polynucleotide encoding the same, and
(c) diagnosing the subject as having the viral infection if the polypeptide or protein of claim 1 (or a fragment or variant thereof) or a polynucleotide encoding the same is determined to be present in the sample in step (b).

183. (canceled)

184. A method of treating a viral infection in a subject, the method comprising
(a) receiving testing results that determined the presence of the polypeptide or protein of claim 1 (or a fragment or variant thereof) or a polynucleotide encoding the same in a sample from the subject,
(b) diagnosing the subject as having the viral infection, and
(c) administering a therapeutic agent to treat the viral infection.

185.-190. (canceled)


SEQUENCE LISTING (550 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 577 aa, Homo sapiens
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 4 - protein, 556 aa, Homo sapiens
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 5 - protein, 325 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 6 - protein, 306 aa, Homo sapiens
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 7 - protein, 20 aa, Homo sapiens
MYRMQLLSCIALSLALVTNS

SEQ ID NO: 8 - protein, 25 aa, Homo sapiens
MGVLLTQRTLLSLVLALLFPSMASM

SEQ ID NO: 9 - protein, 18 aa, Homo sapiens
MASLWLLSCFSLVGAAFG

SEQ ID NO: 10 - protein, 15 aa, Homo sapiens
MNLLLILTFVAAAVA

SEQ ID NO: 11 - protein, 24 aa, Homo sapiens
MALWMRLLPLLALLALWGPDPAAA

SEQ ID NO: 12 - protein, 12 aa, synthetic construct
EPKSCDKTHTCP

SEQ ID NO: 13 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 14 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 106 aa, synthetic construct [sequence omitted]

SEQ ID NO: 16 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 12 aa, synthetic construct
ESKYGPPCPSCP

SEQ ID NO: 23 - protein, 13 aa, synthetic construct
AESKYGPPCPSCP

SEQ ID NO: 24 - protein, 110 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 25 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 106 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 27 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 216 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 223 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 222 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 31 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 228 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 33 - protein, 230 aa, synthetic construct
AESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 229 aa, synthetic construct
AESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 35 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 217 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 216 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 43 - protein, 223 aa, synthetic construct
PCPSCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 222 aa, synthetic construct
PCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 45 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 47 - protein, 230 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 229 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 49 - protein, 8 aa, synthetic construct
GGGGGGGS

SEQ ID NO: 50 - protein, 16 aa, synthetic construct
GGGGGGGSGGGGGGGS

SEQ ID NO: 51 - protein, 24 aa, synthetic construct
GGGGGGGSGGGGGGGSGGGGGGGS

SEQ ID NO: 52 - protein, 5 aa, synthetic construct
GGGGS

SEQ ID NO: 53 - protein, 10 aa, synthetic construct
GGGGSGGGGS

SEQ ID NO: 54 - protein, 15 aa, synthetic construct
GGGGSGGGGSGGGGS

SEQ ID NO: 55 - protein, 4 aa, synthetic construct
GGGS

SEQ ID NO: 56 - protein, 8 aa, synthetic construct
GGGSGGGS

SEQ ID NO: 57 - protein, 12 aa, synthetic construct
GGGSGGGSGGGS

SEQ ID NO: 58 - protein, 447 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGKRAFVVSVAMALLGIYVITNTVNARHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 59 - protein, 464 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMARRLTVASCGSVSLLAAFAAVLLIGCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 60 - protein, 453 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMSNNKILVCAVIILTYTLYTDAYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 61 - protein, 466 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMANVVYVVLVISIMMANIHVSKTYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 62 - protein, 449 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMQGLQLLRGLLCCGVFAAASSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 63 - protein, 454 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVSGAIRTLLMVAVVAVSVRGHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 64 - protein, 452 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 65 - protein, 442 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 66 - protein, 449 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMALAHQLPVWIFSIWILYFTLPLSEERVLPLRGNCKLLLQDTVIPNLLYSMRSIFQDIKPYFQGKDSLNNLLLSGQLLEDLQSPIGCDALSEMIQFYLEEVMPQAEIHHPKHKNSVMQLGETLHTLISQLQECTALFPCKHKSLGAQKIKEEVSKLGQYGIIKAVAEFDIFINYMESYFGVK

SEQ ID NO: 67 - protein, 456 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRRSFGIIVAGAIGTLLMMAVVVLSAHDHEHKEVPPACDPVHGNLAGIFKELRATYASIREGLQKKDTVYYTSLFNDRVLHEMLSPMGCRVTNELMEHYLDGVLPRASHLDYDNSTLNGLHVFASSMQALYQHMLKCPALACTGKTPAWMYFLEVEHKLNPWRGTAKAAAEADLLLNYLETFLLQF

SEQ ID NO: 68 - protein, 453 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVAGAIGTLLMMAVVVFSAHEHKEVPPACDPVHGNLAGIFKELRATYASIREGLQKKDTVYYTSLFNDRVLQEMLSPMGCRVTNELMEHYLDGVLPRALHLDYDNSTLNGLHAFASSMQALYQHMLKCPALACTGKTPAWMYFLEVEHKLNPWRGTAKAAAEADLLLNYLETFLLQF

SEQ ID NO: 69 - protein, 427 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGKRAFVVSVAMALLGIYVITNTVNARHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 70 - protein, 444 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMARRLTVASCGSVSLLAAFAAVLLIGCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 71 - protein, 433 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMSNNKILVCAVIILTYTLYTDAYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 72 - protein, 446 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMANVVYVVLVISIMMANIHVSKTYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 73 - protein, 429 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMQGLQLLRGLLCCGVFAAASSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 74 - protein, 434 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVSGAIRTLLMVAVVAVSVRGHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 75 - protein, 432 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 76 - protein, 422 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 77 - protein, 429 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMALAHQLPVWIFSIWILYFTLPLSEERVLPLRGNCKLLLQDTVIPNLLYSMRSIFQDIKPYFQGKDSLNNLLLSGQLLEDLQSPIGCDALSEMIQFYLEEVMPQAEIHHPKHKNSVMQLGETLHTLISQLQECTALFPCKHKSLGAQKIKEEVSKLGQYGIIKAVAEFDIFINYMESYFGVK

SEQ ID NO: 78 - protein, 436 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRRSFGIIVAGAIGTLLMMAVVVLSAHDHEHKEVPPACDPVHGNLAGIFKELRATYASIREGLQKKDTVYYTSLFNDRVLHEMLSPMGCRVTNELMEHYLDGVLPRASHLDYDNSTLNGLHVFASSMQALYQHMLKCPALACTGKTPAWMYFLEVEHKLNPWRGTAKAAAEADLLLNYLETFLLQF

SEQ ID NO: 79 - protein, 433 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVAGAIGTLLMMAVVVFSAHEHKEVPPACDPVHGNLAGIFKELRATYASIREGLQKKDTVYYTSLFNDRVLQEMLSPMGCRVTNELMEHYLDGVLPRALHLDYDNSTLNGLHAFASSMQALYQHMLKCPALACTGKTPAWMYFLEVEHKLNPWRGTAKAAAEADLLLNYLETFLLQF

SEQ ID NO: 80 - protein, 446 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGKRAFVVSVAMALLGIYVITNTVNARHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 81 - protein, 463 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMARRLTVASCGSVSLLAAFAAVLLIGCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 82 - protein, 452 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMSNNKILVCAVIILTYTLYTDAYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 83 - protein, 465 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMANVVYVVLVISIMMANIHVSKTYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 84 - protein, 448 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMQGLQLLRGLLCCGVFAAASSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 85 - protein, 453 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVSGAIRTLLMVAVVAVSVRGHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 86 - protein, 451 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 87 - protein, 441 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 88 - protein, 448 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMALAHQLPVWIFSIWILYFTLPLSEERVLPLRGNCKLLLQDTVIPNLLYSMRSIFQDIKPYFQGKDSLNNLLLSGQLLEDLQSPIGCDALSEMIQFYLEEVMPQAEIHHPKHKNSVMQLGETLHTLISQLQECTALFPCKHKSLGAQKIKEEVSKLGQYGIIKAVAEFDIFINYMESYFGVK

SEQ ID NO: 89 - protein, 455 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRRSFGIIVAGAIGTLLMMAVVVLSAHDHEHKEVPPACDPVHGNLAGIFKELRATYASIREGLQKKDTVYYTSLFNDRVLHEMLSPMGCRVTNELMEHYLDGVLPRASHLDYDNSTLNGLHVFASSMQALYQHMLKCPALACTGKTPAWMYFLEVEHKLNPWRGTAKAAAEADLLLNYLETFLLQF

SEQ ID NO: 90 - protein, 452 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVAGAIGTLLMMAVVVFSAHEHKEVPPACDPVHGNLAGIFKELRATYASIREGLQKKDTVYYTSLFNDRVLQEMLSPMGCRVTNELMEHYLDGVLPRALHLDYDNSTLNGLHAFASSMQALYQHMLKCPALACTGKTPAWMYFLEVEHKLNPWRGTAKAAAEADLLLNYLETFLLQF

SEQ ID NO: 91 - protein, 426 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGKRAFVVSVAMALLGIYVITNTVNARHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 92 - protein, 443 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMARRLTVASCGSVSLLAAFAAVLLIGCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 93 - protein, 432 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMSNNKILVCAVIILTYTLYTDAYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 94 - protein, 445 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMANVVYVVLVISIMMANIHVSKTYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 95 - protein, 428 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMQGLQLLRGLLCCGVFAAASSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 96 - protein, 433 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVSGAIRTLLMVAVVAVSVRGHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 97 - protein, 431 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 98 - protein, 421 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 99 - protein, 428 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMALAHQLPVWIFSIWILYFTLPLSEERVLPLRGNCKLLLQDTVIPNLLYSMRSIFQDIKPYFQGKDSLNNLLLSGQLLEDLQSPIGCDALSEMIQFYLEEVMPQAEIHHPKHKNSVMQLGETLHTLISQLQECTALFPCKHKSLGAQKIKEEVSKLGQYGIIKAVAEFDIFINYMESYFGVK

SEQ ID NO: 100 - protein, 435 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRRSFGIIVAGAIGTLLMMAVVVLSAHDHEHKEVPPACDPVHGNLAGIFKELRATYASIREGLQKKDTVYYTSLFNDRVLHEMLSPMGCRVTNELMEHYLDGVLPRASHLDYDNSTLNGLHVFASSMQALYQHMLKCPALACTGKTPAWMYFLEVEHKLNPWRGTAKAAAEADLLLNYLETFLLQF

SEQ ID NO: 101 - protein, 432 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRRRRSFGIVVAGAIGTLLMMAVVVFSAHEHKEVPPACDPVHGNLAGIFKELRATYASIREGLQKKDTVYYTSLFNDRVLQEMLSPMGCRVTNELMEHYLDGVLPRALHLDYDNSTLNGLHAFASSMQALYQHMLKCPALACTGKTPAWMYFLEVEHKLNPWRGTAKAAAEADLLLNYLETFLLQF

SEQ ID NO: 102 - protein, 445 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 103 - protein, 425 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 104 - protein, 444 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 105 - protein, 424 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 106 - protein, 518 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRKGEELFTGVVPILVELDGDVNGHKFSVSGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKRPAANDENYAASV

SEQ ID NO: 107 - protein, 498 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSMRKGEELFTGVVPILVELDGDVNGHKFSVSGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSALSKDPNEKRDHMVLLEFVTAAGITHGMDELYKRPAANDENYAASV

SEQ ID NO: 108 - protein, 180 aa, synthetic construct
MGKRAFVVSVAMALLGIYVITNTVNARHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 109 - protein, 197 aa, synthetic construct
MARRLTVASCGSVSLLAAFAAVLLIGCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 110 - protein, 186 aa, synthetic construct
MSNNKILVCAVIILTYTLYTDAYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 111 - protein, 199 aa, synthetic construct
MANVVYVVLVISIMMANIHVSKTYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 112 - protein, 182 aa, synthetic construct
MQGLQLLRGLLCCGVFAAASSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 113 - protein, 187 aa, synthetic construct
MRRRRSFGIVVSGAIRTLLMVAVVAVSVRGHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 114 - protein, 185 aa, synthetic construct
MGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 115 - protein, 175 aa, synthetic construct
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 116 - protein, 182 aa, synthetic construct
MALAHQLPVWIFSIWILYFTLPLSEERVLPLRGNCKLLLQDTVIPNLLYSMRSIFQDIKPYFQGKDSLNNLLLSGQLLEDLQSPIGCDALSEMIQFYLEEVMPQAEIHHPKHKNSVMQLGETLHTLISQLQECTALFPCKHKSLGAQKIKEEVSKLGQYGIIKAVAEFDIFINYMESYFGVK

SEQ ID NO: 117 - protein, 189 aa, synthetic construct
MRRRRRSFGIIVAGAIGTLLMMAVVVLSAHDHEHKEVPPACDPVHGNLAGIFKELRATYASIREGLQKKDTVYYTSLFNDRVLHEMLSPMGCRVTNELMEHYLDGVLPRASHLDYDNSTLNGLHVFASSMQALYQHMLKCPALACTGKTPAWMYFLEVEHKLNPWRGTAKAAAEADLLLNYLETFLLQF

SEQ ID NO: 118 - protein, 186 aa, synthetic construct
MRRRRSFGIVVAGAIGTLLMMAVVVFSAHEHKEVPPACDPVHGNLAGIFKELRATYASIREGLQKKDTVYYTSLFNDRVLQEMLSPMGCRVTNELMEHYLDGVLPRALHLDYDNSTLNGLHAFASSMQALYQHMLKCPALACTGKTPAWMYFLEVEHKLNPWRGTAKAAAEADLLLNYLETFLLQF

SEQ ID NO: 119 - protein, 154 aa, synthetic construct
RHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 120 - protein, 171 aa, synthetic construct
CQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 121 - protein, 164 aa, synthetic construct
YCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 122 - protein, 176 aa, synthetic construct
YCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 123 - protein, 161 aa, synthetic construct
RSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 124 - protein, 157 aa, synthetic construct
HEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 125 - protein, 159 aa, synthetic construct
KGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 126 - protein, 150 aa, synthetic construct
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 127 - protein, 185 aa, synthetic construct
MSKNKILVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKP

SEQ ID NO: 128 - protein, 185 aa, synthetic construct
MSNNKILVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKT

SEQ ID NO: 129 - protein, 185 aa, synthetic construct
MPNNKILVCAVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKS

SEQ ID NO: 130 - protein, 184 aa, synthetic construct
MSNKKILVCVVIILTYTLYTDAYCVEYKESEEDRQQCSSSSFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTK

SEQ ID NO: 131 - protein, 185 aa, synthetic construct
MSNNKILVCVAIILTYTLYTDAYCVEYAESDEDKQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 132 - protein, 185 aa, synthetic construct
MSKNKVLVCFVIILTYTLYTDAYCVEYEESEEDKQQCGSNGGPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 133 - protein, 185 aa, synthetic construct
MSNNKILVCAVIILTYTLYTDAYCVEYAESDEDRQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 134 - protein, 186 aa, synthetic construct
MSNNKILLCVAIILTYTLYTDAYCVEYEESEEDKQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 135 - protein, 185 aa, synthetic construct
MSNNKILVCAVIILTYTLYTDAYCIQYEESEEDKQQCSSSNFPASLPHMLRELRAAFGKVKTFFQMKDQLNSMLLTQSLLDDFKGYLGCQALSEMIRFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 136 - protein, 185 aa, synthetic construct
MSKNKFLVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLREKLKTLRLRLRRCHRFLPCENKSKAVEQVKRVFNMLQERGVYKAMSEFDIFINYIESYMTTKT

SEQ ID NO: 137 - protein, 186 aa, synthetic construct
MSKNKILVCFVIILTYTLYTDAYCVEYEESEEDKQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 138 - protein, 185 aa, synthetic construct
MSNNKILVCVAIILTYTLYTDAYCVEYAESDEDKQQCSGSNFPASLPHMLRELRAAFGKVKTFFQMKDQLNSMLLTQSLLDDFKGYLGCQALSEMIQFYLEEVMPRAENHGPDIKEHVNSLGEKLKTLRLRLRRCHRFLPCENKSKAVEQVKRVFNMLQERGVYKAMSEFDIFINYIESYMTTKM

SEQ ID NO: 139 - protein, 186 aa, synthetic construct
MSNNKILVCVVIILTYTLYTDAYCVEYEESEEDRQQCSGSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 140 - protein, 186 aa, synthetic construct
MSNNKILVCAVIILTYTLYTDAYCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 141 - protein, 186 aa, synthetic construct
MSKNKILVCVAIILTYTLYTDAYCVEYEESDEDKQQCSSSTGAPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 142 - protein, 186 aa, synthetic construct
MSKNKILVCVAIILTYTLYTDAYCVEYEETKEDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 143 - protein, 185 aa, synthetic construct
MSKNKILVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGDYKAMSEFDIFINYIESYMTTKS

SEQ ID NO: 144 - protein, 186 aa, synthetic construct
MSNNKILVCVVIILTYTLYTDAYCVEYEESEEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCEDKSKAVEQVKRVFNMLQERGVYKAMSEFDILINYIESYMTTKM

SEQ ID NO: 145 - protein, 186 aa, synthetic construct
MSNNKILVCAVIILTYTLYTDAYCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMATKM

SEQ ID NO: 146 - protein, 179 aa, synthetic construct
MFRALLLCCLALLAGVWADNRYDGQDGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 147 - protein, 186 aa, synthetic construct
MSKNKILVCVAIILTYTLYTDAYCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 148 - protein, 186 aa, synthetic construct
MSKNKILVCVAIILTYTLYTDAYCVEYEESKEDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 149 - protein, 186 aa, synthetic construct
MSKNKILVCVAIILTYTLYTDAYCVEYLESGEDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 150 - protein, 186 aa, synthetic construct
MSKNKILVCVAIILTYTLYTDAYCVEYLESREDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 151 - protein, 178 aa, synthetic construct
MPGAALLYCLFFVTGVWAESENNCTHFPTSLPHMLHELRAAFSRVKTFFQMKDQLDNMLLNGSLLEDFKGYLGCQALSEMIQFYLEEVMPQAENHSGGGGPDIKEHVNSLGEKLKTLRVRLRRCHRFLPCENKSKAVEQVKSAFSKLQEKGVYKAMSEFDIFINYIEAYMTTKMKNKK

SEQ ID NO: 152 - protein, 186 aa, synthetic construct
MSNNKILVCVAIILTYTLYTDAYCVEYLESDEDKQHCSSSNGASASSPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 153 - protein, 185 aa, synthetic construct
MSNNKILVCVAIILTYTLYTNAYCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLAEKLKTLRLRLRRCHRFLPCENKSKAVEQVKRVFNMLQERGVYKAMSEFDIFINYIESYMTTKS

SEQ ID NO: 154 - protein, 185 aa, synthetic construct
MSNNKILVCVAIILTYTLYTNAYCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLGEKLKTLRLRLRRCHRFLPCENKSKAVAQVKRVFNMLQERGVYKAMSEFDIFINYIESYMTTKS

SEQ ID NO: 155 - protein, 174 aa, synthetic construct
ILVCFVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 156 - protein, 174 aa, synthetic construct
ILVCFVIILTYTLYTDAYCVEYEESEEDRQQCSSSNFPASLPHMLRELRAAFGKVKTFFQMKDQLNSMLLTQSLLDDFKGYLGCQAFSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 157 - protein, 174 aa, synthetic construct
LVCVAIILTYTLYTDAYCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 158 - protein, 177 aa, synthetic construct
MGLRSGLTLQCLVILQCLVMLYLAPACKGVSNCGNLPHMLRDLRDAFSRVKTFFQMKDQLDNILLKESLLEDFKGYLGCQALSEMIQFYLEEVMPQAENQDPHAKEHVNSLGENLKTLRLRLRRCHRFLPCENKSKAVEQVKNAFSKLQEKGVYKAMSEFDIFINYIEAYMTMKIRR

SEQ ID NO: 159 - protein, 174 aa, synthetic construct
LVCVAIILTYTLYTDAYCVEYLESREDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 160 - protein, 174 aa, synthetic construct
ILVCFVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSPGEKLKTLRLRLRRCHRFLPCENKSKAVEQVKRVFNMLQERGVYKAMSEFDIFINYIES

SEQ ID NO: 161 - protein, 186 aa, synthetic construct
MANVIYVVLALNILLSQIHVSNPYCTSCSYRDCTEDEDQKQQCEGGLRSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 162 - protein, 174 aa, synthetic construct
ILVCFVIILTYTLYTDAYCVEYEESEEDRQQCSSSNFPASLPHMPRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 163 - protein, 189 aa, synthetic construct
MANVIYVVLALNILLSQFHVSNPYCTSCSHRDCTEDDEQKQQCEGGSGGLGSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 164 - protein, 170 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPGAKDHVNSLGENLKTLRLRLRRCHRFLPCENKSKAVEQIKNAFNKLQEKGIYKAMSEFDIFINYIEAYMTIKAR

SEQ ID NO: 165 - protein, 170 aa, synthetic construct
MERRLVVTLQCLVLLYLAPECGSTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 166 - protein, 170 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTMKAR

SEQ ID NO: 167 - protein, 171 aa, synthetic construct
MGPRAGLALQCLLLLYLAPACKGVSNCGNLPHMLRDLRDAFSRVKTFFQMKDQLDNILLKESLLEDFKGYLGCQALSEMIQFYLEEVMPQAENQDPNAKEHVNSLGENLKTLRLRLRRCHRFLPCENKSKAVEQVKNAFSKLQEKGVYKAMSEFDIFINYIEAYMTMKTRR

SEQ ID NO: 168 - protein, 170 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTIRLRLRRCHRFLPCENKSKAVEQIKNAFNKLQEKGIYKAMSEFDIFINYIEAYMTIKAR

SEQ ID NO: 169 - protein, 170 aa, synthetic construct
MERRLVVTLQCLVLLYLAPECGGTDQCDNFPQMLRDLRDAFSRVKTFFQTKDEVDSLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 170 - protein, 170 aa, synthetic construct
MERRLMVTLQCLVLLYLAPECGSTDQCDNFPQMLRDLRDAFSRVKTFFQTKDEVDNILLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 171 - protein, 170 aa, synthetic construct
MERRLVVTLQCLVLLYLAPECGGTDQCDNFPQMLRDLRDAFSRVKTFFQTKDEVDNLFLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 172 - protein, 170 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEKVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 173 - protein, 170 aa, synthetic construct
MERRLMVTLQCLVLLYLAPECGSTDQCDNFPQMLRDLRDAFSRVKTFFQTKDAVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 174 - protein, 177 aa, synthetic construct
MELRSGLTLQCLVMLQCLVMLYLAPACKGASNCGNLPHMLRDLRDAFSRVKTFFQMKDQLDNILLKESLLEDFRGYLGCQALSEMIQFYLEEVMPQAENQDPHSKEHVNSLGENLKTLRLRLRRCHRFLPCENKGKAVEQVKNAFSKLQEKGVYKAMSEFDIFINYIEAYMTMKLRR

SEQ ID NO: 175 - protein, 170 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFHIFINYIEAYMTIKAR

SEQ ID NO: 176 - protein, 161 aa, synthetic construct
MERRLVVTLQCLVLLYLAPECGEMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 177 - protein, 171 aa, synthetic construct
MELSLGLTLHFLVFLCLAPACGRAETCGNIPHMLRDLRDAFSRVKTFFQMKDQLDNILLKESLLEDFKGYLGCQALSEMIQFYLEEVMPQAEAMSLKSQEHVNFLGENLNTLRLRLRRCHRFLPCENKSKAVEQVKNAFSKLQEKGVYKAMSEFDIFINYIEAYMTMKLRR

SEQ ID NO: 178 - protein, 204 aa, synthetic construct
MAYGKKIVAASLLVIPAYVVFTNATANNRAQKCFCFDGSNAGNSEETNTAAFQKKCDSEIPESLPYMLRDLRNSSVQTRRYFQEKDEENSPLLTQKLLEDFKGYLGCQALSEMIQFYLEEVMPQAEDSNPSAKDSVTSLGEKLKTLRLRLRRCHRFLPCENKSKAVENLKSKFGDLGNQGVHKAMSEFDIFINYIETYMTTKMK

SEQ ID NO: 179 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLETVFPAGDHVYPGLKTELHSMRSTLESIYKDMRQCPLLGCGDKSVISRLSQEAERKSDNGTRKGLSELDTLFSRLEEYLHSRK

SEQ ID NO: 180 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 181 - protein, 175 aa, synthetic construct
MLSVMVSSSLVLIVFFLGASEEAKPATTTTKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 182 - protein, 176 aa, synthetic construct
MLSVMVSSSLVLIVFFLGASEEAKPATTTTIKNTKPQCRPEDYATRLQDLRVTFHRVKPTLQREDDYSVWLDGTMVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 183 - protein, 176 aa, synthetic construct
MLSVMVSSSLVLIVFFLGASEEAKPATTTTIKNTKPQCRPEDYATRLQDLRVTFHRVKSTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 184 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDMLFSRLEEYLHSRK

SEQ ID NO: 185 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVLPAGDHVYPGLKTELHSMRSTLESIYKDMRQCPLLGCGDKSVISRLSQEAERKSDNGTRKGLSELDTLFSRLEEYLHSRK

SEQ ID NO: 186 - protein, 176 aa, synthetic construct
MLSVMVSSSLVLIVFFLGASEEAKPAATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 187 - protein, 176 aa, synthetic construct
MLSVMVSSSLVLIVFFLGASEEAKPATTTTINNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 188 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMHSTLESIYKDMRQCPLLGCGDKSVISRLSQEAERKSDNGTRKGLSELDTLFSRLEEYLHSRK

SEQ ID NO: 189 - protein, 176 aa, synthetic construct
MLSVMVSSSLVLIIFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 190 - protein, 192 aa, synthetic construct
MGSRPVRVCGSSNLLCLLLLILVVLVAVVIDRGCWASSKPPVDCDPIHGTLSRIIKEVRTGYGSIKQALQSKDTVYYVSLFHESLLREMLSPVGCRVTNELMQHYLDGVLPRAFQCGYDNTTLSGLHSLVSSLDTLYKHMLKCPALACTGQTPAWTQFLETEHKLDPWKGTIKATAEMDLLVNYLETFLAQS

SEQ ID NO: 191 - protein, 176 aa, synthetic construct
MLSVMVSSSLVLIVFFLGASEEAKPATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 192 - protein, 176 aa, synthetic construct
MLSVMVSSSLVLIVFFLGASEEAKPAATTTIKNTKPQCRPEDYATRLQDLRVTFHRVKPTLQHEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 193 - protein, 176 aa, synthetic construct
MLSVMVSSSLVLIVFFLGASEEAKPATTTTIKNTKPQCRPEDYATRLQDLRVTFHRVKPTLQREDDYSVWLDGMVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 194 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVVDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 195 - protein, 176 aa, synthetic construct
MLSVMVSSSLVLIVFFLGASEEAKPATTTTIKNTKPQCRPEDYATRLQDLRITFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 196 - protein, 175 aa, synthetic construct
MLSVMVSSSLVLIVFFLGASEEAKPATTTIKNTKPQCRPEDYATRLQDLRVTFHRIKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 197 - protein, 176 aa, synthetic construct
MLSVMVSSSLVLIVFFLGASEEAKPATTTTIKNTKPQCRPEDYATRLQDLRVTFHRVKPTLQCEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 198 - protein, 177 aa, synthetic construct
MLSVMVFSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 199 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 200 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEILFPAGDHVYPGLKTELHSMRSTLESIYKDMRQCPLLGCGDKSVISRLSQEAERKSDNGTRKGLSELDTLFSRLEEYLHSRK

SEQ ID NO: 201 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 202 - protein, 175 aa, synthetic construct
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPRLKTELHSMRSTLESIYKDMQQCPLLGCGDKSVISRLSQEAERKSDNGTRKGLSELDTLFSRLEEYLHSRK

SEQ ID NO: 203 - protein, 177 aa, synthetic construct
MLSVMVSSSLVLIVFFLGASEEAKPAATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 204 - protein, 178 aa, synthetic construct
MLSVMVSSSLVLIVFFLGASEEAKPAATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 205 - protein, 177 aa, synthetic construct
MLSVMVSSSLVLIVFFLGASEEAKPAATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 206 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIMFPAGDHVYPGLKTELHSMRSTLESIYKDMRQCPLLGCGDKSVISRLSQEAERKSDNGTRKGLSELDTLFSRLEEYLHSRK

SEQ ID NO: 207 - protein, 175 aa, synthetic construct
MLSVMVSSSLVLIVFFLGASEEAKSATTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 208 - protein, 176 aa, synthetic construct
MLSVMVSSSLVLIIFFLGASEEAKPATTTTIKNTKPQCRPEDYATRLQDLRVTFHRVKPTLQREDDYSVWLDGTVVKGCWGCSVMDWLLRRYLEIVFPAGDYVYPGLKTELHSMRSTLESIYKDMRQCPLLGCGDKSVISRLSQEAERKSDNGMRKGLSELDTLFSRLEEYLHSRK

SEQ ID NO: 209 - protein, 175 aa, synthetic construct
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 210 - protein, 177 aa, synthetic construct
MLSVMVSSSLVLIVFFLGASEEAKPATTTTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 211 - protein, 178 aa, synthetic construct
MLSVMVSSSLVLIVFFLGASEEAKPAATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 212 - protein, 178 aa, synthetic construct
MLSVMVSSSLVLIIFFLGASEEAKPATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 213 - protein, 176 aa, synthetic construct
MLSVMVSSSLVLIVFFLGASEEAKPAATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLNELDTLFSRLEEYLHSRK

SEQ ID NO: 214 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 215 - protein, 176 aa, synthetic construct
MLSVMVSSSLVLIVFFLGASEEAKPATTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 216 - protein, 179 aa, synthetic construct
MLSVMVSSSLVLIVFFLGASEEAKPATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 217 - protein, 177 aa, synthetic construct
MLSVMVSSSLVLIVFFLGASEEAKPAATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKSLSELDTLFSRLEEYLHSRK

SEQ ID NO: 218 - protein, 175 aa, synthetic construct
MLSVMVSSSLVLIVFFLGASEKAKSATTTIKNTKPQCRPEDYATRLQDLRVTFHRVKPTLQREDDYSVWLDGTVVKGCWGCSVMDWLLRRYLEIVFPAGDHVYSGLKTELHSMRSTLESIYKDMRQCPLLGCGDKSVISRLSQEAERKSDNGTRKGLSELDTLFSRLEEYLHSRK

SEQ ID NO: 219 - protein, 179 aa, synthetic construct
MLSVMVSSSLVLIVFFLGASEEAKPAATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 220 - protein, 179 aa, synthetic construct
MLSVMVSSSLVLIVFFLGASEEAKPAATTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 221 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMWQCPLLGCGDKSVISRLSQEAERKSDNGTRKGLSELDTLFSRLEEYLHSRK

SEQ ID NO: 222 - protein, 177 aa, synthetic construct
MLSVMVSSSLVLIVFFLGASEEAKPAATTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 223 - protein, 173 aa, synthetic construct
MLSVMVSSSLVLIVFFLGASEEAKPATIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 224 - protein, 180 aa, synthetic construct
MLSVMVSSSLVLIVFFLGASEEAKPAATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 225 - protein, 180 aa, synthetic construct
MLSVMVSSSLVLIVFFLGASEEAKPAATTTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 226 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLRELDTLFSRLEEYLHSRK

SEQ ID NO: 227 - protein, 181 aa, synthetic construct
MLSVMVSSSLVLIVFFLGASEEAKPAATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 228 - protein, 179 aa, synthetic construct
MLSVMVSSSLVLIVFFLGASEEAKPAATTTTTTIKNTKPQCRPEDYATRLQDLRVTFHRVKPTLQREDDYSVWLDGTVVKGCWRCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 229 - protein, 177 aa, synthetic construct
MLSVMVSSSLVMIVFFLGASEEAKPATTTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 230 - protein, 182 aa, synthetic construct
MLSVMVSSSLVLIVFFLGASEEAKPAAATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 231 - protein, 180 aa, synthetic construct
MLSVMVSSSLVLIVFFLGASEEAKPAATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFIRLEEYLHSRK

SEQ ID NO: 232 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGRSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 233 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESICKDMRQRPLLGCGDKSVISRLSQEAERKSDNGTRKGLSELDTLFSRLEEYLHSRK

SEQ ID NO: 234 - protein, 182 aa, synthetic construct
MLSVMVSSSLVLIVFFLGASEEAKPAATTTTTTTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 235 - protein, 176 aa, synthetic construct
MLSVMVSSSLVLIVFFLGASEEAKPATIKNTKPQCRPEDYATRLQDLRVTFHRVKPTLPGHQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 236 - protein, 170 aa, synthetic construct
MKTNTKIILFCYVILSLYVFSCAIASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 237 - protein, 170 aa, synthetic construct
MKTNTKIILFCYVILSLYVFSCAIASAKKCNDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 238 - protein, 171 aa, synthetic construct
MKTNTKIILFCYVIFLSLYVFSCVVASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 239 - protein, 170 aa, synthetic construct
MKTNTKIILFCYVILSLYVFSCVVAYAKKCDDVSFDYILKDLRSEFSKIKSFVQNNDQENMMLLSQSMLNKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 240 - protein, 170 aa, synthetic construct
MKTSTKIILFCYVILSLYVFSCVVASAKKCDDVSFDYILKDLRSEFIKIKSFVQNNDQENMMLLSQSMLDKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 241 - protein, 168 aa, synthetic construct
MKTNTKIILFCYVILYVFSCTVASAKKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 242 - protein, 170 aa, synthetic construct
MKTSTKIILFCYVILSLYVFSCVVASAKKCDDVSFDYILKDLRSEFIKIKSFVQNNDQENMMLLSQSMLDKLTSRIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 243 - protein, 168 aa, synthetic construct
MKTNTKIILFCYVILYLFSCTVASAKKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 244 - protein, 174 aa, synthetic construct
MLSVMVSSSLVLIVFFLGASEEAKPATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKAVISRLSQEAERKSDNGTRKGLSELDTLFSRLEEYLHSRK

SEQ ID NO: 245 - protein, 168 aa, synthetic construct
MKTNTKIILFCYVILYLFSCTVASAKKCDDVSFDYILKDLRSEFSKIKSFVQNNDKENMMLLSQSMLDKLTRCIGCKSLSDMIKFYLNDVLPNAEKIEHIKNKITSIGEKLKSLKERLISCDFLHCENHDEIKAVKTIFNELKDKGIYKAMGEFDIFINHLEKYIVKK

SEQ ID NO: 246 - protein, 150 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKMPLTSCRKKEFTKP

SEQ ID NO: 247 - protein, 185 aa, synthetic construct
MGSRRLSRCSLATAVCLVAIVVAVAAKGRDSKPSPACDPMHGALAGIFKELRTTYRSVRETLQTKDTVYYVSLFHEQLLQEMLSPVGCRVTNELMQHYLDGVLPRAFHCGYDNTTLNALHFLSSSLSTLYQHMLKCPALACTGQTPAWTQFLDTEHKLDPWKGTVKATAEMDLLLNYLETFLLQS

SEQ ID NO: 248 - protein, 159 aa, synthetic construct
MLSVMVSSSLVLIVFLLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCEAERKSDNGTRKGLSELDTLFSRLEEYLHSRK

SEQ ID NO: 249 - protein, 187 aa, synthetic construct
MRRRRSFGVVVVGAIGTLLMMAVVVLSAHDHEHKVPPACDPVHGNLAGIFKELRTIYTSIREGLQKKDTVYYTSLFNDRVLQEMLSPMGCRVTNEIMEHYLDGVLPRASHLDYDNSTLNGLHAFASSMQALYQHMLKCPALACTGKTPAWMYFLEVEHKLNPWRGTAKAAAEADLLLNYLETFLLQF

SEQ ID NO: 250 - protein, 188 aa, synthetic construct
MGSRPARMCGLSNLLCLLLVVLVAVVIHRGCGASKPPVDCDPIHGTLSRIIKEVRTGYGSIKQALQSKDTVYYVSLFHENLLNEMLSPVGCRVTNELMQHYLDGVLPRAFQCGYDNTTLDGLHSLVSSLDALYKHMLKCPALACTGQTPAWTQFLETEHKLDPWKGTIKATAEMDLLVNYLETFLAQS

SEQ ID NO: 251 - protein, 142 aa, synthetic construct
MLSMMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISR

SEQ ID NO: 252 - protein, 139 aa, synthetic construct
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCVSVSVAALSAQR

SEQ ID NO: 253 - protein, 190 aa, synthetic construct
MININILSLLILILSIYANAIIDTCYDDQERERTKSNSISSVTPEMCKGLKQLVSTKLKDARQKEKSVRDYFTSRDNDLDFMLLQGVKETHKKTCGCYVLYLLLSFYGKTIRDTIQSNKHKNLNTELTNLAVSVLSLEDLLEACGITCNPKKDSLLKRIEEYMKEHGDDAIYKVIGEIEFLFQAIEKHVY

SEQ ID NO: 254 - protein, 192 aa, synthetic construct
MINISINILSLLILILSIYANSIIDMCYDDQERERTKSNSISSITPDMCKGLKQLVATKLKDARQKEKLVNSYFTSRDNDLTYMLLQGVRETHKKPCGCYVLYLLLTFYRKTIKDTIQSKKHESINTELTNLAVTVLSLEDLLEACGITCNPKKDSLLKRIEGYTKEHGDDAIYKVIGEIDFLFQAIERHVY

SEQ ID NO: 255 - protein, 191 aa, synthetic construct
MININILSLLILILSIYANAIIDTCYDDQERERTKSNSISSVTPEMCKGLKQLVATKLKDARQKEKLVNDYFTGRDNDLSYMLLQGVRETHKKPCGCYVLYLLLSFYRKTIRDTIQSNKHASINAELTNLAVSVLSLEDLLDACGITCNPKKDSLLKRIEEYMKEHGDDAIYKLIGEIEFLFQAIERHVYT

SEQ ID NO: 256 - protein, 191 aa, synthetic construct
MININILSLLILILSIYANAIIDTYDEDEDEDSIKLSSIGSITPEMCKNLKQLVASKLKDIRQKEKSLRDYFTNLDDELDYMLLQGVGENHKKKCGCYILHLLLKFYSKTIRNTIQSEKHKNVNLELTNIAVSMLALEDLLEKCGITCNPKKDPLLKRIEDYMKQHGDDGVNKAIAELEFLFQMIEKQVYI

SEQ ID NO: 257 - protein, 165 aa, synthetic construct
MARFIYVVLLCLVFDAAQSAAQCRKGTITSRLKMLRTAFEKVREFYEDRDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 258 - protein, 170 aa, synthetic construct
MMLALAMMLMALGPLSTNAMYVQHGSDYCTTTVHADIASAISGMRAEYDSGLGHYFKSLVPHPDNPYDTDDYKYMINNTNSYNCHALQSTINALLGMYGYVDIDEPHQLAMMKLATHTMQAAMLLNKCAKQLGCYHIPFDVETLHEAHPDDVMASLDTALNLMSMVTNEI

SEQ ID NO: 259 - protein, 165 aa, synthetic construct
MARFIYVVLLCLVFDAAQSAAQCRKGTITSRLKMLRTAFEKVREFYEDSDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 260 - protein, 168 aa, synthetic construct
MFQLEGIVLLVYLANWVSPATIKCVGMSTLFNPELIQLRRLFGDGIKDFFQNKDEDLDNAFLNEDVQRELASDCGCDHLMDMLSLYVNDTIPKGMKTEDAPSGLGQMGQLMSSLYRKMDMCWSELGCSHNTRLTLQEYADKKGGWDNKALGESDILFDALELFFSKIK

SEQ ID NO: 261 - protein, 169 aa, synthetic construct
MMTTLALVMTLMALSTNAMYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPNDVMASLDTALNLMSMVTNEI

SEQ ID NO: 262 - protein, 171 aa, synthetic construct
MITLALVMTLMALGPLSTNAMYARRSGDYCTTTVRADIASAISGMRAEYNSGLRDYFKSLVPHPDNPYDTDDYKYMLNNTNSYNCHALQSTINALLGMYGYVDIDESHQLAMMKLATHTMQTAMMLNKCAAQLGCYHIPFDLETLHEAHPDDVMASLDTALNLMSMITNEI

SEQ ID NO: 263 - protein, 169 aa, synthetic construct
MMTTLALVMTLMALATNAMYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPDDVMASLDTALNLMSMVTNEI

SEQ ID NO: 264 - protein, 177 aa, synthetic construct
MSGTSNKKFVFLVAIAVAICMMSSVSSNVHSGTEDNPCTNSKTVLNTLLNQIKQEYINNLLPYYKALTPKPVDVFDDSYTYSIQSTDYNCYTIYETLNFLLGDVFPRATTDATVRLSLAKIATSSQQASMLMNLCKKELACGPAPFDMIKLYHDTKEYGADNIMGTLDTPFQYFVIV

SEQ ID NO: 265 - protein, 170 aa, synthetic construct
MLALAMVLMALGPLSTNAMYVQHGRGDYCTTTVRADIASAISGMRAEYDSGLGHYFKSLVPHPDNPYDTDDYKYMINNTNSYNCHALQSTINALLGMYGYVDIDEPHQLAMMKLATHTMQTAMLLNKCAEQLGCYHIPFDVEILHEAHPDDVMASLDTALNLMSMVTNEI

SEQ ID NO: 266 - protein, 179 aa, synthetic construct
MFLAVLLTATIFFEARGAPATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCRDFLKCEDLPHWHQMAENYKEKPMQGFSEMDFVFQSVEKFLVAKDVKNMKTKRKH

SEQ ID NO: 267 - protein, 170 aa, synthetic construct
MLKQIIVVCIVAMAAVFADDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCRQQLGCGPPPFDAKTLYDDRAEYGADDIMATLDTALAKFKLVLESENVV

SEQ ID NO: 268 - protein, 170 aa, synthetic construct
MLKQIIVVCIVAMAAVFADDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCREQLGCGPPPFDAKTLYDDRAEYGADDIMATLDTALAKFKLVLESENVV

SEQ ID NO: 269 - protein, 171 aa, synthetic construct
MITLALVMTLMALGPLSTNGVHARRRGDYCTTTVRADIASAISGMRAEYNSGLGDYFKSLVPHPDNPYDTDDYKHMIDNANSYNCHALQSTINALLGMYGYVDIDEPHQLAMMKLATHTMQTAMLLNKCAAQLGCYHIPFDLETLREAPPADVMASLDTALNLMSMITNEI

SEQ ID NO: 270 - protein, 180 aa, synthetic construct
MDAQFLLLIVLALPASFAASLSTHYNNYDLTRIATIDKDVCKRVAQHINDDFVNMRKLYETQLKNYFQQLVPNPTDVFKDDSYMYMINGTDYNCHIIYETMRFLSGDVFPFATETEAELQYMWKMMLGVSQLSAYIGNCYQYFKCGPAPFDPQVLYHDRELFHADTVMAYLDTAFSHFTL

SEQ ID NO: 271 - protein, 156 aa, synthetic construct
MKLYFYCIFFYKIIVTISLNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNTVNVLEIKKLLLIDNNCKKLFSEIDIFLTWVMAKI

SEQ ID NO: 272 - protein, 156 aa, synthetic construct
MKLYFYCIFFYKIIVTISLNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNNVNVLEIKKLLLIDNNCKKLFSEIDIFLTWVMAKI

SEQ ID NO: 273 - protein, 134 aa, synthetic construct
MFLAVLLTATIFFEARGAPATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCVSRTQHLCYD

SEQ ID NO: 274 - protein, 179 aa, synthetic construct
MFRASLLCCLVLLAGVWADNKYDSESGDDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 275 - protein, 179 aa, synthetic construct
MFRASLLCCLVLLAGVWADNKYDSESGNDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 276 - protein, 179 aa, synthetic construct
MFRASLLCCLVLLAGVWADNKYDSESGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 277 - protein, 179 aa, synthetic construct
MFRASLLCCLVLLAGVWADNKYDSESGDDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 278 - protein, 179 aa, synthetic construct
MFRALLLCCLALLAGVWADNRYDGQDGNDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 279 - protein, 175 aa, synthetic construct
MLSVMVSSSLVLIVFFLGASEEAKPATTAIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 280 - protein, 176 aa, synthetic construct
MLSVMVSSSLVLIVFFLGAFEEAKPATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 281 - protein, 177 aa, synthetic construct
MLSVMVSSSLVLIVFFLGASEEAKPATTTTTIKNTKPQCRPEDYATRLQDLRVTFYRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 282 - protein, 174 aa, synthetic construct
MLSVMVSSSLVLIVFFLGASEEAKPATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 283 - protein, 171 aa, synthetic construct
MKTNTKIILFCYVIFLSLYVFSCVVASTKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 284 - protein, 148 aa, synthetic construct
MLSVMVSSSLVLIVFFLGASEEAKPATTTIKNTKPQCRPEDYATRLQDLRVTFHRVKPTLQREDDYSVWLDGDHVYPGLKTELHSMRSTLESIYKDMRQCPLLGCGDKSVISRLSQEAERKSDNGTRKGLSELDTLFSRLEEYLHSRK

SEQ ID NO: 285 - protein, 190 aa, synthetic construct
MSNFRILSLLIFSLIVHVNAMIGTCYDEDEEIERLKSNSISSITPGMCRNLKHSVMIRLIDARQIEASIRSYFTDGDNNLSFMLLQGIREISKKKCGCYILNLMLRFYIQTIKHTILSNKHKDMNLELTNLAVTILSLESLLEKCGVTCNPVKDPLLTRIEEYTRKHGDNAIYKTIGELEFLFDAIEKFV

SEQ ID NO: 286 - protein, 165 aa, synthetic construct
MARFIYVVLLCLVFDAAQSAAQCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVIGM

SEQ ID NO: 287 - protein, 165 aa, synthetic construct
MARFIYVVLLCLVFDAAQSAAQCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVNGM

SEQ ID NO: 288 - protein, 165 aa, synthetic construct
MARFIYVVLLCLVFDAAQSAAQCRKGTITIRLKMLRTAFEKVREFYEDRDEEETALASTEHLHGPESCSVIDELITHYTKCVIPAANEEEGADLLSLDTLQFALENVKGLLANCQEEFGCKPPFSMRDYKKQYRQLNKEKNAGMIKAMGELGMLFNGIEERVIGM

SEQ ID NO: 289 - protein, 165 aa, synthetic construct
MARFIYVVLLCLVFDAAQSAAQCRKGTITSRLKMLRTAFEKVREFYEDRDEEETALASTEHLHGPESCSVIDELITHHTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 290 - protein, 125 aa, synthetic construct
MLSVMVSSSLVLIVFLLGASEEAKPATTTIKNTKPQCRPEDYATRLQDLRVTFHRVKPTLVGHVGDHVYPGLKTELHSMRSTLESIYKDMRQCEAERKSDNGTRKGLSELDTLFSRLEEYLHSRK

SEQ ID NO: 291 - protein, 163 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKP

SEQ ID NO: 292 - protein, 163 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKT

SEQ ID NO: 293 - protein, 163 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKS

SEQ ID NO: 294 - protein, 162 aa, synthetic construct
YCVEYKESEEDRQQCSSSSFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTK

SEQ ID NO: 295 - protein, 163 aa, synthetic construct
YCVEYAESDEDKQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 296 - protein, 163 aa, synthetic construct
YCVEYEESEEDKQQCGSNGGPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 297 - protein, 163 aa, synthetic construct
YCVEYAESDEDRQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 298 - protein, 164 aa, synthetic construct
YCVEYEESEEDKQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 299 - protein, 163 aa, synthetic construct
YCIQYEESEEDKQQCSSSNFPASLPHMLRELRAAFGKVKTFFQMKDQLNSMLLTQSLLDDFKGYLGCQALSEMIRFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 300 - protein, 163 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLREKLKTLRLRLRRCHRFLPCENKSKAVEQVKRVFNMLQERGVYKAMSEFDIFINYIESYMTTKT

SEQ ID NO: 301 - protein, 164 aa, synthetic construct
YCVEYEESEEDKQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 302 - protein, 163 aa, synthetic construct
YCVEYAESDEDKQQCSGSNFPASLPHMLRELRAAFGKVKTFFQMKDQLNSMLLTQSLLDDFKGYLGCQALSEMIQFYLEEVMPRAENHGPDIKEHVNSLGEKLKTLRLRLRRCHRFLPCENKSKAVEQVKRVFNMLQERGVYKAMSEFDIFINYIESYMTTKM

SEQ ID NO: 303 - protein, 164 aa, synthetic construct
YCVEYEESEEDRQQCSGSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 304 - protein, 164 aa, synthetic construct
YCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 305 - protein, 164 aa, synthetic construct
YCVEYEESDEDKQQCSSSTGAPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 306 - protein, 164 aa, synthetic construct
YCVEYEETKEDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 307 - protein, 163 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGDYKAMSEFDIFINYIESYMTTKS

SEQ ID NO: 308 - protein, 164 aa, synthetic construct
YCVEYEESEEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCEDKSKAVEQVKRVFNMLQERGVYKAMSEFDILINYIESYMTTKM

SEQ ID NO: 309 - protein, 164 aa, synthetic construct
YCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMATKM

SEQ ID NO: 310 - protein, 161 aa, synthetic construct
DNRYDGQDGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 311 - protein, 164 aa, synthetic construct
YCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 312 - protein, 164 aa, synthetic construct
YCVEYEESKEDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 313 - protein, 164 aa, synthetic construct
YCVEYLESGEDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 314 - protein, 164 aa, synthetic construct
YCVEYLESREDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 315 - protein, 160 aa, synthetic construct
ESENNCTHFPTSLPHMLHELRAAFSRVKTFFQMKDQLDNMLLNGSLLEDFKGYLGCQALSEMIQFYLEEVMPQAENHSGGGGPDIKEHVNSLGEKLKTLRVRLRRCHRFLPCENKSKAVEQVKSAFSKLQEKGVYKAMSEFDIFINYIEAYMTTKMKNKK

SEQ ID NO: 316 - protein, 164 aa, synthetic construct
YCVEYLESDEDKQHCSSSNGASASSPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 317 - protein, 163 aa, synthetic construct
YCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLAEKLKTLRLRLRRCHRFLPCENKSKAVEQVKRVFNMLQERGVYKAMSEFDIFINYIESYMTTKS

SEQ ID NO: 318 - protein, 163 aa, synthetic construct
YCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLGEKLKTLRLRLRRCHRFLPCENKSKAVAQVKRVFNMLQERGVYKAMSEFDIFINYIESYMTTKS

SEQ ID NO: 319 - protein, 157 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 320 - protein, 157 aa, synthetic construct
YCVEYEESEEDRQQCSSSNFPASLPHMLRELRAAFGKVKTFFQMKDQLNSMLLTQSLLDDFKGYLGCQAFSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 321 - protein, 158 aa, synthetic construct
YCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 322 - protein, 148 aa, synthetic construct
VSNCGNLPHMLRDLRDAFSRVKTFFQMKDQLDNILLKESLLEDFKGYLGCQALSEMIQFYLEEVMPQAENQDPHAKEHVNSLGENLKTLRLRLRRCHRFLPCENKSKAVEQVKNAFSKLQEKGVYKAMSEFDIFINYIEAYMTMKIRR

SEQ ID NO: 323 - protein, 158 aa, synthetic construct
YCVEYLESREDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 324 - protein, 157 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSPGEKLKTLRLRLRRCHRFLPCENKSKAVEQVKRVFNMLQERGVYKAMSEFDIFINYIES

SEQ ID NO: 325 - protein, 163 aa, synthetic construct
YCTSCSYRDCTEDEDQKQQCEGGLRSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 326 - protein, 157 aa, synthetic construct
YCVEYEESEEDRQQCSSSNFPASLPHMPRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 327 - protein, 166 aa, synthetic construct
YCTSCSHRDCTEDDEQKQQCEGGSGGLGSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 328 - protein, 147 aa, synthetic construct
TDQCDNFPQMLRDLRDAFSRVKTFFQTKDEVDNLLLKESLLEDFKGYLGCQALSEMIQFYLEEVMPQAENQDPGAKDHVNSLGENLKTLRLRLRRCHRFLPCENKSKAVEQIKNAFNKLQEKGIYKAMSEFDIFINYIEAYMTIKAR

SEQ ID NO: 329 - protein, 147 aa, synthetic construct
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 330 - protein, 147 aa, synthetic construct
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTMKAR

SEQ ID NO: 331 - protein, 148 aa, synthetic construct
VSNCGNLPHMLRDLRDAFSRVKTFFQMKDQLDNILLKESLLEDFKGYLGCQALSEMIQFYLEEVMPQAENQDPNAKEHVNSLGENLKTLRLRLRRCHRFLPCENKSKAVEQVKNAFSKLQEKGVYKAMSEFDIFINYIEAYMTMKTRR

SEQ ID NO: 332 - protein, 147 aa, synthetic construct
TDQCDNFPQMLRDLRDAFSRVKTFFQTKDEVDNLLLKESLLEDFKGYLGCQALSEMIQFYLEEVMPQAENQDPEAKDHVNSLGENLKTIRLRLRRCHRFLPCENKSKAVEQIKNAFNKLQEKGIYKAMSEFDIFINYIEAYMTIKAR

SEQ ID NO: 333 - protein, 147 aa, synthetic construct
TDQCDNFPQMLRDLRDAFSRVKTFFQTKDEVDSLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 334 - protein, 147 aa, synthetic construct
TDQCDNFPQMLRDLRDAFSRVKTFFQTKDEVDNILLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 335 - protein, 147 aa, synthetic construct
TDQCDNFPQMLRDLRDAFSRVKTFFQTKDEVDNLFLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 336 - protein, 147 aa, synthetic construct
TDQCDNFPQMLRDLRDAFSRVKTFFQTKDEVDNLLLKESLLEDFKGYLGCQALSEMIQFYLEKVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 337 - protein, 147 aa, synthetic construct
TDQCDNFPQMLRDLRDAFSRVKTFFQTKDAVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 338 - protein, 148 aa, synthetic construct
ASNCGNLPHMLRDLRDAFSRVKTFFQMKDQLDNILLKESLLEDFRGYLGCQALSEMIQFYLEEVMPQAENQDPHSKEHVNSLGENLKTLRLRLRRCHRFLPCENKGKAVEQVKNAFSKLQEKGVYKAMSEFDIFINYIEAYMTMKLRR

SEQ ID NO: 339 - protein, 147 aa, synthetic construct
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFHIFINYIEAYMTIKAR

SEQ ID NO: 340 - protein, 139 aa, synthetic construct
EMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 341 - protein, 147 aa, synthetic construct
ETCGNIPHMLRDLRDAFSRVKTFFQMKDQLDNILLKESLLEDFKGYLGCQALSEMIQFYLEEVMPQAEAMSLKSQEHVNFLGENLNTLRLRLRRCHRFLPCENKSKAVEQVKNAFSKLQEKGVYKAMSEFDIFINYIEAYMTMKLRR

SEQ ID NO: 342 - protein, 180 aa, synthetic construct
TANNRAQKCFCFDGSNAGNSEETNTAAFQKKCDSEIPESLPYMLRDLRNSSVQTRRYFQEKDEENSPLLTQKLLEDFKGYLGCQALSEMIQFYLEEVMPQAEDSNPSAKDSVTSLGEKLKTLRLRLRRCHRFLPCENKSKAVENLKSKFGDLGNQGVHKAMSEFDIFINYIETYMTTKMK

SEQ ID NO: 343 - protein, 151 aa, synthetic construct
ATTTTIKNTKPQCRPEDYATRLQDLRVTFHRVKPTLQREDDYSVWLDGTVVKGCWGCSVMDWLLRRYLETVFPAGDHVYPGLKTELHSMRSTLESIYKDMRQCPLLGCGDKSVISRLSQEAERKSDNGTRKGLSELDTLFSRLEEYLHSRK

SEQ ID NO: 344 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 345 - protein, 150 aa, synthetic construct
ATTTTKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 346 - protein, 151 aa, synthetic construct
ATTTTIKNTKPQCRPEDYATRLQDLRVTFHRVKPTLQREDDYSVWLDGTMVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 347 - protein, 151 aa, synthetic construct
ATTTTIKNTKPQCRPEDYATRLQDLRVTFHRVKSTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 348 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDMLFSRLEEYLHSRK

SEQ ID NO: 349 - protein, 151 aa, synthetic construct
ATTTTIKNTKPQCRPEDYATRLQDLRVTFHRVKPTLQREDDYSVWLDGTVVKGCWGCSVMDWLLRRYLEIVLPAGDHVYPGLKTELHSMRSTLESIYKDMRQCPLLGCGDKSVISRLSQEAERKSDNGTRKGLSELDTLFSRLEEYLHSRK

SEQ ID NO: 350 - protein, 151 aa, synthetic construct
AATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 351 - protein, 151 aa, synthetic construct
ATTTTINNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 352 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDHVYPGLKTELHSMHSTLESIYKDMRQCPLLGCGDKSVISRLSQEAERKSDNGTRKGLSELDTLFSRLEEYLHSRK

SEQ ID NO: 353 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 354 - protein, 155 aa, synthetic construct
SKPPVDCDPIHGTLSRIIKEVRTGYGSIKQALQSKDTVYYVSLFHESLLREMLSPVGCRVTNELMQHYLDGVLPRAFQCGYDNTTLSGLHSLVSSLDTLYKHMLKCPALACTGQTPAWTQFLETEHKLDPWKGTIKATAEMDLLVNYLETFLAQS

SEQ ID NO: 355 - protein, 151 aa, synthetic construct
ATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 356 - protein, 151 aa, synthetic construct
AATTTIKNTKPQCRPEDYATRLQDLRVTFHRVKPTLQHEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 357 - protein, 151 aa, synthetic construct
ATTTTIKNTKPQCRPEDYATRLQDLRVTFHRVKPTLQREDDYSVWLDGMVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 358 - protein, 151 aa, synthetic construct
ATTTTIKNTKPQCRPEDYATRLQDLRVTFHRVKPTLQREDDYSVWLDGTVVKGCWGCSVVDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 359 - protein, 151 aa, synthetic construct
ATTTTIKNTKPQCRPEDYATRLQDLRITFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 360 - protein, 150 aa, synthetic construct
ATTTIKNTKPQCRPEDYATRLQDLRVTFHRIKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 361 - protein, 151 aa, synthetic construct
ATTTTIKNTKPQCRPEDYATRLQDLRVTFHRVKPTLQCEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 362 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 363 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 364 - protein, 151 aa, synthetic construct
ATTTTIKNTKPQCRPEDYATRLQDLRVTFHRVKPTLQREDDYSVWLDGTVVKGCWGCSVMDWLLRRYLEILFPAGDHVYPGLKTELHSMRSTLESIYKDMRQCPLLGCGDKSVISRLSQEAERKSDNGTRKGLSELDTLFSRLEEYLHSRK

SEQ ID NO: 365 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 366 - protein, 150 aa, synthetic construct
ATTTIKNTKPQCRPEDYATRLQDLRVTFHRVKPTLQREDDYSVWLDGTVVKGCWGCSVMDWLLRRYLEIVFPAGDHVYPRLKTELHSMRSTLESIYKDMQQCPLLGCGDKSVISRLSQEAERKSDNGTRKGLSELDTLFSRLEEYLHSRK

SEQ ID NO: 367 - protein, 152 aa, synthetic construct
AATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 368 - protein, 153 aa, synthetic construct
AATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 369 - protein, 152 aa, synthetic construct
AATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 370 - protein, 152 aa, synthetic construct
ATTTTTIKNTKPQCRPEDYATRLQDLRVTFHRVKPTLQREDDYSVWLDGTVVKGCWGCSVMDWLLRRYLEIMFPAGDHVYPGLKTELHSMRSTLESIYKDMRQCPLLGCGDKSVISRLSQEAERKSDNGTRKGLSELDTLFSRLEEYLHSRK

SEQ ID NO: 371 - protein, 150 aa, synthetic construct
ATTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 372 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDYVYPGLKTELHSMRSTLESIYKDMRQCPLLGCGDKSVISRLSQEAERKSDNGMRKGLSELDTLFSRLEEYLHSRK

SEQ ID NO: 373 - protein, 150 aa, synthetic construct
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 374 - protein, 152 aa, synthetic construct
ATTTTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 375 - protein, 153 aa, synthetic construct
AATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 376 - protein, 153 aa, synthetic construct
ATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 377 - protein, 151 aa, synthetic construct
AATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLNELDTLFSRLEEYLHSRK

SEQ ID NO: 378 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 379 - protein, 151 aa, synthetic construct
ATTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 380 - protein, 154 aa, synthetic construct
ATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 381 - protein, 152 aa, synthetic construct
AATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKSLSELDTLFSRLEEYLHSRK

SEQ ID NO: 382 - protein, 150 aa, synthetic construct
ATTTIKNTKPQCRPEDYATRLQDLRVTFHRVKPTLQREDDYSVWLDGTVVKGCWGCSVMDWLLRRYLEIVFPAGDHVYSGLKTELHSMRSTLESIYKDMRQCPLLGCGDKSVISRLSQEAERKSDNGTRKGLSELDTLFSRLEEYLHSRK

SEQ ID NO: 383 - protein, 153 aa, synthetic construct
ATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 384 - protein, 153 aa, synthetic construct
ATTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 385 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMWQCPLLGCGDKSVISRLSQEAERKSDNGTRKGLSELDTLFSRLEEYLHSRK

SEQ ID NO: 386 - protein, 152 aa, synthetic construct
AATTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 387 - protein, 148 aa, synthetic construct
ATIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 388 - protein, 154 aa, synthetic construct
ATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 389 - protein, 154 aa, synthetic construct
ATTTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 390 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLRELDTLFSRLEEYLHSRK

SEQ ID NO: 391 - protein, 159 aa, synthetic construct
AKPAATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 392 - protein, 153 aa, synthetic construct
ATTTTTTIKNTKPQCRPEDYATRLQDLRVTFHRVKPTLQREDDYSVWLDGTVVKGCWRCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 393 - protein, 152 aa, synthetic construct
ATTTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 394 - protein, 155 aa, synthetic construct
ATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 395 - protein, 154 aa, synthetic construct
ATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFIRLEEYLHSRK

SEQ ID NO: 396 - protein, 152 aa, synthetic construct
ATTTTTIKNTKPQCRPEDYATRLQDLRVTFHRVKPTLQREDDYSVWLDGTVVKGCWGRSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 397 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESICKDMRQRPLLGCGDKSVISRLSQEAERKSDNGTRKGLSELDTLFSRLEEYLHSRK

SEQ ID NO: 398 - protein, 160 aa, synthetic construct
AKPAATTTTTTTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 399 - protein, 151 aa, synthetic construct
ATIKNTKPQCRPEDYATRLQDLRVTFHRVKPTLPGHQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 400 - protein, 149 aa, synthetic construct
CAIASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 401 - protein, 149 aa, synthetic construct
CAIASAKKCNDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 402 - protein, 149 aa, synthetic construct
CVVASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 403 - protein, 149 aa, synthetic construct
CVVAYAKKCDDVSFDYILKDLRSEFSKIKSFVQNNDQENMMLLSQSMLNKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 404 - protein, 149 aa, synthetic construct
CVVASAKKCDDVSFDYILKDLRSEFIKIKSFVQNNDQENMMLLSQSMLDKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 405 - protein, 143 aa, synthetic construct
KKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 406 - protein, 149 aa, synthetic construct
CVVASAKKCDDVSFDYILKDLRSEFIKIKSFVQNNDQENMMLLSQSMLDKLTSRIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 407 - protein, 149 aa, synthetic construct
CTVASAKKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 408 - protein, 149 aa, synthetic construct
ATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKAVISRLSQEAERKSDNGTRKGLSELDTLFSRLEEYLHSRK

SEQ ID NO: 409 - protein, 149 aa, synthetic construct
CTVASAKKCDDVSFDYILKDLRSEFSKIKSFVQNNDKENMMLLSQSMLDKLTRCIGCKSLSDMIKFYLNDVLPNAEKIEHIKNKITSIGEKLKSLKERLISCDFLHCENHDEIKAVKTIFNELKDKGIYKAMGEFDIFINHLEKYIVKK

SEQ ID NO: 410 - protein, 127 aa, synthetic construct
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKMPLTSCRKKEFTKP

SEQ ID NO: 411 - protein, 159 aa, synthetic construct
KGRDSKPSPACDPMHGALAGIFKELRTTYRSVRETLQTKDTVYYVSLFHEQLLQEMLSPVGCRVTNELMQHYLDGVLPRAFHCGYDNTTLNALHFLSSSLSTLYQHMLKCPALACTGQTPAWTQFLDTEHKLDPWKGTVKATAEMDLLLNYLETFLLQS

SEQ ID NO: 412 - protein, 134 aa, synthetic construct
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCEAERKSDNGTRKGLSELDTLFSRLEEYLHSRK

SEQ ID NO: 413 - protein, 160 aa, synthetic construct
AHDHEHKVPPACDPVHGNLAGIFKELRTIYTSIREGLQKKDTVYYTSLFNDRVLQEMLSPMGCRVTNEIMEHYLDGVLPRASHLDYDNSTLNGLHAFASSMQALYQHMLKCPALACTGKTPAWMYFLEVEHKLNPWRGTAKAAAEADLLLNYLETFLLQF

SEQ ID NO: 414 - protein, 156 aa, synthetic construct
ASKPPVDCDPIHGTLSRIIKEVRTGYGSIKQALQSKDTVYYVSLFHENLLNEMLSPVGCRVTNELMQHYLDGVLPRAFQCGYDNTTLDGLHSLVSSLDALYKHMLKCPALACTGQTPAWTQFLETEHKLDPWKGTIKATAEMDLLVNYLETFLAQS

SEQ ID NO: 415 - protein, 117 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISR

SEQ ID NO: 416 - protein, 114 aa, synthetic construct
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCVSVSVAALSAQR

SEQ ID NO: 417 - protein, 170 aa, synthetic construct
IIDTCYDDQERERTKSNSISSVTPEMCKGLKQLVSTKLKDARQKEKSVRDYFTSRDNDLDFMLLQGVKETHKKTCGCYVLYLLLSFYGKTIRDTIQSNKHKNLNTELTNLAVSVLSLEDLLEACGITCNPKKDSLLKRIEEYMKEHGDDAIYKVIGEIEFLFQAIEKHVY

SEQ ID NO: 418 - protein, 172 aa, synthetic construct
NSIIDMCYDDQERERTKSNSISSITPDMCKGLKQLVATKLKDARQKEKLVNSYFTSRDNDLTYMLLQGVRETHKKPCGCYVLYLLLTFYRKTIKDTIQSKKHESINTELTNLAVTVLSLEDLLEACGITCNPKKDSLLKRIEGYTKEHGDDAIYKVIGEIDFLFQAIERHVY

SEQ ID NO: 419 - protein, 171 aa, synthetic construct
IIDTCYDDQERERTKSNSISSVTPEMCKGLKQLVATKLKDARQKEKLVNDYFTGRDNDLSYMLLQGVRETHKKPCGCYVLYLLLSFYRKTIRDTIQSNKHASINAELTNLAVSVLSLEDLLDACGITCNPKKDSLLKRIEEYMKEHGDDAIYKLIGEIEFLFQAIERHVYT

SEQ ID NO: 420 - protein, 171 aa, synthetic construct
IIDTYDEDEDEDSIKLSSIGSITPEMCKNLKQLVASKLKDIRQKEKSLRDYFTNLDDELDYMLLQGVGENHKKKCGCYILHLLLKFYSKTIRNTIQSEKHKNVNLELTNIAVSMLALEDLLEKCGITCNPKKDPLLKRIEDYMKQHGDDGVNKAIAELEFLFQMIEKQVYI

SEQ ID NO: 421 - protein, 146 aa, synthetic construct
AAQCRKGTITSRLKMLRTAFEKVREFYEDRDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 422 - protein, 151 aa, synthetic construct
MYVQHGSDYCTTTVHADIASAISGMRAEYDSGLGHYFKSLVPHPDNPYDTDDYKYMINNTNSYNCHALQSTINALLGMYGYVDIDEPHQLAMMKLATHTMQAAMLLNKCAKQLGCYHIPFDVETLHEAHPDDVMASLDTALNLMSMVTNEI

SEQ ID NO: 423 - protein, 146 aa, synthetic construct
AAQCRKGTITSRLKMLRTAFEKVREFYEDSDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 424 - protein, 149 aa, synthetic construct
ATIKCVGMSTLFNPELIQLRRLFGDGIKDFFQNKDEDLDNAFLNEDVQRELASDCGCDHLMDMLSLYVNDTIPKGMKTEDAPSGLGQMGQLMSSLYRKMDMCWSELGCSHNTRLTLQEYADKKGGWDNKALGESDILFDALELFFSKIK

SEQ ID NO: 425 - protein, 151 aa, synthetic construct
MYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPNDVMASLDTALNLMSMVTNEI

SEQ ID NO: 426 - protein, 151 aa, synthetic construct
MYARRSGDYCTTTVRADIASAISGMRAEYNSGLRDYFKSLVPHPDNPYDTDDYKYMLNNTNSYNCHALQSTINALLGMYGYVDIDESHQLAMMKLATHTMQTAMMLNKCAAQLGCYHIPFDLETLHEAHPDDVMASLDTALNLMSMITNEI

SEQ ID NO: 427 - protein, 151 aa, synthetic construct
MYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPDDVMASLDTALNLMSMVTNEI

SEQ ID NO: 428 - protein, 150 aa, synthetic construct
NVHSGTEDNPCTNSKTVLNTLLNQIKQEYINNLLPYYKALTPKPVDVFDDSYTYSIQSTDYNCYTIYETLNFLLGDVFPRATTDATVRLSLAKIATSSQQASMLMNLCKKELACGPAPFDMIKLYHDTKEYGADNIMGTLDTPFQYFVIV

SEQ ID NO: 429 - protein, 152 aa, synthetic construct
MYVQHGRGDYCTTTVRADIASAISGMRAEYDSGLGHYFKSLVPHPDNPYDTDDYKYMINNTNSYNCHALQSTINALLGMYGYVDIDEPHQLAMMKLATHTMQTAMLLNKCAEQLGCYHIPFDVEILHEAHPDDVMASLDTALNLMSMVTNEI

SEQ ID NO: 430 - protein, 162 aa, synthetic construct
APATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCRDFLKCEDLPHWHQMAENYKEKPMQGFSEMDFVFQSVEKFLVAKDVKNMKTKRKH

SEQ ID NO: 431 - protein, 152 aa, synthetic construct
DDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCRQQLGCGPPPFDAKTLYDDRAEYGADDIMATLDTALAKFKLVLESENVV

SEQ ID NO: 432 - protein, 152 aa, synthetic construct
DDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCREQLGCGPPPFDAKTLYDDRAEYGADDIMATLDTALAKFKLVLESENVV

SEQ ID NO: 433 - protein, 148 aa, synthetic construct
RRRGDYCTTTVRADIASAISGMRAEYNSGLGDYFKSLVPHPDNPYDTDDYKHMIDNANSYNCHALQSTINALLGMYGYVDIDEPHQLAMMKLATHTMQTAMLLNKCAAQLGCYHIPFDLETLREAPPADVMASLDTALNLMSMITNEI

SEQ ID NO: 434 - protein, 162 aa, synthetic construct
ASLSTHYNNYDLTRIATIDKDVCKRVAQHINDDFVNMRKLYETQLKNYFQQLVPNPTDVFKDDSYMYMINGTDYNCHIIYETMRFLSGDVFPFATETEAELQYMWKMMLGVSQLSAYIGNCYQYFKCGPAPFDPQVLYHDRELFHADTVMAYLDTAFSHFTL

SEQ ID NO: 435 - protein, 138 aa, synthetic construct
LNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNTVNVLEIKKLLLIDNNCKKLFSEIDIFLTWVMAKI

SEQ ID NO: 436 - protein, 138 aa, synthetic construct
LNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNNVNVLEIKKLLLIDNNCKKLFSEIDIFLTWVMAKI

SEQ ID NO: 437 - protein, 117 aa, synthetic construct
APATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCVSRTQHLCYD

SEQ ID NO: 438 - protein, 161 aa, synthetic construct
DNKYDSESGDDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 439 - protein, 161 aa, synthetic construct
DNKYDSESGNDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 440 - protein, 161 aa, synthetic construct
DNKYDSESGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 441 - protein, 161 aa, synthetic construct
DNKYDSESGDDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 442 - protein, 161 aa, synthetic construct
DNRYDGQDGNDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 443 - protein, 150 aa, synthetic construct
ATTAIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 444 - protein, 151 aa, synthetic construct
ATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 445 - protein, 152 aa, synthetic construct
ATTTTTIKNTKPQCRPEDYATRLQDLRVTFYRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 446 - protein, 149 aa, synthetic construct
ATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 447 - protein, 145 aa, synthetic construct
STKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 448 - protein, 123 aa, synthetic construct
ATTTIKNTKPQCRPEDYATRLQDLRVTFHRVKPTLQREDDYSVWLDGDHVYPGLKTELHSMRSTLESIYKDMRQCPLLGCGDKSVISRLSQEAERKSDNGTRKGLSELDTLFSRLEEYLHSRK

SEQ ID NO: 449 - protein, 170 aa, synthetic construct
MIGTCYDEDEEIERLKSNSISSITPGMCRNLKHSVMIRLIDARQIEASIRSYFTDGDNNLSFMLLQGIREISKKKCGCYILNLMLRFYIQTIKHTILSNKHKDMNLELTNLAVTILSLESLLEKCGVTCNPVKDPLLTRIEEYTRKHGDNAIYKTIGELEFLFDAIEKFV

SEQ ID NO: 450 - protein, 144 aa, synthetic construct
QCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVIGM

SEQ ID NO: 451 - protein, 144 aa, synthetic construct
QCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVNGM

SEQ ID NO: 452 - protein, 144 aa, synthetic construct
QCRKGTITIRLKMLRTAFEKVREFYEDRDEEETALASTEHLHGPESCSVIDELITHYTKCVIPAANEEEGADLLSLDTLQFALENVKGLLANCQEEFGCKPPFSMRDYKKQYRQLNKEKNAGMIKAMGELGMLFNGIEERVIGM

SEQ ID NO: 453 - protein, 146 aa, synthetic construct
AAQCRKGTITSRLKMLRTAFEKVREFYEDRDEEETALASTEHLHGPESCSVIDELITHHTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 454 - protein, 100 aa, synthetic construct
ATTTIKNTKPQCRPEDYATRLQDLRVTFHRVKPTLVGHVGDHVYPGLKTELHSMRSTLESIYKDMRQCEAERKSDNGTRKGLSELDTLFSRLEEYLHSRK

SEQ ID NO: 455 - protein, 13 aa, synthetic construct
VPRDCGCKPCICT

SEQ ID NO: 456 - protein, 107 aa, synthetic construct
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTK

SEQ ID NO: 457 - protein, 107 aa, synthetic construct
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 458 - protein, 106 aa, synthetic construct
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 459 - protein, 214 aa, synthetic construct
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 460 - protein, 213 aa, synthetic construct
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 461 - protein, 227 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 462 - protein, 227 aa, synthetic construct; identical to SEQ ID NO: 461

SEQ ID NO: 463 - protein, 16 aa, synthetic construct
EPRGPTIKPCPPCKCP

SEQ ID NO: 464 - protein, 134 aa, synthetic construct
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVT

SEQ ID NO: 465 - protein, 83 aa, synthetic construct
LTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 466 - protein, 82 aa, synthetic construct
LTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 467 - protein, 217 aa, synthetic construct
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 468 - protein, 216 aa, synthetic construct
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 469 - protein, 233 aa, synthetic construct
EPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 470 - protein, 232 aa, synthetic construct
EPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 471 - protein, 217 aa, synthetic construct
APNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 472 - protein, 216 aa, synthetic construct
APNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 473 - protein, 233 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 474 - protein, 232 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 475 - protein, 217 aa, synthetic construct
APNAAGGPSVFIFAAKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 476 - protein, 216 aa, synthetic construct
APNAAGGPSVFIFAAKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 477 - protein, 233 aa, synthetic construct
EPRGPTIKPCAACKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 478 - protein, 232 aa, synthetic construct
EPRGPTIKPCAACKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 479 - protein, 457 aa, synthetic construct
MYRMQLLSCIALSLALVTNSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 480 - protein, 448 aa, synthetic construct
MYRMQLLSCIALSLALVTNSVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 481 - protein, 437 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 482 - protein, 428 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSMGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 483 - protein, 421 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 484 - protein, 438 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 485 - protein, 431 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 486 - protein, 443 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 487 - protein, 428 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 488 - protein, 424 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 489 - protein, 426 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 490 - protein, 431 aa, synthetic construct
MYRMQLLSCIALSLALVTNSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 491 - protein, 422 aa, synthetic construct
MYRMQLLSCIALSLALVTNSVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 492 - protein, 417 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 493 - protein, 401 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 494 - protein, 418 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 495 - protein, 411 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 496 - protein, 423 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 497 - protein, 408 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 498 - protein, 404 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 499 - protein, 406 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 500 - protein, 411 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 501 - protein, 402 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 502 - protein, 397 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 503 - protein, 420 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 504 - protein, 437 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 505 - protein, 430 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 506 - protein, 442 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 507 - protein, 427 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 508 - protein, 423 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 509 - protein, 425 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 510 - protein, 416 aa, synthetic construct
MYRMQLLSCIALSLALVTNSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 511 - protein, 400 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 512 - protein, 417 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 513 - protein, 410 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 514 - protein, 422 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 515 - protein, 407 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 516 - protein, 403 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 517 - protein, 405 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 518 - protein, 396 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 519 - protein, 423 aa, synthetic construct
MYRMQLLSCIALSLALVTNSVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 520 - protein, 403 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 521 - protein, 431 aa, synthetic construct
MYRMQLLSCIALSLALVTNSEPRGPTIKPCLLCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 522 - protein, 411 aa, synthetic construct
EPRGPTIKPCLLCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 523 - protein, 431 aa, synthetic construct
MYRMQLLSCIALSLALVTNSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 524 - protein, 411 aa, synthetic construct
EPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 525 - protein, 253 aa, synthetic construct
MYRMQLLSCIALSLALVTNSEPRGPTIKPCPPCKCPAPNAAGGPSVFIFPPKIKDVLMISLSPIVTCVVVDVSEDDPDVQISWFVNNVEVHTAQTQTHREDYNSTLRVVSALPIQHQDWMSGKEFKCKVNNKDLGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 526 - protein, 233 aa, synthetic construct; identical to SEQ ID NO: 473

SEQ ID NO: 527 - protein, 245 aa, synthetic construct
MYRMQLLSCIALSLALVTNSVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSP

SEQ ID NO: 528 - protein, 225 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSP

SEQ ID NO: 529 - protein, 248 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 530 - protein, 228 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 531 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 532 - protein, 330 aa, synthetic construct [sequence omitted]

SEQ ID NO: 533 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 534 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 535 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 536 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 537 - protein, 107 aa, synthetic construct [sequence omitted]

SEQ ID NO: 538 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 539 - protein, 97 aa, synthetic construct
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKI

SEQ ID NO: 540 - protein, 330 aa, synthetic construct
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIEPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 541 - protein, 329 aa, synthetic construct
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIEPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 542 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 543 - protein, 105 aa, synthetic construct
QPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 544 - RNA, 500 nt, synthetic construct
  source 1..500: mol_type = other RNA
  misc_difference 1..500: note = This sequence may encompass 10-500 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 545 - RNA, 200 nt, synthetic construct
  source 1..200: mol_type = other RNA
  misc_difference 1..200: note = This sequence may encompass 10-200 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 546 - RNA, 200 nt, synthetic construct; identical to SEQ ID NO: 545
  source 1..200: mol_type = other RNA
  misc_difference 1..200: note = This sequence may encompass 20-200 nucleotides

SEQ ID NO: 547 - RNA, 200 nt, synthetic construct; identical to SEQ ID NO: 545
  source 1..200: mol_type = other RNA
  misc_difference 1..200: note = This sequence may encompass 30-200 nucleotides

SEQ ID NO: 548 - RNA, 200 nt, synthetic construct; identical to SEQ ID NO: 545
  source 1..200: mol_type = other RNA
  misc_difference 1..200: note = This sequence may encompass 40-200 nucleotides

SEQ ID NO: 549 - RNA, 200 nt, synthetic construct; identical to SEQ ID NO: 545
  source 1..200: mol_type = other RNA
  misc_difference 1..200: note = This sequence may encompass 50-200 nucleotides

SEQ ID NO: 550 - RNA, 500 nt, synthetic construct; identical to SEQ ID NO: 544
  source 1..500: mol_type = other RNA
  misc_difference 1..500: note = This sequence may encompass 10, 20, 30, 40, 50, 60, 70, 80, 90, 100, 200, 300, 400, or 500 nucleotides